SEQUENCE LISTING (162 sequences)

SEQ ID NO: 1 - DNA, 705 bp, Artificial Sequence
  Description of Artificial Sequence Synthetic polynucleotide
atgaggctccctgctcagctcctggggctcctgctgctctggttcccaggttccagatgcgacatccagatgacccagtctccatcttccgtgtctgcatctgtaggagacagagtcaccatcacttgtcgggcgagtcagggtatttacagctggttagcctggtatcagcagaaaccagggaaagcccctaacctcctgatctatactgcatccactttacaaagtggggtcccatcaaggttcagcggcagtggatctgggacagatttcactctcaccatcagcagcctgcaacctgaagattttgcaacttactattgtcaacaggctaacattttcccgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 2 - DNA, 60 bp, Artificial Sequence
  Description of Artificial Sequence Synthetic oligonucleotide
atgaggctccctgctcagctcctggggctcctgctgctctggttcccaggttccagatgc

SEQ ID NO: 3 - DNA, 320 bp, Artificial Sequence
  Description of Artificial Sequence Synthetic polynucleotide
gacatccagatgacccagtctccatcttccgtgtctgcatctgtaggagacagagtcaccatcacttgtcgggcgagtcagggtatttacagctggttagcctggtatcagcagaaaccagggaaagcccctaacctcctgatctatactgcatccactttacaaagtggggtcccatcaaggttcagcggcagtggatctgggacagatttcactctcaccatcagcagcctgcaacctgaagattttgcaacttactattgtcaacaggctaacattttcccgctcactttcggcggagggaccaaggtggagatcaa

SEQ ID NO: 4 - protein, 234 aa, Artificial Sequence
  Description of Artificial Sequence Synthetic polypeptide
MRLPAQLLGLLLLWFPGSRCDIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 20 aa, Artificial Sequence
  Description of Artificial Sequence Synthetic peptide
MRLPAQLLGLLLLWFPGSRC

SEQ ID NO: 6 - protein, 107 aa, Artificial Sequence
  Description of Artificial Sequence Synthetic polypeptide
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIK

SEQ ID NO: 7 - DNA, 1416 bp, Artificial Sequence
  Description of Artificial Sequence Synthetic polynucleotide
atggactggacctggaggatcctcttcttggtggcagcagccacaggagcccactcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctgacagtggtggcacaaactatgcacagaagtttcagggcagggtcaccatgaccagggacacgtccatcagcacagcctacatggagctgaacaggctgagatctgacgacacggccgtgtattactgtgcgagagatcagcccctaggatattgtactaatggtgtatgctcctactttgactactggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 8 - DNA, 1428 bp, Artificial Sequence
  Description of Artificial Sequence Synthetic polynucleotide
atggactggacctggaggatcctcttcttggtggcagcagccacaggagcccactcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctgacagtggtggcacaaactatgcacagaagtttcagggcagggtcaccatgaccagggacacgtccatcagcacagcctacatggagctgaacaggctgagatctgacgacacggccgtgtattactgtgcgagagatcagcccctaggatattgtactaatggtgtatgctcctactttgactactggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 9 - DNA, 1428 bp, Artificial Sequence
  Description of Artificial Sequence Synthetic polynucleotide
atggactggacctggaggatcctcttcttggtggcagcagccacaggagcccactcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctgacagtggtggcacaaactatgcacagaagtttcagggcagggtcaccatgaccagggacacgtccatcagcacagcctacatggagctgaacaggctgagatctgacgacacggccgtgtattactgtgcgagagatcagcccctaggatattgtactaatggtgtatgctcctactttgactactggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcccaaatcttgtgacaaaactcacacatgcccaccgtgcccagcacctgaactcgtggggggaccgtcagtcttcctcctgcccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcctgaggagcagtacaacagcacgctgcgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctctggtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 10 - DNA, 1428 bp, Artificial Sequence
  Description of Artificial Sequence Synthetic polynucleotide
atggactggacctggaggatcctcttcttggtggcagcagccacaggagcccactcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctgacagtggtggcacaaactatgcacagaagtttcagggcagggtcaccatgaccagggacacgtccatcagcacagcctacatggagctgaacaggctgagatctgacgacacggccgtgtattactgtgcgagagatcagcccctaggatattgtactaatggtgtatgctcctactttgactactggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcccaaatcttgtgacaaaactcacacatgcccaccgtgcccagcacctgaactcctggggggaccggatgtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccgaggagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 11 - DNA, 1428 bp, Artificial Sequence
  Description of Artificial Sequence Synthetic polynucleotide
atggactggacctggaggatcctcttcttggtggcagcagccacaggagcccactcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctgacagtggtggcacaaactatgcacagaagtttcagggcagggtcaccatgaccagggacacgtccatcagcacagcctacatggagctgaacaggctgagatctgacgacacggccgtgtattactgtgcgagagatcagcccctaggatattgtactaatggtgtatgctcctactttgactactggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacgccacgtaccgtgtggtcagcgtcctcaccgtcctgcaccaggactggctgaatggcaaggagtacaagtgcaaggtctccaacaaagccctcccagcccccatcgccgctaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 12 - DNA, 57 bp, Artificial Sequence
  Description of Artificial Sequence Synthetic oligonucleotide
atggactggacctggaggatcctcttcttggtggcagcagccacaggagcccactcc

SEQ ID NO: 13 - DNA, 379 bp, Artificial Sequence
  Description of Artificial Sequence Synthetic polynucleotide
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctgacagtggtggcacaaactatgcacagaagtttcagggcagggtcaccatgaccagggacacgtccatcagcacagcctacatggagctgaacaggctgagatctgacgacacggccgtgtattactgtgcgagagatcagcccctaggatattgtactaatggtgtatgctcctactttgactactggggccagggaaccctggtcaccgtctcctcag

SEQ ID NO: 14 - protein, 471 aa, Artificial Sequence
  Description of Artificial Sequence Synthetic polypeptide
MDWTWRILFLVAAATGAHSQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 15 - protein, 475 aa, Artificial Sequence
  Description of Artificial Sequence Synthetic polypeptide
MDWTWRILFLVAAATGAHSQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 475 aa, Artificial Sequence
  Description of Artificial Sequence Synthetic polypeptide
MDWTWRILFLVAAATGAHSQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVEPKSCDKTHTCPPCPAPELVGGPSVFLLPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSREEMTKNQVSLTCLVKGFYPSDIAVEWESNGQPENNYKTTPLVLDSDGSFFLYSKLTVDKSRWQQGNVFSCSVMHEALHNHYTQKSLSLSPGK

SEQ ID NO: 17 - protein, 475 aa, Artificial Sequence
  Description of Artificial Sequence Synthetic polypeptide
MDWTWRILFLVAAATGAHSQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 475 aa, Artificial Sequence
  Description of Artificial Sequence Synthetic polypeptide
MDWTWRILFLVAAATGAHSQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 19 aa, Artificial Sequence
  Description of Artificial Sequence Synthetic peptide
MDWTWRILFLVAAATGAHS

SEQ ID NO: 20 - protein, 126 aa, Artificial Sequence
  Description of Artificial Sequence Synthetic polypeptide
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSS

SEQ ID NO: 21 - protein, 5 aa, Unknown
  Description of Unknown Sortase A enzyme recognition motif sequence
  MOD_RES (3)..(3): Any amino acid
LPXTG

SEQ ID NO: 22 - protein, 456 aa, Artificial Sequence
  Description of Artificial Sequence Synthetic polypeptide
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 7 aa, Artificial Sequence
  Description of Artificial Sequence Synthetic peptide
GYTFTYY

SEQ ID NO: 24 - protein, 8 aa, Artificial Sequence
  Description of Artificial Sequence Synthetic peptide
INPDSGGT

SEQ ID NO: 25 - protein, 19 aa, Artificial Sequence
  Description of Artificial Sequence Synthetic peptide
ARDQPLGYCTNGVCSYFDY

SEQ ID NO: 26 - protein, 214 aa, Artificial Sequence
  Description of Artificial Sequence Synthetic polypeptide
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 6 aa, Artificial Sequence
  Description of Artificial Sequence Synthetic peptide
QGIYSW

SEQ ID NO: 28 - protein, 3 aa, Artificial Sequence
  Description of Artificial Sequence Synthetic peptide
TAS

SEQ ID NO: 29 - protein, 9 aa, Artificial Sequence
  Description of Artificial Sequence Synthetic peptide
QQANIFPLT

SEQ ID NO: 30 - protein, 449 aa, Artificial Sequence
  Description of Artificial Sequence Synthetic polypeptide
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 8 aa, Artificial Sequence
  Description of Artificial Sequence Synthetic peptide
GFTFTDYT

SEQ ID NO: 32 - protein, 8 aa, Artificial Sequence
  Description of Artificial Sequence Synthetic peptide
VNPNSGGS

SEQ ID NO: 33 - protein, 12 aa, Artificial Sequence
  Description of Artificial Sequence Synthetic peptide
ARNLGPSFYFDY

SEQ ID NO: 34 - protein, 214 aa, Artificial Sequence
  Description of Artificial Sequence Synthetic polypeptide
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 6 aa, Artificial Sequence
  Description of Artificial Sequence Synthetic peptide
QDVSIG

SEQ ID NO: 36 - protein, 3 aa, Artificial Sequence
  Description of Artificial Sequence Synthetic peptide
SAS

SEQ ID NO: 37 - protein, 9 aa, Artificial Sequence
  Description of Artificial Sequence Synthetic peptide
QQYYIYPYT

SEQ ID NO: 38 - protein, 444 aa, Artificial Sequence
  Description of Artificial Sequence Synthetic polypeptide
EVQLVESGGGLVQPGGSLRLSCAASGYSFTGYYIHWVRQAPGKGLEWVARVIPNAGGTSYNQKFKGRFTLSVDNSKNTAYLQMNSLRAEDTAVYYCAREGIYWWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 8 aa, Artificial Sequence
  Description of Artificial Sequence Synthetic peptide
GYSFTGYY

SEQ ID NO: 40 - protein, 8 aa, Artificial Sequence
  Description of Artificial Sequence Synthetic peptide
VIPNAGGT

SEQ ID NO: 41 - protein, 7 aa, Artificial Sequence
  Description of Artificial Sequence Synthetic peptide
AREGIYW

SEQ ID NO: 42 - protein, 219 aa, Artificial Sequence
  Description of Artificial Sequence Synthetic polypeptide
DIQMTQSPSSLSASVGDRVTITCRSSQSLVHSNGNTFLHWYQQKPGKAPKLLIYTVSNRFSGVPSRFSGSGSGTDFTLTISSLQPEDFATYFCSQTTHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 11 aa, Artificial Sequence
  Description of Artificial Sequence Synthetic peptide
QSLVHSNGNTF

SEQ ID NO: 44 - protein, 3 aa, Artificial Sequence
  Description of Artificial Sequence Synthetic peptide
TVS

SEQ ID NO: 45 - protein, 9 aa, Artificial Sequence
  Description of Artificial Sequence Synthetic peptide
SQTTHVPWT

SEQ ID NO: 46 - protein, 448 aa, Artificial Sequence
  Description of Artificial Sequence Synthetic polypeptide
QLQLQESGPGLLKPSETLSLTCTVSGGSISSPGYYGGWIRQPPGKGLEWIGSIYKSGSTYHNPSLKSRVTISVDTSKNQFSLKLSSVTAADTAVYYCTRPVVRYFGWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 47 - protein, 10 aa, Artificial Sequence
  Description of Artificial Sequence Synthetic peptide
GGSISSPGYY

SEQ ID NO: 48 - protein, 7 aa, Artificial Sequence
  Description of Artificial Sequence Synthetic peptide
IYKSGST

SEQ ID NO: 49 - protein, 13 aa, Artificial Sequence
  Description of Artificial Sequence Synthetic peptide
TRPVVRYFGWFDP

SEQ ID NO: 50 - protein, 213 aa, Artificial Sequence
  Description of Artificial Sequence Synthetic polypeptide
AIQLTQSPSSLSASVGDRVTITCRASQGISSALAWYQQKPGKAPKLLIYDASNLESGVPSRFSGSGSGTDFTLTISSLQPEDFATYYCQQFNSYPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 6 aa, Artificial Sequence
  Description of Artificial Sequence Synthetic peptide
QGISSA

SEQ ID NO: 52 - protein, 3 aa, Artificial Sequence
  Description of Artificial Sequence Synthetic peptide
DAS

SEQ ID NO: 53 - protein, 8 aa, Artificial Sequence
  Description of Artificial Sequence Synthetic peptide
QQFNSYPT

SEQ ID NO: 54 - protein, 450 aa, Artificial Sequence
  Description of Artificial Sequence Synthetic polypeptide
QVQLVESGGGVVQPGRSLRLSCAASGFTFSSYGMHWVRQAPGKGLEWVAVISYEESNRYHADSVKGRFTISRDNSKITLYLQMNSLRTEDTAVYYCARDGGIAAPGPDYWGQGTLVTVSSASTKGPSVFPLAPASKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 8 aa, Artificial Sequence
  Description of Artificial Sequence Synthetic peptide
GFTFSSYG

SEQ ID NO: 56 - protein, 8 aa, Artificial Sequence
  Description of Artificial Sequence Synthetic peptide
ISYEESNR

SEQ ID NO: 57 - protein, 13 aa, Artificial Sequence
  Description of Artificial Sequence Synthetic peptide
ARDGGIAAPGPDY

SEQ ID NO: 58 - protein, 219 aa, Artificial Sequence
  Description of Artificial Sequence Synthetic polypeptide
DIVMTQSPLSLTVTPGEPASISCRSSQSLLYSNGYNYLDWYLQKPGQSPQVLISLGSNRASGVPDRFSGSGSGTDFTLKISRVEAEDVGVYYCMQARQTPFTFGPGTKVDIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 11 aa, Artificial Sequence
  Description of Artificial Sequence Synthetic peptide
QSLLYSNGYNY

SEQ ID NO: 60 - protein, 3 aa, Artificial Sequence
  Description of Artificial Sequence Synthetic peptide
LGS

SEQ ID NO: 61 - protein, 9 aa, Artificial Sequence
  Description of Artificial Sequence Synthetic peptide
MQARQTPFT

SEQ ID NO: 62 - protein, 447 aa, Artificial Sequence
  Description of Artificial Sequence Synthetic polypeptide
EVQLLESGGGLVQPGGSLRLSCAASGFTFSTYGMHWVRQAPGKGLEWLSYISGGSSYIFYADSVRGRFTISRDNSENALYLQMNSLRAEDTAVYYCARILRGGSGMDLWGQGTLVTVSSASTKGPSVFPLAPSSKSTSGGTAALGCLVKDYFPEPVTVSWNSGALTSGVHTFPAVLQSSGLYSLSSWTVPSSSLGTQTYICNVNHKPSNTKVDKKVEPKSCDKTHTCPPCPAPELLGGPSVFLFPPKPKDTLMISRTPEVTCNAVDVSHEDPEVKFNWYVDGVEVHNAKTKPREEQYNSTYRWSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 8 aa, Artificial Sequence
  Description of Artificial Sequence Synthetic peptide
GFTFSTYG

SEQ ID NO: 64 - protein, 8 aa, Artificial Sequence
  Description of Artificial Sequence Synthetic peptide
ISGGSSYI

SEQ ID NO: 65 - protein, 12 aa, Artificial Sequence
  Description of Artificial Sequence Synthetic peptide
ARILRGGSGMDL

SEQ ID NO: 66 - protein, 217 aa, Artificial Sequence
  Description of Artificial Sequence Synthetic polypeptide
QSVLTQPPSASGTPGQRVTISCTGSSSNIGAGYNVYWYQQLPGTAPKLLIYGNINRPSGVPDRFSGSKSGTSASLAISGLRSEDEADYYCAAWDKSISGLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 67 - protein, 9 aa, Artificial Sequence
  Description of Artificial Sequence Synthetic peptide
SSNIGAGYN

SEQ ID NO: 68 - protein, 3 aa, Artificial Sequence
  Description of Artificial Sequence Synthetic peptide
GNI

SEQ ID NO: 69 - protein, 11 aa, Artificial Sequence
  Description of Artificial Sequence Synthetic peptide
AAWDKSISGLV

SEQ ID NO: 70 - protein, 450 aa, Artificial Sequence
  Description of Artificial Sequence Synthetic polypeptide
QVQLVESGGGVVQPGRSLRLSCAASGFSFSSTYVCWVRQAPGKGLEWIACIYTGDGTNYSASWAKGRFTISKDSSKNTVYLQMNSLRAEDTAVYFCARPDITYGFAINFWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 8 aa, Artificial Sequence
  Description of Artificial Sequence Synthetic peptide
GFSFSSTY

SEQ ID NO: 72 - protein, 8 aa, Artificial Sequence
  Description of Artificial Sequence Synthetic peptide
IYTGDGTN

SEQ ID NO: 73 - protein, 14 aa, Artificial Sequence
  Description of Artificial Sequence Synthetic peptide
ARPDITYGFAINFW

SEQ ID NO: 74 - protein, 215 aa, Artificial Sequence
  Description of Artificial Sequence Synthetic polypeptide
DIQMTQSPSSLSASVGDRVTIKCQASQSISSRLAWYQQKPGKPPKLLIYRASTLASGVPSRFSGSGSGTDFTLTISSLQPEDVATYYCQCTGYGISWPIGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 6 aa, Artificial Sequence
  Description of Artificial Sequence Synthetic peptide
QSISSR

SEQ ID NO: 76 - protein, 3 aa, Artificial Sequence
  Description of Artificial Sequence Synthetic peptide
RAS

SEQ ID NO: 77 - protein, 10 aa, Artificial Sequence
  Description of Artificial Sequence Synthetic peptide
QCTGYGISWP

SEQ ID NO: 78 - protein, 452 aa, Artificial Sequence
  Description of Artificial Sequence Synthetic polypeptide
EVQLQQSGPDLVKPGASVKISCKTSGYTFTEYIMHWVKQSHGKSLEWIGGIIPNNGGTSYNQKFKDKATMTVDKSSSTGYMELRSLTSEDSAVYYCTRREVYGRNYYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 8 aa, Artificial Sequence
  Description of Artificial Sequence Synthetic peptide
GYTFTEYI

SEQ ID NO: 80 - protein, 8 aa, Artificial Sequence
  Description of Artificial Sequence Synthetic peptide
IIPNNGGT

SEQ ID NO: 81 - protein, 15 aa, Artificial Sequence
  Description of Artificial Sequence Synthetic peptide
TRREVYGRNYYALDY

SEQ ID NO: 82 - protein, 214 aa, Artificial Sequence
  Description of Artificial Sequence Synthetic polypeptide
DIQMTQTTSSLSASLGDRVTITCSASQGINNYLNWYQQKPDGTVKLLIYYTSSLHSGVPSRFSGSGSGTDYSLTISNLEPEDIATYYCQQYSNLPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 83 - protein, 6 aa, Artificial Sequence
  Description of Artificial Sequence Synthetic peptide
QGINNY

SEQ ID NO: 84 - protein, 3 aa, Artificial Sequence
  Description of Artificial Sequence Synthetic peptide
YTS

SEQ ID NO: 85 - protein, 9 aa, Artificial Sequence
  Description of Artificial Sequence Synthetic peptide
QQYSNLPYT

SEQ ID NO: 86 - protein, 448 aa, Artificial Sequence
  Description of Artificial Sequence Synthetic polypeptide
QVQLVESGGGVVQPGRSLRLSCAASGFTFSNYGMYWVRQAPGKGLEWVAVIWYDGSNKYYADSVKGRFTISRDNSKNTLYLQMNSLRAEDTAVYYCARDLWGWYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 118 aa, Artificial Sequence
  Description of Artificial Sequence Synthetic polypeptide
QVQLVESGGGVVQPGRSLRLSCAASGFTFSNYGMYWVRQAPGKGLEWVAVIWYDGSNKYYADSVKGRFTISRDNSKNTLYLQMNSLRAEDTAVYYCARDLWGWYFDYWGQGTLVTVSS

SEQ ID NO: 88 - protein, 8 aa, Artificial Sequence
  Description of Artificial Sequence Synthetic peptide
GFTFSNYG

SEQ ID NO: 89 - protein, 8 aa, Artificial Sequence
  Description of Artificial Sequence Synthetic peptide
IWYDGSNK

SEQ ID NO: 90 - protein, 11 aa, Artificial Sequence
  Description of Artificial Sequence Synthetic peptide
ARDLWGWYFDY

SEQ ID NO: 91 - protein, 107 aa, Artificial Sequence
  Description of Artificial Sequence Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRRNWPLTFGGGTKVEIK

SEQ ID NO: 92 - protein, 107 aa, Artificial Sequence; identical to SEQ ID NO: 91
  Description of Artificial Sequence Synthetic polypeptide

SEQ ID NO: 93 - protein, 6 aa, Artificial Sequence
  Description of Artificial Sequence Synthetic peptide
QSVSSY

SEQ ID NO: 94 - protein, 3 aa, Artificial Sequence
  Description of Artificial Sequence Synthetic peptide
DAS

SEQ ID NO: 95 - protein, 9 aa, Artificial Sequence
  Description of Artificial Sequence Synthetic peptide
QQRRNWPLT

SEQ ID NO: 96 - protein, 446 aa, Artificial Sequence
  Description of Artificial Sequence Synthetic polypeptide
EVQLVQSGAEVKKPGESLRISCKGSGDSFTTYWIGWVRQMPGKGLEWMGIIYPGDSDTIYSPSFQGQVTISADKSISTAYLQWSSLKASDTAMYYCTRGDRGVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 116 aa, Artificial Sequence
  Description of Artificial Sequence Synthetic polypeptide
EVQLVQSGAEVKKPGESLRISCKGSGDSFTTYWIGWVRQMPGKGLEWMGIIYPGDSDTIYSPSFQGQVTISADKSISTAYLQWSSLKASDTAMYYCTRGDRGVDYWGQGTLVTVSS

SEQ ID NO: 98 - protein, 8 aa, Artificial Sequence
  Description of Artificial Sequence Synthetic peptide
GDSFTTYW

SEQ ID NO: 99 - protein, 8 aa, Artificial Sequence
  Description of Artificial Sequence Synthetic peptide
IYPGDSDT

SEQ ID NO: 100 - protein, 9 aa, Artificial Sequence
  Description of Artificial Sequence Synthetic peptide
TRGDRGVDY

SEQ ID NO: 101 - protein, 107 aa, Artificial Sequence
  Description of Artificial Sequence Synthetic polypeptide
DIQMTQSPSSLSASVGDRVTITCRASQGISRWLAWYQQKPEKAPKSLIYAASSLQSGVPSRFSGSGSGTDFTLTISGLQPEDFATYYCQQYNSYPRTFGQGTKVEIK

SEQ ID NO: 102 - protein, 107 aa, Artificial Sequence; identical to SEQ ID NO: 101
  Description of Artificial Sequence Synthetic polypeptide

SEQ ID NO: 103 - protein, 6 aa, Artificial Sequence
  Description of Artificial Sequence Synthetic peptide
QGISRW

SEQ ID NO: 104 - protein, 3 aa, Artificial Sequence
  Description of Artificial Sequence Synthetic peptide
AAS

SEQ ID NO: 105 - protein, 9 aa, Artificial Sequence
  Description of Artificial Sequence Synthetic peptide
QQYNSYPRT

SEQ ID NO: 106 - protein, 9 aa, Artificial Sequence
  Description of Artificial Sequence Synthetic peptide
QHFWNTPWT

SEQ ID NO: 107 - protein, 9 aa, Artificial Sequence
  Description of Artificial Sequence Synthetic peptide
QQGHTLPYT

SEQ ID NO: 108 - protein, 7 aa, Artificial Sequence
  Description of Artificial Sequence Synthetic peptide
SNDGYYS

SEQ ID NO: 109 - protein, 8 aa, Artificial Sequence
  Description of Artificial Sequence Synthetic peptide
YYGIYVDY

SEQ ID NO: 110 - protein, 4 aa, Artificial Sequence
  Description of Artificial Sequence Synthetic peptide
FLVY

SEQ ID NO: 111 - protein, 7 aa, Artificial Sequence
  Description of Artificial Sequence Synthetic peptide
NFGILGY

SEQ ID NO: 112 - protein, 9 aa, Artificial Sequence
  Description of Artificial Sequence Synthetic peptide
QQGNTLPPT

SEQ ID NO: 113 - protein, 9 aa, Artificial Sequence
  Description of Artificial Sequence Synthetic peptide
QQHYITPLT

SEQ ID NO: 114 - protein, 9 aa, Artificial Sequence
  Description of Artificial Sequence Synthetic peptide
QQYGNLPYT

SEQ ID NO: 115 - protein, 7 aa, Artificial Sequence
  Description of Artificial Sequence Synthetic peptide
RYYLGVD

SEQ ID NO: 116 - protein, 7 aa, Artificial Sequence
  Description of Artificial Sequence Synthetic peptide
DDSGRFP

SEQ ID NO: 117 - protein, 7 aa, Artificial Sequence
  Description of Artificial Sequence Synthetic peptide
YGYAVDY

SEQ ID NO: 118 - protein, 7 aa, Artificial Sequence
  Description of Artificial Sequence Synthetic peptide
YPNALDY

SEQ ID NO: 119 - protein, 10 aa, Artificial Sequence
  Description of Artificial Sequence Synthetic peptide
GLKSFYAMDH

SEQ ID NO: 120 - protein, 9 aa, Artificial Sequence
  Description of Artificial Sequence Synthetic peptide
QQGKTLPWT

SEQ ID NO: 121 - protein, 9 aa, Artificial Sequence
  Description of Artificial Sequence Synthetic peptide
QQYYSTPRT

SEQ ID NO: 122 - protein, 9 aa, Artificial Sequence
  Description of Artificial Sequence Synthetic peptide
GQSYNYPPT

SEQ ID NO: 123 - protein, 9 aa, Artificial Sequence
  Description of Artificial Sequence Synthetic peptide
WQDTHFPHV

SEQ ID NO: 124 - protein, 107 aa, Artificial Sequence; identical to SEQ ID NO: 101
  Description of Artificial Sequence Synthetic polypeptide

SEQ ID NO: 125 - protein, 116 aa, Artificial Sequence; identical to SEQ ID NO: 97
  Description of Artificial Sequence Synthetic polypeptide

SEQ ID NO: 126 - protein, 118 aa, Artificial Sequence
  Description of Artificial Sequence Synthetic polypeptide
QIVESGGGLVQPKESLKISCTASGFTFSNAAIYWVRQTPGKGLEWVGRIRTRPSKYATDYADSVRGRFTISRDDSKSMVYLQMDNLRTEDTAMYYCTPRATEDVPFYWGQGVMVTVSS

SEQ ID NO: 127 - protein, 109 aa, Artificial Sequence
  Description of Artificial Sequence Synthetic polypeptide
DIVMTQTPSSQAVSAGEKVTMNCKSSQSVLYDENKKNYLAWYQQKSGQSPKLLIYWASTGESGVPDRFIGSGSGTDFTLTISSVQAEDLAVYYCQQYYDFPPTFGGGTK

SEQ ID NO: 128 - protein, 448 aa, Artificial Sequence
  Description of Artificial Sequence Synthetic polypeptide
QVQLQESGPGLVKPSETLSLTCVVSGGSISSSNWWSWVRQPPGKGLEWIGEIYHSGSPDYNPSLKSRVTISVDKSRNQFSLKLSSVTAADTAVYYCAKVSTGGFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - protein, 448 aa, Artificial Sequence
  Description of Artificial Sequence Synthetic polypeptide
QVQLQESGPGLVKPSETLSLTCVVSGGSISSSNWWSWVRQPPGKGLEWIGEIYHSGSPNYNPSLKSRVTISVDKSKNQFSLKLSSVTAADTAVYYCARSSSGGFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 130 - protein, 448 aa, Artificial Sequence
  Description of Artificial Sequence Synthetic polypeptide
QVQLQESGPGLVKPSETLSLTCVVSGGSISSSNWWSWVRQPPGKGLEWIGEIYHSGSPNYNPSLKSRVTISVDKSKNQFSLKLSSVTAADTAVYYCARQTTAGSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - protein, 214 aa, Artificial Sequence
  Description of Artificial Sequence Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 132 - protein, 450 aa, Artificial Sequence
  Description of Artificial Sequence Synthetic polypeptide
QVQLVESGGGVVQPGRSLRLSCAASGFTLSSYGMHWVRQAPGKGLEWVAVIWYDGNDKYYADSVKGRFTISRDNSKNTLYLQVNSLRAEDTAVYYCARGTGTPYWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 450 aa, Artificial Sequence
  Description of Artificial Sequence Synthetic polypeptide
EVQLVESGGGLVQPGGSLRLSCAASGFTFSNYLMNWVRQAPGKGLEWVANIEQDGSEKYYVDSVKGRFTISRDNAKNSLYLQMNSLRAEDTAVYFCARDGDTAMITFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 134 - protein, 452 aa, Artificial Sequence
  Description of Artificial Sequence Synthetic polypeptide
QVQLQESGPGLVKPSQTLSLTCTVSGGSISSGGYYWNWIRQHPGKGLEWIGYIYYSGNTYYNPSLKSRVTISVDTSKNQFSLKLSSVTAADAAVYHCARGYGDYGGGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 215 aa, Artificial Sequence
  Description of Artificial Sequence Synthetic polypeptide
EIVLTQSPATLSLSPGERATLSCRASQSVNNYLAWYQQKPGQAPRLLIYDASNRATGIPARFSGSGSGTDFTLTISSLEPEDFAVYYCQQRSTWPPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 136 - protein, 214 aa, Artificial Sequence
  Description of Artificial Sequence Synthetic polypeptide
DIQMTQSPSSVSASVGDRVTITCRASQGIRRWLAWYQQKPGKAPKLLIYAASSLQRGVPSRFSGSGSGTDFTLTISSLQPEDFATYYCQQANSFPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 137 - protein, 214 aa, Artificial Sequence
  Description of Artificial Sequence Synthetic polypeptide
DIQMTQSPSSLSASVGDRVTITCQASQDISNYLNWYQQKPGKAPKFLIYDVSNLETGVPSRFSGSGSGTDFTFTISSLQPEDIATYYCQQYDNLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 138 - protein, 25 aa, Artificial Sequence
  Description of Artificial Sequence Synthetic peptide
  MISC_FEATURE (1)..(25): This sequence may encompass 2-5 ′Gly Gly Gly Gly Ser′ repeating units
GGGGSGGGGSGGGGSGGGGSGGGGS

SEQ ID NO: 139 - protein, 25 aa, Artificial Sequence
  Description of Artificial Sequence Synthetic peptide
  MISC_FEATURE (1)..(25): This sequence may encompass 3-5 ′Gly Gly Gly Gly Ser′ repeating units
GGGGSGGGGSGGGGSGGGGSGGGGS

SEQ ID NO: 140 - protein, 4 aa, Artificial Sequence
  Description of Artificial Sequence Synthetic peptide
GFLG

SEQ ID NO: 141 - protein, 4 aa, Artificial Sequence
  Description of Artificial Sequence Synthetic peptide
ALAL

SEQ ID NO: 142 - protein, 114 aa, Artificial Sequence [sequence omitted]

SEQ ID NO: 143 - protein, 112 aa, Artificial Sequence [sequence omitted]

SEQ ID NO: 144 - protein, 121 aa, Artificial Sequence
  Description of Artificial Sequence Synthetic polypeptide
QLQLQESGPGLLKPSETLSLTCTVSGGSISSPGYYGGWIRQPPGKGLEWIGSIYKSGSTYHNPSLKSRVTISVDTSKNQFSLKLSSVTAADTAVYYCTRPVVRYFGWFDPWGQGTLVTVSS

SEQ ID NO: 145 - protein, 106 aa, Artificial Sequence
  Description of Artificial Sequence Synthetic polypeptide
AIQLTQSPSSLSASVGDRVTITCRASQGISSALAWYQQKPGKAPKLLIYDASNLESGVPSRFSGSGSGTDFTLTISSLQPEDFATYYCQQFNSYPTFGQGTKVEIK

SEQ ID NO: 146 - protein, 120 aa, Artificial Sequence
  Description of Artificial Sequence Synthetic polypeptide
QVQLVESGGGVVQPGRSLRLSCAASGFTFSSYGMHWVRQAPGKGLEWVAVISYEESNRYHADSVKGRFTISRDNSKITLYLQMNSLRTEDTAVYYCARDGGIAAPGPDYWGQGTLVTVSS

SEQ ID NO: 147 - protein, 112 aa, Artificial Sequence
  Description of Artificial Sequence Synthetic polypeptide
DIVMTQSPLSLTVTPGEPASISCRSSQSLLYSNGYNYLDWYLQKPGQSPQVLISLGSNRASGVPDRFSGSGSGTDFTLKISRVEAEDVGVYYCMQARQTPFTFGPGTKVDIR

SEQ ID NO: 148 - protein, 119 aa, Artificial Sequence
  Description of Artificial Sequence Synthetic polypeptide
EVQLLESGGGLVQPGGSLRLSCAASGFTFSTYGMHWVRQAPGKGLEWLSYISGGSSYIFYADSVRGRFTISRDNSENALYLQMNSLRAEDTAVYYCARILRGGSGMDLWGQGTLVTVSS

SEQ ID NO: 149 - protein, 111 aa, Artificial Sequence
  Description of Artificial Sequence Synthetic polypeptide
QSVLTQPPSASGTPGQRVTISCTGSSSNIGAGYNVYWYQQLPGTAPKLLIYGNINRPSGVPDRFSGSKSGTSASLAISGLRSEDEADYYCAAWDKSISGLVFGGGTKLTVL

SEQ ID NO: 150 - protein, 120 aa, Artificial Sequence
  Description of Artificial Sequence Synthetic polypeptide
QVQLVESGGGVVQPGRSLRLSCAASGFSFSSTYVCWVRQAPGKGLEWIACIYTGDGTNYSASWAKGRFTISKDSSKNTVYLQMNSLRAEDTAVYFCARPDITYGFAINFWGPGTLVTVSS

SEQ ID NO: 151 - protein, 108 aa, Artificial Sequence
  Description of Artificial Sequence Synthetic polypeptide
DIQMTQSPSSLSASVGDRVTIKCQASQSISSRLAWYQQKPGKPPKLLIYRASTLASGVPSRFSGSGSGTDFTLTISSLQPEDVATYYCQCTGYGISWPIGGGTKVEIK

SEQ ID NO: 152 - protein, 117 aa, Artificial Sequence
  Description of Artificial Sequence Synthetic polypeptide
EVQLQQSGPDLVKPGASVKISCKTSGYTFTEYIMHWVKQSHGKSLEWIGGIIPNNGGTSYNQKFKDKATMTVDKSSSTGYMELRSLTSEDSAVYYCTRREVYGRNYYALDYWGQGTL

SEQ ID NO: 153 - protein, 107 aa, Artificial Sequence
  Description of Artificial Sequence Synthetic polypeptide
DIQMTQTTSSLSASLGDRVTITCSASQGINNYLNWYQQKPDGTVKLLIYYTSSLHSGVPSRFSGSGSGTDYSLTISNLEPEDIATYYCQQYSNLPYTFGGGTKLEIK

SEQ ID NO: 154 - protein, 331 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNEGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 155 - DNA, 1371 bp, Artificial Sequence
  Description of Artificial Sequence Synthetic polynucleotide
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctgacagtggtggcacaaactatgcacagaagtttcagggcagggtcaccatgaccagggacacgtccatcagcacagcctacatggagctgaacaggctgagatctgacgacacggccgtgtattactgtgcgagagatcagcccctaggatattgtactaatggtgtatgctcctactttgactactggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcccaaatcttgtgacaaaactcacacatgcccaccgtgcccagcacctgaactcgtggggggaccgtcagtcttcctcctgcccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcctgaggagcagtacaacagcacgctgcgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctctggtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 156 - DNA, 1371 bp, Artificial Sequence
  Description of Artificial Sequence Synthetic polynucleotide
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctgacagtggtggcacaaactatgcacagaagtttcagggcagggtcaccatgaccagggacacgtccatcagcacagcctacatggagctgaacaggctgagatctgacgacacggccgtgtattactgtgcgagagatcagcccctaggatattgtactaatggtgtatgctcctactttgactactggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcccaaatcttgtgacaaaactcacacatgcccaccgtgcccagcacctgaactcctggggggaccggatgtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccgaggagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 157 - DNA, 1371 bp, Artificial Sequence
  Description of Artificial Sequence Synthetic polynucleotide
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctgacagtggtggcacaaactatgcacagaagtttcagggcagggtcaccatgaccagggacacgtccatcagcacagcctacatggagctgaacaggctgagatctgacgacacggccgtgtattactgtgcgagagatcagcccctaggatattgtactaatggtgtatgctcctactttgactactggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacgccacgtaccgtgtggtcagcgtcctcaccgtcctgcaccaggactggctgaatggcaaggagtacaagtgcaaggtctccaacaaagccctcccagcccccatcgccgctaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 158 - DNA, 1371 bp, Artificial Sequence
  Description of Artificial Sequence Synthetic polynucleotide
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctgacagtggtggcacaaactatgcacagaagtttcagggcagggtcaccatgaccagggacacgtccatcagcacagcctacatggagctgaacaggctgagatctgacgacacggccgtgtattactgtgcgagagatcagcccctaggatattgtactaatggtgtatgctcctactttgactactggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 159 - protein, 456 aa, Artificial Sequence
  Description of Artificial Sequence Synthetic polypeptide
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVEPKSCDKTHTCPPCPAPELVGGPSVFLLPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSREEMTKNQVSLTCLVKGFYPSDIAVEWESNGQPENNYKTTPLVLDSDGSFFLYSKLTVDKSRWQQGNVFSCSVMHEALHNHYTQKSLSLSPGK

SEQ ID NO: 160 - protein, 456 aa, Artificial Sequence
  Description of Artificial Sequence Synthetic polypeptide
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 161 - protein, 456 aa, Artificial Sequence; identical to SEQ ID NO: 22
  Description of Artificial Sequence Synthetic polypeptide

SEQ ID NO: 162 - protein, 456 aa, Artificial Sequence
  Description of Artificial Sequence Synthetic polypeptide
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2020/0199242 A1
(43) Pub. Date: Jun. 25, 2020

(54) CONSTRUCT-PEPTIDE COMPOSITIONS AND METHODS OF USE THEREOF

(71) Applicant: SILVERBACK THERAPEUTICS, INC., Seattle, WA (US)

(72) Inventor: Peter Armstrong Thompson, Seattle, WA (US)

(21) Appl. No.: 16/478,825

(22) PCT Filed: Feb. 2, 2018
(86) PCT No.: PCT/US2018/016757

§ 371 Date: Jul. 17, 2019

Related U.S. Application Data
(60) Provisional application No. 62/453,998, filed on Feb. 2, 2017.

(51) Int. Cl. C07K 16/28; C07K 14/47; A61P 37/04

(52) U.S. Cl. CPC C07K 16/2878; A61K 2039/505; A61P 37/04; C07K 14/4748

(57) ABSTRACT

Construct-peptide compositions are disclosed. Construct-peptide compositions conjugated to immune stimulatory compounds are also provided. Additionally provided are the methods of preparation and use of the construct-peptide compositions and construct-peptide compositions conjugated to immune stimulatory compound. This includes methods for treating disorders, such as cancer.

### CROSS-REFERENCE

This application claims the benefit of U.S. Provisional Patent Application No. 62/453,998, filed Feb. 2, 2017, the entire disclosure of which is incorporated by reference herein.

### SEQUENCE LISTING

The instant application contains a Sequence Listing which has been submitted electronically in ASCII format and is hereby incorporated by reference in its entirety. Said ASCII copy, created on Feb. 2, 2018, is named 50358-705_601_SL.txt and is 196,871 bytes in size.

### BACKGROUND

One of the leading causes of death in the United States is cancer. The conventional methods of cancer treatment, like chemotherapy, surgery, or radiation therapy, tend to be either highly toxic or nonspecific to a cancer, or both, resulting in limited efficacy and harmful side effects. However, the immune system has the potential to be a powerful, specific tool in fighting cancers. In many cases tumors can specifically express genes whose products are required for inducing or maintaining the malignant state. These proteins may serve as antigen markers for the development and establishment of more specific anti-cancer immune response. The boosting of this specific immune response has the potential to be a powerful anti-cancer treatment that can be more effective than conventional methods of cancer treatment and can have fewer side effects.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

In some aspects, a composition may comprise an immune-stimulatory compound connected to a construct by a first linker. The construct may comprise an antigen binding domain, wherein the antigen binding domain may specifically bind a target antigen. The construct may additionally comprise an Fc domain, wherein a K_{d} for binding of the Fc domain to an Fc receptor in the presence of the immune-stimulatory compound may be no greater than about 100 times a K_{d} for binding of the Fc domain to the Fc receptor in the absence of the immune stimulatory compound. The construct may also comprise a peptide comprising an antigenic epitope of a cancer sequence, and wherein the peptide may be connected to the construct.

In some embodiments the peptide is connected to the construct as a fusion protein or by a second linker at the C-terminus of the Fc domain. In some embodiments, the peptide comprises a non-synonymous mutation, neoantigen, splice variant of a tumor specific epitope, or a tumor specific epitope. In some embodiments, the peptide comprises a non-synonymous mutation. In some embodiments, the peptide comprises a mutation selected from the group consisting of: a V157F, G154V, R176G, P278A, Y220C, G245S, R248Q, or R273H mutation in p53; a G466V or V600E mutation in BRAF; a E79Q mutation in NFE2L2; a G719A mutation in EGFR; a G12D, G12V, G12C, or G12R mutation in KRAS; a G12V, Q61L, or Q61R mutation in HRAS; a G12D, G12S, G13D, Q61K, or Q61R mutation in NRAS; a Q311E mutation in C3orf59; a E805G mutation in ERBB2IP; a A359D mutation in NUP98; a E426K mutation in GPD2; a E1179K mutation in PLEC; a P274S mutation in XPO7; a Q418K mutation in AKAP2; a F67V mutation in CASP8; a S1002I mutation in ITGB4; a P293L mutation in TUBGCP2; a N1702S mutation in RNF213; a R653H mutation in SKIV2L; a A48T mutation in H3F3B; a R243Q mutation in API5; a E572K mutation in RNF10; a G566E mutation in PHLPP1; and a R6H mutation in 2FYVE27.

In some embodiments, the peptide comprises 50 amino acids. In some embodiments, a non-synonymous mutation in the peptide is a centrally located amino acid in the amino acid sequence of the peptide. In some embodiments, the peptide comprising a non-synonymous mutation is from a cancer. In some embodiments, the peptide binds to an MHC class I molecule.

In some embodiments, the target antigen is a tumor associated antigen or an antigen expressed on an immune cell. In some embodiments, the target antigen is a tumor associated antigen. In some embodiments, the peptide binds to an MHC class II molecule. In some embodiments, the target antigen is an antigen expressed on an immune cell.

In some embodiments, the target antigen is a tumor associated antigen and has an amino acid sequence to an amino acid sequence of an antigen selected from the group consisting of: CD5, CD19, CD20, CD25, CD37, CD30, CD33, CD45, CAMPATH-1, HLD-DR, GD2, GD3, GM2, Ley, carcinoembryonic antigen (CEA), TAG-72, EpCAM, MUC1, MUC15, folate-binding protein, A33, G250, prostate-specific membrane antigen (PSMA), ferritin, CA-125, CA19-9, epidermal growth factor, p185HER2, IL-2 receptor, fibroblast activation protein (FAP), tenascin, a metalloproteinase, endosialin, vascular endothelial growth factor, avB3, WT1, LMP2, HPV E6, HPV E7, EGFRvIII (de2-7 EGFR), Her-2/neu, idiotype, MAGE A3, p53 nonmutant, NY-ESO-1, MelanA/MART1, Ras mutant, gp100, p53 mutant, PR1, bcr-abl, tyrosinase, survivin, P5A, hTERT, a Sarcoma translocation breakpoint fusion protein, EphA2, PAP, ML-IAP, AFP, ERG, NA17, PAX3, ALK, androgen receptor, cyclin B1, polysialic acid, MYCN, RhoC, TRP-2, fucosyl GM1, mesothelin (MSLN), PSCA, MAGE A1, sLe(animal), CYP1B1, PLAV1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, B7H3, Legumain, Tie 3, Page4, VEGFR2, MAD-CT-1, PDGFR-B, MAD-CT-2, ROR2, CMET, HER3, EPCAM, CA6, NAPI2B, TROP2, CLDN18.2, RON, LY6E, FRA, DLL3, PTK7, LIV1, ROR1, CMET, HER3, EPCAM, Fos-related antigen 1, VEGFR, endoglin, PD-L1, CD204, CD206, CD301, VTCN1, or VISTA.

In some embodiments, the peptide is a polymer of peptides. In some embodiments, the polymer of peptides comprises at least two different peptides. In some embodiments, the peptide is immunogenic.

In some embodiments, the target antigen is an antigen expressed on an immune cell. In some embodiments, the immune cell is an antigen presenting cell. In some embodiments, an amino acid sequence of the antigen expressed on an immune cell is selected from the group consisting of CD40, DEC-205, DICR, DNGR-1, BDCA-2, CD36 mannose scavenger receptor 1, CLEC12A, DC-SIGN, OX40L, 4-1BBL, CD36, CD204, MARCO, CLEC9A, Dectin 1, Dectin 2, CLEC10A, CD206, CD64, CD32a, CD16a, HVEM, and CD32b.

In some embodiments, the antigen binding domain is a CD40 agonist, a DEC-205 agonist, DICR agonist, DNGR-1 agonist, BDCA-2 agonist, CD36 mannose scavenger receptor 1 agonist, CLEC12A agonist, DC-SIGN agonist, OX40L agonist, 4-1BBL agonist, CD36 agonist, CD204 agonist, MARCO agonist, CLEC9A agonist, Dectin 1 agonist, Dectin 2 agonist, CLEC10A agonist, CD206 agonist, CD64 agonist, CD32a agonist, CD16a agonist, HVEM agonist, or CD32b agonist.

In some embodiments, the antigen binding domain comprises: a) HC CDR1 comprising an amino acid sequence of SEQ ID NO: 23, HC CDR2 comprising an amino acid sequence of SEQ ID NO: 24, a HC CDR3 comprising an amino acid sequence of SEQ ID NO: 25, LC CDR1 comprising an amino acid sequence of SEQ ID NO: 27, LC CDR1 comprising an amino acid sequence of SEQ ID NO: 28, and LC CDR3 comprising an amino acid sequence of SEQ ID NO: 29; b) HC CDR1 comprising an amino acid sequence of SEQ ID NO: 88, HC CDR2 comprising an amino acid sequence of SEQ ID NO: 89, a HC CDR3 comprising an amino acid sequence of SEQ ID NO: 90, LC CDR1 comprising an amino acid sequence of SEQ ID NO: 93, LC CDR1 comprising an amino acid sequence of SEQ ID NO: 94, and LC CDR3 comprising an amino acid sequence of SEQ ID NO: 95; c) HC CDR1 comprising an amino acid sequence of SEQ ID NO: 98, HC CDR2 comprising an amino acid sequence of SEQ ID NO: 99, a HC CDR3 comprising an amino acid sequence of SEQ ID NO: 100, LC CDR1 comprising an amino acid sequence of SEQ ID NO: 103, LC CDR1 comprising an amino acid sequence of SEQ ID NO: 104, and LC CDR3 comprising an amino acid sequence of SEQ ID NO: 105; d) HC CDR1 comprising an amino acid sequence of SEQ ID NO: 106, HC CDR2 comprising an amino acid sequence of SEQ ID NO: 107, a HC CDR3 comprising an amino acid sequence of SEQ ID NO: 108, LC CDR1 comprising an amino acid sequence of SEQ ID NO: 115, LC CDR1 comprising an amino acid sequence of SEQ ID NO: 116, and LC CDR3 comprising an amino acid sequence of SEQ ID NO: 117; e) HC CDR1 comprising an amino acid sequence of SEQ ID NO: 109, HC CDR2 comprising an amino acid sequence of SEQ ID NO: 110, a HC CDR3 comprising an amino acid sequence of SEQ ID NO: 111, LC CDR1 comprising an amino acid sequence of SEQ ID NO: 118, LC CDR1 comprising an amino acid sequence of SEQ ID NO: 119, and LC CDR3 comprising an amino acid sequence of SEQ ID NO: 120; or f) HC CDR1 comprising an amino acid sequence of SEQ ID NO: 112, HC CDR2 comprising an amino acid sequence of SEQ ID NO: 113, a HC CDR3 comprising an amino acid sequence of SEQ ID NO: 114, LC CDR1 comprising an amino acid sequence of SEQ ID NO: 121, LC CDR1 comprising an amino acid sequence of SEQ ID NO: 122, and LC CDR3 comprising an amino acid sequence of SEQ ID NO: 123.

In some embodiments, the construct is an antibody. In some embodiments, the K_{d} for binding of the Fc domain to the Fc receptor in the presence of the immune-stimulatory compound is no greater than about two times, five times, ten times, or fifty times a K_{d} for binding of the Fc domain to the Fc receptor in an absence of the immune-stimulatory compound. In some embodiments, the Fc domain is a human Fc domain. In some embodiments, the Fc domain is selected from a group consisting of a human IgG1 Fc domain, a human IgG2 Fc domain, a human IgG3 Fc domain, and a human IgG4 Fc domain. In some embodiments, the Fc domain binds an Fc receptor with the same affinity as a wild type IgG1 Fc domain.

In some embodiments, the Fc domain comprises one or more amino acid substitutions that increase the affinity of the Fc domain to an Fc receptor compared to the affinity of a reference Fc domain to the Fc receptor in the absence of the one or more amino acid substitutions. In some embodiments, the Fc domain has at least one amino acid residue change as compared to a wildtype Fc domain, wherein the at least one amino acid residue is: F243L, R292P, Y300L, L235V, and P396L, wherein numbering of amino acid residues in the Fc domain is according to the EU index as in Kabat; S239D and 1332E, wherein numbering of amino acid residues in the Fc domain is according to the EU index as in Kabat; or S298A, E333A, and K334A, wherein numbering of amino acid residues in the Fc domain is according to the EU index as in Kabat.

In some embodiments, the Fc domain comprises one or more amino acid substitutions that reduce the affinity of the Fc domain to an Fc receptor compared to the affinity of a reference Fc domain to the Fc receptor in the absence of the one or more amino acid substitutions and wherein the target antigen is an antigen expressed on an immune cell.

In some embodiments, a K_{d} for binding of the Fc domain to an Fc receptor in the presence of the peptide and the immune stimulatory compound is no greater than about 100 times a K_{d} for binding of the Fc domain to the Fc receptor in the absence of the peptide and the immune-stimulatory compound. In some embodiments, the K_{d} for binding of the Fc domain to an Fc receptor in the presence of the peptide and the immune stimulatory compound is no greater than 500 nM, no greater than 100 nM, no greater than 50 nM, no greater than 25 nM, no greater than 10 nM, no greater than 5 nM, no greater than 1 nM, or no greater than 0.1 nM.

In some embodiments, the immune-stimulatory compound is a damage-associated molecular pattern molecule or a pathogen-associated molecular pattern molecule. In some embodiments, the damage-associated molecular pattern molecule is HMGP1, S100A8/S100A9, ATP, uric acid, APP, SAA, granulysin, or eosinophil-derived neurotoxin.

In some embodiments, the immune-stimulatory compound is a toll-like receptor agonist, STING agonist, or RIG-I agonist. In some embodiments, the immune-stimulatory compound is a TLR1 agonist, a TLR2 agonist, a TLR3 agonist, a TLR4 agonist, a TLR5 agonist, a TLR6 agonist, a TLR7 agonist, a TLR8 agonist, a TLR9 agonist, or a TLR10 agonist.

In some embodiments, the immune-stimulatory compound is selected from a group consisting of: CpG oligonucleotide, Poly G10, Poly G3, Poly I:C, Lipopolysaccharide, zymosan, Flagellin, Pam3CSK4, PamCysPamSK4, dsRNA, a diacylated lipopeptide, a triacylated lipoprotein, lipoteichoic acid, a peptidoglycan, a cyclic dinucleotide, a 5′ppp-dsRNA, S-27609, CL307, UC-IV150, imiquimod, gardiquimod, resiquimod, motolimod, VTS-1463GS-9620, GSK2245035, TMX-101, TMX-201, TMX-202, isatoribine, AZD8848, MEDI9197, 3M-051, 3M-852, 3M-052, 3M-854A, S-34240, KU34B, CL663, SB9200, SB11285, or 8-substituted 2-amino-3H-benzo[b]azepine-4-carbozamide.

In some embodiments, the immune-stimulatory compound is an inhibitor of TGFβ, β-Catenin, PI3K-β, STAT3, IL-10, IDO, or TDO. In some embodiments, the immune-stimulatory compound is LY2109761, GSK263771, iCRT3, iCRT5, iCRT14, LY2090314, CGX-1321, PRI-724, BC21, ZINCO2092166, LGK974, IWP2, LY3022859, LY364947, SB431542, AZD8186, SD-208, indoximod (NLG8189), F001287, GDC-0919, epacadostat (INCB024360), RG70099, 1-methyl-L-tryptophan, methylthiohydantoin tryptophan, brassinin, annulin B, exiguamine A, PIM, LM10, INCB023843, or 8-substituted imidazo[1,5-a]pyridine.

In some embodiments, a molar ratio of the immune-stimulatory compound to the construct is less than 8.

In some embodiments, a composition mixture may comprise at least two different compositions disclosed herein.

In some aspects, the at least two different compositions each comprise a different peptide.

In some aspects, a pharmaceutical composition may comprise the compositions or the composition mixtures and a pharmaceutically acceptable carrier.

In some embodiments, a method of treating a subject in need thereof may comprise administering a therapeutic dose of the compositions or composition mixtures or the pharmaceutical compositions. In some embodiments, the subject has cancer. In some embodiments, the composition, composition mixture, or pharmaceutical composition is administered intravenously, cutaneously, subcutaneously, or injected at a site of affliction.

In some embodiments, a method producing the peptide may comprise sequencing the genome or transcriptome of a cancer cell to produce a cancer cell sequence; comparing the cancer cell sequence to a sequence from a normal cell to identify a mutation in the cancer cell sequence; and generating the antigenic peptide with at least 80% sequence identity to the cancer cell sequence with the mutation, wherein the mutation is present in the peptide. In some embodiments, the cancer cell and the normal cell are from one subject. In some embodiments, the cancer cell sequence is clonally represented within a cancer from a patient. In some embodiments, the cancer cell sequence contains a driver mutation of a cancer.

In some embodiments, the peptide is immunogenic. In some embodiments, the peptide binds to MHC. In some embodiments, the peptide binds to MHC with a Kd of no greater than 500 nM, no greater than 100 nM, no greater than 50 nM, no greater than 25 nM, no greater than 10 nM, no greater than 5 nM, no greater than 1 nM, or no greater than 0.1 nM. In some embodiments, the MHC is MHC class I or MHC class II. In some embodiments, the method is used to produce a peptide library for a cancer type.

In one embodiment, a composition comprises a construct comprising an antigen binding domain, wherein said antigen binding domain specifically binds an antigen; and a peptide with at least 80% sequence identity to a cancer sequence, wherein said peptide is connected to said construct.

In some aspects, said peptide is connected to said construct as a fusion protein. In some aspects, said peptide is connected to said construct by a first linker.

In some aspects, the composition further comprises an immune-stimulatory compound.

In various aspects, the composition further comprises a second linker attaching said construct to said immune-stimulatory compound.

In some aspects, said peptide is an antigenic peptide. In some aspects, said peptide comprises: a V157F, G154V, R176G, P278A, Y220C, G245S, R248Q, or R273H mutation in p53; a G466V or V600E mutation of BRAF; a E79Q mutation in NFE2L2; a G719A mutation in EGFR; a G12D, G12V, or G12C mutation in KRAS; a G12V, Q61L, or Q61R mutation in HRAS; a G12D, G12S, G13D, Q61K, or Q61R mutation in NRAS; a Q311E mutation in C3orf59; a E805G mutation in ERBB2IP; a A359D mutation in NUP98; a E426K mutation in GPD2; a E1179K mutation in PLEC; a P274S mutation in XPO7; a Q418K mutation in AKAP2; a F67V mutation in CASP8; a S1002I mutation in ITGB4; a P293L mutation in TUBGCP2; a N1702S mutation in RNF213; a R653H mutation in SKIV2L; a A48T mutation in H3F3B; a R243Q mutation in API5; a E572K mutation in RNF10; a G566E mutation in PHLPP1; or a R6H mutation in 2FYVE27.

In some aspects, said peptide comprises 25 amino acids. In some aspects, said peptide comprises 15-30 amino acids. In some embodiments, the peptide comprises 50 amino acids. In some aspects, said peptide has a non-synonymous nucleic acid mutation for a centrally located amino acid. In some aspects, the non-synonymous mutation is from a cancer. In some aspects, said non-synonymous mutation is from a cancer of a patient being treated with said composition.

In some aspects, said antigen binding domain is from an antibody or non-antibody scaffold. In some aspects, said antigen binding domain is at least 80% homologous to an antigen binding domain from an antibody or non-antibody scaffold. In some aspects, said antigen binding domain is from DARPins, affimers, avimers, knottins, monobodies, or affinity clamps. In some aspects, said antigen binding domain is at least 80% homologous to an antigen binding domain from DARPins, affimers, avimers, knottins, monobodies, or affinity clamps.

In various aspects, said antigen binding domain recognizes a single antigen.

In some aspects, a K_{d} for binding of said antigen binding domain to said antigen in a presence of said peptide is less than about 500 nM and no greater than about 100 times a K_{d} for binding of said antigen binding domain to said antigen in the absence of said peptide. In some aspects, a K_{d} for binding of said antigen binding domain to said antigen in a presence of said peptide is no greater than 500 nM, no greater than 100 nM, no greater than 50 nM, no greater than 25 nM, no greater than 10 nM, no greater than 5 nM, no greater than 1 nM, or no greater than 0.1 nM. In various aspects, a K_{d} for binding of said antigen binding domain to said antigen in a presence of said peptide and said immune-stimulatory compound is less than about 500 nM and no greater than about 100 times a K_{d} for binding of said antigen binding domain to said antigen in the absence of said peptide and said immune stimulatory compound. In some aspects, a K_{d} for binding of said antigen binding domain to said antigen in a presence of said peptide and said immune stimulatory compound is no greater than 500 nM, no greater than 100 nM, no greater than 50 nM, no greater than 25 nM, no greater than 10 nM, no greater than 5 nM, no greater than 1 nM, or no greater than 0.1 nM.

In some aspects, said antigen binding domain recognizes two or more antigens.

In some aspects, said antigen is a peptide presented in a major histocompatibility complex by a cell. In some aspects, said presented peptide is at least 80% homologous to CD5, CD19, CD20, CD25, CD37, CD30, CD33, CD45, CAMPATH-1, HLD-DR, carcinoembryonic antigen (CEA), TAG-72, EpCAM, MUC1, MUC15, folate-binding protein, A33, G250, prostate-specific membrane antigen (PSMA), ferritin, GD2, GD3, GM2, Le^{y}, CA-125, CA19-9, epidermal growth factor, p185HER2, IL-2 receptor, fibroblast activation protein (FAP), tenascin, a metalloproteinase, endosialin, vascular endothelial growth factor, avB3, WT1, LMP2, HPV E6, HPV E7, EGFRvIII (de2-7 EGFR), Her-2/neu, idiotype, MAGE A3, p53 nonmutant, NY-ESO-1, MelanA/MART1, Ras mutant, gp100, p53 mutant, PR1, bcr-abl, tyrosinase, survivin, PSA, hTERT, a Sarcoma translocation breakpoint fusion protein, EphA2, PAP, ML-IAP, AFP, ERG, NA17, PAX3, ALK, androgen receptor, cyclin B1, polysialic acid, MYCN, RhoC, TRP-2, fucosyl GM1, mesothelin (MSLN), PSCA, MAGE A1, sLe(animal), CYP1B1, PLAV1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, B7H3, Legumain, Tie 3, Page4, VEGFR2, MAD-CT-1, PDGFR-B, MAD-CT-2, ROR2, CMET, HER3, EPCAM, CA6, NAPI2B, TROP2, CLDN18.2, RON, LY6E, FRA, DLL3, PTK7, LIV1, ROR1, Fos-related antigen 1, VEGFR, endoglin, PD-L1, CD204, CD206, CD301, VTCN1, or VISTA.

In some aspects, said antigen is expressed on an immune cell. In some aspects, said antigen is expressed on an antigen-presenting cell. In some aspects, said antigen is expressed on a dendritic cell, a macrophage, or a B-cell. In some aspects, said antigen is at least 80% homologous to CD40. In some aspects, said antigen is at least 80% homologous to DEC-205, DICR, DNGR-1, BDCA-2, CD36 mannose scavenger receptor 1, CLEC12A, DC-SIGN, OX40L, 4-1BBL, CD36, CD204, MARCO, CLEC9A, Dectin 1, Dectin 2, CLEC10A, CD206, CD64, CD32a, CD16a, HVEM, or CD32b. In some aspects, said antigen binding domain is a CD40 agonist. In some aspects, said antigen binding domain is a DEC-205 agonist, DICR agonist, DNGR-1 agonist, BDCA-2 agonist, CD36 mannose scavenger receptor 1 agonist, CLEC12A agonist, DC-SIGN agonist, OX40L agonist, 4-1BBL agonist, CD36 agonist, CD204 agonist, MARCO agonist, CLEC9A agonist, Dectin 1 agonist, Dectin 2 agonist, CLEC10A agonist, CD206 agonist, CD64 agonist, CD32a agonist, CD16a agonist, HVEM agonist, or CD32b agonist.

In various aspects, said construct is an antibody. In some aspects, said construct is an antibody comprising an Fc domain. In some aspects, said construct is a human antibody or a humanized antibody. In some aspects, said construct comprises: a light chain sequence that is at least 80%, 90%, or 100% homologous to SEQ ID NO: 4; a light chain sequence that is at least 80%, 90%, or 100% homologous to SEQ ID NO: 26; a light chain sequence that is at least 80%, 90%, or 100% homologous to SEQ ID NO: 91; a light chain sequence that is at least 80%, 90%, or 100% homologous to SEQ ID NO: 101; a light chain sequence that is at least 80%, 90%, or 100% homologous to SEQ ID NO: 131; a light chain sequence that is at least 80%, 90%, or 100% homologous to SEQ ID NO: 135; a light chain sequence that is at least 80%, 90%, or 100% homologous to SEQ ID NO: 136; or a light chain sequence that is at least 80%, 90%, or 100% homologous to SEQ ID NO: 137. In some aspects, said construct comprises: a light chain variable domain sequence that is at least 80%, 90%, or 100% homologous to SEQ ID NO: 6; a light chain variable domain sequence that is at least 80%, 90%, or 100% homologous to SEQ ID NO: 92; a light chain variable domain sequence that is at least 80%, 90%, or 100% homologous to SEQ ID NO: 102; a light chain variable domain sequence that is at least 80%, 90%, or 100% homologous to SEQ ID NO: 125; or a light chain variable domain sequence that is at least 80%, 90%, or 100% homologous to SEQ ID NO: 127. In various aspects, said construct comprises: a heavy chain sequence that is at least 80%, 90%, or 100% homologous to SEQ ID NO: 15; a heavy chain sequence that is at least 80%, 90%, or 100% homologous to SEQ ID NO: 16; a heavy chain sequence that is at least 80%, 90%, or 100% homologous to SEQ ID NO: 17; a heavy chain sequence that is at least 80%, 90%, or 100% homologous to SEQ ID NO: 18; a heavy chain sequence that is at least 80%, 90%, or 100% homologous to SEQ ID NO: 22; a heavy chain sequence that is at least 80%, 90%, or 100% homologous to SEQ ID NO: 86; a heavy chain sequence that is at least 80%, 90%, or 100% homologous to SEQ ID NO: 96; a heavy chain sequence that is at least 80%, 90%, or 100% homologous to SEQ ID NO: 128; a heavy chain sequence that is at least 80%, 90%, or 100% homologous to SEQ ID NO: 129; a heavy chain sequence that is at least 80%, 90%, or 100% homologous to SEQ ID NO: 130; a heavy chain sequence that is at least 80%, 90%, or 100% homologous to SEQ ID NO: 132; a heavy chain sequence that is at least 80%, 90%, or 100% homologous to SEQ ID NO: 133; or a heavy chain sequence that is at least 80%, 90%, or 100% homologous to SEQ ID NO: 134. In some aspects, said construct comprises: a heavy chain variable domain that is at least 80%, 90%, or 100% homologous to SEQ ID NO: 20; a heavy chain variable domain that is at least 80%, 90%, or 100% homologous to SEQ ID NO: 87; a heavy chain variable domain that is at least 80%, 90%, or 100% homologous to SEQ ID NO: 97; a heavy chain variable domain that is at least 80%, 90%, or 100% homologous to SEQ ID NO: 124; or a heavy chain variable domain that is at least 80%, 90%, or 100% homologous to SEQ ID NO: 126. In some aspects, said antigen binding domain comprises at least 80%, 90%, or 100% homology to: HC CDR1 comprising an amino acid sequence of SEQ ID NO: 23, HC CDR2 comprising an amino acid sequence of SEQ ID NO: 24, a HC CDR3 comprising an amino acid sequence of SEQ ID NO: 25, LC CDR1 comprising an amino acid sequence of SEQ ID NO: 27, LC CDR1 comprising an amino acid sequence of SEQ ID NO: 28, and LC CDR3 comprising an amino acid sequence of SEQ ID NO: 29; HC CDR1 comprising an amino acid sequence of SEQ ID NO: 88, HC CDR2 comprising an amino acid sequence of SEQ ID NO: 89, a HC CDR3 comprising an amino acid sequence of SEQ ID NO: 90, LC CDR1 comprising an amino acid sequence of SEQ ID NO: 93, LC CDR1 comprising an amino acid sequence of SEQ ID NO: 94, and LC CDR3 comprising an amino acid sequence of SEQ ID NO: 95; HC CDR1 comprising an amino acid sequence of SEQ ID NO: 98, HC CDR2 comprising an amino acid sequence of SEQ ID NO: 99, a HC CDR3 comprising an amino acid sequence of SEQ ID NO: 100, LC CDR1 comprising an amino acid sequence of SEQ ID NO: 103, LC CDR1 comprising an amino acid sequence of SEQ ID NO: 104, and LC CDR3 comprising an amino acid sequence of SEQ ID NO: 105; HC CDR1 comprising an amino acid sequence of SEQ ID NO: 106, HC CDR2 comprising an amino acid sequence of O: 108, LC CDR1 comprising an amino acid sequence of SEQ ID NO: 115, LC CDR1 comprising an amino acid sequence of SEQ ID NO: 116, and LC CDR3 comprising an amino acid sequence of SEQ ID NO: 117; HC CDR1 comprising an amino acid sequence of SEQ ID NO: 109, HC CDR2 comprising an amino acid sequence of SEQ ID NO: 110, a HC CDR3 comprising an amino acid sequence of SEQ ID NO: 111, LC CDR1 comprising an amino acid sequence of SEQ ID NO: 118, LC CDR1 comprising an amino acid sequence of SEQ ID NO: 119, and LC CDR3 comprising an amino acid sequence of SEQ ID NO: 120; or HC CDR1 comprising an amino acid sequence of SEQ ID NO: 112, HC CDR2 comprising an amino acid sequence of SEQ ID NO: 113, a HC CDR3 comprising an amino acid sequence of SEQ ID NO: 114, LC CDR1 comprising an amino acid sequence of SEQ ID NO: 121, LC CDR1 comprising an amino acid sequence of SEQ ID NO: 122, and LC CDR3 comprising an amino acid sequence of SEQ ID NO: 123.

In various aspects, said construct comprises an Fc domain. In various aspects, said construct comprises an Fc domain, wherein said Fc domain specifically binds an Fc receptor in the presence of said immune-stimulatory compound. In some aspects, said construct comprises an Fc domain, wherein said Fc domain binding to an Fc receptor in the presence of said immune-stimulatory compound results in Fc-receptor-mediated signaling. In various aspects, said construct comprises an Fc domain, wherein said Fc domain specifically binds an Fc receptor in the presence of said peptide. In some aspects, said construct comprises an Fc domain, wherein said Fc domain binding to an Fc receptor in the presence of said peptide results in Fc-receptor-mediated signaling. In some aspects, said Fc domain is from a non-antibody scaffold or an antibody. In some aspects, said Fc domain is at least 80% homologous to an Fc domain from a non-antibody scaffold or an antibody. In various aspects, said Fc domain is a human Fc domain.

In some aspects, the Fc domain is an IgG region. In some aspects, the Fc domain is an IgG1 Fc region. In some aspects, the Fc domain is an Fc domain variant comprising one or more amino acid substitutions in an IgG region as compared to an amino acid sequence of a wild-type IgG region. In some aspects, the Fc domain variant has increased affinity to one or more Fcγ receptors as compared to the wild-type IgG region. In some aspects, the Fc domain is a non-antibody scaffold. In some aspects, said Fc domain is selected from a group consisting of a human IgG1 Fc domain, a human IgG2 Fc domain, a human IgG3 Fc domain, and a human IgG4 Fc domain. In some aspects, said Fc domain is an Fc domain variant comprising at least one amino acid residue changes as compared to a wild type sequence of said Fc domain. In some aspects, wherein said Fc domain binds an Fc receptor with altered affinity as compared to a wild type Fc domain. In some aspects, said Fc receptor is selected from a group consisting of CD16a, CD16b, CD32a, CD32b, and CD64. In some aspects, said Fc receptor is a CD16a F158 variant or a CD16a V158 variant. In some aspects, said Fc domain binds an Fc receptor with higher affinity than a wild type Fc domain. In some aspects, Fc receptor is selected from a group consisting of: CD16a, CD16b, CD32a, CD32b, or CD64. In some aspects, said Fc receptor is a CD16a F158 variant or a CD16a V158 variant. In some aspects, said Fc domain has at least one amino acid residue change as compared to wildtype, wherein said at least one amino acid residue is F243L, R292P, Y300L, L235V, and P396L, wherein numbering of amino acid residues in said Fc domain is according to the EU index as in Kabat. In some aspects, said Fc domain has at least one amino acid residue change as compared to wildtype, wherein said at least one amino acid residue is S239D and I332E, wherein numbering of amino acid residues in said Fc domain is according to the EU index as in Kabat. In some aspects, said Fc domain has at least one amino acid residue change as compared to wildtype, wherein said at least one amino acid residue is S298A, E333A, and K334A, wherein numbering of amino acid residues in said Fc domain is according to the EU index as in Kabat.

In various aspects, a K_{d} for binding of said Fc domain to an Fc receptor in the presence of said peptide is no greater than about 100 times a K_{d} for binding of said Fc domain to said Fc receptor in the absence of said peptide. In some aspects, said K_{d} for binding of said Fc domain to an Fc receptor in the presence of said peptide is no greater than 500 nM, no greater than 100 nM, no greater than 50 nM, no greater than 25 nM, no greater than 10 nM, no greater than 5 nM, no greater than 1 nM, or no greater than 0.1 nM. In some aspects, a K_{d} for binding of said Fc domain to an Fc receptor in the presence of said peptide and said immune stimulatory compound is no greater than about 100 times a K_{d} for binding of said Fc domain to said Fc receptor in the absence of said peptide and said immune-stimulatory compound. In some aspects, said Kd for binding of said Fc domain to an Fc receptor in the presence of said peptide and said immune stimulatory compound is no greater than 500 nM, no greater than 100 nM, no greater than 50 nM, no greater than 25 nM, no greater than 10 nM, no greater than 5 nM, no greater than 1 nM, or no greater than 0.1 nM.

In some aspects, said immune-stimulatory compound is a damage-associated molecular pattern molecule. In some aspects, said damage-associated molecular pattern molecule is HMGP1, S100A8/S100A9, ATP, uric acid, APP, SAA, granulysin, or eosinophil-derived neurotoxin.

In some aspects, said immune-stimulatory compound is a pathogen-associated molecular pattern molecule. In some aspects, said immune-stimulatory compound is a toll-like receptor agonist. In some aspects, said immune-stimulatory compound is a STING agonist. In some aspects, said immune-stimulatory agonist is a RIG-I agonist. In some aspects, said immune-stimulatory compound is a TLR1 agonist, a TLR2 agonist, a TLR3 agonist, a TLR4 agonist, a TLR5 agonist, a TLR6 agonist, a TLR7 agonist, a TLR8 agonist, a TLR9 agonist, or a TLR10 agonist. In some aspects, said immune-stimulatory compound is selected from a group consisting of: CpG oligonucleotide, Poly G10, Poly G3, Poly I:C, Lipopolysaccharide, zymosan, Flagellin, Pam3CSK4, PamCysPamSK4, dsRNA, a diacylated lipopeptide, a triacylated lipoprotein, lipoteichoic acid, a peptidoglycan, a cyclic dinucleotide, a 5′ppp-dsRNA, S-27609, CL307, UC-IV150, imiquimod, gardiquimod, resiquimod, motolimod, VTS-1463GS-9620, GSK2245035, TMX-101, TMX-201, TMX-202, isatoribine, AZD8848, MEDI9197, 3M-051, 3M-852, 3M-052, 3M-854A, S-34240, KU34B, CL663, SB9200, SB11285, or 8-substituted 2-amino-3H-benzo[b]azepine-4-carbozamide. In some aspects, said immune-stimulatory compound comprises one or more rings selected from carbocyclic and heterocyclic rings. In some aspects, the immune-stimulatory compound acts to attentuate or reverse the activity of an immunosuppressive pathway. In some aspects, the immune-stimulatory compound is an inhibitor of TGFβ, β-Catenin, PI3K-β, STAT3, IL-10, IDO, or TDO. In some aspects, the immune-stimulatory compound is LY2109761, GSK263771, iCRT3, iCRT5, iCRT14, LY2090314, CGX-1321, PRI-724, BC21, ZINCO2092166, LGK974, IWP2, LY3022859, LY364947, SB431542, AZD8186, SD-208, indoximod (NLG8189), F001287, GDC-0919, epacadostat (INCB024360), RG70099, 1-methyl-L-tryptophan, methylthiohydantoin tryptophan, brassinin, annulin B, exiguamine A, PIM, LM10, INCB023843, or 8-substituted imidazo[1,5-a]pyridine.

In some aspects, said first linker is conjugated to a carboxyl terminus of a light chain of said construct. In some aspects, said first linker is conjugated to a carboxyl terminus of a heavy chain of said construct. In some aspects, said first linker is conjugated to an amino acid residue of said construct by a THIOMAB linker, or a Sortase A-catalyzed linker. In various aspects, said first linker is conjugated to said antibody construct via a sulfhydryl group. In some aspects, said first linker is conjugated to said antibody construct via a primary amine. In some aspects, said first linker is conjugated to said construct via a hinge cysteine, a CL lysine, an engineered cysteine in a light chain, an engineered light chain glutamine, or an unnatural amino acid engineered into a light chain or heavy chain. In some aspects, said first linker is conjugated to said construct at an amino acid residue, wherein a conjugation at said amino acid residue does not interfere with said Fc domain binding to an Fc receptor. In some aspects, said first linker is conjugated to said construct at an amino acid residue, wherein a conjugation at said amino acid residue does not interfere with Fc-receptor-mediated signaling resulting from said Fc domain binding to an Fc receptor. In some aspects, said first linker is conjugated at the C-terminal amino acid or N-terminal amino acid of said peptide. In some aspects, said peptide is covalently bound to said linker by a bond to lysine amino acid residue

In various aspects, said first linker is a cleavable linker. In some aspects, said first linker is a valine-citrulline linker. In some aspects, said first linker is a valine-citrulline linker containing a pentafluorophenyl group. In some aspects, said first linker is a valine-citrulline linker containing a succinimide group. In some aspects, said first linker is a valine-citrulline linker containing a para aminobenzoic acid group. In some aspects, said first linker is a valine-citrulline linker containing a para aminobenzoic acid group and a pentafluorophenyl group. In some aspects, said first linker is a valine-citrulline linker containing a para aminobenzoic acid group and a succinimide group.

In some aspects, said first linker is a non-cleavable linker. In some aspects, said first linker is a maleimidocaproyl linker. In some aspects, said first linker is a combination of a maleimidocaproyl group and one or more polyethylene glycol molecules. In some aspects, said first linker is a maleimide-PEG4 linker. In some aspects, said first linker is a maleimidocaproyl linker containing a succinimide group. In some aspects, said first linker is a maleimidocaproyl linker containing a pentafluorophenyl group. In some aspects, said first linker is a combination of a maleimidocaproyl linker containing a succinimide group and one or more polyethylene glycol molecules. In some aspects, said first linker is a combination of a maleimidocaproyl linker containing a pentafluorophenyl group and one or more polyethylene glycol molecules.

In some aspects, said second linker is not conjugated at any amino acid residue selected from a group consisting of: 221, 222, 224, 227, 228, 230, 231, 223, 233, 234, 235, 236, 237, 238, 239, 240, 241, 243, 244, 245, 246, 247, 249, 250, 258, 262, 263, 264, 265, 266, 267, 268, 269, 270, 271, 272, 273, 274, 275, 276, 278, 280, 281, 283, 285, 286, 288, 290, 291, 292, 293, 294, 295, 296, 297, 298, 299, 300, 302, 305, 313, 317, 318, 320, 322, 323, 324, 325, 326, 327, 328, 329, 330, 331, 332, 333, 334, 335 336, 396, or 428, wherein numbering of amino acid residues in said Fc domain is according to the EU index as in Kabat. In some aspects, said second linker is conjugated to an amino acid residue of said antibody construct by a THIOMAB linker, or a Sortase A-catalyzed linker.

In some aspects, said second linker is conjugated to said antibody construct via a sulfhydryl group. In some aspects, said second linker is conjugated to said antibody construct via a primary amine. In some aspects, said second is conjugated to said antibody construct via a hinge cysteine, a CL lysine, an engineered cysteine in a light chain, an engineered light chain glutamine, or an unnatural amino acid engineered into a light chain or heavy chain. In some aspects, said second linker is conjugated to said antibody construct at an amino acid residue, wherein a conjugation at said amino acid residue does not interfere with said Fc domain binding to said Fc receptor. In some aspects, said second linker is conjugated to said antibody at an amino acid residue, wherein a conjugation at said amino acid residue does not interfere with Fc-receptor-mediated signaling resulting from said Fc domain binding to an Fc receptor. In some aspects, said second linker is conjugated to said immune-stimulatory compound via an exocyclic nitrogen or carbon atom of said immune-stimulatory compound. In some aspects, said immune-stimulatory compound is covalently bound to said second linker by a bond to an exocyclic carbon or nitrogen atom on said immune-stimulatory compound.

In some aspects, said second linker is a cleavable linker. In some aspects, said second linker is a valine-citrulline linker. In some aspects, said second linker is a valine-citrulline linker containing a pentafluorophenyl group. In some aspects, said second linker is a valine-citrulline linker containing a succinimide group. In some aspects, said second linker is a valine-citrulline linker containing a para aminobenzoic acid group. In some aspects, said second linker is a valine-citrulline linker containing a para aminobenzoic acid group and a pentafluorophenyl group. In some aspects, said second linker is a valine-citrulline linker containing a para aminobenzoic acid group and a succinimide group.

In various aspects, said second linker is a non-cleavable linker. In some aspects, said second linker is a maleimidocaproyl linker. In some aspects, said second linker is a combination of a maleimidocaproyl group and one or more polyethylene glycol molecules. In some aspects, said second linker is a maleimide-PEG4 linker. In some aspects, said second linker is a maleimidocaproyl linker containing a succinimide group. In some aspects, said second linker is a maleimidocaproyl linker containing a pentafluorophenyl group. In some aspects, said second linker is a combination of a maleimidocaproyl linker containing a succinimide group and one or more polyethylene glycol molecules. In some aspects, said second linker is a combination of a maleimidocaproyl linker containing a pentafluorophenyl group and one or more polyethylene glycol molecules.

In some aspects, a molar ratio of said immune-stimulatory compound to said construct is less than 8.

In various aspects, said composition is formulated to treat tumors.

In some aspects, said composition is in a pharmaceutical formulation.

In some embodiments, a method of treating a subject in need thereof comprises administering said composition of any one of preceding embodiments.

In various embodiments, a method of treating cancer comprises administering said composition of any one of the preceding embodiments.

In some embodiments, a method producing said peptide of any one of the preceding embodiments comprises sequencing the genome or transcriptome of a cancer cell to produce a cancer cell sequence; comparing said cancer cell sequence to a sequence from a normal cell to identify a mutation in said cancer cell sequence; and generating said peptide with at least 80% sequence identity to said cancer cell sequence with said mutation, wherein said mutation is present in said peptide. In some aspects, said cancer cell and said normal cell are from one subject. In some aspects, said cancer cell sequence is clonally represented within a cancer from a patient. In some aspects, said cancer cell sequence contains a driver mutation of a cancer. In some aspects, said peptide is immunogenic. In various aspects, said peptide binds to MHC. In some aspects, said MHC is MHC class I or MHC class II. In some aspects, said peptide binds to MHC with a K_{d} of no greater than 500 nM, no greater than 100 nM, no greater than 50 nM, no greater than 25 nM, no greater than 10 nM, no greater than 5 nM, no greater than 1 nM, or no greater than 0.1 nM In some aspects, said method is used to produce a peptide library for a cancer type.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative aspects, in which the principles of the disclosure are utilized, and the accompanying drawings of which:

FIG. 1A illustrates a DNA sequence (SEQ ID NO: 1) of a light chain of a human CD40 monoclonal antibody SBT-040. Furthermore, SEQ ID NO: 1 illustrates a DNA sequence containing a signal sequence (SEQ ID NO: 2) as shown in FIG. 1B and a variable domain sequence (SEQ ID NO: 3) as shown in FIG. 1C.

FIG. 1B illustrates a DNA sequence of a signal sequence (SEQ ID NO: 2) of a light chain of a human CD40 monoclonal antibody SBT-040.

FIG. 1C illustrates a DNA sequence of a variable domain (SEQ ID NO: 3) in a light chain of a human CD40 monoclonal antibody SBT-040.

FIG. 2A illustrates an amino acid sequence (SEQ ID NO: 4) of a light chain of a human CD40 monoclonal antibody SBT-040. Furthermore, SEQ ID NO: 4 illustrates an amino acid sequence containing a signal sequence (SEQ ID NO: 5) as shown in FIG. 2B and a variable domain sequence (SEQ ID NO: 6) as shown in FIG. 2C.

FIG. 2B illustrates an amino acid sequence of a signal sequence (SEQ ID NO: 5) of a light chain of a human CD40 monoclonal antibody SBT-040.

FIG. 2C illustrates an amino acid sequence of a variable domain (SEQ ID NO: 6) in a light chain of a human CD40 monoclonal antibody SBT-040.

FIG. 3A illustrates a DNA sequence (SEQ ID NO: 7) of a wildtype IgG2 isotype heavy chain of a human CD40 monoclonal antibody SBT-040, wherein this heavy chain of the SBT-040 antibody can also be referred to as SBT-040-G2. Furthermore, SEQ ID NO: 7 illustrates a DNA sequence containing a signal sequence (SEQ ID NO: 12) as shown in FIG. 3F and a variable domain sequence (SEQ ID NO: 13) as shown in FIG. 3G.

FIG. 3B illustrates a DNA sequence (SEQ ID NO: 8) of a wildtype IgG1 isotype heavy chain of a human CD40 monoclonal antibody SBT-040, wherein this heavy chain of the SBT-040 antibody can also be referred to as SBT-040-G1WT. Furthermore, SEQ ID NO: 8 illustrates a DNA sequence containing a signal sequence (SEQ ID NO: 12) as shown in FIG. 3F and a variable domain sequence (SEQ ID NO: 13) as shown in FIG. 3G.

FIG. 3C illustrates a DNA sequence (SEQ ID NO: 9) of an IgG1 isotype heavy chain of a human CD40 monoclonal antibody SBT-040 containing DNA nucleotide modifications corresponding to L235V, F243L, R292P, Y300L, and P396L amino acid residue modifications of a wildtype IgG1 Fc domain, wherein this heavy chain of the SBT-040 antibody can also be referred to as SBT-040-G1VLPLL. The modified DNA nucleotides corresponding to the L235V, F243L, R292P, Y300L, and P396L amino acid residue modifications are in bold. Furthermore, SEQ ID NO: 9 illustrates a DNA sequence containing a signal sequence (SEQ ID NO: 12) as shown in FIG. 3F and a variable domain sequence (SEQ ID NO: 13) as shown in FIG. 3G.

FIG. 3D illustrates a DNA sequence (SEQ ID NO: 10) of an IgG isotype heavy chain of a human CD40 monoclonal antibody SBT-040 containing DNA nucleotide modifications corresponding to S239D and 1332E amino acid residue modifications of a wildtype IgG1 Fc domain, wherein this heavy chain of the SBT-040 antibody can also be referred to as SBT-040-G1DE. The modified DNA nucleotides corresponding to the S239D and 1332E amino acid residue modifications are in bold. Furthermore, SEQ ID NO: 10 illustrates a DNA sequence containing a signal sequence (SEQ ID NO: 12) as shown in FIG. 3F and a variable domain sequence (SEQ ID NO: 13) as shown in FIG. 3G.

FIG. 3E illustrates a DNA sequence (SEQ ID NO: 11) of an IgG1 isotype heavy chain of human CD40 monoclonal antibody SBT-040 containing DNA nucleotide modifications corresponding to S298A, E333A, and K334A amino acid residue modifications of a wildtype IgG1 Fc domain, wherein this heavy chain of the SBT-040 antibody can also be referred to as SBT-040-G1AAA. The modified DNA nucleotides corresponding to the S298A, E333A, and K334A amino acid residue modifications are in bold. Furthermore, SEQ ID NO: 11 illustrates a DNA sequence containing a signal sequence (SEQ ID NO: 12) as shown in FIG. 3F and a variable domain sequence (SEQ ID NO: 13) as shown in FIG. 3G.

FIG. 3F illustrates a DNA sequence of a signal sequence (SEQ ID NO: 12) of a heavy chain of a human CD40 monoclonal antibody SBT-040.

FIG. 3G illustrates a DNA sequence of a variable domain (SEQ ID NO: 13) in a heavy chain of a human CD40 monoclonal antibody SBT-040.

FIG. 4A illustrates an amino acid sequence (SEQ ID NO: 14) of a wildtype IgG2 isotype heavy chain of a human CD40 monoclonal antibody SBT-040, wherein this heavy chain of the SBT-040 antibody can also be referred to as SBT-040-G2. Furthermore, SEQ ID NO: 14 illustrates an amino acid sequence containing a signal sequence (SEQ ID NO: 19) as shown in FIG. 4F and a variable domain sequence (SEQ ID NO: 20) as shown in FIG. 4G.

FIG. 4B illustrates an amino acid sequence (SEQ ID NO: 15) of a wildtype IgG1 isotype heavy chain of a human CD40 monoclonal antibody SBT-040, wherein this heavy chain of the SBT-040 antibody can also be referred to as SBT-040-G1WT. Furthermore, SEQ ID NO: 15 illustrates an amino acid sequence containing a signal sequence (SEQ ID NO: 19) as shown in FIG. 4F and a variable domain sequence (SEQ ID NO: 20) as shown in FIG. 4G.

FIG. 4C illustrates an amino acid sequence (SEQ ID NO: 16) of an IgG1 isotype heavy chain of a human CD40 monoclonal antibody SBT-040 containing L235V, F243L, R292P, Y300L, and P396L amino acid residue modifications of a wildtype IgG1 Fc domain, wherein this heavy chain of the SBT-040 antibody can also be referred to as SBT-040-G1VLPLL. The amino acid residues corresponding to the L235V, F243L, R292P, Y300L, and P396L amino acid residue modifications are in bold. Furthermore, SEQ ID NO: 16 illustrates an amino acid sequence containing a signal sequence (SEQ ID NO: 19) as shown in FIG. 4F and a variable domain sequence (SEQ ID NO: 150) as shown in FIG. 4G.

FIG. 4D illustrates an amino acid sequence (SEQ ID NO: 17) of an IgG1 isotype heavy chain of a human CD40 monoclonal antibody SBT-040 containing S239D and 1332 amino acid residue modifications of a wildtype IgG1 Fc domain, wherein this heavy chain of the SBT-040 antibody can also be referred to as SBT-040-G1DE. The amino acid residues corresponding to the S239D and 1332E amino acid residue modifications are in bold. Furthermore, SEQ ID NO: 17 illustrates an amino acid sequence containing a signal sequence (SEQ ID NO: 19) as shown in FIG. 4F and a variable domain sequence (SEQ ID NO: 20) as shown in FIG. 4G.

FIG. 4E illustrates an amino acid sequence (SEQ ID NO: 18) of an IgG1 isotype heavy chain of a human CD40 monoclonal antibody SBT-040 containing S298A, E333A, and K334A amino acid residue modifications of a wildtype IgG1 Fc domain, wherein this heavy chain of the SBT-040 antibody can also be referred to as SBT-040-G1AAA. The amino acid residues corresponding to the S298A, E333A, and K334A amino acid modifications are in bold. Furthermore, SEQ ID NO: 11 illustrates an amino acid sequence containing a signal sequence (SEQ ID NO: 12) as shown in FIG. 4F and a variable domain sequence (SEQ ID NO: 13) as shown in FIG. 4G.

FIG. 4F illustrates an amino acid sequence of a signal sequence (SEQ ID NO: 12) of a heavy chain of a human CD40 monoclonal antibody SBT-040.

FIG. 4G illustrates an amino acid sequence of a variable domain (SEQ ID NO: 13) in a heavy chain of a human CD40 monoclonal antibody SBT-040.

FIG. 5 illustrates a CLUSTAL O(1.2.1) multiple DNA sequence alignment of the DNA sequences of SBT-040-G1VLPLL (SEQ ID NO: 9), SBT-040-G1AAA (SEQ ID NO: 11), SBT-040-G1WT (SEQ ID NO: 8), and SBT-040-G1DE (SEQ ID NO: 10). The SBT-040-G1 VLPLL sequence is a DNA sequence of an IgG1 isotype heavy chain of a human CD40 monoclonal antibody SBT-040 containing DNA nucleotide modifications corresponding to L235V, F243L, R292P, Y300L, and P396L amino acid residue modifications of a wild type IgG1 Fc domain. The modified DNA nucleotides corresponding to the L235V, F243L, R292P, Y300L, and P396L amino acid residue modifications are in bold. The SBT-040-G1AAA sequence is a DNA sequence of an IgG1 isotype heavy chain of a human CD40 monoclonal antibody SBT-040 containing DNA nucleotide modifications corresponding to S298A, E333A, and K334A amino acid residue modifications of a wild type IgG1 Fc domain. The modified DNA nucleotides corresponding to the S298A, E333A, and K334A amino acid residue modifications are boxed. The SBT-040-G1WT sequence is a DNA sequence of an IgG1 isotype heavy chain of a human CD40 monoclonal antibody SBT-040. The SBT-040-G1AAA sequence is a DNA sequence of an IgG1 isotype heavy chain of a human CD40 monoclonal antibody SBT-040 containing DNA nucleotide modifications corresponding to S239D and 1332E amino acid residue modifications of a wild type IgG1 Fc domain. The modified DNA nucleotides corresponding to the S239D and 1332E amino acid residue modifications are in bold italics. SEQ ID NO: 155 (CAGGTGCAGCTGGTGCAGTCTGGGGCTGAGGTGAAGAAGCCTGGGGCCTCAGTGAA GGTCTCCTGCAAGGCTTCTGGATACACCTTCACCGGCTACTATATGCACTGGGTGCG ACAGGCCCCTGGACAAGGGCTTGAGTGGATGGGATGGATCAACCCTGACAGTGGTG GCACAAACTATGCACAGAAGTTTCAGGGCAGGGTCACCATGACCAGGGACACGTCC ATCAGCACAGCCTACATGGAGCTGAACAGGCTGAGATCTGACGACACGGCCGTGTA TTACTGTGCGAGAGATCAGCCCCTAGGATATTGTACTAATGGTGTATGCTCCTACIT GACTACTGGGGCCAGGGAACCCTGGTCACCGTCTCCTCAGCCTCCACCAAGGGCCCA TCGGTCTTCCCCCTGGCGCCCTGCTCCAGGAGCACCTCCGAGAGCACAGCGGCCCTG GGCTGCCTGGTCAAGGACTACTTCCCCGAACCGGTGACGGTGTCGTGGAACTCAGGC GCTCTGACCAGCGGCGTGCACACCTTCCCAGCTGTCCTACAGTCCTCAGGACTCTAC TCCCTCAGCAGCGTGGTGACCGTGCCCTCCAGCAACTTCGGCACCCAGACCTACACC TGCAACGTAGATCACAAGCCCAGCAACACCAAGGTGGACAAGACAGTTGAGCCCAA ATCTTGTGACAAAACTCACACATGCCCACCGTGCCCAGCACCTGAACTCGTGGGGGG ACCGTCAGTCTTCCTCCTGCCCCCAAAACCCAAGGACACCCTCATGATCTCCCGGAC CCCTGAGGTCACATGCGTGGTGGTGGACGTGAGCCACGAAGACCCTGAGGTCAAGT TCAACTGGTACGTGGACGGCGTGGAGGTGCATAATGCCAAGACAAAGCCGCCTGAG GAGCAGTACAACAGCACGCTGCGTGTGGTCAGCGTCCTCACCGTCCTGCACCAGGAC TGGCTGAATGGCAAGGAGTACAAGTGCAAGGTCTCCAACAAAGCCCTCCCAGCCCC CATCGAGAAAACCATCTCCAAAGCCAAAGGGCAGCCCCGAGAACCACAGGTGTACA CCCTGCCCCCATCCCGGGAGGAGATGACCAAGAACCAGGTCAGCCTGACCTGCCTG GTCAAAGGCTTCTATCCCAGCGACATCGCCGTGGAGTGGGAGAGCAATGGGCAGCC GGAGAACAACTACAAGACCACGCCTCTGGTGCTGGACTCCGACGGCTCCTTCTTCCT CTATAGCAAGCTCACCGTGGACAAGAGCAGGTGGCAGCAGGGGAACGTCTTCTCAT GCCCGTGATGCATGAGGCTCTGCACAACCACTACACGCAGAAGAGCCTCTCCCTGT CCCCGGGTAAATGA) is SBT-040-G1VLPLL without the leader sequence. SEQ ID NO: 156 (CAGGTGCAGCTGGTGCAGTCTGGGGCTGAGGTGAAGAAGCCTGGGGCCTCAGTGAA GGTCTCCTGCAAGGCTTCTGGATACACCTTCACCGGCTACTATATGCACTGGGTGCG ACAGGCCCCTGGACAAGGGCTTGAGTGGATGGGATGGATCAACCCTGACAGTGGTG GCACAAACTATGCACAGAAGTTTCAGGGCAGGGTCACCATGACCAGGGACACGTCC ATCAGCACAGCCTACATGGAGCTGAACAGGCTGAGATCTGACGACACGGCCGTGTA TTACTGTGCGAGAGATCAGCCCCTAGGATATTGTACTAATGGTGTATGCTCCTACTTT GACTACTGGGGCCAGGGAACCCTGGTCACCGTCTCCTCAGCCTCCACCAAGGGCCCA TCGGTCTTCCCCCTGGCGCCCTGCTCCAGGAGCACCTCCGAGAGCACAGCGGCCCTG GGCTGCCTGGTCAAGGACTACTTCCCCGAACCGGTGACGGTGTCGTGGAACTCAGGC GCTCTGACCAGCGGCGTGCACACCTTCCCAGCTGTCCTACAGTCCTCAGGACTCTAC TCCCTCAGCAGCGTGGTGACCGTGCCCTCCAGCAACTTCGGCACCCAGACCTACACC TGCAACGTAGATCACAAGCCCAGCAACACCAAGGTGGACAAGACAGTTGAGCCCAA ATCTTGTGACAAAACTCACACATGCCCACCGTGCCCAGCACCTGAACTCCTGGGGGG ACCGGATGTCTTCCTCTTCCCCCCAAAACCCAAGGACACCCTCATGATCTCCCGGAC CCCTGAGGTCACATGCGTGGTGGTGGACGTGAGCCACGAAGACCCTGAGGTCAAGT TCAACTGGTACGTGGACGGCGTGGAGGTGCATAATGCCAAGACAAAGCCGCGGGAG GAGCAGTACAACAGCACGTACCGTGTGGTCAGCGTCCTCACCGTCCTGCACCAGGAC TGGCTGAATGGCAAGGAGTACAAGTGCAAGGTCTCCAACAAAGCCCTCCCAGCCCC CGAGGAGAAAACCATCTCCAAAGCCAAAGGGCAGCCCCGAGAACCACAGGTGTACA CCCTGCCCCCATCCCGGGAGGAGATGACCAAGAACCAGGTCAGCCTGACCTGCCTG GTCAAAGGCTTCTATCCCAGCGACATCGCCGTGGAGTGGGAGAGCAATGGGCAGCC GGAGAACAACTACAAGACCACGCCTCCCGTGCTGGACTCCGACGGCTCCTTCTTCCT CTATAGCAAGCTCACCGTGGACAAGAGCAGGTGGCAGCAGGGGAACGTCTTCTCAT GCCCGTGATGCATGAGGCTCTGCACAACCACTACACGCAGAAGAGCCTCTCCCTGT CCCCGGGTAAATGA) is SBT-040-G1DE without the leader sequence. SEQ ID NO: 157 (CAGGTGCAGCTGGTGCAGTCTGGGGCTGAGGTGAAGAAGCCTGGGGCCTCAGTGAA GGTCTCCTGCAAGGCTTCTGGATACACCTTCACCGGCTACTATATGCACTGGGTGCG ACAGGCCCCTGGACAAGGGCTTGAGTGGATGGGATGGATCAACCCTGACAGTGGTG GCACAAACTATGCACAGAAGTTTCAGGGCAGGGTCACCATGACCAGGGACACGTCC ATCAGCACAGCCTACATGGAGCTGAACAGGCTGAGATCTGACGACACGGCCGTGTA TTACTGTGCGAGAGATCAGCCCCTAGGATATTGTACTAATGGTGTATGCTCCTACTTT GACTACTGGGGCCAGGGAACCCTGGTCACCGTCTCCTCAGCCTCCACCAAGGGCCCA TCGGTCTTCCCCCTGGCGCCCTGCTCCAGGAGCACCTCCGAGAGCACAGCGGCCCTG GGCTGCCTGGTCAAGGACTACTTCCCCGAACCGGTGACGGTGTCGTGGAACTCAGGC GCTCTGACCAGCGGCGTGCACACCTTCCCAGCTGTCCTACAGTCCTCAGGACTCTAC TCCCTCAGCAGCGTGGTGACCGTGCCCTCCAGCAACTTCGGCACCCAGACCTACACC TGCAACGTAGATCACAAGCCCAGCAACACCAAGGTGGACAAGACAGTTGAGCCCAA ATCTTGTGACAAAACTCACACATGCCCACCGTGCCCAGCACCTGAACTCCTGGGGGG ACCGTCAGTCTTCCTCTTCCCCCCAAAACCCAAGGACACCCTCATGATCTCCCGGAC CCCTGAGGTCACATGCGTGGTGGTGGACGTGAGCCACGAAGACCCTGAGGTCAAGT TCAACTGGTACGTGGACGGCGTGGAGGTGCATAATGCCAAGACAAAGCCGCGGGAG GAGCAGTACAACGCCACGTACCGTGTGGTCAGCGTCCTCACCGTCCTGCACCAGGAC TGGCTGAATGGCAAGGAGTACAAGTGCAAGGTCTCCAACAAAGCCCTCCCAGCCCC CATCGCCGCTACCATCTCCAAAGCCAAAGGGCAGCCCCGAGAACCACAGGTGTACA CCCTGCCCCCATCCCGGGAGGAGATGACCAAGAACCAGGTCAGCCTGACCTGCCTG GTCAAAGGCTTCTATCCCAGCGACATCGCCGTGGAGTGGGAGAGCAATGGGCAGCC GGAGAACAACTACAAGACCACGCCTCCCGTGCTGGACTCCGACGGCTCCTTCTTCCT CTATAGCAAGCTCACCGTGGACAAGAGCAGGTGGCAGCAGGGGAACGTCTTCTCAT GCCCGTGATGCATGAGGCTCTGCACAACCACTACACGCAGAAGAGCCTCTCCCTGT CCCCGGGTAAATGA) is SBT-040-G1AAA without the leader sequence. SEQ ID NO: 158 (CAGGTGCAGCTGGTGCAGTCTGGGGCTGAGGTGAAGAAGCCTGGGGCCTCAGTGAA GGTCTCCTGCAAGGCTTCTGGATACACCTTCACCGGCTACTATATGCACTGGGTGCG ACAGGCCCCTGGACAAGGGCTTGAGTGGATGGGATGGATCAACCCTGACAGTGGTG GCACAAACTATGCACAGAAGTTTCAGGGCAGGGTCACCATGACCAGGGACACGTCC ATCAGCACAGCCTACATGGAGCTGAACAGGCTGAGATCTGACGACACGGCCGTGTA TTACTGTGCGAGAGATCAGCCCCTAGGATATTGTACTAATGGTGTATGCTCCTACTTT GACTACTGGGGCCAGGGAACCCTGGTCACCGTCTCCTCAGCCTCCACCAAGGGCCCA TCGGTCTTCCCCCTGGCGCCCTGCTCCAGGAGCACCTCCGAGAGCACAGCGGCCCTG GGCTGCCTGGTCAAGGACTACTTCCCCGAACCGGTGACGGTGTCGTGGAACTCAGGC GCTCTGACCAGCGGCGTGCACACCTTCCCAGCTGTCCTACAGTCCTCAGGACTCTAC TCCCTCAGCAGCGTGGTGACCGTGCCCTCCAGCAACTTCGGCACCCAGACCTACACC TGCAACGTAGATCACAAGCCCAGCAACACCAAGGTGGACAAGACAGTTGAGCCCAA ATCTTGTGACAAAACTCACACATGCCCACCGTGCCCAGCACCTGAACTCCTGGGGGG ACCGTCAGTCTTCCTCTTCCCCCCAAAACCCAAGGACACCCTCATGATCTCCCGGAC CCCTGAGGTCACATGCGTGGTGGTGGACGTGAGCCACGAAGACCCTGAGGTCAAGT TCAACTGGTACGTGGACGGCGTGGAGGTGCATAATGCCAAGACAAAGCCGCGGGAG GAGCAGTACAACAGCACGTACCGTGTGGTCAGCGTCCTCACCGTCCTGCACCAGGAC TGGCTGAATGGCAAGGAGTACAAGTGCAAGGTCTCCAACAAAGCCCTCCCAGCCCC CATCGAGAAAACCATCTCCAAAGCCAAAGGGCAGCCCCGAGAACCACAGGTGTACA CCCTGCCCCCATCCCGGGAGGAGATGACCAAGAACCAGGTCAGCCTGACCTGCCTG GTCAAAGGCTTCTATCCCAGCGACATCGCCGTGGAGTGGGAGAGCAATGGGCAGCC GGAGAACAACTACAAGACCACGCCTCCCGTGCTGGACTCCGACGGCTCCTTCTTCCT CTATAGCAAGCTCACCGTGGACAAGAGCAGGTGGCAGCAGGGGAACGTCTTCTCAT GCCCGTGATGCATGAGGCTCTGCACAACCACTACACGCAGAAGAGCCTCTCCCTGT CCCCGGGTAAATGA) is SBT-040-G1WT without the leader sequence.

FIG. 6 illustrates a CLUSTAL O(1.2.1) multiple amino acid sequence alignment of the amino acid sequences of SBT-040-G1VLPLL (SEQ ID NO: 16), SBT-040-G1AAA (SEQ ID NO: 18), SBT-040-G1WT (SEQ ID NO: 15), and SBT-040-G1DE (SEQ ID NO: 17). The SBT-040-G1VLPLL sequence is an amino acid sequence of an IgG1 isotype heavy chain of a human CD40 monoclonal antibody SBT-040 containing L235V, F243L, R292P, Y300L, and P396L amino acid residue modifications of a wild type IgG1 Fc domain. The L235V, F243L, R292P, Y300L, and P396L amino acid residue modifications are in bold. The SBT-040-G1AAA sequence is an amino acid sequence of an IgG1 isotype heavy chain of a human CD40 monoclonal antibody SBT-040 containing S298A, E333A, and K334A amino acid residue modifications of a wild type IgG1 Fc domain. The S298A, E333A, and K334A amino acid residue modifications are italics. The SBT-040-G1WT sequence is an amino acid sequence of an IgG1 isotype heavy chain of a human CD40 monoclonal antibody SBT-040. The SBT-040-G1 AAA sequence is an amino acid sequence of an IgG1 isotype heavy chain of a human CD40 monoclonal antibody SBT-040 containing S239D and 1332E amino acid residue modifications bold italics. Additionally, the hinge region of each amino acid sequence is differentiated from other regions of the amino acid sequence by brackets. The left bracket indicates the upper portion of the hinge region (UH). The four residues between the brackets are the middle portion of the hinge region. The right bracket indicates the lower portion of the hinge region (LH). SEQ ID NO: 143 (QVQLVQSGAEVKKPGASVKVSCKASGYTFTGYYMHWVRQAPGQGLEWMGWINPDSG GTNYAQKFQGRVTMTRDTSISTAYMELNRLRSDDTAVYYCARDQPLGYCTNGVCSYFD YWGQGTLVTVSSASTKGPS VFPLAPCSRSTSESTAALGCLVKDYFPEPVTVSWNSGALTS GVHTFPAVLQSSGLYSLSSVVTVPSSNFGTQTYTCNVDHKPSNTKVDKTVEPKSCDKTH TCPPCPAPELVGGPSVFLLPPKPKDTLMISRTPEVTCVVVDVSHEDPEVKFNWYVDGVE VHNAKTKPPEEQYNSTLRVVSVLTVLHQDWLNGKEYKCKVSNKALPAPIEKTISKAKG QPREPQVYTLPPSREEMTKNQVSLTCLVKGFYPSDIAVEWESNGQPENNYKTTPLVLDS DGSFFLYSKLTVDKSRWQQGNVFSCSVMHEALHNHYTQKSLSLSPGK) is the sequence of SBT-040-G1VLPLL without the leader sequence. SEQ ID NO: 144 (QVQLVQSGAEVKKPGASVKVSCKASGYTFTGYYMHWVRQAPGQGLEWMGWINPDSG GTNYAQKFQGRVTMTRDTSISTAYMELNRLRSDDTAVYYCARDQPLGYCTNGVCSYFD YWGQGTLVTVSSASTKGPS VFPLAPCSRSTSESTAALGCLVKDYFPEPVTVSWNSGALTS GVHTFPAVLQSSGLYSLSSVVTVPSSNFGTQTYTCNVDHKPSNTKVDKTVEPKSCDKTH TCPPCPAPELLGGPSVFLFPPKPKDTLMISRTPEVTCVVVDVSHEDPEVKFNWYVDGVEV HNAKTKPREEQYNATYRVVSVLTVLHQDWLNGKEYKCKVSNKALPAPIAATISKAKGQ PREPQVYTLPPSREEMTKNQVSLTCLVKGFYPSDIAVEWESNGQPENNYKTTPPVLDSD GSFFLYSKLTVDKSRWQQGNVFSCSVMHEALHNHYTQKSLSLSPGK) is the sequence of SBT-040-G1AAA without the leader sequence. SEQ ID NO: 145 (QVQLVQSGAEVKKPGASVKVSCKASGYTFTGYYMHWVRQAPGQGLEWMGWINPDSG GTNYAQKFQGRVTMTRDTSISTAYMELNRLRSDDTAVYYCARDQPLGYCTNGVCSYFD YWGQGTLVTVSSASTKGPS VFPLAPCSRSTSESTAALGCLVKDYFPEPVTVSWNSGALTS GVHTFPAVLQSSGLYSLSSVVTVPSSNFGTQTYTCNVDHKPSNTKVDKTVEPKSCDKTH TCPPCPAPELLGGPSVFLFPPKPKDTLMISRTPEVTCVVVDVSHEDPEVKFNWYVDGVEV HNAKTKPREEQYNSTYRVVSVLTVLHQDWLNGKEYKCKVSNKALPAPIEKTISKAKGQ PREPQVYTLPPSREEMTKNQVSLTCLVKGFYPSDIAVEWESNGQPENNYKTTPPVLDSD GSFFLYSKLTVDKSRWQQGNVFSCSVMHEALHNHYTQKSLSLSPGK) is the sequence of SBT-040-G1WT without the leader sequence. SEQ ID NO: 146 (QVQLVQSGAEVKKPGASVKVSCKASGYTFTGYYMHWVRQAPGQGLEWMGWINPDSG GTNYAQKFQGRVTMTRDTSISTAYMELNRLRSDDTAVYYCARDQPLGYCTNGVCSYFD YWGQGTLVTVSSASTKGPS VFPLAPCSRSTSESTAALGCLVKDYFPEPVTVSWNSGALTS GVHTFPAVLQSSGLYSLSSVVTVPSSNFGTQTYTCNVDHKPSNTKVDKTVEPKSCDKTH TCPPCPAPELLGGPDVFLFPPKPKDTLMISRTPEVTCVVVDVSHEDPEVKFNWYVDGVE VHNAKTKPREEQYNSTYRVVSVLTVLHQDWLNGKEYKCKVSNKALPAPEEKTISKAKG QPREPQVYTLPPSREEMTKNQVSLTCLVKGFYPSDIAVEWESNGQPENNYKTTPPVLDS DGSFFLYSKLTVDKSRWQQGNVFSCSVMHEALHNHYTQKSLSLSPGK) is the sequence of SBT-040-G1DE without the leader sequence. SEQ ID NO: 159 (QVQLVQSGAEVKKPGASVKVSCKASGYTFTGYYMHWVRQAPGQGLEWMGWINPDSG GTNYAQKFQGRVTMTRDTSISTAYMELNRLRSDDTAVYYCARDQPLGYCTNGVCSYFD YWGQGTLVTVSSASTKGPS VFPLAPCSRSTSESTAALGCLVKDYFPEPVTVSWNSGALTS GVHTFPAVLQSSGLYSLSSVVTVPSSNFGTQTYTCNVDHKPSNTKVDKTVEPKSCDKTH TCPPCPAPELVGGPSVFLLPPKPKDTLMISRTPEVTCVVVDVSHEDPEVKFNWYVDGVE VHNAKTKPPEEQYNSTLRVVSVLTVLHQDWLNGKEYKCKVSNKALPAPIEKTISKAKG QPREPQVYTLPPSREEMTKNQVSLTCLVKGFYPSDIAVEWESNGQPENNYKTTPLVLDS DGSFFLYSKLTVDKSRWQQGNVFSCSVMHEALHNHYTQKSLSLSPGK) is the sequence of SBT-040-G1VLPLL without the leader sequence. SEQ ID NO: 160 (QVQLVQSGAEVKKPGASVKVSCKASGYTFTGYYMHWVRQAPGQGLEWMGWINPDSG GTNYAQKFQGRVTMTRDTSISTAYMELNRLRSDDTAVYYCARDQPLGYCTNGVCSYFD YWGQGTLVTVSSASTKGPS VFPLAPCSRSTSESTAALGCLVKDYFPEPVTVSWNSGALTS GVHTFPAVLQSSGLYSLSSVVTVPSSNFGTQTYTCNVDHKPSNTKVDKTVEPKSCDKTH TCPPCPAPELLGGPSVFLFPPKPKDTLMISRTPEVTCVVVDVSHEDPEVKFNWYVDGVEV HNAKTKPREEQYNATYRVVSVLTVLHQDWLNGKEYKCKVSNKALPAPIAATISKAKGQ PREPQVYTLPPSREEMTKNQVSLTCLVKGFYPSDIAVEWESNGQPENNYKTTPPVLDSD GSFFLYSKLTVDKSRWQQGNVFSCSVMHEALHNHYTQKSLSLSPGK) is the sequence of SBT-040-G1AAA without the leader sequence. SEQ ID NO: 161 (QVQLVQSGAEVKKPGASVKVSCKASGYTFTGYYMHWVRQAPGQGLEWMGWINPDSG GTNYAQKFQGRVTMTRDTSISTAYMELNRLRSDDTAVYYCARDQPLGYCTNGVCSYFD YWGQGTLVTVSSASTKGPS VFPLAPCSRSTSESTAALGCLVKDYFPEPVTVSWNSGALTS GVHTFPAVLQSSGLYSLSSVVTVPSSNFGTQTYTCNVDHKPSNTKVDKTVEPKSCDKTH TCPPCPAPELLGGPSVFLFPPKPKDTLMISRTPEVTCVVVDVSHEDPEVKFNWYVDGVEV HNAKTKPREEQYNSTYRVVSVLTVLHQDWLNGKEYKCKVSNKALPAPIEKTISKAKGQ PREPQVYTLPPSREEMTKNQVSLTCLVKGFYPSDIAVEWESNGQPENNYKTTPPVLDSD GSFFLYSKLTVDKSRWQQGNVFSCSVMHEALHNHYTQKSLSLSPGK) is the sequence of SBT-040-G1WT without the leader sequence. SEQ ID NO: 162 (QVQLVQSGAEVKKPGASVKVSCKASGYTFTGYYMHWVRQAPGQGLEWMGWINPDSG GTNYAQKFQGRVTMTRDTSISTAYMELNRLRSDDTAVYYCARDQPLGYCTNGVCSYFD YWGQGTLVTVSSASTKGPS VFPLAPCSRSTSESTAALGCLVKDYFPEPVTVSWNSGALTS GVHTFPAVLQSSGLYSLSSVVTVPSSNFGTQTYTCNVDHKPSNTKVDKTVEPKSCDKTH TCPPCPAPELLGGPDVFLFPPKPKDTLMISRTPEVTCVVVDVSHEDPEVKFNWYVDGVE VHNAKTKPREEQYNSTYRVVSVLTVLHQDWLNGKEYKCKVSNKALPAPEEKTISKAKG QPREPQVYTLPPSREEMTKNQVSLTCLVKGFYPSDIAVEWESNGQPENNYKTTPPVLDS DGSFFLYSKLTVDKSRWQQGNVFSCSVMHEALHNHYTQKSLSLSPGK) is the sequence of SBT-040-G1DE without the leader sequence.

FIG. 7 illustrates a schematic of an antibody. The antibody comprises two heavy chains as shown in gray and two light chains as shown in light gray. A portion of the heavy chains contain Fc domains (705 and 720). The antibody has two antigen binding sites (710 and 715).

FIG. 8 illustrates a schematic of an exemplary construct-peptide composition. The construct is an antibody. The antibody comprises two heavy chains as shown in gray and two light chains as shown in light gray. The antibody has two antigen binding sites (810 and 815), and a portion of the heavy chains contain Fc domains (805 and 820). The peptides form a composition with the antibody (880 and 885). The peptides contain a nonsynonymous mutation (890 and 895).

FIG. 9 illustrates a schematic of an exemplary construct-peptide composition conjugated to immune-stimulatory compounds. The construct is an antibody. The antibody comprises two heavy chains as shown in gray and two light chains as shown in light gray. The antibody has two antigen binding sites (910 and 915), and a portion of the heavy chains contain Fc domains (905 and 920). The immune-stimulatory compounds (930 and 940) are conjugated to the antibody by linkers (960 and 970). The peptides (980 and 985) form a composition with the antibody. The peptides contain a nonsynonymous mutation (990 and 995).

FIG. 10 illustrates a schematic of an exemplary construct-peptide composition. The construct comprises Fc regions of an antibody with the heavy chains shown in gray, and two scaffolds as shown in light gray. The construct comprises two antigen binding sites (1010 and 1015) in the scaffolds, and a portion of the heavy chains contain Fc domains (1005 and 1020). The peptides (1080 and 1085) form a composition with the construct. The peptides contain a nonsynonymous mutation (1090 and 1095).

FIG. 11 illustrates a schematic of an exemplary construct-peptide composition conjugated to immune-stimulatory compounds. The construct comprises Fc regions of an antibody with the heavy chains shown in gray, and two scaffolds as shown in light gray. The construct comprises two antigen binding sites (1110 and 1115) in the scaffolds, and a portion of the heavy chains contain Fc domains (1105 and 1120). The immune-stimulatory compounds (1130 and 1140) are conjugated to the construct by linkers (1160 and 1170). The peptides (1180 and 1185) form a composition with the construct. The peptides contain a nonsynonymous mutation (1190 and 1195).

FIG. 12 illustrates a schematic of an exemplary construct-peptide composition. The construct comprises the F(ab′)2 regions of an antibody with heavy chains shown in gray and light chains shown in light gray, and two scaffolds as shown in dark gray. The construct comprises two antigen binding sites (1210 and 1215), and a portion of two scaffolds contain Fc domains (1240 and 1245). The peptides (1280 and 1285) form a composition with the construct. The peptides contain a nonsynonymous mutation (1290 and 1295).

FIG. 13 illustrates a schematic of an exemplary construct-peptide composition conjugated to immune-stimulatory compounds. The construct contains the F(ab′)2 regions of an antibody with heavy chains shown in gray and light chains shown in light gray, and two scaffolds as shown in dark gray. The construct comprises two antigen binding sites (1310 and 1315), and a portion of two scaffolds contain Fc domains (1320 and 1345). The immune-stimulatory compounds (1330 and 1340) are conjugated to the construct by linkers (1360 and 1370). The peptides (1380 and 1385) form a composition with the construct. The peptides contain a nonsynonymous mutation (1390 and 1395).

FIG. 14 illustrates a schematic of an exemplary construct-peptide composition. The construct contains two scaffolds as shown in light gray and two scaffolds as shown in dark gray. The construct comprises two antigen binding sites (1410 and 1415) in the light gray scaffolds, and a portion of the two dark gray scaffolds contain Fc domains (1440 and 1445). The peptides (1480 and 1485) form a composition with the construct. The peptides contain a nonsynonymous mutation (1490 and 1495).

FIG. 15 illustrates a schematic of an exemplary construct-peptide composition conjugated to immune-stimulatory compounds. The construct contains two scaffolds as shown in light gray and two scaffolds as shown in dark gray. The construct comprises two antigen binding sites (1510 and 1515), and a portion of the two dark gray scaffolds contain Fc domains (1520 and 1545). The immune-stimulatory compounds (1530 and 1540) are conjugated to the construct by linkers (1560 and 1570). The peptides (1580 and 1585) form a composition with the construct. The peptides contain a nonsynonymous mutation (1590 and 1595).

FIG. 16 is the two-dimensional structure of the heavy chain of Dacetuzumab. Figure discloses SEQ ID NO: 142.

FIG. 17 is the two-dimensional structure of the light chain of Dacetuzumab. Figure discloses SEQ ID NO: 143.

FIG. 18 is the two-dimensional structure of the heavy chain of Bleselumab. Figure discloses SEQ ID NO: 144.

FIG. 19 is the two-dimensional structure of the light chain of Bleselumab. Figure discloses SEQ ID NO: 145.

FIG. 20 is the two-dimensional structure of the heavy chain of Lucatumumab. Figure discloses SEQ ID NO: 146.

FIG. 21 is the two-dimensional structure of the light chain of Lucatumumab. Figure discloses SEQ ID NO: 147.

FIG. 22 is the two-dimensional structure of the heavy chain of ADC-1013. Figure discloses SEQ ID NO: 148.

FIG. 23 is the two-dimensional structure of the light chain of ADC-1013. Figure discloses SEQ ID NO: 149.

FIG. 24 is the two-dimensional structure of the heavy chain of humanized rabbit antibody APX005. Figure discloses SEQ ID NO: 150.

FIG. 25 is the two-dimensional structure of the light chain of humanized rabbit antibody APX005. Figure discloses SEQ ID NO: 151.

FIG. 26 is the two-dimensional structure of the heavy chain of Chi Lob 7/4. Figure discloses SEQ ID NO: 152.

FIG. 27 is the two-dimensional structure of the light chain of Chi Lob 7/4. Figure discloses SEQ ID NO: 153.

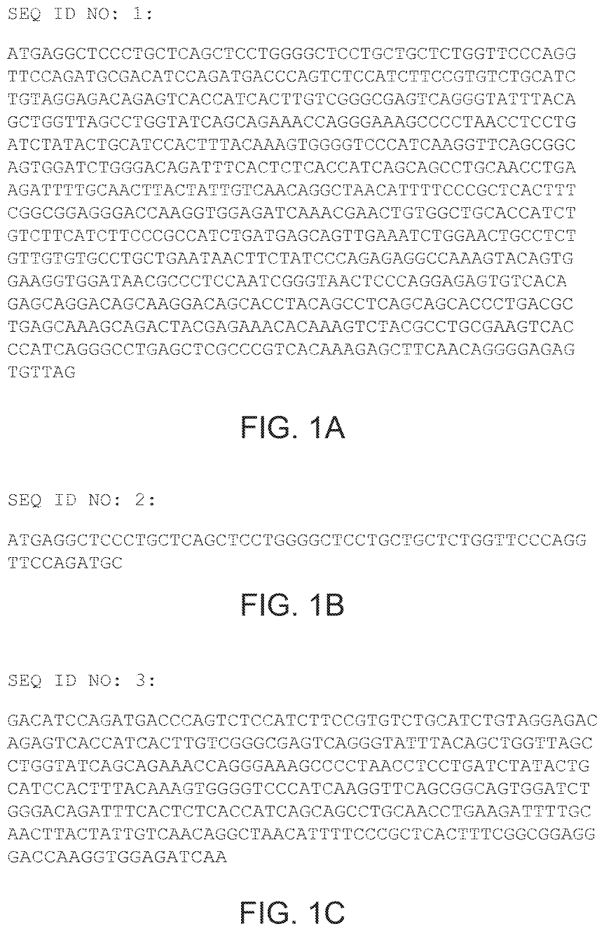

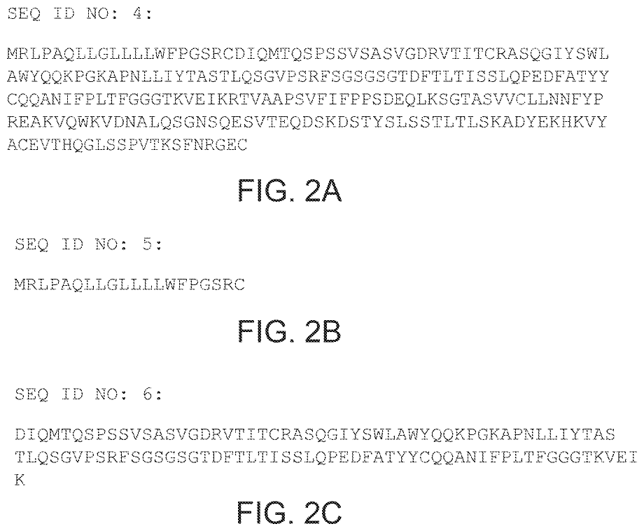

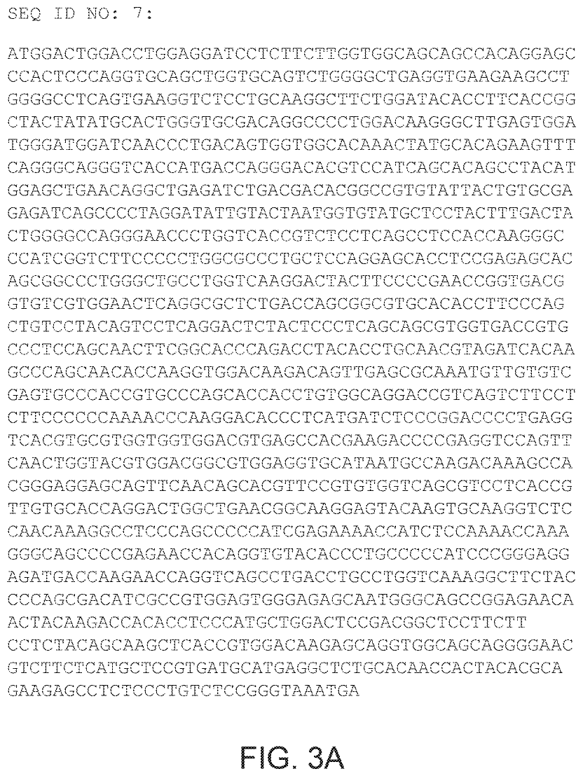

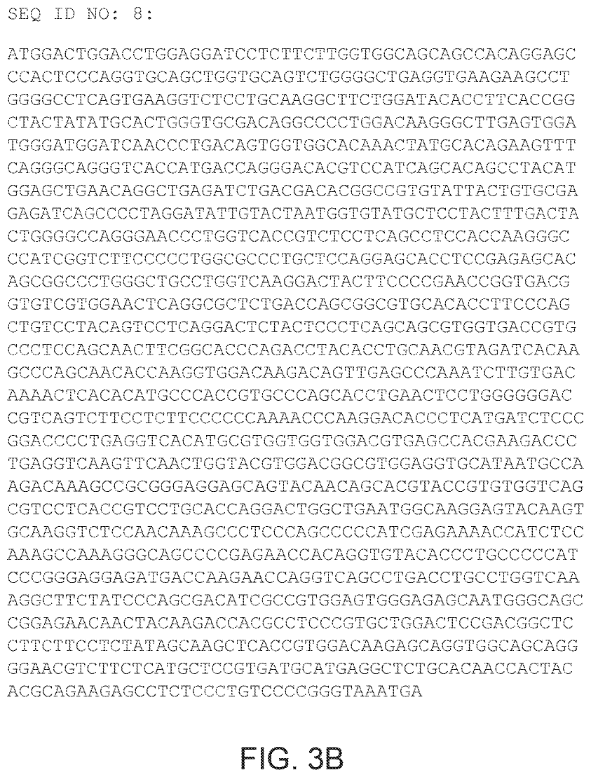

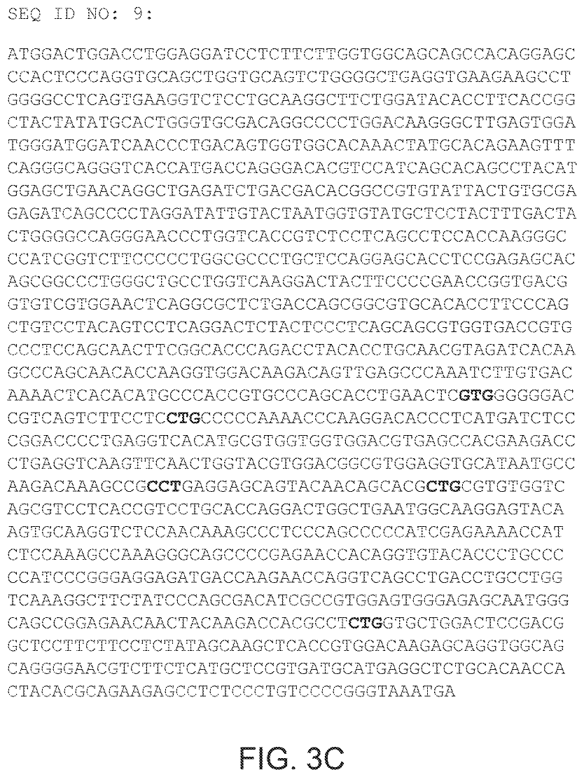

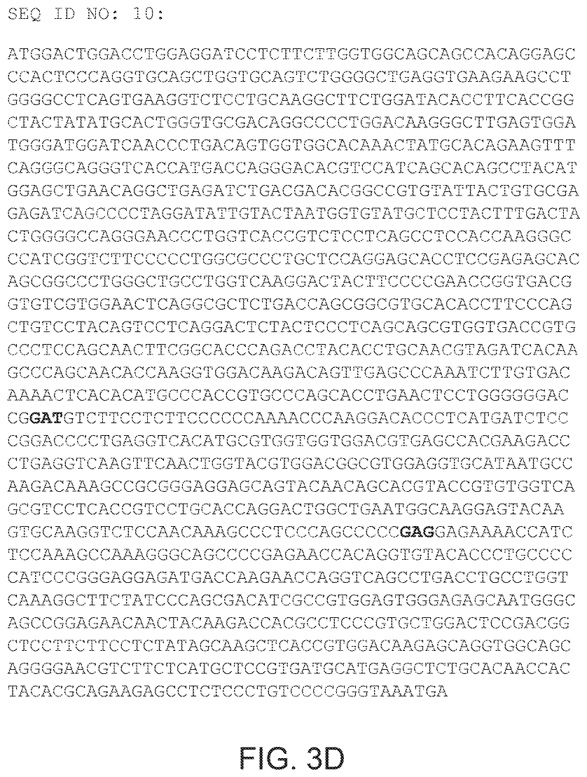

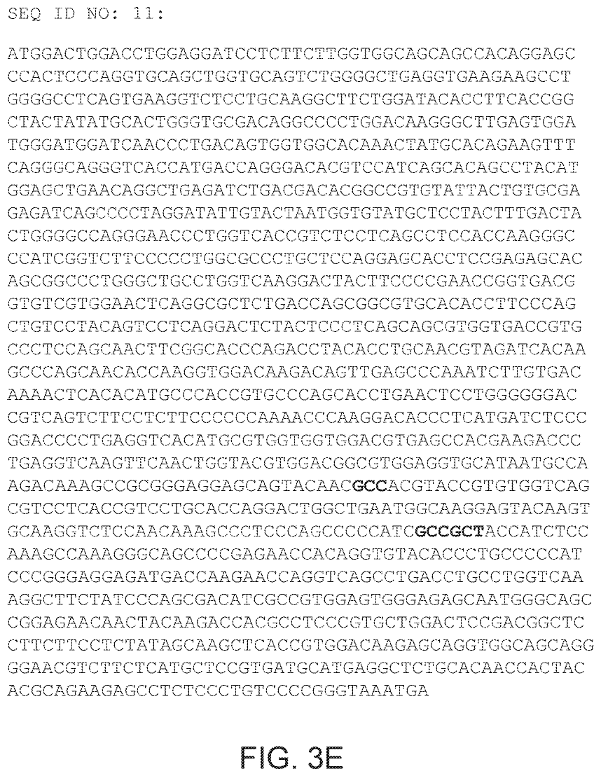

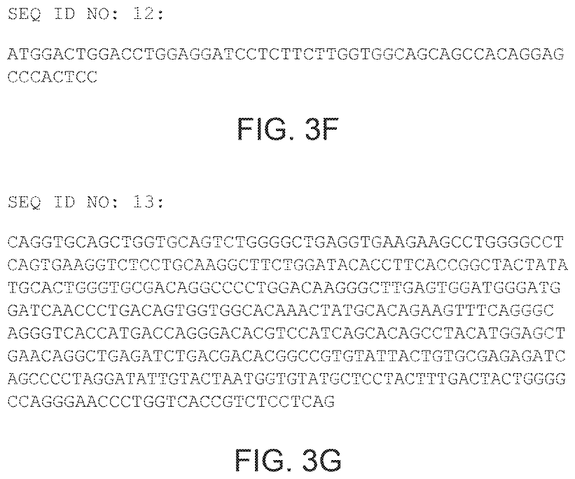

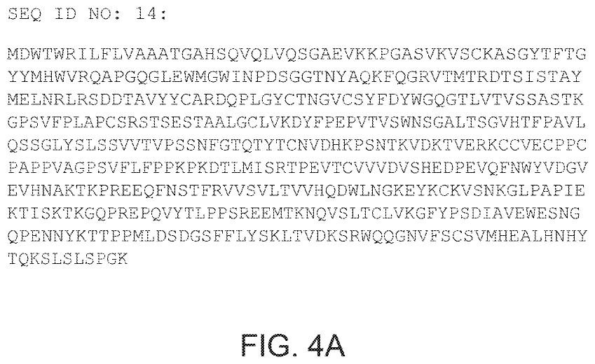

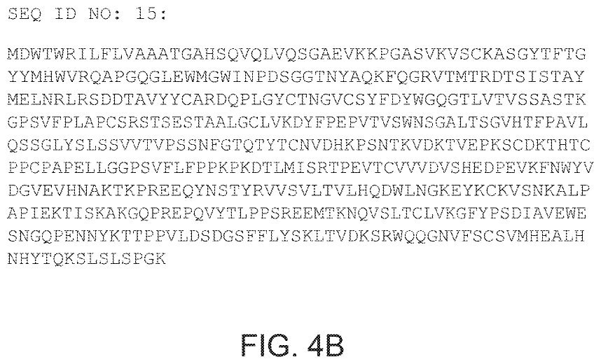

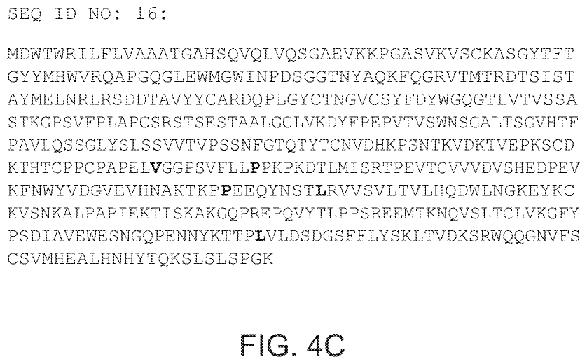

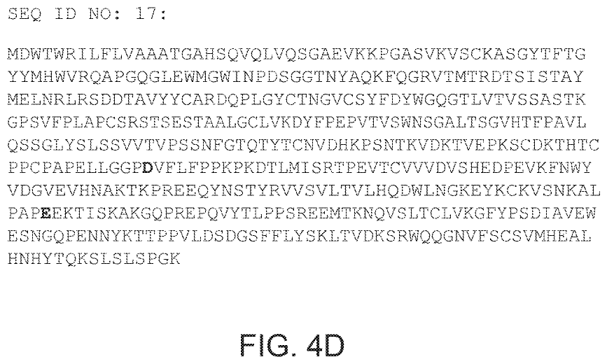

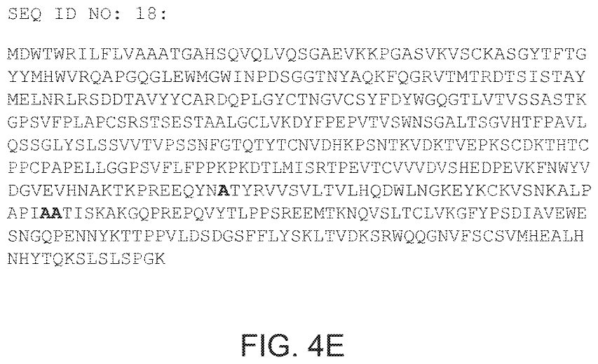

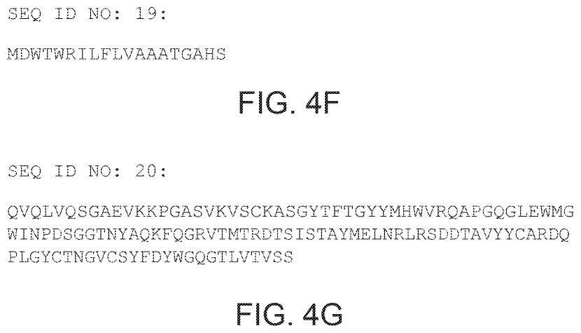

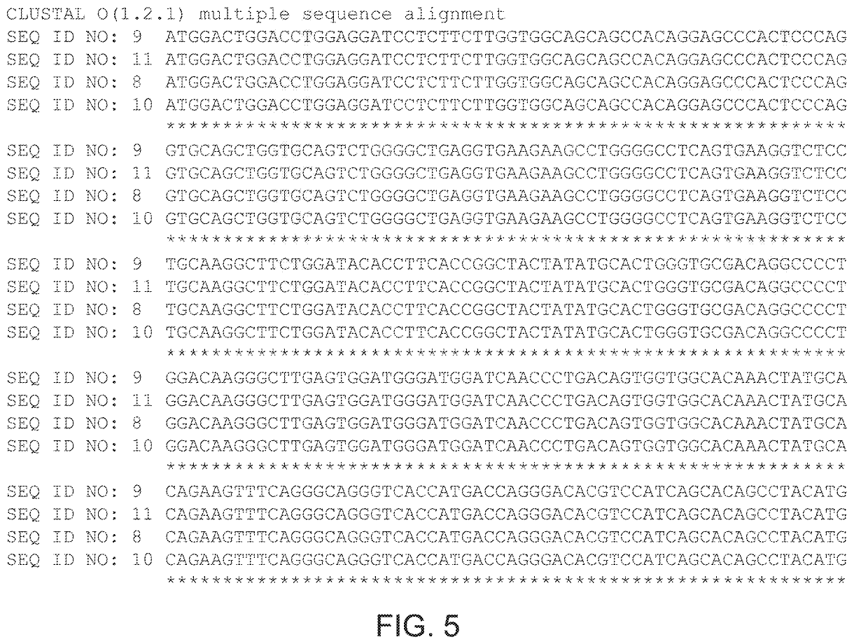

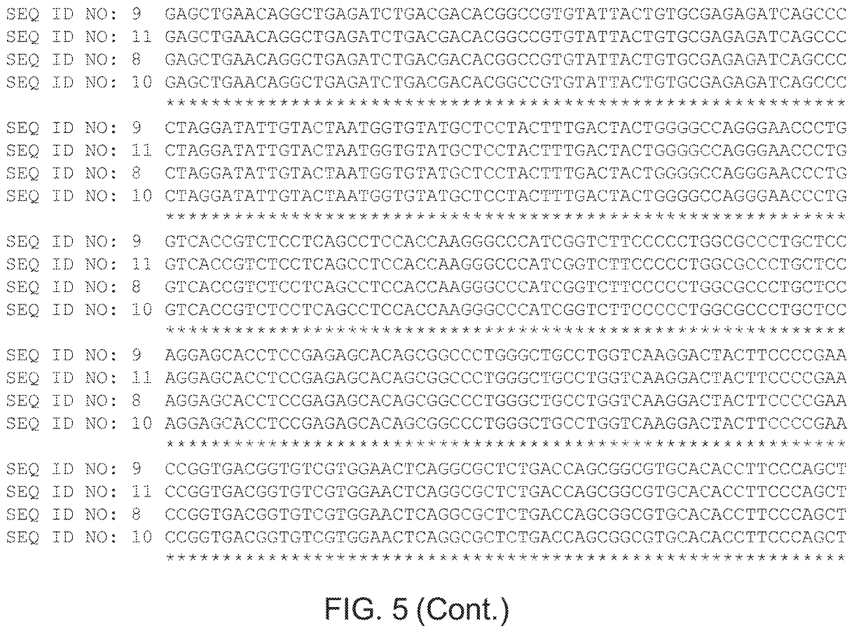

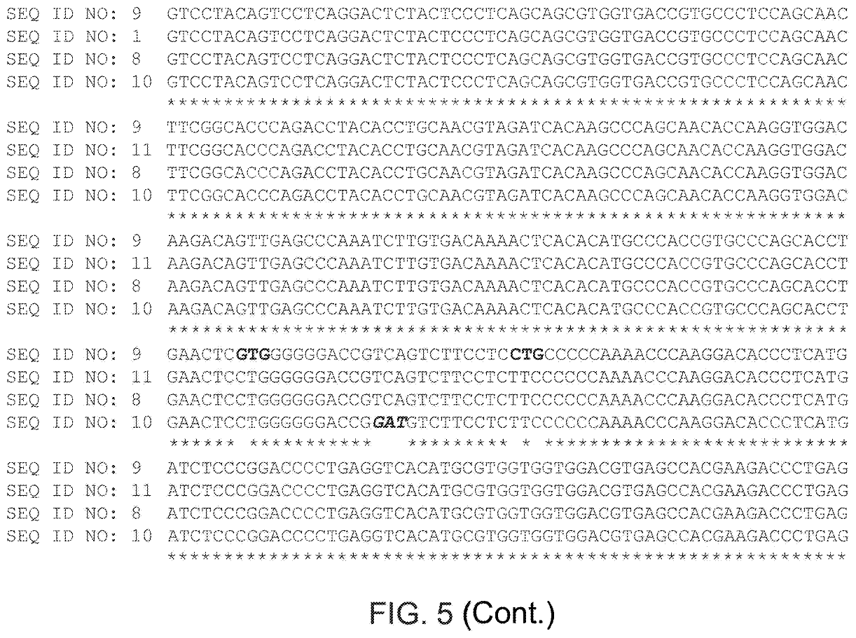

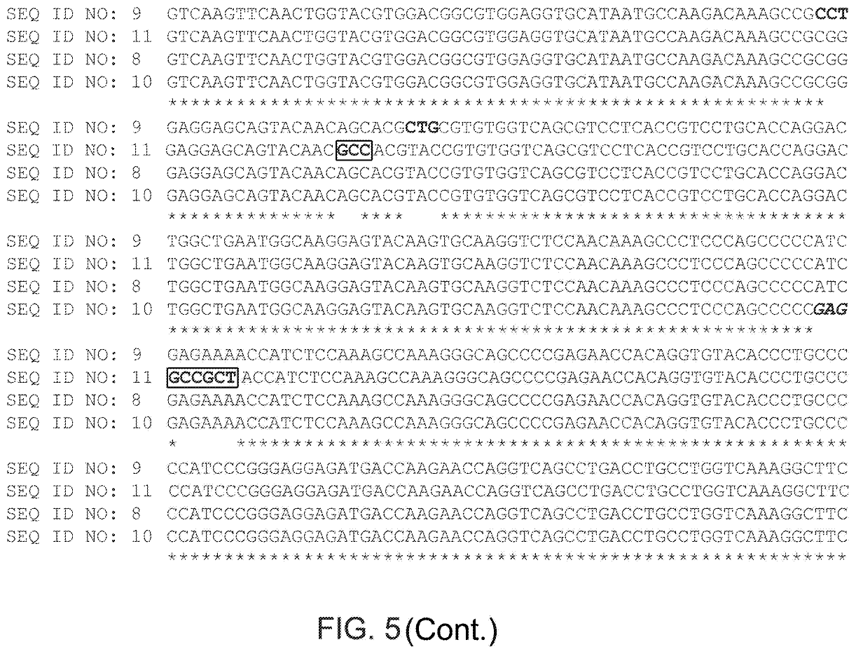

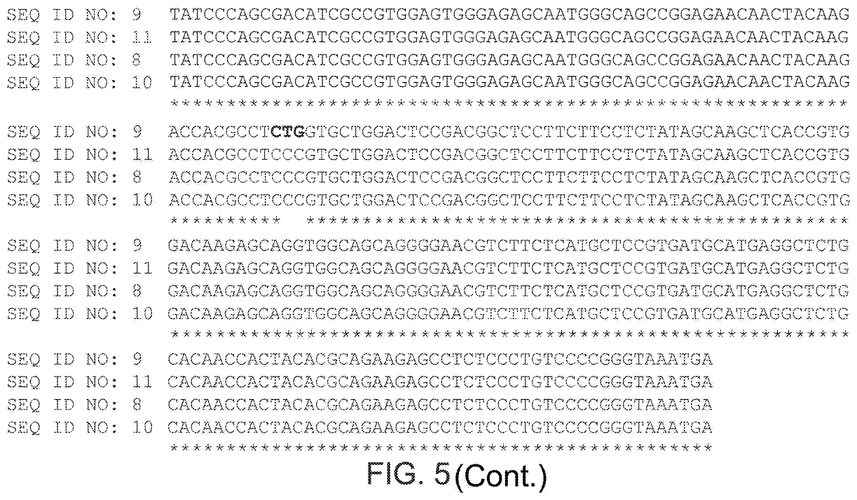

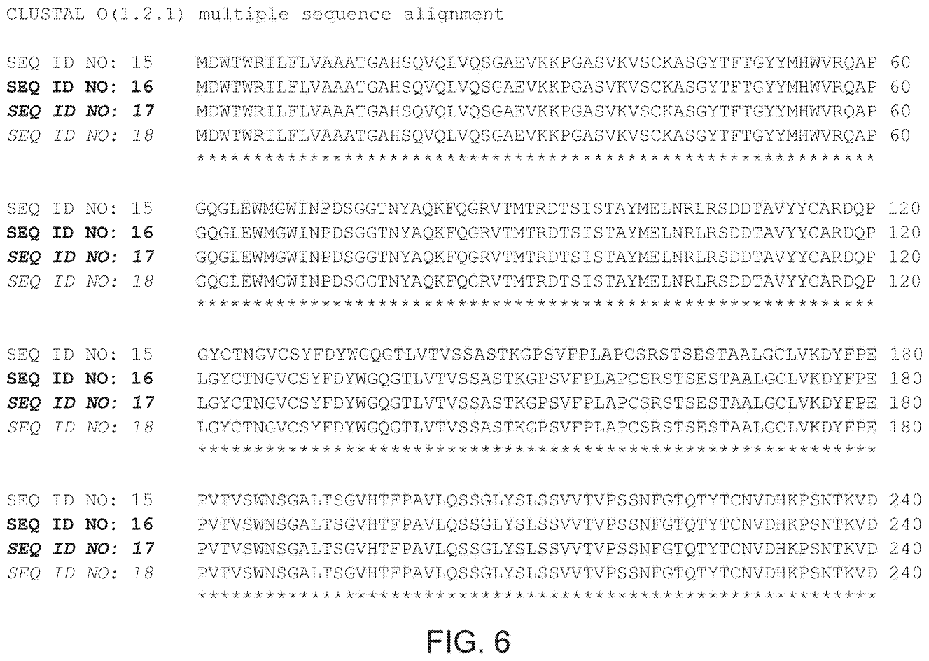

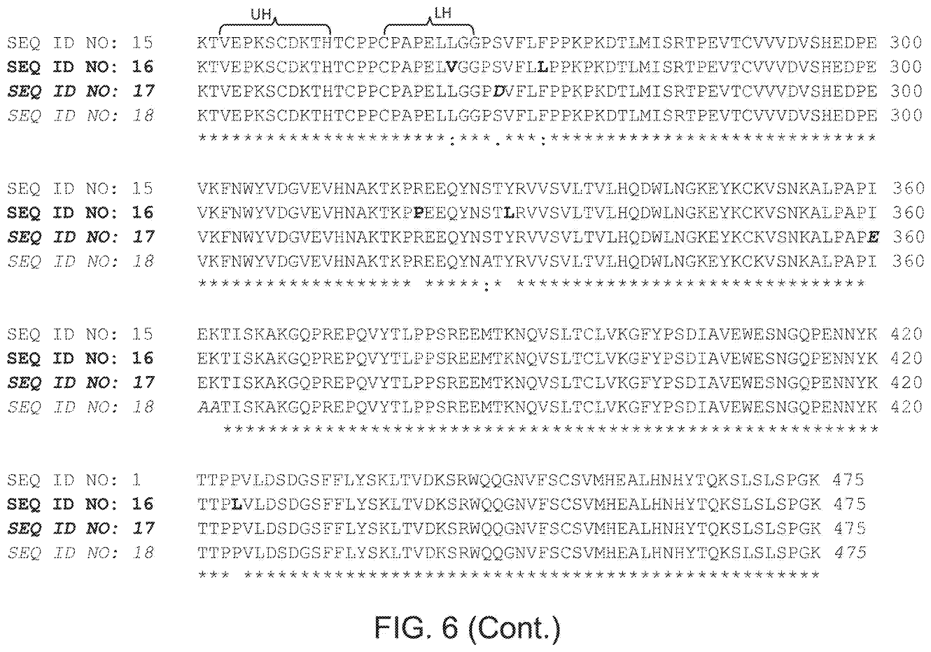

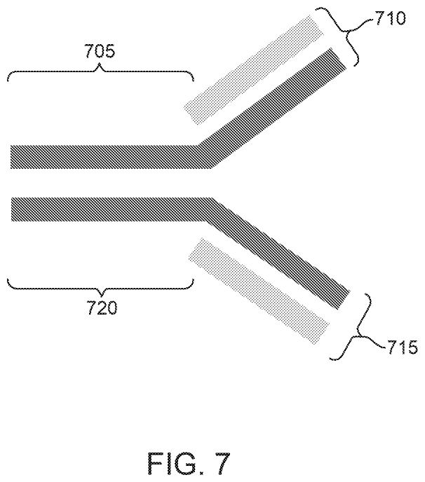

Additional aspects and advantages of the present disclosure will become apparent to those skilled in this art from the following detailed description, wherein illustrative aspects of the present disclosure are shown and described. As will be appreciated, the present disclosure is capable of other and different aspects, and its several details are capable of modifications in various respects, all without departing from the disclosure. Accordingly, the drawings and descriptions are to be regarded as illustrative in nature, and not as restrictive.

### DETAILED DESCRIPTION

Cancer is one of the leading causes of death in the United States. Conventional methods of cancer treatment like chemotherapy, surgery, or radiation therapy, can be limited in their efficacy since they are often nonspecific to the cancer. In many cases, tumors can specifically express genes whose products are required for inducing or maintaining the malignant state. These proteins can serve as antigen markers for the development and establishment of efficient anti-cancer treatments.

As used herein, “homologous” or “homology” refers to the similarity between a DNA, RNA, nucleotide, amino acid, or protein sequence to another DNA, RNA, nucleotide, amino acid, or protein sequence. Homology can be expressed in terms of a percentage of sequence identity of a first sequence to a second sequence. Percent (%) sequence identity with respect to a reference DNA sequence can be the percentage of DNA nucleotides in a candidate sequence that are identical with the DNA nucleotides in the reference DNA sequence after aligning the sequences. Percent (%) sequence identity with respect to a reference amino acid sequence can be the percentage of amino acid residues in a candidate sequence that are identical with the amino acid residues in the reference amino acid sequence after aligning the sequences and introducing gaps, if necessary, to achieve the maximum percent sequence identity, and not considering any conservative substitutions as part of the sequence identity.

As used herein, the term “antibody” refers to an immunoglobulin molecule that specifically binds to, or is immunologically reactive toward, a specific antigen. The term antibody can include, for example, polyclonal, monoclonal, genetically engineered, and antigen binding fragments thereof. An antibody can be, for example, murine, chimeric, humanized, heteroconjugate, bispecific, diabody, triabody, or tetrabody. An antigen binding fragment can include, for example, Fab′, F(ab′)₂, Fab, Fv, rIgG, scFv, hcAbs (heavy chain antibodies), a single domain antibody, VHH, VNAR, sdAbs, or nanobody.

As used herein, “recognize” refers to the association or binding between an antigen binding domain and an antigen.

As used herein, an “antigen” refers to an antigenic substance that can elicit an immune response in a host. An antigen can be a protein, polysaccharide, lipid, or glycolipid, which can be recognized by an immune cell, such as a T cell or a B cell. Exposure of immune cells to one or more of these antigens can elicit a rapid cell division and differentiation response resulting in the formation of clones of the exposed T cells and B cells. B cells can differentiate into plasma cells which in turn can produce antibodies which selectively bind to the antigens.

As used herein, a “tumor antigen” can be an antigenic substance associated with a tumor or cancer cell, and can trigger an immune response in a host.

As used herein, an “antibody construct” refers to a construct that contains an antigen binding domain and an Fc domain.

As used herein, a “Fc domain” can be an Fc domain from an antibody or from a non-antibody that can bind to an Fc receptor, such as an Fcγ receptor or an FcRn receptor.

As used herein, a “target binding domain” refers to a construct that contains an antigen binding domain from an antibody or from a non-antibody that can bind to the antigen.

As used herein, an “antigen binding domain” refers to a binding domain from an antibody or from a non-antibody that can specifically bind to an antigen. Antigen binding domains can be numbered when there is more than one antigen binding domain in a given conjugate or antibody construct (e.g., first antigen binding domain, second antigen binding domain, third antigen binding domain, etc.).

As used herein, a “target antigen binding domain” refers to an antigen binding domain of a conjugate or construct that specifically binds an antigen.

As used herein, an “immune cell” refers to a T cell, B cell, NK cell, NKT cell, or an antigen presenting cell. In some embodiments, an immune cell is a T cell, B cell, NK cell, or NKT cell. In some embodiments, an immune cell is an antigen presenting cell. In some embodiments, an immune cell is not an antigen presenting cell.

As used herein, the abbreviations for the natural L-enantiomeric amino acids are conventional and can be as follows: alanine (A, Ala); arginine (R, Arg); asparagine (N, Asn); aspartic acid (D, Asp); cysteine (C, Cys); glutamic acid (E, Glu); glutamine (Q, Gln); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val). Unless otherwise specified, X can indicate any amino acid. In some aspects, X can be asparagine (N), glutamine (Q), histidine (H), lysine (K), or arginine (R).

As used herein, the term “mutation” in the context of a peptide or protein, refers to a change in amino acid sequence, for example by insertion, deletion or substitution. A “non-synonymous mutation” refers to a missense substitution in an amino acid sequence.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

The term “C_{x-y}” when used in conjunction with a chemical moiety, such as alkyl, alkenyl, or alkynyl is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, etc.

The terms “C_{x-y}alkenyl” and “C_{x-y}alkynyl” refer to substituted or unsubstituted unsaturated aliphatic groups analogous in length and possible substitution to the alkyls described above, but that contain at least one double or triple bond respectively.

The term “carbocycle” as used herein refers to a saturated, unsaturated or aromatic ring in which each atom of the ring is carbon. Carbocycle includes 3- to 10-membered monocyclic rings, 6- to 12-membered bicyclic rings, and 6- to 12-membered bridged rings. Each ring of a bicyclic carbocycle may be selected from saturated, unsaturated, and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary carbocycles include cyclopentyl, cyclohexyl, cyclohexenyl, adamantyl, phenyl, indanyl, and naphthyl.

The term “heterocycle” as used herein refers to a saturated, unsaturated or aromatic ring comprising one or more heteroatoms. Exemplary heteroatoms include N, O, Si, P, B, and S atoms. Heterocycles include 3- to 10-membered monocyclic rings, 6- to 12-membered bicyclic rings, and 6- to 12-membered bridged rings. Each ring of a bicyclic heterocycle may be selected from saturated, unsaturated, and aromatic rings wherein at least one of the rings includes a heteroatom. In an exemplary embodiment, an aromatic ring, e.g., pyridyl, may be fused to a saturated or unsaturated ring, e.g., cyclohexane, cyclopentane, morpholine, piperidine or cyclohexene. The term “heteroaryl” includes aromatic single ring structures, preferably 5- to 7-membered rings, more preferably 5- to 6-membered rings, whose ring structures include at least one heteroatom, preferably one to four heteroatoms, more preferably one or two heteroatoms. The term “heteroaryl” also include polycyclic ring systems having two or more cyclic rings in which two or more carbons are common to two adjoining rings wherein at least one of the rings is heteroaromatic, e.g., the other cyclic rings can be aromatic or non-aromatic carbocyclic, or heterocyclic. Heteroaryl groups include, for example, pyrrole, furan, thiophene, imidazole, oxazole, thiazole, pyrazole, pyridine, pyrazine, pyridazine, and pyrimidine, and the like.

The term “substituted” refers to moieties having substituents replacing a hydrogen on one or more carbons or substitutable heteroatoms, e.g., —NH—, of the structure. It will be understood that “substitution” or “substituted with” includes the implicit proviso that such substitution is in accordance with permitted valence of the substituted atom and the substituent, and that the substitution results in a stable compound. i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N (R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N (R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N (R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

It will be understood by those skilled in the art that substituents can themselves be substituted, if appropriate. Unless specifically stated as “unsubstituted,” references to chemical moieties herein are understood to include substituted variants. For example, reference to a “heteroaryl” group or moiety implicitly includes both substituted and unsubstituted variants.

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, chemical entities described herein are intended to include all Z-, E- and tautomeric forms as well.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

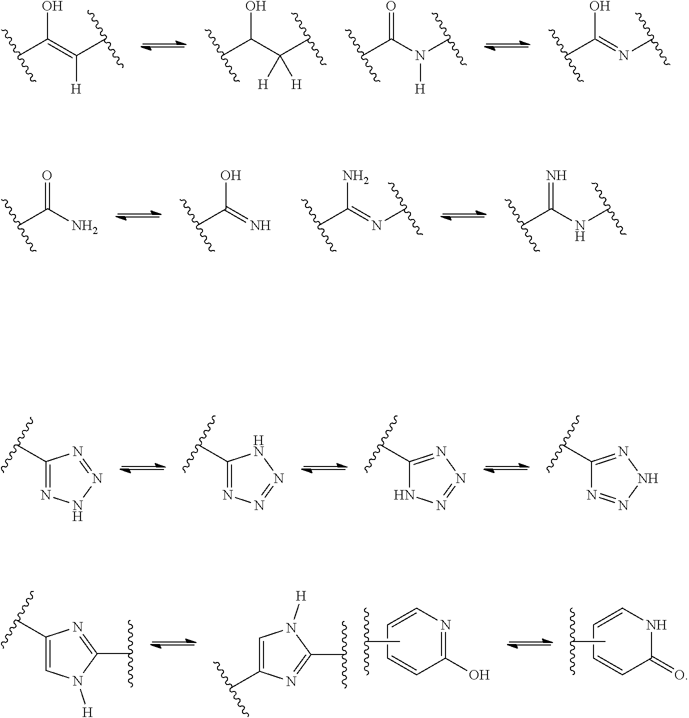

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³S, ³⁵S, ³⁶S, ³⁵Cl, ³⁷Cl, ⁷⁹Br, ⁸¹Br, ¹²⁵I are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods. Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Compounds of the present invention also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. In some aspects, a pharmaceutical composition may comprise the compositions or the composition mixtures and a pharmaceutically acceptable carrier.

An antigen can elicit an immune response. An antigen can be a protein, polysaccharide, lipid, or glycolipid, which can be recognized by an immune cell, such as a T cell or a B cell. Exposure of immune cells to one or more of these antigens can elicit a rapid cell division and differentiation response resulting in the formation of clones of the exposed T cells and B cells. B cells can differentiate into plasma cells which in turn can produce antibodies which selectively bind to the antigens. The antigen can be bound by the antigen binding domain. The antigen bound by the antigen binding domain can bind to a tumor associated antigen or to an antigen expressed on an immune cell, for example, an APC. The antigen can be a target antigen of an antigen binding domain.

Tumor antigens can include, for example, (i) viral tumor antigens, which can be identical for any viral tumor of this type, (ii) carcinogenic tumor antigens, which can be specific for patients and for the tumors, (iii) isoantigens of the transplantation type or tumor-specific transplantation antigens, which can be different in all individual types of tumor but can be the same in different tumors caused by the same virus; and (iv) embryonic antigens.

As a result of the discovery of tumor antigens, tumor antigens have become important in the development of new cancer treatments that can specifically target the cancer. This has led to the development of antibodies directed against these tumor antigens.

In addition to the development of antibodies against tumor antigens for cancer treatment, antibodies that target immune cells to boost the immune response have also been developed. For example, an anti-CD40 antibody that is a CD40 agonist can be used to activate dendritic cells to enhance the immune response.

Cluster of Differentiation 40 (CD40) is a member of the Tumor Necrosis Factor Receptor (TNF-R) family. CD40 can be a 50 kDa cell surface glycoprotein that can be constitutively expressed in normal cells, such as monocytes, macrophages, B lymphocytes, dendritic cells, endothelial cells, smooth muscle cells, fibroblasts and epithelium, and in tumor cells, including B-cell lymphomas and many types of solid tumors. Expression of CD40 can be increased in antigen presenting cells in response to IL-1βp, IFN-γ, GM-CSF, and LPS induced signaling events.

Humoral and cellular immune responses can be regulated, in part, by CD40. For example, in the absence of CD40 activation by its cognate binding partner, CD40 Ligand (CD40L), antigen presentation can result in tolerance. However, CD40 activation can ameliorate tolerance. In addition, CD40 activation can positively impact immune responses by enhancing antigen presentation by antigen presenting cells (APC), increasing cytokine and chemokine secretion, stimulating expression of and signaling by co-stimulatory molecules, and activating the cytolytic activity of different types of immune cells. Accordingly, the interaction between CD40 and CD40L can be essential to maintain proper humoral and cellular immune responses.

The intracellular effects of CD40 and CD40L interaction can include association of the CD40 cytoplasmic domain with TRAFs (TNF-R associated factors), which can lead to the activation of NFκB and Jun/API pathways. While the response to activation of NFκB and Jun/API pathways can be cell type-specific, often such activation can lead to increased production and secretion of cytokines, including IL-6, IL-8, IL-12, IL-15; increased production and secretion of chemokines, including MIP1α and β and RANTES; and increased expression of cellular adhesion molecules, including ICAM. While the effects of cytokines, chemokines and cellular adhesion molecules can be widespread, such effects can include enhanced survival and activation of T cells.

In addition to the enhanced immune responses induced by CD40 activation, CD40 activation can also be involved in chemokine- and cytokine-mediated cellular migration and differentiation; activation of immune cells, including monocytes; activation of and increased cytolytic activity of immune cells, including cytolytic T lymphocytes and natural killer cells; induction of CD40-positive tumor cell apoptosis and enhanced immunogenicity of CD40-positive tumors. In addition, CD40 can initiate and enhance immune responses by many different mechanisms, including, inducing antigen-presenting cell maturation and increased expression of costimulatory molecules, increasing production of and secretion of cytokines, and enhancing effector functions.

CD40 activation can be effective for inducing immune-mediated antitumor responses. For example, CD40 activation reverses host immune tolerance to tumor-specific antigens which leads to enhanced antitumor responses by T cells. Such antitumor activity can also occur in the absence of immune cells. Similarly, antitumor effects can occur in response to anti-CD40 antibody-mediated activation of CD40 and can be independent of antibody-dependent cellular cytotoxicity. In addition to other CD40-mediated mechanisms of antitumor effects, CD40L-stimulation can cause dendritic cell maturation and stimulation. CD40L-stimulated dendritic cells can contribute to the antitumor response. Furthermore, vaccination strategies including CD40 can result in regression of CD40-positive and CD40-negative tumors.

CD40 activating antibodies (e.g., anti-CD40 activating monoclonal antibodies) can be useful for treatment of tumors. This can occur through one or more mechanisms, including cell activation, antigen presentation, production of cytokines and chemokines, amongst others. For example, CD40 antibodies activate dendritic cells, leading to processing and presentation of tumor antigens as well as enhanced immunogenicity of CD40-positive tumor cells. Not only can enhanced immunogenicity result in activation of CD40-positive tumor specific CD4⁺ and CD8⁺ T cells, but further antitumor activity can include recruitment and activation monocytes, and enhanced cytolytic activity of cytotoxic lymphocytes and natural killer cells, as well as induction of apoptosis or stimulation of a humoral response so as to directly target tumor cells. In addition, tumor cell debris, including tumor-specific antigens, can be presented to other cells of the immune system by CD40-activated antigen presenting cells.

Since CD40 can be important in an immune response, there is a need for enhanced CD40 meditated signaling events to provide reliable and rapid treatment options to patients suffering from diseases which may be ameliorated by treatment with CD40-targeted therapeutic strategies.

Dendritic cells (DCs) are antigen-presenting cells that can initiate both primary and secondary T and B cell responses, and can potentiate CD4⁺ and CD8⁺ T cell responses. DCs can be myeloid or plasmacytoid, and can secrete IL-2 or IFN-α, respectively. DCs are a heterogeneous cell population and can be found in various locations of the body. Subsets of human DCs can include, for example, CD141/BDCA3⁺ DCs and CD1c/BDCA1⁺ DCs, which can be found in the blood, spleen, and tonsils. CD141/BDCA3⁺ DCs can be a relatively rare subset of DCs. Human CD1c/BDCA1⁺ DCs can be found in blood. Plasmacytoid DCs can be found in blood and lymphoid tissue, and human dermal DCs can include, for example, CD1a⁺CD14⁻ DCsCD1a⁻CD14⁺ DCs, and Langerhans cells.

DCs can be identified using multiple different DC markers, which can include, for example, BDCA-2 (CLEC4C), BDCA-3 (thrombomodulin), BDCA-4 (neuropilin), BST-2, CD207, CLEC12A, DCAR1, DCIR, DCIR2, DNGR-1 (CLEC9A), CD36 mannose scavenger receptor 1, DC-SIGN, DEC-205, Dectin 1, Dectin 2, MGL, and Siglec-H. DEC-205 is a type I cell surface protein that can be expressed primarily by DCs. DEC-205 can have a single transmembrane domain with a short cytoplasmic tail, in which DEC-205 can have ten extracellular contiguous C-type lectin domains, and can have a cysteine rich N-terminal domain followed by a fibronectin type II domain and ten carbohydrate recognition domains (CRDs). DEC-205 can be found in lymphoid tissues, bone marrow-derived DC, Langerhan's cells, macrophages, and T cells. High levels of DEC-205 can be associated with the antigen-presenting function of DCs. Antibodies that can bind DEC-205 include, for example, 3D6-2F4, 3D6-4C8, 3G9-2D2, 5A8-1F1, 2D3-1F5-2A9, 3C7-3A3, 5D12-5G1, 1G6-1G6, 5C3-2-3F6, 1E6-3D10, NLDC-145, and 3A4-1C10.

The HER2/neu (human epidermal growth factor receptor 2/receptor tyrosine-protein kinase erbB-2) is part of the human epidermal growth factor family. Overexpression of this protein has shown to play an important role in the progression of cancer, for example, breast cancer. The HER2/neu protein functions as a receptor tyrosine kinase and autophosphorylates upon dimerization with binding partners. HER2/neu can activate several signaling pathways including, for example, mitogen-activated protein kinase, phosphoinositide 3-kinase, phospholipase Cγ, protein kinase C, and signal transducer and activator of transcription (STAT). Several compounds have been developed to inhibit HER2/neu including for example, the monoclonal antibody trastuzumab and the monoclonal antibody pertuzumab.

In addition to being recognized by tumor-antigen specific antibodies, tumor antigens can be recognized by T cells. When activated, T cells can rapidly expand, and then can efficiently and specifically kill cells that express a tumor antigen. However, the activation of these T cells can be complex. T cells require multiple signals for activation, such as requiring the T cell receptor (TCR) of the T cell bind to its cognate antigen in the context of a Major Histocompatibility Complex (MHC) molecule or Human Leukocyte Antigen (HLA) molecule. For cancer, if antigen-presenting cells do not present a tumor antigen associated with the cancer, then the T cells will not become activated to specifically kill that cancer. Therefore, the ability of T cells to contribute to the anti-cancer response can be limited by the presentation of tumor antigen by antigen-presenting cells. Accordingly, there is a need for methods that increase the presentation of tumor antigens by antigen-presenting cells to activate T cells, which can greatly contribute to the anti-cancer immune response. The presently described construct-peptide composition can be utilized to enhance tumor-antigen presentation for activation of T cells and boosting of anti-cancer immune responses.

The anti-cancer response can be further boosted by use of immune-stimulatory compounds with the presently described construct-peptide composition. Immune-stimulatory molecular motifs, such as Pathogen-Associated Molecular Pattern molecules, (PAMPs) can be recognized by receptors of the innate immune system, such as Toll-like receptors (TLRs), Nod-like receptors, C-type lectins, and RIG-I-like receptors. These receptors can be transmembrane and intra-endosomal proteins which can prime activation of the immune system in response to infectious agents such as pathogens. Similar to other protein families, TLRs can have many isoforms, including TLR4, TLR7 and TLR8. Several agonists targeting activation of different TLRs can be used in various immunotherapies, including vaccine adjuvants and in cancer immunotherapies. TLR agonists can range from simple molecules to complex macromolecules. Likewise, the sizes of TLR agonists can range from small to large. TLR agonists can be synthetic or biosynthetic agonists. TLR agonists can also be PAMPs. Additional immune-stimulatory compounds, such as cytosolic DNA and unique bacterial nucleic acids called cyclic dinucleotides, can be recognized by Interferon Regulatory Factor (IRF) or stimulator of interferon genes (STING), which can act a cytosolic DNA sensor. Compounds recognized by Interferon Regulatory Factor (IRF) can play a role in immunoregulation by TLRs and other pattern recognition receptors.

Imiquimod, a synthetic TLR7 agonist, is currently approved for human therapeutic applications. Contained in a cream and marketed under the brand name Aldara, imiquimod serves as a topical treatment for a variety of indications with immune components, such as, actinic keratosis, genital warts, and basal cell carcinomas. In addition, imiquimod is indicated as a candidate adjuvant for enhancing adaptive immune responses when applied topically at an immunization site.

Another type of immune stimulatory molecular motif, damage-associated molecular pattern molecules (DAMPs), can initiate and maintain an immune response occurring as part of the non-infectious inflammatory response. DAMPs can be specially localized proteins that, when detected by the immune system in a location other than where DAMPs should be located, activate the immune system. Often, DAMPs can be nuclear or cytosolic proteins and upon release from the nucleus or cytosol, DAMP proteins can become denatured through oxidation. Examples of DAMP proteins can include chromatin-associated protein high-mobility group box 1 (HMGB1), S100 molecules of the calcium modulated family of proteins and glycans, such as hyaluronan fragments, and glycan conjugates. DAMPs can also be nucleic acids, such as DNA, when released from tumor cells following apoptosis or necrosis. Examples of additional DAMP nucleic acids can include RNA and purine metabolites, such as ATP, adenosine and uric acid, present outside of the nucleus or mitochondria.

Therapeutic application of DAMPs can focus on indications with an immune component, such as arthritis, cancer, ischemia-reperfusion injury, myocardial infarction and stroke. In these indications, the mechanism of action for DAMP therapeutic effects can include the prevention of DAMP release using therapeutic strategies, such as proapoptotic interventions, platinum and ethyl pyruvate, extracellular neutralization or blockade of DAMP release or signaling using therapeutic strategies such as anti-HMGB1, rasburiaspect and sRAGE, as well as direct or indirect blockade of DAMP receptors, and downstream signaling events, using therapeutic strategies such as RAGE small molecule antagonists; TLR4 antagonists and antibodies to DAMP-R.

Additionally, the immune response elicited by TLR agonists can further be enhanced when co-administered with a CD40-agonist antibody. For example, co-administration of a TLR agonist such as poly IC:LC with a CD40-agonist antibody can synergize to stimulate a greater CD8⁺ T cell response than either agonist alone.

Therefore, by conjugating immune stimulatory compounds to the construct-peptide compositions presently described, the anti-cancer immune responses can be further enhanced.

In some embodiments, a composition mixture may comprise at least two different compositions disclosed herein.

### Construct of the Construct-Peptide Composition

A construct can contain an antigen binding domain. An antigen binding domain can be a domain that can specifically bind to an antigen. An antigen binding domain can be a domain that can specifically bind to a target antigen. An antigen binding domain can be an antigen-binding portion of an antibody or an antibody fragment. An antigen binding domain can be one or more fragments of an antibody that can retain the ability to specifically bind to an antigen. An antigen binding domain can be one or more fragments of an antibody that can retain the ability to specifically bind to a target antigen. An antigen binding domain can be any antigen binding fragment. An antigen binding domain typically recognizes a single antigen. A construct can comprise two antigen binding domains. A construct can comprise three, four, five, six, seven, eight, nine, ten, or more antigen binding domains. A construct can contain two antigen binding domains in which each antigen binding domain can recognize the same antigen. A construct can contain two antigen binding domains in which each antigen binding domain can recognize the same target antigen. A construct can contain two antigen binding domains in which each antigen binding domain can recognize different antigens. A construct can contain two antigen binding domains in which each antigen binding domain can recognize different target antigens. A construct can comprise an antigen binding domain in a scaffold. An antigen binding domain can be in a scaffold, in which the scaffold is a supporting framework for the antigen binding domain. An antigen binding domain can be in a non-antibody scaffold. An antigen binding domain can be in an antibody scaffold. An antigen binding domain can be in an antibody. A construct can comprise an antigen binding domain in a scaffold. A construct can comprise an Fc fusion product. In some embodiments, a construct is a Fc fusion protein product.

A construct can comprise an antigen binding domain and an Fc domain, wherein the Fc domain can be covalently attached to the antigen binding domain. A construct can comprise a target antigen binding domain and Fc domain, wherein the Fc domain can be covalently attached to the target antigen binding domain. A construct can comprise an antigen binding domain and Fc domain, wherein the Fc domain is covalently attached to the antigen binding domain as an Fc domain-antigen binding domain fusion protein. A construct can comprise an antigen binding domain and Fc domain, wherein the Fc domain is covalently attached to the antigen binding domain by a linker. A construct can comprise a target antigen binding domain and Fc domain, wherein the Fc domain is covalently attached to the target antigen binding domain as an Fc domain-target antigen binding domain fusion protein. A construct can comprise a target antigen binding domain and Fc domain, wherein the Fc domain is covalently attached to the target antigen binding domain via a linker.

The antigen binding domain of a construct can be selected from any domain that binds the antigen including, but not limited to, from a monoclonal antibody, a polyclonal antibody, a recombinant antibody, or a functional fragment thereof, for example, a heavy chain variable domain (VH) and a light chain variable domain (VL), a Fab′, F(ab′)2, Fab, Fv, rIgG, scFv, hcAbs (heavy chain antibodies), a single domain antibody, VHH, VNAR, sdAbs, or nanobody. The antigen binding domain of a construct can be at least 80% homologous to an antigen binding domain selected from, but not limited to, a monoclonal antibody, a polyclonal antibody, a recombinant antibody, or a functional fragment thereof, for example, a heavy chain variable domain (VH) and a light chain variable domain (VL), from a non-antibody scaffold, such as a DARPin, an affimer, an avimer, a knottin, a monobody, lipocalin, an anticalin, ‘T-body’, an affibody, a peptibody, an affinity clamp, an ectodomain, a receptor ectodomain, a receptor, a cytokine, an immunocytokine, a TCR, a recombinant TCR, a ligand, or a centryin.

An antigen binding domain of a construct, for example an antigen binding domain from a monoclonal antibody, can comprise a light chain and a heavy chain. In one aspect, the monoclonal antibody binds to CD40 and comprises the light chain of an anti-CD40 antibody and the heavy chain of an anti-CD40 antibody, which bind a CD40 antigen. In another aspect, the monoclonal antibody binds to a tumor antigen comprises the light chain of a tumor antigen antibody and the heavy chain of a tumor antigen antibody, which bind the tumor antigen.

A construct can be an antibody. An antibody molecule can consist of two identical light protein chains and two identical heavy protein chains, all held together covalently by precisely located disulfide linkages. The N-terminal regions of the light and heavy chains together can form the antigen recognition site of each antibody. Structurally, various functions of an antibody can be confined to discrete protein domains (i.e., regions). The sites that can recognize and can bind antigen consist of three complementarity determining regions (CDRs) that can lie within the variable heavy chain regions and variable light chain regions at the N-terminal ends of the two heavy and two light chains. The constant domains can provide the general framework of the antibody and may not be involved directly in binding the antibody to an antigen, but can be involved in various effector functions, such as participation of the antibody in antibody-dependent cellular cytotoxicity.

The domains of natural light chain variable regions and heavy chain variable regions can have the same general structures, and each domain can comprise four framework regions, whose sequences can be somewhat conserved, connected by three hyper-variable regions or CDRs. The four framework regions can largely adopt a β-sheet conformation and the CDRs can form loops connecting, and in some aspects forming part of, the β-sheet structure. The CDRs in each chain can be held in close proximity by the framework regions and, with the CDRs from the other chain, can contribute to the formation of the antigen binding site.

In some embodiments, the construct is an antibody. An antibody of an antibody construct can comprise an antibody of any type, which can be assigned to different classes of immunoglobins, e.g., IgA, IgD, IgE, IgG, and IgM. Several different classes can be further divided into isotypes, e.g., IgG1, IgG2, IgG3, IgG4, IgA1, and IgA2. An antibody can further comprise a light chain and a heavy chain, often more than one chain. The heavy-chain constant regions (Fc) that corresponds to the different classes of immunoglobulins can be α, δ, ε, γ, and μ, respectively. The light chains can be one of either kappa or κ and lambda or λ, based on the amino acid sequences of the constant domains. The Fc region can comprise an Fc domain. An Fc receptor can bind to an Fc domain. An antibody construct can also comprise any fragment or recombinant form thereof, including but not limited to a scFv, Fab, variable Fc fragment, domain antibody, and any other fragment thereof that can specifically bind to an antigen.

An antibody can comprise an antigen binding domain which refers to a portion of an antibody comprising the antigen recognition portion, i.e., an antigenic determining variable region of an antibody sufficient to confer recognition and specific binding of the antigen recognition portion to a target, such as an antigen, i.e., at an epitope. Examples of antibody binding domains can include, but are not limited to, Fab, variable Fv fragment and other fragments, combinations of fragments or types of fragments known or knowable to one of ordinary skill in the art.

An antigen binding domain of an antibody can comprise one or more light chain (LC) CDRs (LC CDRs) and one or more heavy chain (HC) CDRs (HC CDRs), one or more LC CDRs or one or more HC CDRs. For example, an antibody binding domain of an antibody can comprise one or more of the following: a light chain complementary determining region 1 (LC CDR1), a light chain complementary determining region 2 (LC CDR2), or a light chain complementary determining region 3 (LC CDR3). For another example, an antibody binding domain can comprise one or more of the following: a heavy chain complementary determining region 1 (HC CDR1), a heavy chain complementary determining region 2 (HC CDR2), or a heavy chain complementary determining region 3 (HC CDR3). In some embodiments an antibody binding domain comprises all of the following: a light chain complementary determining region 1 (LC CDR1), a light chain complementary determining region 2 (LC CDR2), a light chain complementary determining region 3 (LC CDR3), a heavy chain complementary determining region 1 (HC CDR1), a heavy chain complementary determining region 2 (HC CDR2), and a heavy chain complementary determining region 3 (HC CDR3). Unless stated otherwise, the CDRs described herein can be defined according to the IMGT (the international ImMunoGeneTics information system). An antigen binding domain can comprise only the heavy chain of an antibody (e.g., does not include any other portion of the antibody). An antigen binding domain can comprise only the variable domain of the heavy chain of an antibody. Alternatively, an antigen binding domain can comprise only the light chain of an antibody. An antigen binding domain can comprise only the variable light chain of an antibody.

An antibody construct can comprise an antibody fragment, such as a Fab, a Fab′, a F(ab′)2 or an Fv fragment. An antibody used herein can be “humanized.” Humanized forms of non-human (e.g., murine) antibodies can be intact (full length) chimeric immunoglobulins, immunoglobulin chains or antigen binding fragments thereof (such as Fv, Fab, Fab′, F(ab′)2 or other target-binding subdomains of antibodies), which can contain sequences derived from non-human immunoglobulin. In general, the humanized antibody can comprise substantially all of at least one, and typically two, variable domains, in which all or substantially all of the CDR regions correspond to those of a non-human immunoglobulin and all or substantially all of the framework (FR) regions are those of a human immunoglobulin sequence. The humanized antibody can also comprise at least a portion of an immunoglobulin constant region (Fc), typically that of a human immunoglobulin consensus sequence.

An antibody described herein can be a human antibody. As used herein, “human antibodies” can include antibodies having, for example, the amino acid sequence of a human immunoglobulin and include antibodies isolated from human immunoglobulin libraries or from animals transgenic for one or more human immunoglobulins and that typically do not express endogenous immunoglobulins. Human antibodies can be produced using transgenic mice which are incapable of expressing functional endogenous immunoglobulins, but which can express human immunoglobulin genes. Completely human antibodies that recognize a selected epitope can be generated using guided selection. In this approach, a selected non-human monoclonal antibody, e.g., a mouse antibody, is used to guide the selection of a completely human antibody recognizing the same epitope

An antibody described herein can be a bispecific antibody or a dual variable domain antibody (DVD). Bispecific and DVD antibodies are monoclonal, often human or humanized, antibodies that have binding specificities for at least two different antigens.

An antibody described herein can be derivatized or otherwise modified. For example, derivatized antibodies can be modified by glycosylation, acetylation, pegylation, phosphorylation, amidation, derivatization by known protecting/blocking groups, proteolytic cleavage, or the like.

An antibody described herein can be a derivatized antibody. For example, derivatized antibodies can be modified by glycosylation, acetylation, pegylation, phosphorylation, amidation, derivatization by known protecting/blocking groups, proteolytic cleavage, linkage to a cellular ligand or other protein.

An antibody described herein can have a sequence that has been modified to alter at least one constant region-mediated biological effector function relative to the corresponding wild type sequence. For example, in some embodiments, the antibody can be modified to reduce at least one constant region-mediated biological effector function relative to an unmodified antibody, e.g., reduced binding to the Fc receptor (FcR). FcR binding can be reduced by, for example, mutating the immunoglobulin constant region segment of the antibody at particular regions necessary for FcR interactions.

An antibody described herein can be modified to acquire or improve at least one constant region-mediated biological effector function relative to an unmodified antibody, e.g., to enhance FcγR interactions. For example, an antibody with a constant region that binds FcγRIIA, FcγRIIB and/or FcγRIIIA with greater affinity than the corresponding wild type constant region can be produced according to the methods described herein.

A construct can comprise an antibody with modifications occurring at least at one amino acid residue. Modifications can be substitutions, additions, mutations, deletions, or the like. An antibody modification can be an insertion of an unnatural amino acid.

A construct can comprise a light chain of an amino acid sequence having at least one, two, three, four, five, six, seven, eight, nine or ten modifications but not more than 40, 35, 30, 25, 20, 15 or 10 modifications of the amino acid sequence relative to the natural or original amino acid sequence. An antibody construct can comprise a heavy chain of an amino acid sequence having at least one, two, three, four, five, six, seven, eight, nine or ten modifications but not more than 40, 35, 30, 25, 20, 15 or 10 modifications of the amino acid sequence relative to the natural or original amino acid sequence.

A construct can comprise an Fc domain of an IgG1 isotype. A construct can comprise an Fc domain of an IgG2 isotype. A construct can comprise an Fc domain of an IgG3 isotype. A construct can comprise an Fc domain of an IgG4 isotype. A construct can have a hybrid isotype comprising constant regions from two or more isotypes.

In some an embodiment, the Fc domain is a human Fc domain. In some embodiments, the Fc domain is selected from a group consisting of a human IgG1 Fc domain, a human IgG2 Fc domain, a human IgG3 Fc domain, and a human IgG4 Fc domain. In some embodiments, the Fc domain binds an Fc receptor with the same affinity as a wild type IgG1 Fc domain.

An antibody construct described herein can have a sequence that has been modified to alter at least one constant region-mediated biological effector function relative to the corresponding wild type sequence. For example, in some embodiments, the antibody construct can be modified to increase or decrease at least one constant region-mediated biological effector function relative to an unmodified antibody construct, e.g., increased binding to an Fc receptor (FcR). FcR binding can be reduced or increased by, for example, mutating the immunoglobulin constant region segment of the antibody at particular regions necessary for FcR interactions.

An antibody construct described herein can be modified to acquire or improve at least one constant region-mediated biological effector function relative to an unmodified antibody construct, e.g., to enhance FcγR interactions. For example, an antibody construct with a constant region that binds FcγRIIA, FcγRIIB and/or FcγRIIIA with greater affinity than the corresponding wild type constant region can be produced according to the methods described herein.

An antibody construct described herein can bind to tumor cells, such as an antibody against a cell surface receptor or a tumor antigen. An antibody construct described herein can bind to CD40, such as an antibody that can be a CD40 agonist and bind to CD40.

A construct can comprise an anti-CD40 antibody. A construct can comprise an antibody light chain. A light chain can be a light chain of an anti-CD40 antibody which can bind a CD40 antigen. A light chain of an anti-CD40 antibody can be expressed from a DNA sequence comprising ATGAGGCTCCCTGCTCAGCTCCTGGGGCTCCTGCTGCTCTGGTTCCCAGGTTCCAGAT GCGACATCCAGATGACCCAGTCTCCATCTTCCGTGTCTGCATCTGTAGGAGACAGAG TCACCATCACTTGTCGGGCGAGTCAGGGTATTTACAGCTGGTTAGCCTGGTATCAGC AGAAACCAGGGAAAGCCCCTAACCTCCTGATCTATACTGCATCCACTTTACAAAGTG GGGTCCCATCAAGGTTCAGCGGCAGTGGATCTGGGACAGATTTCACTCTCACCATCA GCAGCCTGCAACCTGAAGATTTTGCAACTTACTATTGTCAACAGGCTAACATTTTCCC GCTCACTTTCGGCGGAGGGACCAAGGTGGAGATCAAACGAACTGTGGCTGCACCAT CTGTCTTCATCTTCCCGCCATCTGATGAGCAGTTGAAATCTGGAACTGCCTCTGTTGT GTGCCTGCTGAATAACTTCTATCCCAGAGAGGCCAAAGTACAGTGGAAGGTGGATA ACGCCCTCCAATCGGGTAACTCCCAGGAGAGTGTCACAGAGCAGGACAGCAAGGAC AGCACCTACAGCCTCAGCAGCACCCTGACGCTGAGCAAAGCAGACTACGAGAAACA CAAAGTCTACGCCTGCGAAGTCACCCATCAGGGCCTGAGCTCGCCCGTCACAAAGA GCTTCAACAGGGGAGAGTGTTAG (SEQ ID NO: 1). A light chain of an anti-CD40 antibody can be expressed from DNA sequence comprising greater than 70%, greater than 75%, greater than 80%, greater than 85%, greater than 90%, greater than 95%, or greater than 99% homology to SEQ ID NO: 1. A variable region of a light chain of an anti-CD40 antibody can be expressed from a DNA sequence comprising GACATCCAGATGACCCAGTCTCCATCTTCCGTGTCTGCATCTGTAGGAGACAGAGTC ACCATCACTTGTCGGGCGAGTCAGGGTATTTACAGCTGGTTAGCCTGGTATCAGCAG AAACCAGGGAAAGCCCCTAACCTCCTGATCTATACTGCATCCACTTTACAAAGTGGG GTCCCATCAAGGTTCAGCGGCAGTGGATCTGGGACAGATTTCACTCTCACCATCAGC AGCCTGCAACCTGAAGATTTTGCAACTTACTATTGTCAACAGGCTAACATTTTCCCGC TCACTTTCGGCGGAGGGACCAAGGTGGAGATCAA (SEQ ID NO: 3). A variable region of a light chain of an anti-CD40 antibody can be expressed from a DNA sequence comprising greater than 70%, greater than 75%, greater than 80%, greater than 85%, greater than 90%, greater than 95% or greater than 99% homology to SEQ ID NO: 3. Additionally, anti-CD40 antibodies expressed from SEQ ID NO: 1, or expressed from a DNA sequence comprising greater than 70% homology to SEQ ID NO: 1 can have a dissociation constant (K_{d}) for CD40 that is less than 10 nM. Anti-CD40 antibodies expressed from SEQ ID NO: 1, or expressed from a DNA sequence comprising greater than 70% homology to SEQ ID NO: 1 can have a dissociation constant (K_{d}) for CD40 that is less than 1 nM, less than 100 pM, less than 10 pM, less than 1 pM, or less than 0.1 pM. The anti-CD40 light chain can be expressed with any anti-CD40 heavy chain or fragment thereof. The anti-CD40 light chain can also expressed with any anti-CD40 heavy chain or fragment thereof to form an anti-CD40 antibody or fragment thereof. The anti-CD40 antibody or fragment thereof can be purified, and can be combined with a pharmaceutically acceptable carrier. The anti-CD40 antibody can be a construct.

A light chain of an anti-CD40 antibody can comprise an amino acid sequence MRLPAQLLGLLLLWFPGSRCDIQMTQSPSSVSASVGDRVTITCRASQGIYSWLAWYQQK PGKAPNLLIYTASTLQSGVPSRFSGSGSGTDFTLTISSLQPEDFATYYCQQANIFPLTFGGG TKVEIKRTVAAPSVFIFPPSDEQLKSGTASVVCLLNNFYPREAKVQWKVDNALQSGNSQ ESVTEQDSKDSTYSLSSTLTLSKADYEKHKVYACEVTHQGLSSPVTKSFNRGEC (SEQ ID NO: 4). A light chain of an anti-CD40 antibody can comprise an amino sequence with greater than 70%, greater than 75%, greater than 80%, greater than 85%, greater than 90%, greater than 95% or greater than 99% homology to SEQ ID NO: 4. A variable region of a light chain of an anti-CD40 antibody can comprise an amino acid sequence DIQMTQSPSSVSASVGDRVTITCRASQGIYSWLAWYQQKPGKAPNLLIYTASTLQSGVPS RFSGSGSGTDFTLTISSLQPEDFATYYCQQANIFPLTFGGGTKVEIK (SEQ ID NO: 6). A variable region of a light chain of an anti-CD40 antibody can comprise an amino acid sequence with greater than 70%, greater than 75%, greater than 80%, greater than 85%, greater than 90%, greater than 95% or greater than 99% homology to SEQ ID NO: 6. Additionally, anti-CD40 antibodies comprising SEQ ID NO: 4, or comprising an amino acid sequence with greater than 70% homology to SEQ ID NO: 4 can have a dissociation constant (K_{d}) for CD40 that is less than 10 nM. Anti-CD40 antibodies comprising SEQ ID NO: 4, or comprising an amino acid sequence with greater than 70% homology to SEQ ID NO: 4 can have a dissociation constant (K_{d}) for CD40 that is less than 1 nM, less than 100 pM, less than 10 pM, less than 1 pM, or less than 0.1 pM. The anti-CD40 light chain can be purified, and can be combined with a pharmaceutically acceptable carrier. The anti-CD40 light chain can be combined with any anti-CD40 heavy chain or fragment thereof. The anti-CD40 light chain can also be combined with any anti-CD40 heavy chain or fragment thereof to form an anti-CD40 antibody or fragment thereof. The anti-CD40 antibody or fragment thereof can be purified, and can be combined with a pharmaceutically acceptable carrier. The anti-CD40 antibody can be a construct. Additionally, one skilled in the art would recognize that these same concepts could apply to anti-CD40 antibodies created for use in the veterinary sciences and/or in laboratory animals.

A construct can comprise an antibody light chain. A light chain can be a light chain of an anti-CD40 antibody which can bind a CD40 antigen. A light chain of an anti-CD40 antibody can be SBT-040 VL-Ck. SBT-040 VL-Ck can comprise an amino acid sequence DIQMTQSPSSVSASVGDRVTITCRASQGIYSWLAWYQQKPGKAPNLLIYTASTLQSGVPS RFSGSGSGTDFTLTISSLQPEDFATYYCQQANIFPLTFGGGTKVEIKRTVAAPSVFIFPPSD EQLKSGTASVVCLLNNFYPREAKVQWKVDNALQSGNSQESVTEQDSKDSTYSLSSTLTL SKADYEKHKVYACEVTHQGLSSPVTKSFNRGEC (SEQ ID NO: 26). SBT-040 VL-Ck can comprise an amino acid sequence with greater than 70%, greater than 75%, greater than 80%, greater than 85%, greater than 90%, greater than 95% or greater than 99% homology to SEQ ID NO: 26.

A light chain of an anti-CD40 antibody can comprise a CDR. A light chain of an anti-CD40 antibody can comprise a CDR with an amino acid sequence QGIYSW (SEQ ID NO: 27). A light chain of an anti-CD40 antibody can comprise a CDR with an amino acid sequence TAS (SEQ ID NO: 28). A light chain of an anti-CD40 antibody can comprise a CDR with an amino acid sequence QQANIFPLT (SEQ ID NO: 29). A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 27. A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 28. A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 29.

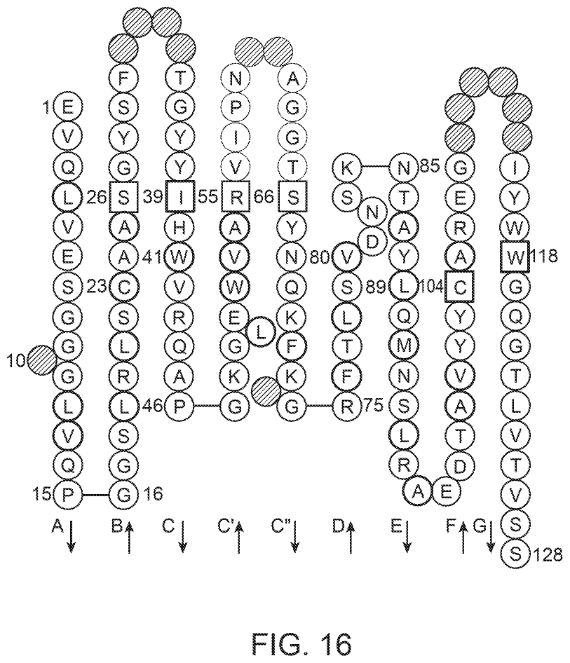

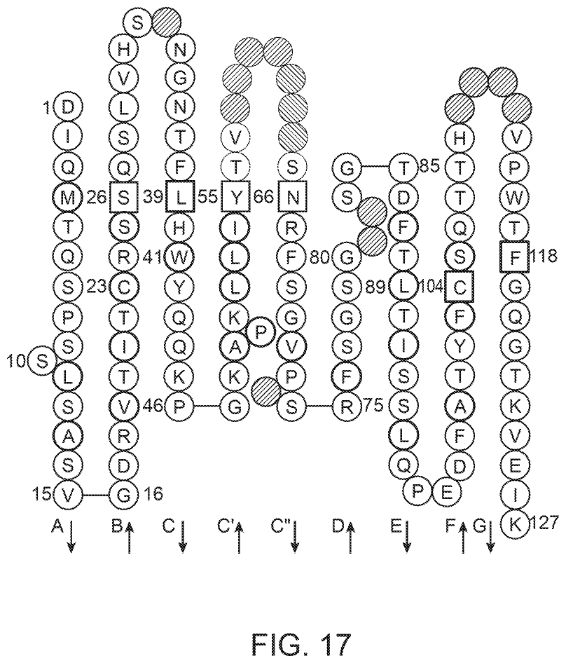

A construct can comprise an antibody heavy chain. A heavy chain can be a heavy chain of an anti-CD40 antibody which can bind a CD40 antigen. A heavy chain of an anti-CD40 antibody can be an IgG1 isotype. A heavy chain of an anti-CD40 antibody can be Dacetuzumab. Dacetuzumab can comprise an amino acid sequence EVQLVESGGGLVQPGGSLRLSCAASGYSFTGYYIHWVRQAPGKGLEWVARVIPNAGGT SYNQKFKGRFTLSVDNSKNTAYLQMNSLRAEDTAVYYCAREGIYWWGQGTLVTVSSA STKGPSVFPLAPSSKSTSGGTAALGCLVKDYFPEPVTVSWNSGALTSGVHTFPAVLQSSG LYSLSSVVTVPSSSLGTQTYICNVNHKPSNTKVDKKVEPKSCDKTHTCPPCPAPELLGGP SVFLFPPKPKDTLMISRTPEVTCVVVDVSHEDPEVKFNWYVDGVEVHNAKTKPREEQY NSTYRVVSVLTVLHQDWLNGKEYKCKVSNKALPAPIEKTISKAKGQPREPQVYTLPPSR EEMTKNQVSLTCLVKGFYPSDIAVEWESNGQPENNYKTTPPVLDSDGSFFLYSKLTVDK SRWQQGNVFSCSVMHEALHNHYTQKSLSLSPGK (SEQ ID NO: 38). Dacetuzumab can comprise an amino acid sequence with greater than 70%, greater than 75%, greater than 80%, greater than 85%, greater than 90%, greater than 95% or greater than 99% homology to SEQ ID NO: 38. A heavy chain of an anti-CD40 antibody can comprise a CDR. A heavy chain of an anti-CD40 antibody can comprise a CDR with an amino acid sequence GYSFTGYY (SEQ ID NO: 39). A heavy chain of an anti-CD40 antibody can comprise a CDR with an amino acid sequence VIPNAGGT (SEQ ID NO: 40). A heavy chain of an anti-CD40 antibody can comprise a CDR with an amino acid sequence AREGIYW (SEQ ID NO: 41). A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 39. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 40. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 41. The two-dimensional structure of the dacetuzumab heavy chain is shown in FIG. 16.

A construct can comprise an antibody light chain. A light chain can be a light chain of an anti-CD40 antibody which can bind a CD40 antigen. A light chain of an anti-CD40 antibody can be dacetuzumab. Dacetuzumab can comprise an amino acid sequence DIQMTQSPSSLSASVGDRVTITCRSSQSLVHSNGNTFLHWYQQKPGKAPKLLIYTVSNRF SGVPSRFSGSGSGTDFTLTISSLQPEDFATYFCSQTTHVPWTFGQGTKVEIKRTVAAPSVFI FPPSDEQLKSGTASVVCLLNNFYPREAKVQWKVDNALQSGNSQESVTEQDSKDSTYSLS STLTLSKADYEKHKVYACEVTHQGLSSPVTKSFNRGEC (SEQ ID NO: 42). Dacetuzumab can comprise an amino acid sequence with greater than 70%, greater than 75%, greater than 80%, greater than 85%, greater than 90%, greater than 95% or greater than 99% homology to SEQ ID NO: 42. A light chain of an anti-CD40 antibody can comprise a CDR. A light chain of an anti-CD40 antibody can comprise a CDR with an amino acid sequence QSLVHSNGNTF (SEQ ID NO: 43). A light chain of an anti-CD40 antibody can comprise a CDR with an amino acid sequence TVS (SEQ ID NO: 44). A light chain of an anti-CD40 antibody can comprise a CDR with an amino acid sequence SQTTHVPWT (SEQ ID NO: 45). A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 43. A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 44. A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 45. The two-dimensional structure of the Dacetuzumab light chain is shown in FIG. 17.

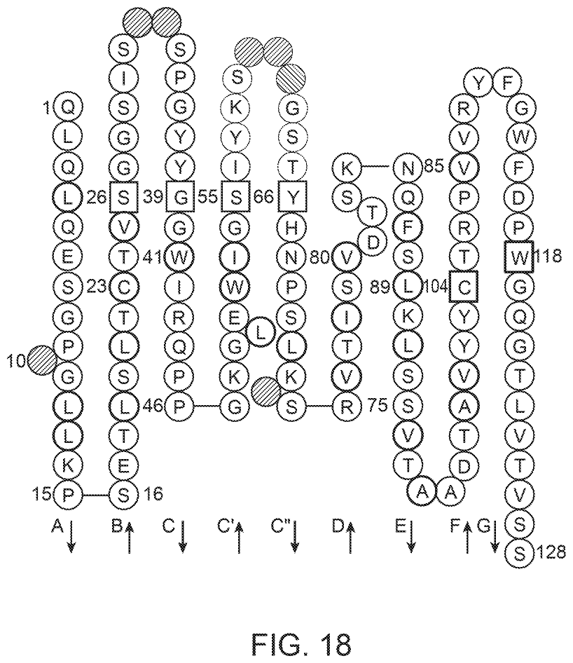

A construct can comprise an antibody heavy chain. A heavy chain can be a heavy chain of an anti-CD40 antibody which can bind a CD40 antigen. A heavy chain of an anti-CD40 antibody can be an IgG4 isotype. A heavy chain of an anti-CD40 antibody can be Bleselumab. Bleselumab can comprise an amino acid sequence QLQLQESGPGLLKPSETLSLTCTVSGGSISSPGYYGGWIRQPPGKGLEWIGSIYKSGSTYH NPSLKSRVTISVDTSKNQFSLKLSSVTAADTAVYYCTRPVVRYFGWFDPWGQGTLVTVS SASTKGPSVFPLAPCSRSTSESTAALGCLVKDYFPEPVTVSWNSGALTSGVHTFPAVLQS SGLYSLSSVVTVPSSSLGTKTYTCNVDHKPSNTKVDKRVESKYGPPCPPCPAPEFEGGPS VFLFPPKPKDTLMISRTPEVTCVVVDVSQEDPEVQFNWYVDGVEVHNAKTKPREEQFNS TYRVVSVLTVLHQDWLNGKEYKCKVSNKGLPSSIEKTISKAKGQPREPQVYTLPPSQEE MTKNQVSLTCLVKGFYPSDIAVEWESNGQPENNYKTTPPVLDSDGSFFLYSRLTVDKSR WQEGNVFSCSVMHEALHNHYTQKSLSLSLGK (SEQ ID NO: 46). Bleselumab can comprise an amino acid sequence with greater than 70%, greater than 75%, greater than 80%, greater than 85%, greater than 90%, greater than 95% or greater than 99% homology to SEQ ID NO: 46. A heavy chain of an anti-CD40 antibody can comprise a CDR. A heavy chain of an anti-CD40 antibody can comprise a CDR with an amino acid sequence GGSISSPGYY (SEQ ID NO: 47). A heavy chain of an anti-CD40 antibody can comprise a CDR with an amino acid sequence IYKSGST (SEQ ID NO: 48). A heavy chain of an anti-CD40 antibody can comprise a CDR with an amino acid sequence TRPVVRYFGWFDP (SEQ ID NO: 49). A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 47. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 48. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 49. The two-dimensional structure of the bleselumab heavy chain is shown in FIG. 18.

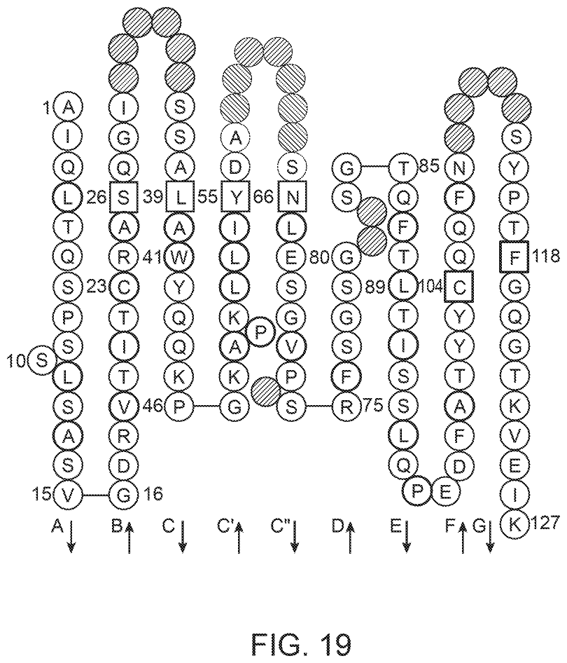

A construct can comprise an antibody light chain. A light chain can be a light chain of an anti-CD40 antibody which can bind a CD40 antigen. A light chain of an anti-CD40 antibody can be Bleselumab. Bleselumab can comprise an amino acid sequence AIQLTQSPSSLSASVGDRVTITCRASQGISSALAWYQQKPGKAPKLLIYDASNLESGVPSR FSGSGSGTDFTLTISSLQPEDFATYYCQQFNSYPTFGQGTKVEIKRTVAAPSVFIFPPSDEQ LKSGTASVVCLLNNFYPREAKVQWKVDNALQSGNSQESVTEQDSKDSTYSLSSTLTLSK ADYEKHKVYACEVTHQGLSSPVTKSFNRGEC (SEQ ID NO: 50). Bleselumab can comprise an amino acid sequence with greater than 70%, greater than 75%, greater than 80%, greater than 85%, greater than 90%, greater than 95% or greater than 99% homology to SEQ ID NO: 50. A light chain of an anti-CD40 antibody can comprise a CDR. A light chain of an anti-CD40 antibody can comprise a CDR with an amino acid sequence QGISSA (SEQ ID NO: 51). A light chain of an anti-CD40 antibody can comprise a CDR with an amino acid sequence DAS (SEQ ID NO: 52). A light chain of an anti-CD40 antibody can comprise a CDR with an amino acid sequence QQFNSYPT (SEQ ID NO: 53). A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 51. A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 52. A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 53. The two-dimensional structure of the bleselumab light chain is shown in FIG. 19.

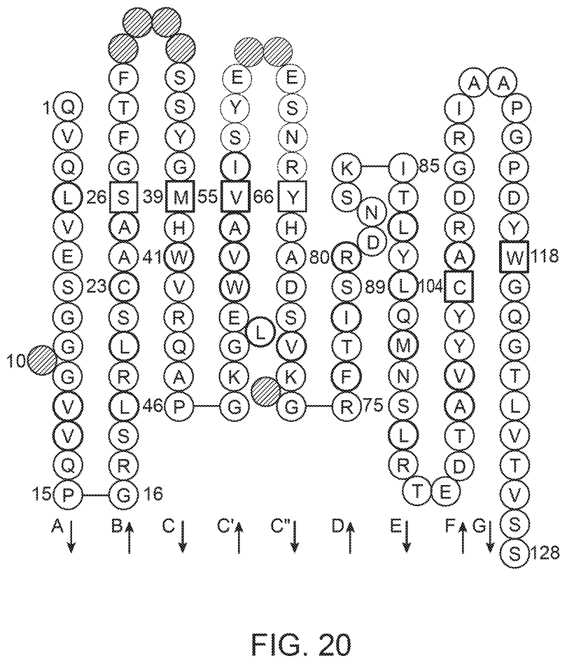

A construct can comprise an antibody heavy chain. A heavy chain can be a heavy chain of an anti-CD40 antibody which can bind a CD40 antigen. A heavy chain of an anti-CD40 antibody can be an IgG1 isotype. Lucatumumab can comprise an amino acid sequence QVQLVESGGGVVQPGRSLRLSCAASGFTFSSYGMHWVRQAPGKGLEWVAVISYEESNR YHADSVKGRFTISRDNSKITLYLQMNSLRTEDTAVYYCARDGGIAAPGPDYWGQGTLV TVSSASTKGPSVFPLAPASKSTSGGTAALGCLVKDYFPEPVTVSWNSGALTSGVHTFPAV LQSSGLYSLSSVVTVPSSSLGTQTYICNVNHKPSNTKVDKRVEPKSCDKTHTCPPCPAPE LLGGPSVFLFPPKPKDTLMISRTPEVTCVVVDVSHEDPEVKFNWYVDGVEVHNAKTKPR EEQYNSTYRVVSVLTVLHQDWLNGKEYKCKVSNKALPAPIEKTISKAKGQPREPQVYTL PPSREEMTKNQVSLTCLVKGFYPSDIAVEWESNGQPENNYKTTPPVLDSDGSFFLYSKLT VDKSRWQQGNVFSCSVMHEALHNHYTQKSLSLSPGK (SEQ ID NO: 54). Lucatumumab can comprise an amino acid sequence with greater than 70%, greater than 75%, greater than 80%, greater than 85%, greater than 90%, greater than 95% or greater than 99% homology to SEQ ID NO: 54. A heavy chain of an anti-CD40 antibody can comprise a CDR. A heavy chain of an anti-CD40 antibody can comprise a CDR with an amino acid sequence GFTFSSYG (SEQ ID NO: 55). A heavy chain of an anti-CD40 antibody can comprise a CDR with an amino acid sequence ISYEESNR (SEQ ID NO: 56). A heavy chain of an anti-CD40 antibody can comprise a CDR with an amino acid sequence ARDGGIAAPGPDY (SEQ ID NO: 57). A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 55. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 56. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 57. The two-dimensional structure of the lucatumumab heavy chain is shown in FIG. 20.

A construct can comprise an antibody light chain. A light chain can be a light chain of an anti-CD40 antibody which can bind a CD40 antigen. A light chain of an anti-CD40 antibody can be Lucatumumab. Lucatumumab can comprise an amino acid sequence

(SEQ ID NO: 58)
DIVMTQSPLSLTVTPGEPASISCRSSQSLLYSNGYNYLDWYLQKPGQSP
QVLISLGSNRASGVPDRFSGSGSGTDFTLKISRVEAEDVGVYYCMQARQ
TPFTFGPGTKVDIRRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC.

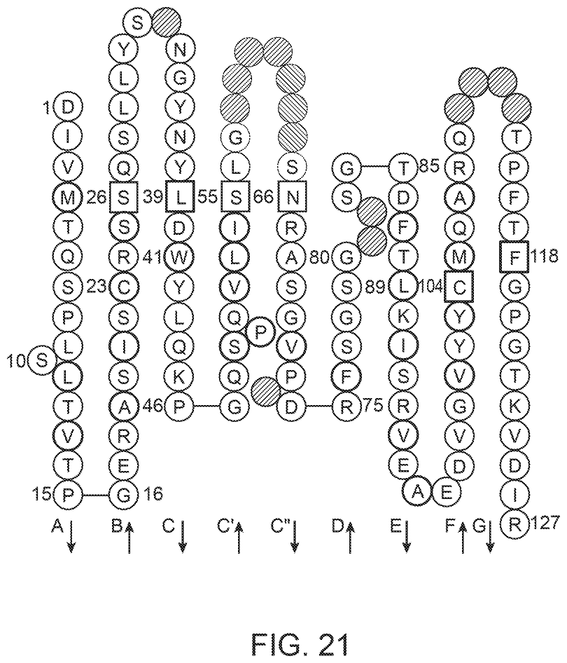

Lucatumumab can comprise an amino acid sequence with greater than 70%, greater than 75%, greater than 80%, greater than 85%, greater than 90%, greater than 95% or greater than 99% homology to SEQ ID NO: 58. A light chain of an anti-CD40 antibody can comprise a CDR with an amino acid sequence LGS (SEQ ID NO: 60). A light chain of an anti-CD40 antibody can comprise a CDR with an amino acid sequence MQARQTPFT (SEQ ID NO: 61). A light chain CDR of an anti-CD40 antibody can comprise an amino acid sequence with greater than 70%, greater than 75%, greater than 80%, greater than 85%, greater than 90%, greater than 95% or greater than 99% homology to QSLLYSNGYNY (SEQ ID NO: 59). A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 60. A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 61. The two-dimensional structure of the lucatumumab light chain is shown in FIG. 21.

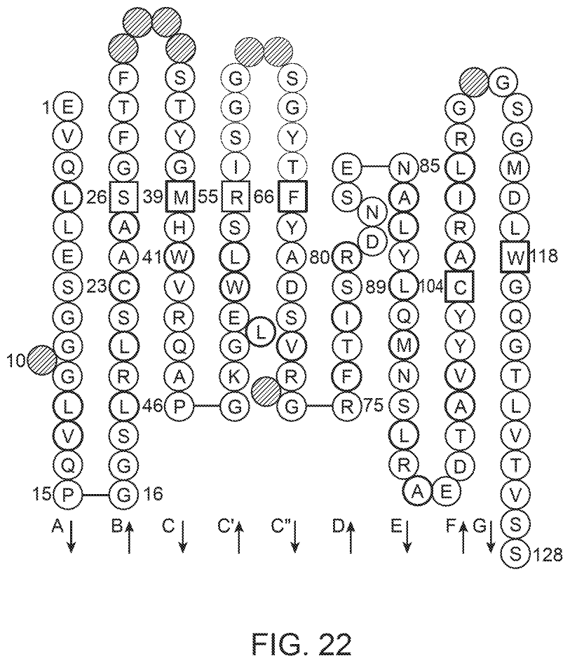

A construct can comprise an antibody heavy chain. A heavy chain can be a heavy chain of an anti-CD40 antibody which can bind a CD40 antigen. A heavy chain of an anti-CD40 antibody can be an IgG1 isotype. A heavy chain of an anti-CD40 antibody can be ADC-1013. ADC-1013 can comprise an amino acid sequence EVQLLESGGGLVQPGGSLRLSCAASGFTFSTYGMHWVRQAPGKGLEWLSYISGGSSYIF YADSVRGRFTISRDNSENALYLQMNSLRAEDTAVYYCARILRGGSGMDLWGQGTLVTV SSASTKGPSVFPLAPSSKSTSGGTAALGCLVKDYFPEPVTVSWNSGALTSGVHTFPAVLQ SSGLYSLSSWTVPSSSLGTQTYICNVNHKPSNTKVDKKVEPKSCDKTHTCPPCPAPELLG GPSVFLFPPKPKDTLMISRTPEVTCNAVDVSHEDPEVKFNWYVDGVEVHNAKTKPREEQ YNSTYRWSVLTVLHQDWLNGKEYKCKVSNKALPAPIEKTISKAKGQPREPQVYTLPPSR DELTKNQVSLTCLVKGFYPSDIAVEWESNGQPENNYKTTPPVLDSDGSFFLYSKLTVDK SRWQQGNVFSCSVMHEALHNHYTQKSLSLSPGK (SEQ ID NO: 62). ADC-1013 can comprise an amino acid sequence with greater than 70%, greater than 75%, greater than 80%, greater than 85%, greater than 90%, greater than 95% or greater than 99% homology to SEQ ID NO: 62. A heavy chain of an anti-CD40 antibody can comprise a CDR. A heavy chain of an anti-CD40 antibody can comprise a CDR with an amino acid sequence GFTFSTYG (SEQ ID NO: 63). A heavy chain of an anti-CD40 antibody can comprise a CDR with an amino acid sequence ISGGSSYI (SEQ ID NO: 64). A heavy chain of an anti-CD40 antibody can comprise a CDR with an amino acid sequence ARILRGGSGMDL (SEQ ID NO: 65). A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 63. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 64. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 65. The two-dimensional structure of the ADC-1013 heavy chain is shown in FIG. 22.

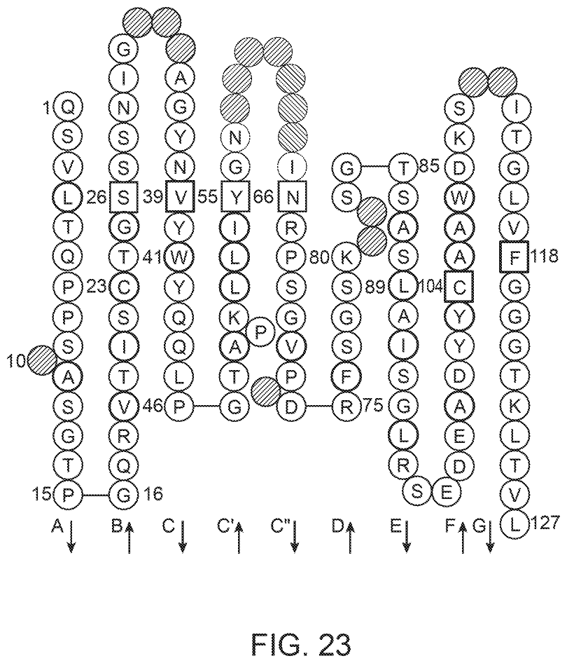

A construct can comprise an antibody light chain. A light chain can be a light chain of an anti-CD40 antibody which can bind a CD40 antigen. A light chain of an anti-CD40 antibody can be ADC-1013. ADC-1013 can comprise an amino acid sequence QSVLTQPPSASGTPGQRVTISCTGSSSNIGAGYNVYWYQQLPGTAPKLLIYGNINRPSGVP DRFSGSKSGTSASLAISGLRSEDEADYYCAAWDKSISGLVFGGGTKLTVLGQPKAAPSVT LFPPSSEELQANKATLVCLISDFYPGAVTVAWKADSSPVKAGVETTTPSKQSNNKYAAS SYLSLTPEQWKSHRSYSCQVTHEGSTVEKTVAPTECS (SEQ ID NO: 66). ADC-1013 can comprise an amino acid sequence with greater than 70%, greater than 75%, greater than 80%, greater than 85%, greater than 90%, greater than 95% or greater than 99% homology to SEQ ID NO: 66. A light chain of an anti-CD40 antibody can comprise a CDR. A light chain of an anti-CD40 antibody can comprise a CDR with an amino acid sequence SSNIGAGYN (SEQ ID NO: 67). A light chain of an anti-CD40 antibody can comprise a CDR with an amino acid sequence GNI (SEQ ID NO: 68). A light chain of an anti-CD40 antibody can comprise a CDR with an amino acid sequence AAWDKSISGLV (SEQ ID NO: 69). A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 67. A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 68. A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 69. The two-dimensional structure of the ADC-1013 light chain is shown in FIG. 23.

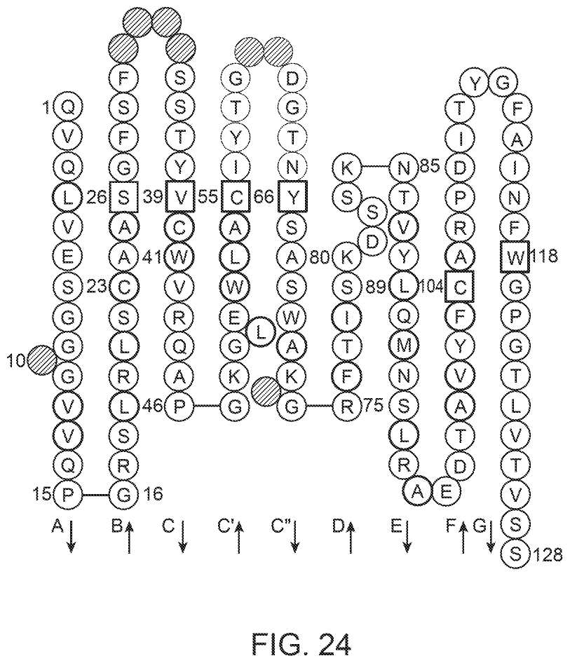

A construct can comprise an antibody heavy chain. A heavy chain can be a heavy chain of an anti-CD40 antibody which can bind a CD40 antigen. A heavy chain of an anti-CD40 antibody can be the humanized rabbit antibody APX005. APX005 can comprise an amino acid sequence QVQLVESGGGVVQPGRSLRLSCAASGFSFSSTYVCWVRQAPGKGLEWIACIYTGDGTN YSASWAKGRFTISKDSSKNTVYLQMNSLRAEDTAVYFCARPDITYGFAINFWGPGTLVT VSSASTKGPSVFPLAPSSKSTSGGTAALGCLVKDYFPEPVTVSWNSGALTSGVHTFPAVL QSSGLYSLSSVVTVPSSSLGTQTYICNVNHKPSNTKVDKKVEPKSCDKTHTCPPCPAPEL LGGPSVFLFPPKPKDTLMISRTPEVTCVVVDVSHEDPEVKFNWYVDGVEVHNAKTKPRE EQYNSTYRVVSVLTVLHQDWLNGKEYKCKVSNKALPAPIEKTISKAKGQPREPQVYTLP PSREEMTKNQVSLTCLVKGFYPSDIAVEWESNGQPENNYKTTPPVLDSDGSFFLYSKLTV DKSRWQQGNVFSCSVMHEALHNHYTQKSLSLSPGK (SEQ ID NO: 70). APX005 can comprise an amino acid sequence with greater than 70%, greater than 75%, greater than 80%, greater than 85%, greater than 90%, greater than 95% or greater than 99% homology to SEQ ID NO: 70. A heavy chain of an anti-CD40 antibody can comprise a CDR. A heavy chain of an anti-CD40 antibody can comprise a CDR with an amino acid sequence GFSFSSTY (SEQ ID NO: 71). A heavy chain of an anti-CD40 antibody can comprise a CDR with an amino acid sequence IYTGDGTN (SEQ ID NO: 72). A heavy chain of an anti-CD40 antibody can comprise a CDR with an amino acid sequence ARPDITYGFAINFW (SEQ ID NO: 73). A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 71. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 72. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 73. The two-dimensional structure of the APX005 heavy chain is shown in FIG. 24.

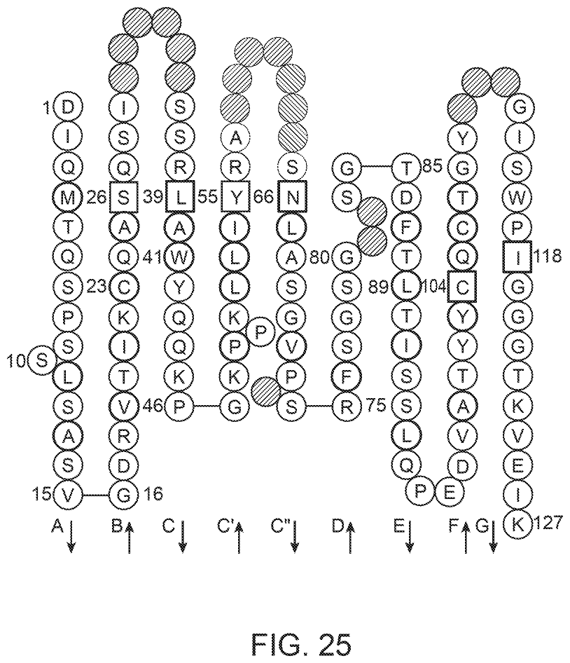

A construct can comprise an antibody light chain. A light chain can be a light chain of an anti-CD40 antibody which can bind a CD40 antigen. A light chain of an anti-CD40 antibody can be the humanized rabbit antibody APX005. APX005 can comprise an amino acid sequence DIQMTQSPSSLSASVGDRVTIKCQASQSISSRLAWYQQKPGKPPKLLIYRASTLASGVPSR FSGSGSGTDFTLTISSLQPEDVATYYCQCTGYGISWPIGGGTKVEIKRTVAAPSVFIFPPSD EQLKSGTASVVCLLNNFYPREAKVQWKVDNALQSGNSQESVTEQDSKDSTYSLSSTLTL SKADYEKHKVYACEVTHQGLSSPVTKSFNRGEC (SEQ ID NO: 74). APX005 can comprise an amino acid sequence with greater than 70%, greater than 75%, greater than 80%, greater than 85%, greater than 90%, greater than 95% or greater than 99% homology to SEQ ID NO: 74. A light chain of an anti-CD40 antibody can comprise a CDR. A light chain of an anti-CD40 antibody can comprise a CDR with an amino acid sequence QSISSR (SEQ ID NO: 75). A light chain of an anti-CD40 antibody can comprise a CDR with an amino acid sequence RAS (SEQ ID NO: 76). A light chain of an anti-CD40 antibody can comprise a CDR with an amino acid sequence QCTGYGISWP (SEQ ID NO: 77). A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 75. A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 76. A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 77. The two-dimensional structure of the APX005 light chain is shown in FIG. 25.

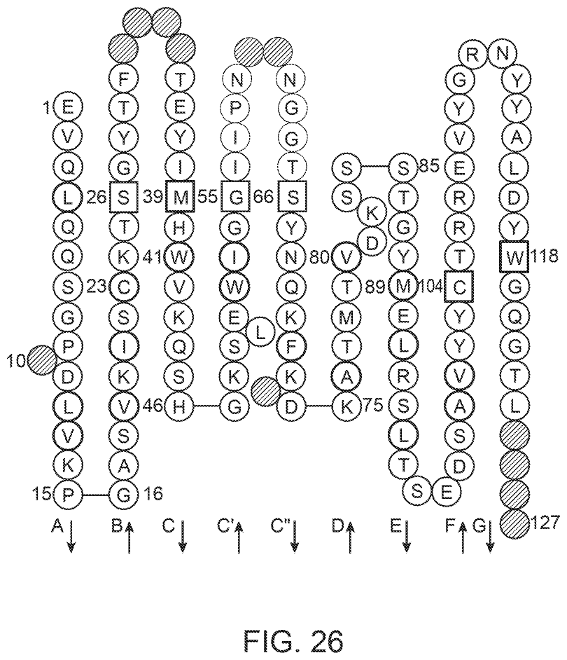

A construct can comprise an antibody heavy chain. A heavy chain can be a heavy chain of an anti-CD40 antibody which can bind a CD40 antigen. A heavy chain of an anti-CD40 antibody can be Chi Lob 7/4. Chi Lob 7/4 can comprise an amino acid sequence EVQLQQSGPDLVKPGASVKISCKTSGYTFTEYIMHWVKQSHGKSLEWIGGIIPNNGGTSY NQKFKDKATMTVDKSSSTGYMELRSLTSEDSAVYYCTRREVYGRNYYALDYWGQGTL VTVSSASTKGPSVFPLAPSSKSTSGGTAALGCLVKDYFPEPVTVSWNSGALTSGVHTFPA VLQSSGLYSLSSVVTVPSSSLGTQTYICNVNHKPSNTKVDKKVEPKSCDKTHTCPPCPAP ELLGGPSVFLFPPKPKDTLMISRTPEVTCVVVDVSHEDPEVKFNWYVDGVEVHNAKTKP REEQYNSTYRVVSVLTVLHQDWLNGKEYKCKVSNKALPAPIEKTISKAKGQPREPQVYT LPPSREEMTKNQVSLTCLVKGFYPSDIAVEWESNGQPENNYKTTPPVLDSDGSFFLYSKL TVDKSRWQQGNVFSCSVMHEALHNHYTQKSLSLSPGK (SEQ ID NO: 78). Chi Lob 7/4 can comprise an amino acid sequence with greater than 70%, greater than 75%, greater than 80%, greater than 85%, greater than 90%, greater than 95% or greater than 99% homology to SEQ ID NO: 78. A heavy chain of an anti-CD40 antibody can comprise a CDR. A heavy chain of an anti-CD40 antibody can comprise a CDR with an amino acid sequence GYTFTEYI (SEQ ID NO: 79). A heavy chain of an anti-CD40 antibody can comprise a CDR with an amino acid sequence IIPNNGGT (SEQ ID NO: 80). A heavy chain of an anti-CD40 antibody can comprise a CDR with an amino acid sequence TRREVYGRNYYALDY (SEQ ID NO: 81). A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 79. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 80. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 81. The two-dimensional structure of the Chi Lob 7/4 heavy chain is shown in FIG. 26.

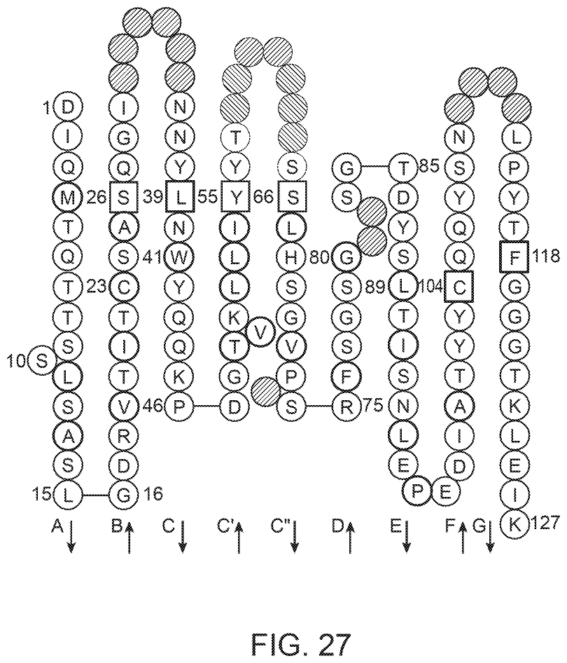

A construct can comprise an antibody light chain. A light chain can be a light chain of an anti-CD40 antibody which can bind a CD40 antigen. A light chain of an anti-CD40 antibody can be Chi Lob 7/4. Chi Lob 7/4 can comprise an amino acid sequence DIQMTQTTSSLSASLGDRVTITCSASQGINNYLNWYQQKPDGTVKLLIYYTSSLHSGVPS RFSGSGSGTDYSLTISNLEPEDIATYYCQQYSNLPYTFGGGTKLEIKRTVAAPSVFIFPPSD EQLKSGTASVVCLLNNFYPREAKVQWKVDNALQSGNSQESVTEQDSKDSTYSLSSTLTL SKADYEKHKVYACEVTHQGLSSPVTKSFNRGEC (SEQ ID NO: 82). Chi Lob 7/4 can comprise an amino acid sequence with greater than 70%, greater than 75%, greater than 80%, greater than 85%, greater than 90%, greater than 95% or greater than 99% homology to SEQ ID NO: 82. A light chain of an anti-CD40 antibody can comprise a CDR. A light chain of an anti-CD40 antibody can comprise a CDR with an amino acid sequence QGINNY (SEQ ID NO: 83). A light chain of an anti-CD40 antibody can comprise a CDR with an amino acid sequence YTS (SEQ ID NO: 84). A light chain of an anti-CD40 antibody can comprise a CDR with an amino acid sequence QQYSNLPYT (SEQ ID NO: 85). A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 83. A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 84. A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 85. The two-dimensional structure of the Chi Lob 7/4 light chain is shown in FIG. 27.

A construct can comprise an antibody heavy chain. A heavy chain can be a heavy chain of an anti-CD40 antibody which can bind a CD40 antigen. A heavy chain of an anti-CD40 antibody can be an IgG1 isotype. A heavy chain of an anti-CD40 antibody can be SBT-040-G1WT. SBT-040-G1WT can be expressed from a DNA sequence comprising ATGGACTGGACCTGGAGGATCCTCTTCTTGGTGGCAGCCACAGGAGCCCACTCC CAGGTGCAGCTGGTGCAGTCTGGGGCTGAGGTGAAGAAGCCTGGGGCCTCAGTGAA GGTCTCCTGCAAGGCTTCTGGATACACCTTCACCGGCTACTATATGCACTGGGTGCG ACAGGCCCCTGGACAAGGGCTTGAGTGGATGGGATGGATCAACCCTGACAGTGGTG GCACAAACTATGCACAGAAGTTTCAGGGCAGGGTCACCATGACCAGGGACACGTCC ATCAGCACAGCCTACATGGAGCTGAACAGGCTGAGATCTGACGACACGGCCGTGTA TTACTGTGCGAGAGATCAGCCCCTAGGATATTGTACTAATGGTGTATGCTCCTACTTT GACTACTGGGGCCAGGGAACCCTGGTCACCGTCTCCTCAGCCTCCACCAAGGGCCCA TCGGTCTTCCCCCTGGCGCCCTGCTCCAGGAGCACCTCCGAGAGCACAGCGGCCCTG GGCTGCCTGGTCAAGGACTACTTCCCCGAACCGGTGACGGTGTCGTGGAACTCAGGC GCTCTGACCAGCGGCGTGCACACCTTCCCAGCTGTCCTACAGTCCTCAGGACTCTAC TCCCTCAGCAGCGTGGTGACCGTGCCCTCCAGCAACTTCGGCACCCAGACCTACACC TGCAACGTAGATCACAAGCCCAGCAACACCAAGGTGGACAAGACAGTTGAGCCCAA ATCTTGTGACAAAACTCACACATGCCCACCGTGCCCAGCACCTGAACTCCTGGGGGG ACCGTCAGTCTTCCTCTTCCCCCCAAAACCCAAGGACACCCTCATGATCTCCCGGAC CCCTGAGGTCACATGCGTGGTGGTGGACGTGAGCCACGAAGACCCTGAGGTCAAGT TCAACTGGTACGTGGACGGCGTGGAGGTGCATAATGCCAAGACAAAGCCGCGGGAG GAGCAGTACAACAGCACGTACCGTGTGGTCAGCGTCCTCACCGTCCTGCACCAGGAC TGGCTGAATGGCAAGGAGTACAAGTGCAAGGTCTCCAACAAAGCCCTCCCAGCCCC CATCGAGAAAACCATCTCCAAAGCCAAAGGGCAGCCCCGAGAACCACAGGTGTACA CCCTGCCCCCATCCCGGGAGGAGATGACCAAGAACCAGGTCAGCCTGACCTGCCTG GTCAAAGGCTTCTATCCCAGCGACATCGCCGTGGAGTGGGAGAGCAATGGGCAGCC GGAGAACAACTACAAGACCACGCCTCCCGTGCTGGACTCCGACGGCTCCTTCTTCCT CTATAGCAAGCTCACCGTGGACAAGAGCAGGTGGCAGCAGGGGAACGTCTTCTCAT GCCCGTGATGCATGAGGCTCTGCACAACCACTACACGCAGAAGAGCCTCTCCCTGT CCCCGGGTAAATGA (SEQ ID NO: 8). SBT-040-G1WT can be expressed from a DNA sequence comprising greater than 70%, greater than 75%, greater than 80%, greater than 85%, greater than 90%, greater than 95% or greater than 99% homology to SEQ ID NO: 8. A variable region of SBT-040-G1WT can be expressed from a DNA sequence comprising CAGGTGCAGCTGGTGCAGTCTGGGGCTGAGGTGAAGAAGCCTGGGGCCTCAGTGAA GGTCTCCTGCAAGGCTTCTGGATACACCTTCACCGGCTACTATATGCACTGGGTGCG ACAGGCCCCTGGACAAGGGCTTGAGTGGATGGGATGGATCAACCCTGACAGTGGTG GCACAAACTATGCACAGAAGTTTCAGGGCAGGGTCACCATGACCAGGGACACGTCC ATCAGCACAGCCTACATGGAGCTGAACAGGCTGAGATCTGACGACACGGCCGTGTA TTACTGTGCGAGAGATCAGCCCCTAGGATATTGTACTAATGGTGTATGCTCCTACTTT GACTACTGGGGCCAGGGAACCCTGGTCACCGTCTCCTCAG (SEQ ID NO: 13). A variable region of SBT-040-G1WT can be expressed from a DNA sequence comprising greater than 70%, greater than 75%, greater than 80%, greater than 85%, greater than 90%, greater than 95% or greater than 99% homology to SEQ ID NO: 13. Additionally, anti-CD40 antibodies comprising SBT-040-G1WT expressed from SEQ ID NO: 8, or expressed from a DNA sequence comprising greater than 70% homology to SEQ ID NO: 8 can have a dissociation constant (K_{d}) for CD40 that is less than 10 nM. Anti-CD40 antibodies comprising SBT-040-G1WT expressed from DNA sequence comprising SEQ ID NO: 8, or comprising greater than 70% homology to SEQ ID NO: 8 can have a dissociation constant (K_{d}) for CD40 that is less than 1 nM, less than 100 pM, less than 10 pM, less than 1 pM, or less than 0.1 pM. SBT-040-G1WT can be expressed with any anti-CD40 light chain or fragment thereof. SBT-040-G1WT can also be expressed with any anti-CD40 light chain or fragment thereof to form an anti-CD40 antibody or fragment thereof. The anti-CD40 antibody or fragment thereof can be purified, and can be combined with a pharmaceutically acceptable carrier. The anti-CD40 antibody can be a construct. Additionally, one skilled in the art would recognize that these same concepts could apply to constructs comprising anti-CD40 antibodies created for use in the veterinary sciences and/or in laboratory animals.

SBT-040-G1WT can comprise an amino acid sequence MDWTWRILFLVAAATGAHSQVQLVQSGAEVKKPGASVKVSCKASGYTFTGYYMHWV RQAPGQGLEWMGWINPDSGGTNYAQKFQGRVTMTRDTSISTAYMELNRLRSDDTAVY YCARDQPLGYCTNGVCSYFDYWGQGTLVTVSSASTKGPSVFPLAPCSRSTSESTAALGC LVKDYFPEPVTVSWNSGALTSGVHTFPAVLQSSGLYSLSSVVTVPSSNFGTQTYTCNVD HKPSNTKVDKTVEPKSCDKTHTCPPCPAPELLGGPSVFLFPPKPKDTLMISRTPEVTCVV VDVSHEDPEVKFNWYVDGVEVHNAKTKPREEQYNSTYRVVSVLTVLHQDWLNGKEY KCKVSNKALPAPIEKTISKAKGQPREPQVYTLPPSREEMTKNQVSLTCLVKGFYPSDIAV EWESNGQPENNYKTTPPVLDSDGSFFLYSKLTVDKSRWQQGNVFSCSVMHEALHNHYT QKSLSLSPGK (SEQ ID NO: 15). SBT-040-G1WT can comprise an amino acid sequence with greater than 70%, greater than 75%, greater than 80%, greater than 85%, greater than 90%, greater than 95% or greater than 99% homology to SEQ ID NO: 15. SBT-040-G1WT can comprise an amino acid sequence QVQLVQSGAEVKKPGASVKVSCKASGYTFTGYYMHWVRQAPGQGLEWMGWINPDSG GTNYAQKFQGRVTMTRDTSISTAYMELNRLRSDDTAVYYCARDQPLGYCTNGVCSYFD YWGQGTLVTVSS (SEQ ID NO: 20). A variable region of SBT-040-G1WT can comprise an amino acid sequence with greater than 70%, greater than 75%, greater than 80%, greater than 85%, greater than 90%, greater than 95% or greater than 99% homology to SEQ ID NO: 20. Additionally, anti-CD40 antibodies comprising SBT-040-G1WT with SEQ ID NO: 15 or with an amino acid sequence with greater than 70% homology to SEQ ID NO: 15 can have a dissociation constant (K_{d}) for CD40 that is less than 10 nM. Anti-CD40 antibodies comprising SBT-040-G1WT with SEQ ID NO: 15 or with an amino acid sequence with greater than 70% homology to SEQ ID NO: 15 can have a dissociation constant (K_{d}) for CD40 that is less than 1 nM, less than 100 pM, less than 10 pM, less than 1 pM, or less than 0.1 pM. SBT-040-G1WT can be purified. SBT-040-G1WT can be combined with any anti-CD40 light chain or fragment thereof to form an anti-CD40 antibody or fragment thereof. The anti-CD40 antibody or fragment thereof can be purified, and can be combined with a pharmaceutically acceptable carrier. The anti-CD40 antibody can be a construct. Additionally, one skilled in the art would recognize that these same concepts could apply to constructs comprising anti-CD40 antibodies created for use in the veterinary sciences and/or in laboratory animals.

A construct can comprise an antibody heavy chain. A heavy chain can be a heavy chain of an anti-CD40 antibody which can bind a CD40 antigen. A heavy chain of an anti-CD40 antibody can be an IgG1 isotype. A heavy chain of an anti-CD40 antibody can be SBT-040 VH-hIgG1 wt. SBT-040 VH-hIgG1 wt can comprise an amino acid sequence with greater than 70%, greater than 75%, greater than 80%, greater than 85%, greater than 90%, greater than 95% or greater than 99% homology to an amino acid sequence QVQLVQSGAEVKKPGASVKVSCKASGYTFTGYYMHWVRQAPGQGLEWMGWINPDSG GTNYAQKFQGRVTMTRDTSISTAYMELNRLRSDDTAVYYCARDQPLGYCTNGVCSYFD YWGQGTLVTVSSASTKGPS VFPLAPCSRSTSESTAALGCLVKDYFPEPVTVSWNSGALTS GVHTFPAVLQSSGLYSLSSVVTVPSSNFGTQTYTCNVDHKPSNTKVDKTVEPKSCDKTH TCPPCPAPELLGGPSVFLFPPKPKDTLMISRTPEVTCVVVDVSHEDPEVKFNWYVDGVEV HNAKTKPREEQYNSTYRVVSVLTVLHQDWLNGKEYKCKVSNKALPAPIEKTISKAKGQ PREPQVYTL PPSREEMTKNQVSLTCLVKGFYPSDIAVEWESNGQPENNYKTTPPVLDSDGSFFLYSKLT VDKSRWQQGNVFSCSVMHEALHNHYTQKSLSLSPGK (SEQ ID NO: 22). A heavy chain of an anti-CD40 antibody can comprise a CDR. A heavy chain of an anti-CD40 antibody can comprise a CDR with an amino acid sequence GYTFTYY (SEQ ID NO: 23). A heavy chain of an anti-CD40 antibody can comprise a CDR with an amino acid sequence INPDSGGT (SEQ ID NO: 24). A heavy chain of an anti-CD40 antibody can comprise a CDR with an amino acid sequence ARDQPLGYCTNGVCSYFDY (SEQ ID NO: 25). A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 23. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 24. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 25.

A heavy chain of an anti-CD40 antibody can be an IgG2 isotype. A heavy chain of an anti-CD40 antibody can be SBT-040-G2. SBT-040-G2 be expressed from a DNA sequence comprising ATGGACTGGACCTGGAGGATCCTCTTCTTGGTGGCAGCCACAGGAGCCCACTCC CAGGTGCAGCTGGTGCAGTCTGGGGCTGAGGTGAAGAAGCCTGGGGCCTCAGTGAA GGTCTCCTGCAAGGCTTCTGGATACACCTTCACCGGCTACTATATGCACTGGGTGCG ACAGGCCCCTGGACAAGGGCTTGAGTGGATGGGATGGATCAACCCTGACAGTGGTG GCACAAACTATGCACAGAAGTTTCAGGGCAGGGTCACCATGACCAGGGACACGTCC ATCAGCACAGCCTACATGGAGCTGAACAGGCTGAGATCTGACGACACGGCCGTGTA TTACTGTGCGAGAGATCAGCCCCTAGGATATTGTACTAATGGTGTATGCTCCTACTTT GACTACTGGGGCCAGGGAACCCTGGTCACCGTCTCCTCAGCCTCCACCAAGGGCCCA TCGGTCTTCCCCCTGGCGCCCTGCTCCAGGAGCACCTCCGAGAGCACAGCGGCCCTG GGCTGCCTGGTCAAGGACTACTTCCCCGAACCGGTGACGGTGTCGTGGAACTCAGGC GCTCTGACCAGCGGCGTGCACACCTTCCCAGCTGTCCTACAGTCCTCAGGACTCTAC TCCCTCAGCAGCGTGGTGACCGTGCCCTCCAGCAACTTCGGCACCCAGACCTACACC TGCAACGTAGATCACAAGCCCAGCAACACCAAGGTGGACAAGACAGTTGAGCGCAA ATGTTGTGTCGAGTGCCCACCGTGCCCAGCACCACCTGTGGCAGGACCGTCAGTCTT CCTCTTCCCCCCAAAACCCAAGGACACCCTCATGATCTCCCGGACCCCTGAGGTCAC GTGCGTGGTGGTGGACGTGAGCCACGAAGACCCCGAGGTCCAGTTCAACTGGTACG TGGACGGCGTGGAGGTGCATAATGCCAAGACAAAGCCACGGGAGGAGCAGTTCAAC AGCACGTTCCGTGTGGTCAGCGTCCTCACCGTTGTGCACCAGGACTGGCTGAACGGC AAGGAGTACAAGTGCAAGGTCTCCAACAAAGGCCTCCCAGCCCCCATCGAGAAAAC CATCTCCAAAACCAAAGGGCAGCCCCGAGAACCACAGGTGTACACCCTGCCCCCAT CCCGGGAGGAGATGACCAAGAACCAGGTCAGCCTGACCTGCCTGGTCAAAGGCTTC TACCCCAGCGACATCGCCGTGGAGTGGGAGAGCAATGGGCAGCCGGAGAACAACTA CAAGACCACACCTCCCATGCTGGACTCCGACGGCTCCTTCTTCCTCTACAGCAAGCT CACCGTGGACAAGAGCAGGTGGCAGCAGGGGAACGTCTTCTCATGCTCCGTGATGC ATGAGGCTCTGCACAACCACTACACGCAGAAGAGCCTCTCCCTGTCTCCGGGTAAAT GA (SEQ ID NO: 7). SBT-040-G2 can be expressed from a DNA sequence comprising greater than 70%, greater than 75%, greater than 80%, greater than 85%, greater than 90%, greater than 95% or greater than 99% homology to SEQ ID NO: 7. A variable region of SBT-040-G2 can be expressed from a DNA sequence comprising SEQ ID NO: 13. A variable region of SBT-040-G2 can be expressed from a DNA sequence comprising greater than 70%, greater than 75%, greater than 80%, greater than 85%, greater than 90%, greater than 95% or greater than 99% homology to SEQ ID NO: 13. Additionally, anti-CD40 antibodies comprising SBT-040-G2 expressed from SEQ ID NO: 7, or expressed from a DNA sequence comprising greater than 70% homology to SEQ ID NO: 7 can have a dissociation constant (K_{d}) for CD40 that is less than 10 nM. Anti-CD40 antibodies comprising SBT-040-G2 expressed from DNA sequence comprising SEQ ID NO: 7, or comprising greater than 70% homology to SEQ ID NO: 7 can have a dissociation constant (K_{d}) for CD40 that is less than 1 nM, less than 100 pM, less than 10 pM, less than 1 pM, or less than 0.1 pM. SBT-040-G2 can be expressed with any anti-CD40 light chain or fragment thereof. SBT-040-G2 can also be expressed with any anti-CD40 light chain or fragment thereof to form an anti-CD40 antibody or fragment thereof. The anti-CD40 antibody or fragment thereof can be purified, and can be combined with a pharmaceutically acceptable carrier. The anti-CD40 antibody can be a construct. Additionally, one skilled in the art would recognize that these same concepts could apply to constructs comprising anti-CD40 antibodies created for use in the veterinary sciences and/or in laboratory animals.

SBT-040-G2 can comprise an amino acid sequence MDWTWRILFLVAAATGAHSQVQLVQSGAEVKKPGASVKVSCKASGYTFTGYYMHWV RQAPGQGLEWMGWINPDSGGTNYAQKFQGRVTMTRDTSISTAYMELNRLRSDDTAVY YCARDQPLGYCTNGVCSYFDYWGQGTLVTVSSASTKGPSVFPLAPCSRSTSESTAALGC LVKDYFPEPVTVSWNSGALTSGVHTFPAVLQSSGLYSLSSVVTVPSSNFGTQTYTCNVD HKPSNTKVDKTVERKCCVECPPCPAPPVAGPSVFLFPPKPKDTLMISRTPEVTCVVVDVS HEDPEVQFNWYVDGVEVHNAKTKPREEQFNSTFRVVSVLTVVHQDWLNGKEYKCKVS NKGLPAPIEKTISKTKGQPREPQVYTLPPSREEMTKNQVSLTCLVKGFYPSDIAVEWESN GQPENNYKTTPPMLDSDGSFFLYSKLTVDKSRWQQGNVFSCSVMHEALHNHYTQKSLS LSPGK (SEQ ID NO: 14). SBT-040-G2 can comprise an amino acid sequence with greater than 70%, greater than 75%, greater than 80%, greater than 85%, greater than 90%, greater than 95% or greater than 99% homology to SEQ ID NO: 14. SBT-040-G1WT can comprise an amino acid sequence SEQ ID NO: 20. A variable region of SBT-040-G2 can comprise an amino acid sequence with greater than 70%, greater than 75%, greater than 80%, greater than 85%, greater than 90%, greater than 95% or greater than 99% homology to SEQ ID NO: 20. Additionally, anti-CD40 antibodies comprising SBT-040-G2 with SEQ ID NO: 14 or with an amino acid sequence with greater than 70% homology to SEQ ID NO: 14 can have a dissociation constant (K_{d}) for CD40 that is less than 10 nM. Anti-CD40 antibodies comprising SBT-040-G2 with SEQ ID NO: 14 or with an amino acid sequence with greater than 70% homology to SEQ ID NO: 14 can have a dissociation constant (K_{d}) for CD40 that is less than 1 nM, less than 100 pM, less than 10 pM, less than 1 pM, or less than 0.1 pM. SBT-040-G2 can be purified. SBT-040-G2 can be combined with any anti-CD40 light chain or fragment thereof to form an anti-CD40 antibody or fragment thereof. The anti-CD40 antibody or fragment thereof can be purified, and can be combined with a pharmaceutically acceptable carrier. The anti-CD40 antibody can be a construct. Additionally, one skilled in the art would recognize that these same concepts could apply to constructs comprising anti-CD40 antibodies created for use in the veterinary sciences and/or in laboratory animals.

A construct can comprise an antibody heavy chain. A heavy chain can be a heavy chain of an anti-DEC205 antibody which can bind a DEC205 antigen. An anti-DEC205 antibody can comprise an anti-DEC205 variant 1 heavy chain. An anti-DEC205 variant 1 antibody or heavy chain can comprise an amino acid sequence with greater than 70%, greater than 75%, greater than 80%, greater than 85%, greater than 90%, greater than 95% or greater than 99% homology to amino acid sequence QVQLVESGGGVVQPGRSLRLSCAASGFTFSNYGMYWVRQAPGKGLEWVAVIWYDGSN KYYADSVKGRFTISRDNSKNTLYLQMNSLRAEDTAVYYCARDLWGWYFDYWGQGTL VTVSSASTKGPSVFPLAPSSKSTSGGTAALGCLVKDYFPEPVTVSWNSGALTSGVHTFPA VLQSSGLYSLSSVVTVPSSSLGTQTYICNVNHKPSNTKVDKKVEPKSCDKTHTCPPCPAP ELLGGPSVFLFPPKPKDTLMISRTPEVTCVVVDVSHEDPEVKFNWYVDGVEVHNAKTKP REEQYNSTYRVVSVLTVLHQDWLNGKEYKCKVSNKALPAPIEKTISKAKGQPREPQVYT LPPSREEMTKNQVSLTCLVKGFYPSDIAVEWESNGQPENNYKTTPPVLDSDGSFFLYSKL TVDKSRWQQGNVFSCSVMHEALHNHYTQKSLSLSPGK (SEQ ID NO: 86). An anti-DEC205 variant 1 antibody or heavy chain variable region can comprise an amino acid sequence with greater than 70%, greater than 75%, greater than 80%, greater than 85%, greater than 90%, greater than 95% or greater than 99% homology to amino acid sequence QVQLVESGGGVVQPGRSLRLSCAASGFTFSNYGMYWVRQAPGKGLEWVAVIWYDGSN KYYADSVKGRFTISRDNSKNTLYLQMNSLRAEDTAVYYCARDLWGWYFDYWGQGTL VTVSS (SEQ ID NO: 87). A heavy chain of an anti-DEC205 variant 1 antibody can comprise a CDR. A heavy chain of an anti-DEC205 variant 1 antibody can comprise a CDR with amino acid sequence GFTFSNYG (SEQ ID NO: 88). A heavy chain of an anti-DEC205 variant 1 antibody can comprise a CDR with amino acid sequence IWYDGSNK (SEQ ID NO: 89). A heavy chain of an anti-DEC205 variant 1 antibody can comprise a CDR with amino acid sequence ARDLWGWYFDY (SEQ ID NO: 90). A heavy chain CDR of an anti-DEC205 variant 1 antibody can comprise an amino acid sequence with greater than 70%, greater than 75%, greater than 80%, greater than 85%, greater than 90%, greater than 95% or greater than 99% homology to SEQ ID NO: 88. A heavy chain CDR of an anti-DEC205 variant 1 antibody can comprise an amino acid sequence with greater than 70%, greater than 75%, greater than 80%, greater than 85%, greater than 90%, greater than 95% or greater than 99% homology to SEQ ID NO: 89. A heavy chain CDR of an anti-DEC205 variant 1 antibody can comprise an amino acid sequence with greater than 70%, greater than 75%, greater than 80%, greater than 85%, greater than 90%, greater than 95% or greater than 99% homology to SEQ ID NO: 90.

A construct can comprise an antibody light chain. A light chain can be a light chain of an anti-DEC205 antibody which can bind a DEC205 antigen. An anti-DEC205 antibody can comprise an anti-DEC205 variant 1 light chain. An anti-DEC205 variant 1 antibody or light chain can comprise an amino acid sequence with greater than 70%, greater than 75%, greater than 80%, greater than 85%, greater than 90%, greater than 95% or greater than 99% homology to amino acid sequence EIVLTQSPATLSLSPGERATLSCRASQSVSSYLAWYQQKPGQAPRLLIYDASNRATGIPAR FSGSGSGTDFTLTISSLEPEDFAVYYCQQRRNWPLTFGGGTKVEIK (SEQ ID NO: 91). An anti-DEC205 variant 1 antibody or light chain variable domain can comprise an amino acid sequence with greater than 70%, greater than 75%, greater than 80%, greater than 85%, greater than 90%, greater than 95% or greater than 99% homology to amino acid sequence EIVLTQSPATLSLSPGERATLSCRASQSVSSYLAWYQQKPGQAPRLLIYDASNRATGIPAR FSGSGSGTDFTLTISSLEPEDFAVYYCQQRRNWPLTFGGGTKVEIK (SEQ ID NO: 92). A light chain of an anti-DEC205 variant 1 antibody can comprise a CDR. A light chain of an anti-DEC205 variant 1 antibody can comprise a CDR with amino acid sequence QSVSSY (SEQ ID NO: 93). A light chain of an anti-DEC205 variant 1 antibody can comprise a CDR with amino acid sequence DAS (SEQ ID NO: 94). A light chain of an anti-DEC205 variant 1 antibody can comprise a CDR with amino acid sequence QQRRNWPLT (SEQ ID NO: 95). A light chain CDR of an anti-anti-DEC205 variant 1 antibody can comprise an amino acid sequence with greater than 70%, greater than 75%, greater than 80%, greater than 85%, greater than 90%, greater than 95% or greater than 99% homology to SEQ ID NO: 93. A light chain CDR of an anti-anti-DEC205 variant 1 antibody can comprise an amino acid sequence with greater than 70%, greater than 75%, greater than 80%, greater than 85%, greater than 90%, greater than 95% or greater than 99% homology to SEQ ID NO: 94. A light chain CDR of an anti-anti-DEC205 variant 1 antibody can comprise an amino acid sequence with greater than 70%, greater than 75%, greater than 80%, greater than 85%, greater than 90%, greater than 95% or greater than 99% homology to SEQ ID NO: 95.

A construct can comprise an antibody heavy chain. A heavy chain can be a heavy chain of an anti-DEC205 antibody which can bind a DEC205 antigen. An anti-DEC205 antibody can comprise an anti-DEC205 variant 2 heavy chain. An anti-DEC205 variant 2 antibody or heavy chain can comprise an amino acid sequence with greater than 70%, greater than 75%, greater than 80%, greater than 85%, greater than 90%, greater than 95% or greater than 99% homology to amino acid sequence EVQLVQSGAEVKKPGESLRISCKGSGDSFTTYWIGWVRQMPGKGLEWMGIIYPGDSDTI YSPSFQGQVTISADKSISTAYLQWSSLKASDTAMYYCTRGDRGVDYWGQGTLVTVSSA STKGPSVFPLAPSSKSTSGGTAALGCLVKDYFPEPVTVSWNSGALTSGVHTFPAVLQSSG LYSLSSVVTVPSSSLGTQTYICNVNHKPSNTKVDKKVEPKSCDKTHTCPPCPAPELLGGP SVFLFPPKPKDTLMISRTPEVTCVVVDVSHEDPEVKFNWYVDGVEVHNAKTKPREEQY NSTYRVVSVLTVLHQDWLNGKEYKCKVSNKALPAPIEKTISKAKGQPREPQVYTLPPSR EEMTKNQVSLTCLVKGFYPSDIAVEWESNGQPENNYKTTPPVLDSDGSFFLYSKLTVDK SRWQQGNVFSCSVMHEALHNHYTQKSLSLSPGK (SEQ ID NO: 96). An anti-DEC205 variant 2 antibody or heavy chain variable region can comprise an amino acid sequence with greater than 70%, greater than 75%, greater than 80%, greater than 85%, greater than 90%, greater than 95% or greater than 99% homology to amino acid sequence EVQLVQSGAEVKKPGESLRISCKGSGDSFTTYWIGWVRQMPGKGLEWMGIIYPGDSDTI YSPSFQGQVTISADKSISTAYLQWSSLKASDTAMYYCTRGDRGVDYWGQGTLVTVSS (SEQ ID NO: 97). A heavy chain of an anti-DEC205 variant 1 antibody can comprise a CDR. A heavy chain of an anti-DEC205 variant 2 antibody can comprise a CDR with amino acid sequence GDSFTTYW (SEQ ID NO: 98). A heavy chain of an anti-DEC205 variant 2 antibody can comprise a CDR with amino acid sequence IYPGDSDT (SEQ ID NO: 99). A heavy chain of an anti-DEC205 variant 2 antibody can comprise a CDR with amino acid sequence TRGDRGVDY (SEQ ID NO: 100). A heavy chain CDR of an anti-anti-DEC205 variant 2 antibody can comprise an amino acid sequence with greater than 70%, greater than 75%, greater than 80%, greater than 85%, greater than 90%, greater than 95% or greater than 99% homology to SEQ ID NO: 98. A heavy chain CDR of an anti-anti-DEC205 variant 2 antibody can comprise an amino acid sequence with greater than 70%, greater than 75%, greater than 80%, greater than 85%, greater than 90%, greater than 95% or greater than 99% homology to SEQ ID NO: 99. A heavy chain CDR of an anti-anti-DEC205 variant 2 antibody can comprise an amino acid sequence with greater than 70%, greater than 75%, greater than 80%, greater than 85%, greater than 90%, greater than 95% or greater than 99% homology to SEQ ID NO: 100.

A construct can comprise an antibody light chain. A light chain can be a light chain of an anti-DEC205 antibody which can bind a DEC205 antigen. An anti-DEC205 antibody can comprise an anti-DEC205 variant 2 light chain. An anti-DEC205 variant 2 antibody or light chain can comprise an amino acid sequence with greater than 70%, greater than 75%, greater than 80%, greater than 85%, greater than 90%, greater than 95% or greater than 99% homology to amino acid sequence

(SEQ ID NO: 101)
DIQMTQSPSSLSASVGDRVTITCRASQGISRWLAWYQQKPEKAPKSLIY
AASSLQSGVPSRFSGSGSGTDFTLTISGLQPEDFATYYCQQYNSYPRTF
GQGTKVEIK.

An anti-DEC205 variant 2 antibody or light chain variable region can comprise an amino acid sequence with greater than 70%, greater than 75%, greater than 80%, greater than 85%, greater than 90%, greater than 95% or greater than 99% homology to amino acid sequence

(SEQ ID NO: 102)
DIQMTQSPSSLSASVGDRVTITCRASQGISRWLAWYQQKPEKAPKSLIY
AASSLQSGVPSRFSGSGSGTDFTLTISGLQPEDFATYYCQQYNSYPRTF
GQGTKVEIK.

A light chain of an anti-DEC205 variant 2 antibody can comprise a CDR. A light chain of an anti-DEC205 variant 2 antibody can comprise a CDR with amino acid sequence QGISRW (SEQ ID NO: 103). A light chain of an anti-DEC205 variant 2 antibody can comprise a CDR with amino acid sequence AAS (SEQ ID NO: 104). A light chain of an anti-DEC205 variant 2 antibody can comprise a CDR with amino acid sequence QQYNSYPRT (SEQ ID NO: 105). A light chain CDR of an anti-anti-DEC205 variant 2 antibody can comprise an amino acid sequence with greater than 70%, greater than 75%, greater than 80%, greater than 85%, greater than 90%, greater than 95% or greater than 99% homology to SEQ ID NO: 103. A light chain CDR of an anti-anti-DEC205 variant 2 antibody can comprise an amino acid sequence with greater than 70%, greater than 75%, greater than 80%, greater than 85%, greater than 90%, greater than 95% or greater than 99% homology to SEQ ID NO: 104. A light chain CDR of an anti-anti-DEC205 variant 2 antibody can comprise an amino acid sequence with greater than 70%, greater than 75%, greater than 80%, greater than 85%, greater than 90%, greater than 95% or greater than 99% homology to SEQ ID NO: 105.

A construct can comprise an antibody heavy chain. A heavy chain can be a heavy chain of an anti-DC-SIGN antibody which can bind a DC-SIGN antigen. An anti-DC-SIGN antibody can comprise an anti-DC-SIGN heavy chain. An anti-DC-SIGN antibody can comprise an anti-DC-SIGN heavy chain variable region. A heavy chain of an anti-DC-SIGN antibody can comprise a variant 1 CDR with amino acid sequence QHFWNTPWT (SEQ ID NO: 106). A heavy chain of an anti-DC-SIGN antibody can comprise a variant 1 CDR with amino acid sequence QQGHTLPYT (SEQ ID NO: 107). A heavy chain of an anti-DC-SIGN antibody can comprise a variant 1 CDR with amino acid sequence SNDGYYS (SEQ ID NO: 108). A heavy chain of an anti-DC-SIGN antibody can comprise a variant 2 CDR with amino acid sequence YYGIYVDY SEQ ID (NO: 109). A heavy chain of an anti-DC-SIGN antibody can comprise a variant 2 CDR with FLVY amino acid sequence (SEQ ID NO: 110). A heavy chain of an anti-DC-SIGN antibody can comprise a variant 2 CDR with amino acid sequence NFGILGY (SEQ ID NO: 111). A heavy chain of an anti-DC-SIGN antibody can comprise a variant 3 CDR with amino acid sequence QQGNTLPPT (SEQ ID NO: 112). A heavy chain of an anti-DC-SIGN antibody can comprise a variant 3 CDR with amino acid sequence QQHYITPLT (SEQ ID NO: 113). A heavy chain of an anti-DC-SIGN antibody can comprise a variant 3 CDR with amino acid sequence QQYGNLPYT (SEQ ID NO: 114). A heavy chain variant 1 CDR of an anti-DC-SIGN antibody can comprise an amino acid sequence with greater than 70%, greater than 75%, greater than 80%, greater than 85%, greater than 90%, greater than 95% or greater than 99% homology to SEQ ID NO: 106. A heavy chain variant 1 CDR of an anti-DC-SIGN antibody can comprise an amino acid sequence with greater than 70%, greater than 75%, greater than 80%, greater than 85%, greater than 90%, greater than 95% or greater than 99% homology to SEQ ID NO: 107. A heavy chain variant 1 CDR of an anti-DC-SIGN antibody can comprise an amino acid sequence with greater than 70%, greater than 75%, greater than 80%, greater than 85%, greater than 90%, greater than 95% or greater than 99% homology to SEQ ID NO: 108. A heavy chain variant 2 CDR of an anti-DC-SIGN antibody can comprise an amino acid sequence with greater than 70%, greater than 75%, greater than 80%, greater than 85%, greater than 90%, greater than 95% or greater than 99% homology to SEQ ID NO: 109. A heavy chain variant 2 CDR of an anti-DC-SIGN antibody can comprise an amino acid sequence with greater than 70%, greater than 75%, greater than 80%, greater than 85%, greater than 90%, greater than 95% or greater than 99% homology to SEQ ID NO: 110. A heavy chain variant 2 CDR of an anti-DC-SIGN antibody can comprise an amino acid sequence with greater than 70%, greater than 75%, greater than 80%, greater than 85%, greater than 90%, greater than 95% or greater than 99% homology to SEQ ID NO: 111. A heavy chain variant 3 CDR of an anti-DC-SIGN antibody can comprise an amino acid sequence with greater than 70%, greater than 75%, greater than 80%, greater than 85%, greater than 90%, greater than 95% or greater than 99% homology to SEQ ID NO: 112. A heavy chain variant 3 CDR of an anti-DC-SIGN antibody can comprise an amino acid sequence with greater than 70%, greater than 75%, greater than 80%, greater than 85%, greater than 90%, greater than 95% or greater than 99% homology to SEQ ID NO: 113. A heavy chain variant 3 CDR of an anti-DC-SIGN antibody can comprise an amino acid sequence with greater than 70%, greater than 75%, greater than 80%, greater than 85%, greater than 90%, greater than 95% or greater than 99% homology to SEQ ID NO: 114.

A construct can comprise an antibody light chain. A light chain can be a light chain of an anti-DC-SIGN antibody which can bind a DC-SIGN antigen. An anti-DC-SIGN antibody can comprise an anti-DC-SIGN light chain. An anti-DC-SIGN antibody can comprise an anti-DC-SIGN light chain variable region. A light chain of an anti-DC-SIGN antibody can comprise a variant 1 CDR with amino acid sequence RYYLGVD (SEQ ID NO: 115). A light chain of an anti-DC-SIGN antibody can comprise a variant 1 CDR with amino acid sequence DDSGRFP (SEQ ID NO: 116). A light chain of an anti-DC-SIGN antibody can comprise a variant 1 CDR with amino acid sequence YGYAVDY (SEQ ID NO: 117). A light chain of an anti-DC-SIGN antibody can comprise a variant 2 CDR with YPNALDY amino acid sequence (SEQ ID NO: 118). A light chain of an anti-DC-SIGN antibody can comprise a variant 2 CDR with amino acid sequence GLKSFYAMDH (SEQ ID NO: 119). A light chain of an anti-DC-SIGN antibody can comprise a variant 2 CDR with amino acid sequence QQGKTLPWT (SEQ ID NO: 120). A light chain of an anti-DC-SIGN antibody can comprise a variant 3 CDR with amino acid sequence QQYYSTPRT (SEQ ID NO: 121). A light chain of an anti-DC-SIGN antibody can comprise a variant 3 CDR with amino acid sequence GQSYNYPPT (SEQ ID NO: 122). A light chain of an anti-DC-SIGN antibody can comprise a variant 3 CDR with amino acid sequence WQDTHFPHV (SEQ ID NO: 123). A light chain variant 1 CDR of an anti-DC-SIGN antibody can comprise an amino acid sequence with greater than 70%, greater than 75%, greater than 80%, greater than 85%, greater than 90%, greater than 95% or greater than 99% homology to SEQ ID NO: 115. A light chain variant 1 CDR of an anti-DC-SIGN antibody can comprise an amino acid sequence with greater than 70%, greater than 75%, greater than 80%, greater than 85%, greater than 90%, greater than 95% or greater than 99% homology to SEQ ID NO: 116. A light chain variant 1 CDR of an anti-DC-SIGN antibody can comprise an amino acid sequence with greater than 70%, greater than 75%, greater than 80%, greater than 85%, greater than 90%, greater than 95% or greater than 99% homology to SEQ ID NO: 117. A light chain variant 2 CDR of an anti-DC-SIGN antibody can comprise an amino acid sequence with greater than 70%, greater than 75%, greater than 80%, greater than 85%, greater than 90%, greater than 95% or greater than 99% homology to SEQ ID NO: 118. A light chain variant 2 CDR of an anti-DC-SIGN antibody can comprise an amino acid sequence with greater than 70%, greater than 75%, greater than 80%, greater than 85%, greater than 90%, greater than 95% or greater than 99% homology to SEQ ID NO: 119. A light chain variant 2 CDR of an anti-DC-SIGN antibody can comprise an amino acid sequence with greater than 70%, greater than 75%, greater than 80%, greater than 85%, greater than 90%, greater than 95% or greater than 99% homology to SEQ ID NO: 120. A light chain variant 3 CDR of an anti-DC-SIGN antibody can comprise an amino acid sequence with greater than 70%, greater than 75%, greater than 80%, greater than 85%, greater than 90%, greater than 95% or greater than 99% homology to SEQ ID NO: 121. A light chain variant 3 CDR of an anti-DC-SIGN antibody can comprise an amino acid sequence with greater than 70%, greater than 75%, greater than 80%, greater than 85%, greater than 90%, greater than 95% or greater than 99% homology to SEQ ID NO: 122. A light chain variant 3 CDR of an anti-DC-SIGN antibody can comprise an amino acid sequence with greater than 70%, greater than 75%, greater than 80%, greater than 85%, greater than 90%, greater than 95% or greater than 99% homology to SEQ ID NO: 123.

A construct can comprise an antibody heavy chain. A heavy chain can be a heavy chain of an anti-CD36 mannose scavenger receptor 1 antibody which can bind a CD36 mannose scavenger receptor lantigen. An anti-CD36 mannose scavenger receptor 1 antibody can comprise an anti-CD36 mannose scavenger receptor 1 heavy chain. An anti-CD36 mannose scavenger receptor 1 antibody can comprise an anti-CD36 mannose scavenger receptor 1 heavy chain variable region comprising amino acid sequence

(SEQ ID NO: 124)
DIQMTQSPSSLSASVGDRVTITCRASQGISRWLAWYQQKPEKAPKSLIY
AASSLQSGVPSRFSGSGSGTDFTLTISGLQPEDFATYYCQQYNSYPRTF
GQGTKVEIK.

An anti-CD36 mannose scavenger receptor 1 heavy chain variable region can comprise a V_{H} sequence having at least 80% sequence identity to an amino acid sequence of SEQ ID NO: 124. A heavy chain of an anti-CD36 antibody mannose scavenger receptor 1 can comprise an anti-CD36 CDR.

A construct can comprise an antibody light chain. A light chain can be a light chain of an anti-CD36 mannose scavenger receptor 1 antibody which can bind a CD36 mannose scavenger receptor lantigen. An anti-CD36 mannose scavenger receptor 1 antibody can comprise an anti-CD36 mannose scavenger receptor 1 light chain. An anti-CD36 mannose scavenger receptor 1 antibody can comprise an anti-CD36 mannose scavenger receptor 1 light chain variable region comprising amino acid sequence EVQLVQSGAEVKKPGESLRISCKGSGDSFTTYWIGWVRQMPGKGLEWMGIIYPGDSDTI YSPSFQGQVTISADKSISTAYLQWSSLKASDTAMYYCTRGDRGVDYWGQGTLVTVSS (SEQ ID NO: 125). An anti-CD36 mannose scavenger receptor 1 light chain variable region can comprise a V_{H} sequence having at least 80% sequence identity to an amino acid sequence of SEQ ID NO: 125. A heavy chain of an anti-CD36 mannose scavenger receptor 1 antibody can comprise an anti-CD36 mannose scavenger receptor 1 CDR.

A construct can comprise an antibody heavy chain. A heavy chain can be a heavy chain of an anti-DNGR-1 antibody which can bind a DNGR-1 antigen. An anti-DNGR-1 antibody can comprise an anti-DNGR-1 heavy chain. An anti-DNGR-1 antibody can comprise an anti-DNGR-1 heavy chain variable region comprising amino acid sequence QIVESGGGLVQPKESLKISCTASGFTFSNAAIYWVRQTPGKGLEWVGRIRTRPSKYATDY ADSVRGRFTISRDDSKSMVYLQMDNLRTEDTAMYYCTPRATEDVPFYWGQGVMVTVS S (SEQ ID NO: 126). An anti-DNGR-1 heavy chain variable region can comprise a V_{H} sequence having at least 80% sequence identity to an amino acid sequence of SEQ ID NO: 126. A heavy chain of an anti-DNGR-1 can comprise an anti-DNGR-1 CDR.

A construct can comprise an antibody light chain. A light chain can be a light chain of an anti-DNGR-1antibody which can bind a DNGR-1 antigen. An anti-DNGR-1 antibody can comprise an anti-DNGR-1 light chain. An anti-DNGR-1 antibody can comprise an anti-DNGR-1 light chain variable region comprising amino acid sequence DIVMTQTPSSQAVSAGEKVTMNCKSSQSVLYDENKKNYLAWYQQKSGQSPKLLIYWAS TGESGVPDRFIGSGSGTDFTLTISSVQAEDLAVYYCQQYYDFPPTFGGGTK (SEQ ID NO: 127). An anti-DNGR-1 light chain variable region can comprise a V_{H} sequence having at least 80% sequence identity to an amino acid sequence of SEQ ID NO: 127. A heavy chain of an anti-DNGR-1 can comprise an anti-DNGR-1 CDR.

A construct can comprise an antibody heavy chain. A heavy chain can be a heavy chain of an anti-CLEC12A antibody which can bind a CLEC12A antigen. A heavy chain of an anti-CLEC12A antibody can be an anti-CLEC12 variant 1 antibody. An anti-CLEC12 variant 1 antibody or heavy chain can comprise an amino acid sequence with greater than 70%, greater than 75%, greater than 80%, greater than 85%, greater than 90%, greater than 95% or greater than 99% homology to amino acid sequence QVQLQESGPGLVKPSETLSLTCVVSGGSISSSNWWSWVRQPPGKGLEWIGEIYHSGSPDY NPSLKSRVTISVDKSRNQFSLKLSSVTAADTAVYYCAKVSTGGFFDYWGQGTLVTVSSA STKGPSVFPLAPSSKSTSGGTAALGCLVKDYFPEPVTVSWNSGALTSGVHTFPAVLQSSG LYSLSSVVTVPSSSLGTQTYICNVNHKPSNTKVDKRVEPKSCDKTHTCPPCPAPELLGGP SVFLFPPKPKDTLMISRTPEVTCVVVDVSHEDPEVKFNWYVDGVEVHNAKTKPREEQY NSTYRVVSVLTVLHQDWLNGKEYKCKVSNKALPAPIEKTISKAKGQPREPQVYTLPPSR EEMTKNQVSLTCLVKGFYPSDIAVEWESNGQPENNYKTTPPVLDSDGSFFLYSKLTVDK SRWQQGNVFSCSVMHEALHNHYTQKSLSLSPGK (SEQ ID NO: 128). A heavy chain of an anti-CLEC12A antibody can be an anti-CLEC12A variant 2 antibody. An anti-CLEC12A variant 2 antibody or heavy chain can comprise an amino acid sequence with greater than 70%, greater than 75%, greater than 80%, greater than 85%, greater than 90%, greater than 95% or greater than 99% homology to amino acid sequence QVQLQESGPGLVKPSETLSLTCVVSGGSISSSNWWSWVRQPPGKGLEWIGEIYHSGSPNY NPSLKSRVTISVDKSKNQFSLKLSSVTAADTAVYYCARSSSGGFFDYWGQGTLVTVSSA STKGPSVFPLAPSSKSTSGGTAALGCLVKDYFPEPVTVSWNSGALTSGVHTFPAVLQSSG LYSLSSVVTVPSSSLGTQTYICNVNHKPSNTKVDKRVEPKSCDKTHTCPPCPAPELLGGP SVFLFPPKPKDTLMISRTPEVTCVVVDVSHEDPEVKFNWYVDGVEVHNAKTKPREEQY NSTYRVVSVLTVLHQDWLNGKEYKCKVSNKALPAPIEKTISKAKGQPREPQVYTLPPSR EEMTKNQVSLTCLVKGFYPSDIAVEWESNGQPENNYKTTPPVLDSDGSFFLYSKLTVDK SRWQQGNVFSCSVMHEALHNHYTQKSLSLSPGK (SEQ ID NO: 129). A heavy chain of an anti-CLEC12A antibody can be an anti-CLEC12A variant 3 antibody. An anti-CLEC12A variant 3 antibody or heavy chain can comprise an amino acid sequence with greater than 70%, greater than 75%, greater than 80%, greater than 85%, greater than 90%, greater than 95% or greater than 99% homology to amino acid sequence QVQLQESGPGLVKPSETLSLTCVVSGGSISSSNWWSWVRQPPGKGLEWIGEIYHSGSPNY NPSLKSRVTISVDKSKNQFSLKLSSVTAADTAVYYCARQTTAGSFDYWGQGTLVTVSSA STKGPSVFPLAPSSKSTSGGTAALGCLVKDYFPEPVTVSWNSGALTSGVHTFPAVLQSSG LYSLSSVVTVPSSSLGTQTYICNVNHKPSNTKVDKRVEPKSCDKTHTCPPCPAPELLGGP SVFLFPPKPKDTLMISRTPEVTCVVVDVSHEDPEVKFNWYVDGVEVHNAKTKPREEQY NSTYRVVSVLTVLHQDWLNGKEYKCKVSNKALPAPIEKTISKAKGQPREPQVYTLPPSR EEMTKNQVSLTCLVKGFYPSDIAVEWESNGQPENNYKTTPPVLDSDGSFFLYSKLTVDK SRWQQGNVFSCSVMHEALHNHYTQKSLSLSPGK (SEQ ID NO: 130). An anti-CLEC12 antibody can comprise an anti-CLEC12A heavy chain variable region. A heavy chain of an anti-CLEC12A antibody can comprise an anti-CLEC12A CDR.

A construct can comprise an antibody light chain. A light chain can be a light chain of an anti-CLEC12A antibody which can bind a CLEC12A antigen. A light chain of an anti-CLEC12A antibody can be an anti-CLEC12 variant 1 antibody. An anti-CLEC12 variant 1 antibody or light chain can comprise an amino acid sequence with greater than 70%, greater than 75%, greater than 80%, greater than 85%, greater than 90%, greater than 95% or greater than 99% homology to amino acid sequence amino acid sequence DIQMTQSPSSLSASVGDRVTITCRASQSISSYLNWYQQKPGKAPKLLIYAASSLQSGVPSR FSGSGSGTDFTLTISSLQPEDFATYYCQQSYSTPPTFGQGTKVEIKRTVAAPSVFIFPPSDE QLKSGTASVVCLLNNFYPREAKVQWKVDNALQSGNSQESVTEQDSKDSTYSLSSTLTLS KADYEKHKVYACEVTHQGLSSPVTKSFNRGEC (SEQ ID NO: 131). An anti-CLEC12 antibody can comprise an anti-CLEC12A light chain variable region. A light chain of an anti-CLEC12A antibody can comprise an anti-CLEC12A CDR.

A construct can comprise an antibody heavy chain. A heavy chain can be a heavy chain of an anti-BDCA-2 antibody which can bind a BDCA-2 antigen. A heavy chain of an anti-BDCA-2 antibody can be an anti-BDCA-2 variant 1 antibody. An anti-BDCA-2 variant 1 antibody or heavy chain can comprise an amino acid sequence with greater than 70%, greater than 75%, greater than 80%, greater than 85%, greater than 90%, greater than 95% or greater than 99% homology to amino acid sequence QVQLVESGGGVVQPGRSLRLSCAASGFTLSSYGMHWVRQAPGKGLEWVAVIWYDGND KYYADSVKGRFTISRDNSKNTLYLQVNSLRAEDTAVYYCARGTGTPYWYFDLWGRGTL VTVSSASTKGPSVFPLAPSSKSTSGGTAALGCLVKDYFPEPVTVSWNSGALTSGVHTFPA VLQSSGLYSLSSVVTVPSSSLGTQTYICNVNHKPSNTKVDKKVEPKSCDKTHTCPPCPAP ELLGGPSVFLFPPKPKDTLMISRTPEVTCVVVDVSHEDPEVKFNWYVDGVEVHNAKTKP REEQYNSTYRVVSVLTVLHQDWLNGKEYKCKVSNKALPAPIEKTISKAKGQPREPQVYT LPPSRDELTKNQVSLTCLVKGFYPSDIAVEWESNGQPENNYKTTPPVLDSDGSFFLYSKL TVDKSRWQQGNVFSCSVMHEALHNHYTQKSLSLSPGK (SEQ ID NO: 132). A heavy chain of an anti-BDCA-2 antibody can be an anti-BDCA-2 variant 2 antibody. An anti-BDCA-2 variant 2 antibody or heavy chain can comprise an amino acid sequence with greater than 70%, greater than 75%, greater than 80%, greater than 85%, greater than 90%, greater than 95% or greater than 99% homology to amino acid sequence EVQLVESGGGLVQPGGSLRLSCAASGFTFSNYLMNWVRQAPGKGLEWVANIEQDGSEK YYVDSVKGRFTISRDNAKNSLYLQMNSLRAEDTAVYFCARDGDTAMITFDFWGQGTLV TVSSASTKGPSVFPLAPSSKSTSGGTAALGCLVKDYFPEPVTVSWNSGALTSGVHTFPAV LQSSGLYSLSSVVTVPSSSLGTQTYICNVNHKPSNTKVDKKVEPKSCDKTHTCPPCPAPE LLGGPSVFLFPPKPKDTLMISRTPEVTCVVVDVSHEDPEVKFNWYVDGVEVHNAKTKPR EEQYNSTYRVVSVLTVLHQDWLNGKEYKCKVSNKALPAPIEKTISKAKGQPREPQVYTL PPSRDELTKNQVSLTCLVKGFYPSDIAVEWESNGQPENNYKTTPPVLDSDGSFFLYSKLT VDKSRWQQGNVFSCSVMHEALHNHYTQKSLSLSPGK (SEQ ID NO: 133). A heavy chain of an anti-BDCA-2 antibody can be an anti-BDCA-2 variant 3 antibody. An anti-BDCA-2 variant 3 antibody or heavy chain can comprise an amino acid sequence with greater than 70%, greater than 75%, greater than 80%, greater than 85%, greater than 90%, greater than 95% or greater than 99% homology to amino acid sequence QVQLQESGPGLVKPSQTLSLTCTVSGGSISSGGYYWNWIRQHPGKGLEWIGYIYYSGNT YYNPSLKSRVTISVDTSKNQFSLKLSSVTAADAAVYHCARGYGDYGGGYFDYWGQGTL VTVSSASTKGPSVFPLAPSSKSTSGGTAALGCLVKDYFPEPVTVSWNSGALTSGVHTFPA VLQSSGLYSLSSVVTVPSSSLGTQTYICNVNHKPSNTKVDKKVEPKSCDKTHTCPPCPAP ELLGGPSVFLFPPKPKDTLMISRTPEVTCVVVDVSHEDPEVKFNWYVDGVEVHNAKTKP REEQYNSTYRVVSVLTVLHQDWLNGKEYKCKVSNKALPAPIEKTISKAKGQPREPQVYT LPPSRDELTKNQVSLTCLVKGFYPSDIAVEWESNGQPENNYKTTPPVLDSDGSFFLYSKL TVDKSRWQQGNVFSCSVMHEALHNHYTQKSLSLSPGK (SEQ ID NO: 134). An anti-BDCA-2 antibody can comprise an anti-BDCA-2 heavy chain variable region. A heavy chain of an anti-BDCA-2 antibody can comprise an anti-BDCA-2 CDR.

A construct can comprise an antibody light chain. A light chain can be a light chain of an anti-BDCA-2 antibody which can bind a BDCA-2 antigen. A light chain of an anti-BDCA-2 antibody can be an anti-BDCA-2 variant 1 light chain. An anti-BDCA-2 variant 1 antibody or light chain can comprise an amino acid sequence with greater than 70%, greater than 75%, greater than 80%, greater than 85%, greater than 90%, greater than 95% or greater than 99% homology to amino acid sequence EIVLTQSPATLSLSPGERATLSCRASQSVNNYLAWYQQKPGQAPRLLIYDASNRATGIPA RFSGSGSGTDFTLTISSLEPEDFAVYYCQQRSTWPPYTFGQGTKLEIKRTVAAPSVFIFPPS DEQLKSGTASVVCLLNNFYPREAKVQWKVDNALQSGNSQESVTEQDSKDSTYSLSSTLT LSKADYEKHKVYACEVTHQGLSSPVTKSFNRGEC (SEQ ID NO: 135). A light chain of an anti-BDCA-2 antibody can be an anti-BDCA-2 variant 2 antibody. An anti-BDCA-2 variant 2 antibody or light chain can comprise an amino acid sequence with greater than 70%, greater than 75%, greater than 80%, greater than 85%, greater than 90%, greater than 95% or greater than 99% homology to amino acid sequence DIQMTQSPSSVSASVGDRVTITCRASQGIRRWLAWYQQKPGKAPKLLIYAASSLQRGVP SRFSGSGSGTDFTLTISSLQPEDFATYYCQQANSFPWTFGQGTKVEIKRTVAAPSVFIFPPS DEQLKSGTASVVCLLNNFYPREAKVQWKVDNALQSGNSQESVTEQDSKDSTYSLSSTLT LSKADYEKHKVYACEVTHQGLSSPVTKSFNRGEC (SEQ ID NO: 136). A light chain of an anti-BDCA-2 antibody can be an anti-BDCA-2 variant 3 antibody. An anti-BDCA-2 variant 3 antibody or light chain can comprise an amino acid sequence with greater than 70%, greater than 75%, greater than 80%, greater than 85%, greater than 90%, greater than 95% or greater than 99% homology to amino acid sequence DIQMTQSPSSLSASVGDRVTITCQASQDISNYLNWYQQKPGKAPKFLIYDVSNLETGVPS RFSGSGSGTDFTFTISSLQPEDIATYYCQQYDNLPYTFGQGTKLEIKRTVAAPSVFIFPPSD EQLKSGTASVVCLLNNFYPREAKVQWKVDNALQSGNSQESVTEQDSKDSTYSLSSTLTL SKADYEKHKVYACEVTHQGLSSPVTKSFNRGEC (SEQ ID NO: 137). An anti-BDCA-2 can comprise an anti-BDCA-2 light chain variable region. A light chain of an anti-BDCA-2 antibody can comprise an anti-BDCA-2 CDR.

A construct can comprise an antibody with modifications occurring at least at one amino acid residue. Modifications can be substitutions, additions, mutations, deletions, or the like. An antibody modification can be an insertion of an unnatural amino acid.

A construct can comprise a light chain of an amino acid sequence having at least one, two, three, four, five, six, seven, eight, nine, or ten modifications but not more than 40, 35, 30, 25, 20, 15 or 10 modifications of the amino acid sequence relative to the natural or original amino acid sequence. A construct can comprise a heavy chain of an amino acid sequence having at least one, two, three, four, five, six, seven, eight, nine or ten modifications but not more than 40, 35, 30, 25, 20, 15 or 10 modifications of the amino acid sequence relative to the natural or original amino acid sequence. A heavy chain can be the heavy chain of an anti-CD40 antibody which can bind to the CD40 antigen.

A construct can be an IgG1 isotype. A construct can be an IgG2 isotype. A construct can be an IgG3 isotype. A construct can be an IgG4 isotype. A construct can be of a hybrid isotype comprising constant regions from two or more isotypes. A construct can be an anti-CD40 antibody, in which the anti-CD40 antibody can be a monoclonal human antibody comprising a wild-type sequence of an IgG1 isoform, in particular, at an Fc region of the antibody.

Constructs disclosed herein can be non-natural, designed, and/or engineered. Constructs disclosed herein can be non-natural, designed, and/or engineered scaffolds comprising an antigen binding domain. Constructs disclosed herein can be non-natural, designed, and/or engineered antibodies. Constructs can be monoclonal antibodies. Antibody constructs can be human antibodies. Constructs can be humanized antibodies. Antibody constructs can be monoclonal humanized antibodies. Constructs can be recombinant antibodies.

Sequences that can be used to produce antibodies for constructs can include leader sequences. Any of the sequences provided herein can be used with or without a leader sequence in a construct or conjugate as described herein. Leader sequences can be signal sequences. Leader sequences useful with the compositions and methods described herein can include, but are not limited to, a DNA sequence comprising ATGAGGCTCCCTGCTCAGCTCCTGGGGCTCCTGCTGCTCTGGTTCCCAGGTTCCAGAT GC (SEQ ID NO: 2) or ATGGACTGGACCTGGAGGATCCTCTTCTTGGTGGCAGCCACAGGAGCCCACTCC (SEQ ID NO: 12), or an amino acid sequence comprising MRLPAQLLGLLLLWFPGSRC (SEQ ID NO: 5) and MDWTWRILFLVAAATGAHS (SEQ ID NO: 19). A leader sequence can comprise a DNA sequence with greater than 70%, greater than 75%, greater than 80%, greater than 85%, greater than 90%, greater than 95% or greater than 99% homology to SEQ ID NO: 2 or SEQ ID NO: 12. A leader sequence can comprise an amino acid sequence with greater than 70%, greater than 75%, greater than 80%, greater than 85%, greater than 90%, greater than 95% or greater than 99% homology to SEQ ID NO: 5 or SEQ ID NO: 19. Additionally, one skilled in the art would recognize that these same concepts can apply to anti-CD40 antibodies created for use in the veterinary sciences and/or in laboratory animals.

An antigen binding domain of a construct can be selected in order to recognize an antigen. An antigen binding domain of a construct can be selected in order to recognize a target antigen. For example, an antigen can be a cell surface marker on target cells associated with a disease or condition. For example, a target antigen can be a cell surface marker on target cells associated with a disease or condition. An antigen can be expressed on an immune cell. A target antigen can be expressed on an immune cell. An antigen can be a protein or fragment thereof. A target antigen can be a protein or fragment thereof. An antigen can be expressed on an antigen-presenting cell. A target antigen can be expressed on an antigen-presenting cell. An antigen can be expressed on a dendritic cell, a macrophage, or a B cell. A target antigen can be expressed on a dendritic cell, a macrophage, or a B cell. An antigen can be CD40 and an antigen binding domain can recognize a CD40 antigen. A target antigen can be CD40 and an antigen binding domain can recognize a CD40 antigen. An antigen can be or can be at least 80% homologous to CD40, OX40L, 4-1BBL, CD36, CD204, MARCO, CLEC9A, Dectin 1, Dectin 2, CLEC10A, CD206, CD64, CD32a, CD16a, HVEM, or CD32b. A target antigen can be or can be at least 80% homologous to CD40, OX40L, 4-1BBL, CD36, CD204, MARCO, CLEC9A, Dectin 1, Dectin 2, CLEC10A, CD206, CD64, CD32a, CD16a, HVEM, or CD32b. In some embodiments, an amino acid sequence of the antigen expressed on an immune cell is selected from the group consisting of CD40, DEC-205, DICR, DNGR-1, BDCA-2, CD36 mannose scavenger receptor 1, CLEC12A, DC-SIGN, OX40L, 4-1BBL, CD36, CD204, MARCO, CLEC9A, Dectin 1, Dectin 2, CLEC10A, CD206, CD64, CD32a, CD16a, HVEM, and CD32b.

A target antigen can be a tumor antigen. Herein, a tumor antigen can be used interchangeably with a tumor associated antigen. A tumor antigen can be any antigen listed on tumor antigen databases, such as TANTIGEN, or peptide databases for T cell-defined tumor antigens, such as the Cancer Immunity Peptide database. A tumor antigen can also be any antigen listed in the review by Chen (Chen, Cancer Immun 2004 [updated 2004 Mar. 10; cited 2004 Apr. 1]). The antigen binding domain can recognize a ‘tumor antigen’. An antigen can be or can be at least 80% homologous to CD5, CD19, CD20, CD25, CD37, CD30, CD33, CD45, CAMPATH-1, HLD-DR, carcinoembryonic antigen (CEA), TAG-72, EpCAM, MUC1, MUC15, folate-binding protein, A33, G250, prostate-specific membrane antigen (PSMA), ferritin, GD2, GD3, GM2, Le^{y}, CA-125, CA19-9, epidermal growth factor, p185HER2, IL-2 receptor, fibroblast activation protein (FAP), tenascin, a metalloproteinase, endosialin, vascular endothelial growth factor, avB3, WT1, LMP2, HPV E6, HPV E7, EGFRvIII (de2-7 EGFR), Her-2/neu, idiotype, MAGE A3, p53 nonmutant, NY-ESO-1, MelanA/MART1, Ras mutant, gp100, p53 mutant, PR1, bcr-abl, tyrosinase, survivin, PSA, hTERT, a Sarcoma translocation breakpoint fusion protein, EphA2, PAP, ML-IAP, AFP, ERG, NA17, PAX3, ALK, androgen receptor, cyclin B1, polysialic acid, MYCN, RhoC, TRP-2, fucosyl GM1, mesothelin (MSLN), PSCA, MAGE A1, sLe(animal), CYP1B1, PLAV1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, B7H3, Legumain, Tie 3, Page4, VEGFR2, MAD-CT-1, PDGFR-B, MAD-CT-2, ROR2, CMET, HER3, EPCAM, CA6, NAPI2B, TROP2, CLDN18.2, RON, LY6E, FRA, DLL3, PTK7, LIV1, ROR1, Fos-related antigen 1, VEGFR, endoglin, PD-L1, CD204, CD206, CD301, VTCN1, or VISTA. An antigen binding domain can be capable of recognizing a single antigen. An antigen binding domain can be capable of recognizing two or more different antigens.

In some embodiments, the target antigen is a tumor associated antigen and has an amino acid sequence to an amino acid sequence of an antigen selected from the group consisting of: CD5, CD19, CD20, CD25, CD37, CD30, CD33, CD45, CAMPATH-1, HLD-DR, GD2, GD3, GM2, Ley, carcinoembryonic antigen (CEA), TAG-72, EpCAM, MUC1, MUC15, folate-binding protein, A33, G250, prostate-specific membrane antigen (PSMA), ferritin, CA-125, CA19-9, epidermal growth factor, p185HER2, IL-2 receptor, fibroblast activation protein (FAP), tenascin, a metalloproteinase, endosialin, vascular endothelial growth factor, avB3, WT1, LMP2, HPV E6, HPV E7, EGFRvIII (de2-7 EGFR), Her-2/neu, idiotype, MAGE A3, p53 nonmutant, NY-ESO-1, MelanA/MART1, Ras mutant, gp100, p53 mutant, PR1, bcr-abl, tyrosinase, survivin, PSA, hTERT, a Sarcoma translocation breakpoint fusion protein, EphA2, PAP, ML-IAP, AFP, ERG, NA17, PAX3, ALK, androgen receptor, cyclin B1, polysialic acid, MYCN, RhoC, TRP-2, fucosyl GM1, mesothelin (MSLN), PSCA, MAGE A1, sLe(animal), CYP1B1, PLAV1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, B7H3, Legumain, Tie 3, Page4, VEGFR2, MAD-CT-1, PDGFR-B, MAD-CT-2, ROR2, CMET, HER3, EPCAM, CA6, NAPI2B, TROP2, CLDN18.2, RON, LY6E, FRA, DLL3, PTK7, LIV1, ROR1, CMET, HER3, EPCAM, Fos-related antigen 1, VEGFR, endoglin, PD-L1, CD204, CD206, CD301, VTCN1, or VISTA.

A construct includes an Fc region with an Fc domain. An Fc domain is a structure that binds to Fc receptors. A construct can comprise an Fc domain. Fc domains can be bound by Fc receptors (FcRs). Fc domains can be from antibodies. An Fc domain can be at least 80% homologous to an Fc domain from an antibody. An Fc region can be in a scaffold. An Fc region with an Fc domain can be in an antibody scaffold. An Fc region with an Fc domain can be in a non-antibody scaffold. A construct can comprise an Fc region with an Fc domain in an antibody scaffold. A construct can comprise an Fc region with an Fc domain in a non-antibody scaffold. An Fc domain can be in a scaffold. An Fc domain can be in an antibody scaffold. An Fc domain can be in a non-antibody scaffold. A construct can comprise an Fc domain in an antibody scaffold. A construct can comprise an Fc domain in a non-antibody scaffold. Fc domains of antibodies, including those of the present disclosure, can be bound by Fc receptors (FcRs). Fc domains can be a portion of the Fc region of an antibody. FcRs bind to an Fc domain of an antibody. FcRs bind to an Fc domain of an antibody bound to an antigen. FcRs are organized into classes (e.g., gamma (γ), alpha (α) and epsilon (ε)) based on the class of antibody that the FcR recognizes. The FcαR class binds to IgA and includes several isoforms, FcαRI (CD89) and FcαμR. The FcγR class binds to IgG and includes several isoforms, FcγRI (CD64), FcγRIIA (CD32a), FcγRIIB (CD32b), FcγRIIIA (CD16a), and FcγRIIIB (CD16b). An FcγRIIIA (CD16a) can be an FcγRIIIA (CD16a) F158 variant. An FcγRIIIA (CD16a) can be an FcγRIIIA (CD16a) or a V158 variant. Each FcγR isoform can differ in affinity to the Fc region of the IgG antibody. For example, FcγRI binds to IgG with greater affinity than FcγRII or FcγRIII. The affinity of a particular FcγR isoform to IgG can be controlled, in part, by a glycan (e.g., oligosacccharide) at position CH₂ 84.4 of the IgG antibody. For example, fucose containing CH₂ 84.4 glycans can reduce IgG affinity for FcγRIIIA. In addition, G0 glucans can have increased affinity for FcγRIIIA due to the lack of galactose and terminal GlcNAc moiety.

Binding of an Fc domain to an FcR can enhance an immune response. FcR-mediated signaling that can result from an Fc region binding to an FcR can lead to the maturation of immune cells. FcR-mediated signaling that can result from an Fc domain binding to an FcR can lead to the maturation of dendritic cells. FcR-mediated signaling that can result from an Fc domain binding to an FcR can lead to more efficient immune cell antigen uptake and processing. FcR-mediated signaling that can result from an Fc region binding to an FcR can lead to more efficient dendritic cell antigen uptake and processing. FcR-mediated signaling that can result from an Fc region binding to an FcR can increase antigen presentation. FcR-mediated signaling that can result from an Fc region binding to an FcR can increase antigen presentation by immune cells. FcR-mediated signaling that can result from an Fc region binding to an FcR can increase antigen presentation by antigen presenting cells. FcR-mediated signaling that can result from an Fc domain binding to an FcR can increase antigen presentation by dendritic cells. FcR-mediated signaling that can result from an Fc domain binding to an FcR can promote the expansion and activation of T cells. FcR-mediated signaling that can result from an Fc domain binding to an FcR can promote the expansion and activation of CD8⁺ T cells. FcR-mediated signaling that can result from an Fc domain binding to an FcR can influence immune cell regulation of T cell responses. FcR-mediated signaling that can result from an Fc domain binding to an FcR can influence immune cell regulation of T cell responses. FcR-mediated signaling that can result from an Fc domain binding to an FcR can influence dendritic cell regulation of T cell responses. FcR-mediated signaling that can result from an Fc domain binding to an FcR can influence functional polarization of T cells (e.g., polarization can be toward a T_{H}1 cell response).

The profile of FcRs on a DC can impact the ability of the DC to respond upon stimulation. For example, most DC can express both CD32A and CD32B, which can have opposing effects on IgG-mediated maturation and function of DCs: binding of IgG to CD32A can mature and activate DCs in contrast with CD32B, which can mediate inhibition due to phosphorylation of immunoreceptor tyrosine-based inhibition motif (ITIM), after CD32B binding of IgG. Therefore, the activity of these two receptors can establish a threshold of DC activation. Furthermore, difference in functional avidity of these receptors for IgG can shift their functional balance. Hence, altering the Fc domain binding to FcRs can also shift their functional balance, allowing for manipulation (either enhanced activity or enhanced inhibition) of the DC immune response.

A modification in the amino acid sequence of the construct can alter the recognition and binding of an FcR for the Fc domain. For example, a modification of the amino acid sequence of the Fc domain in a construct can increase the binding affinity and/or avidity of the Fc domain for FcRs. This increase in binding affinity and/or avidity can specific for a type of FcR. However, such modifications can still allow for FcR-mediated signaling. A modification can be a substitution of an amino acid at a residue (e.g., wildtype) for a different amino acid at that residue. For example, a wildtype Fc domain or wildtype IgG1 Fc domain can comprise ASTKGPSVFPLAPSSKSTSGGTAALGCLVKDYFPEPVTVSWNSGALTSGVHTFPAVLQSS GLYSLSSVVTVPSSSLGTQTYICNVNHKPSNTKVDKKVEPKSCDKTHTCPPCPAPELLGG PSVFLFPPKPKDTLMISRTPEVTCVVVDVSHEDPEVKFNWYVDGVEVHNAKTKPREEQY NSTYRVVSVLTVLHQDWLNEGKEYKCKVSNKALPAPIEKTISKAKGQPREPQVYTLPPS REEMTKNQVSLTCLVKGFYPSDIAVEWESNGQPENNYKTTPPVLDSDGSFFLYSKLTVD KSRWQQGNVFSCSVMHEALHNHYTQKSLSLSPGK (SEQ ID NO: 154), and a modified Fc domain can comprise a substitution of an amino acid in comparison with SEQ ID NO: 154. A modification can permit binding of an FcR to a site on the Fc domain that the FcR may not otherwise bind to. A modification can increase binding affinity of an FcR to the Fc domain of a construct that the FcR may have reduced binding affinity for. A modification can decrease binding affinity of an FcR to a site on the Fc domain of a construct that the FcR may have increased binding affinity for. A modification can increase the subsequent FcR-mediated signaling after Fc binding to an FcR. A construct can comprise an Fc region with at least one amino acid change as compared to the sequence of the wild-type Fc region.

A construct can comprise an Fc domain with at least one amino acid change as compared to the sequence of the wild-type Fc domain. An amino acid change in an Fc domain can allow the construct to bind to at least one Fc receptor with greater affinity compared to a wild-type Fc region. An amino acid change in an Fc domain of a construct can allow the antibody to bind to at least one Fc receptor with greater affinity compared to a wild-type Fc domain. An Fc domain variant can comprise an amino acid sequence having at least one, two, three, four, five, six, seven, eight, nine, or ten modifications but not more than 40, 35, 30, 25, 20, 15, or 10 modifications of the amino acid sequence relative to the natural or original amino acid sequence. An Fc domain variant can comprise an amino acid sequence having at least one, two, three, four, five, six, seven, eight, nine, or ten modifications but not more than 40, 35, 30, 25, 20, 15, or 10 modifications of the amino acid sequence relative to the natural or original amino acid sequence. An Fc region can contain an Fc domain. An Fc region can be an Fc domain. An Fc region can be an Fc region of an anti-CD40 antibody. An Fc domain can be an Fc domain of an anti-CD40 antibody. An Fc region can be an Fc region of an anti-tumor antigen antibody. An Fc domain can be an Fc domain of an anti-tumor antigen antibody. An Fc region can be an Fc region of an anti-DEC205 antibody. An Fc domain can be an Fc domain of an anti-DEC205 antibody. An Fc region can be an Fc region of an anti-DICR antibody. An Fc domain can be an Fc domain of an anti-DICR antibody. An Fc region can be an Fc region of an anti-DNGR-1 antibody. An Fc domain can be an Fc domain of an anti-DNGR-1 antibody. An Fc region can be an Fc region of an anti-BDCA-2 antibody. An Fc domain can be an Fc domain of an anti-BDCA-2 antibody. An Fc region can be an Fc region of an anti-CD36 mannose scavenger receptor 1 antibody. An Fc domain can be an Fc domain of an anti-CD36 mannose scavenger receptor 1 antibody. An Fc region can be an Fc region of an anti-CLEC12A antibody. An Fc domain can be an Fc domain of an anti-CLEC12A antibody. An Fc region can be an Fc region of an anti-DC-SIGN antibody. An Fc domain can be an Fc domain of an anti-DC-SIGN antibody.

Compositions targeting tumor antigens may have Fc domains or regions that have a receptor binding affinity of a wildtype Fc domain. Alternatively, compositions targeting tumor antigens may have Fc domains or regions that have an increased receptor binding affinity as compared to the affinity of a wildtype Fc domain.

A construct can be a monoclonal antibody comprising a sequence of the IgG1 isoform that has been modified from a wildtype IgG1 sequence to increase Fc receptor binding. A modification can comprise a substitution at one or more one amino acid residues of an Fc domain such as at 5 different amino acid residues including L235V/F243L/R292P/Y300L/P396L (IgG1VLPLL). The numbering of amino acids residues described herein is according to the EU index of Kabat. This modification can be located in a portion of an antibody construct which can include an Fc domain and in particular, can be located in a portion of the Fc domain that can bind to Fc receptors. A modification can comprise a substitution at one or more amino acid residues such as at 2 different amino acid residues of an Fc domain, including S239D/I332E (IgGDE). This modification can be located in a portion of an antibody sequence which includes an Fc domain of the antibody and in particular, are located in portions of the Fc domain that can bind to Fc receptors. A modification can comprise a substitution at one or more amino acid residues such as at 3 different amino acid residues of an Fc domain including S298A/E333A/K334A (IgG1AAA). The modification can be located in a portion of an antibody sequence which includes an Fc domain of the antibody and in particular, can be located in portions of the Fc domain that can bind Fc receptors.

In some embodiments, the Fc domain comprises one or more amino acid substitutions that increase the affinity of the Fc domain to an Fc receptor compared to the affinity of a reference Fc domain to the Fc receptor in the absence of the one or more amino acid substitutions. In some embodiments, the Fc domain has at least one amino acid residue change as compared to a wildtype Fc domain, wherein the at least one amino acid residue is: F243L, R292P, Y300L, L235V, and P396L, wherein numbering of amino acid residues in the Fc domain is according to the EU index as in Kabat; S239D and I332E, wherein numbering of amino acid residues in the Fc domain is according to the EU index as in Kabat; or S298A, E333A, and K334A, wherein numbering of amino acid residues in the Fc domain is according to the EU index as in Kabat. In some embodiments, the Fc domain comprises one or more amino acid substitutions that reduce the affinity of the Fc domain to an Fc receptor compared to the affinity of a reference Fc domain to the Fc receptor in the absence of the one or more amino acid substitutions and wherein the target antigen is an antigen expressed on an immune cell.

Binding of Fc receptors to an Fc domain can be affected by amino acid substitutions. For example, binding of some Fc receptors to an Fc domain variant comprising the IgG1 VLPLL modifications can be enhanced compared to wild-type by as result of the L235V/F243L/R292P/Y300L/P396L amino acid modifications. However, binding of other Fc receptors to the Fc domain variant comprising the IgG1VLPLL modifications can be reduced compared to wild-type by the L235V/F243L/R292P/Y300L/P396L amino acid modifications. For example, the binding affinities of the Fc domain variant comprising the IgG1VLPLL modifications to FcγRIIIA and to FcγRIIA can be enhanced compared to wild-type whereas the binding affinity of the Fc domain variant comprising the IgG1VLPLL modifications to FcγRIIB can be reduced compared to wild-type. Binding of Fc receptors to an Fc domain variant comprising the IgG1DE modifications can be enhanced compared to wild-type as a result of the S239D/I332E amino acid modification. However, binding of some Fc receptors to the Fc domain variant comprising the IgGDE modifications can be reduced compared to wild-type by S239D/I332E amino acid modification. For example, the binding affinities of the Fc domain variant comprising the IgGDE modifications to FcγRIIIA and to FcγRIIB can be enhanced compared to wild-type. Binding of Fc receptors to an Fc domain variant comprising the IgG1AAA modifications can be enhanced compared to wild-type as a result of the S298A/E333A/K334A amino acid modification. However, binding of some Fc receptors to Fc domain variant comprising the IgG1AAA modifications can be reduced compared to wild-type by S298A/E333A/K334A amino acid modification. Binding affinities of the Fc domain variant comprising the IgG1AAA modifications to FcγRIIIA can be enhanced compared to wild-type whereas the binding affinity of the Fc domain variant comprising the IgG1AAA modifications to FcγRIIB can be reduced compared to wildtype.

In some embodiments, the heavy chain of a human IgG2 antibody can be mutated at cysteines at positions 127, 232, and/or 233. In some embodiments, the light chain of a human IgG2 antibody can be mutated at a cysteine at position 214. The mutations in the heavy and light chains of the human IgG2 antibody can be from a cysteine residue to a serine residue.

An Fc domain can be from an antibody. An Fc domain can be from an IgG antibody. An Fc domain can be from an IgG1, IgG2, or IgG4 antibody. An Fc domain can be at least 80% homologous to an Fc domain from an antibody. An Fc domain can be a portion of the Fc domain of an antibody. An antibody construct can comprise an Fc domain in an antibody. An antibody construct can comprise an Fc domain in a scaffold. An antibody construct can comprise an Fc domain in an antibody scaffold. An antibody construct can comprise an Fc domain in a non-antibody scaffold. An antibody construct can comprise an Fc domain covalently attached to an antigen binding domain.

A construct can be a monoclonal anti-DEC205 human antibody comprising a sequence of the IgG1 isoform that has been modified from the wildtype IgG1 sequence. A modification can comprise a substitution at more than one amino acid residue such as at 5 different amino acid residues including L235V/F243L/R292P/Y300L/P396L (DEC205-G1VLPLL). The numbering of amino acids residues described herein can be according to the EU index as in Kabat. The 5 amino acid residues can be located in a portion of an antibody sequence which can encode an Fc region of the antibody and in particular, can be located in portions of the Fc region that can bind to Fc receptors (i.e., the Fc domain). A modification can comprise a substitution at more than one amino acid residue such as at 2 different amino acid residues including S239D/I332E (DEC205-G1DE). The 2 amino acid residues can be located in a portion of an antibody sequence which encodes an Fc region of the antibody and in particular, are located in portions of the Fc region that can bind to Fc receptors (i.e., the Fc domain). A modification can comprise a substitution at more than one amino acid residue such as at 3 different amino acid residues including S298A/E333A/K334A (DEC205-G1AAA). The 3 amino acid residues can be located in a portion of an antibody sequence which can encode an Fc region of the antibody and in particular, can be located in portions of the Fc region that can bind Fc receptors (i.e., the Fc domain).

A construct can be a monoclonal anti-DICR human antibody comprising a sequence of the IgG1 isoform that has been modified from the wildtype IgG1 sequence. A modification can comprise a substitution at more than one amino acid residue such as at 5 different amino acid residues including L235V/F243L/R292P/Y300L/P396L (DICR-G1VLPLL). The numbering of amino acids residues described herein can be according to the EU index as in Kabat. The 5 amino acid residues can be located in a portion of an antibody sequence which can encode an Fc region of the antibody and in particular, can be located in portions of the Fc region that can bind to Fc receptors (i.e., the Fc domain). A modification can comprise a substitution at more than one amino acid residue such as at 2 different amino acid residues including S239D/I332E (DICR-G1DE). The 2 amino acid residues can be located in a portion of an antibody sequence which encodes an Fc region of the antibody and in particular, are located in portions of the Fc region that can bind to Fc receptors (i.e., the Fc domain). A modification can comprise a substitution at more than one amino acid residue such as at 3 different amino acid residues including S298A/E333A/K334A (DICR-G1AAA). The 3 amino acid residues can be located in a portion of an antibody sequence which can encode an Fc region of the antibody and in particular, can be located in portions of the Fc region that can bind Fc receptors (i.e., the Fc domain).

A construct can be a monoclonal anti-DNGR-1 human antibody comprising a sequence of the IgG1 isoform that has been modified from the wildtype IgG1 sequence. A modification can comprise a substitution at more than one amino acid residue such as at 5 different amino acid residues including L235V/F243L/R292P/Y300L/P396L (DNGR-1-G1VLPLL). The numbering of amino acids residues described herein can be according to the EU index as in Kabat. The 5 amino acid residues can be located in a portion of an antibody sequence which can encode an Fc region of the antibody and in particular, can be located in portions of the Fc region that can bind to Fc receptors (i.e., the Fc domain). A modification can comprise a substitution at more than one amino acid residue such as at 2 different amino acid residues including S239D/I332E (DNGR-1-G1DE). The 2 amino acid residues can be located in a portion of an antibody sequence which encodes an Fc region of the antibody and in particular, are located in portions of the Fc region that can bind to Fc receptors (i.e., the Fc domain). A modification can comprise a substitution at more than one amino acid residue such as at 3 different amino acid residues including S298A/E333A/K334A (DNGR-1-G1AAA). The 3 amino acid residues can be located in a portion of an antibody sequence which can encode an Fc region of the antibody and in particular, can be located in portions of the Fc region that can bind Fc receptors (i.e., the Fc domain).

A construct can be a monoclonal anti-CD36 mannose scavenger receptor 1 human antibody comprising a sequence of the IgG1 isoform that has been modified from the wildtype IgG1 sequence. A modification can comprise a substitution at more than one amino acid residue such as at 5 different amino acid residues including L235V/F243L/R292P/Y300L/P396L (CD36-G1VLPLL). The numbering of amino acids residues described herein can be according to the EU index as in Kabat. The 5 amino acid residues can be located in a portion of an antibody sequence which can encode an Fc region of the antibody and in particular, can be located in portions of the Fc region that can bind to Fc receptors (i.e., the Fc domain). A modification can comprise a substitution at more than one amino acid residue such as at 2 different amino acid residues including S239D/I332E (CD36-G1DE). The 2 amino acid residues can be located in a portion of an antibody sequence which encodes an Fc region of the antibody and in particular, are located in portions of the Fc region that can bind to Fc receptors (i.e., the Fc domain). A modification can comprise a substitution at more than one amino acid residue such as at 3 different amino acid residues including S298A/E333A/K334A (CD36-G1AAA). The 3 amino acid residues can be located in a portion of an antibody sequence which can encode an Fc region of the antibody and in particular, can be located in portions of the Fc region that can bind Fc receptors (i.e., the Fc domain).

A construct can be a monoclonal anti-CLEC12A human antibody comprising a sequence of the IgG1 isoform that has been modified from the wildtype IgG1 sequence. A modification can comprise a substitution at more than one amino acid residue such as at 5 different amino acid residues including L235V/F243L/R292P/Y300L/P396L (CLEC12A-G1VLPLL). The numbering of amino acids residues described herein can be according to the EU index as in Kabat. The 5 amino acid residues can be located in a portion of an antibody sequence which can encode an Fc region of the antibody and in particular, can be located in portions of the Fc region that can bind to Fc receptors (i.e., the Fc domain). A modification can comprise a substitution at more than one amino acid residue such as at 2 different amino acid residues including S239D/I332E (CLEC12A-G1DE). The 2 amino acid residues can be located in a portion of an antibody sequence which encodes an Fc region of the antibody and in particular, are located in portions of the Fc region that can bind to Fc receptors (i.e., the Fc domain). A modification can comprise a substitution at more than one amino acid residue such as at 3 different amino acid residues including S298A/E333A/K334A (CLEC12A-G1AAA). The 3 amino acid residues can be located in a portion of an antibody sequence which can encode an Fc region of the antibody and in particular, can be located in portions of the Fc region that can bind Fc receptors (i.e., the Fc domain).

A construct can be a monoclonal anti-DC-SIGN human antibody comprising a sequence of the IgG1 isoform that has been modified from the wildtype IgG1 sequence. A modification can comprise a substitution at more than one amino acid residue such as at 5 different amino acid residues including L235V/F243L/R292P/Y300L/P396L (DC-SIGN-G1VLPLL). The numbering of amino acids residues described herein can be according to the EU index as in Kabat. The 5 amino acid residues can be located in a portion of an antibody sequence which can encode an Fc region of the antibody and in particular, can be located in portions of the Fc region that can bind to Fc receptors (i.e., the Fc domain). A modification can comprise a substitution at more than one amino acid residue such as at 2 different amino acid residues including S239D/I332E (DC-SIGN-G1DE). The 2 amino acid residues can be located in a portion of an antibody sequence which encodes an Fc region of the antibody and in particular, are located in portions of the Fc region that can bind to Fc receptors (i.e., the Fc domain). A modification can comprise a substitution at more than one amino acid residue such as at 3 different amino acid residues including S298A/E333A/K334A (DC-SIGN-G1AAA). The 3 amino acid residues can be located in a portion of an antibody sequence which can encode an Fc region of the antibody and in particular, can be located in portions of the Fc region that can bind Fc receptors (i.e., the Fc domain).

A construct can be a heavy chain of an anti-CD40 antibody. A heavy chain of an anti-CD40 antibody can be SBT-040-G1VLPLL. SBT-040-G1VLPLL be expressed from a DNA sequence comprising ATGGACTGGACCTGGAGGATCCTCTTCTTGGTGGCAGCCACAGGAGCCCACTCC CAGGTGCAGCTGGTGCAGTCTGGGGCTGAGGTGAAGAAGCCTGGGGCCTCAGTGAA GGTCTCCTGCAAGGCTTCTGGATACACCTTCACCGGCTACTATATGCACTGGGTGCG ACAGGCCCCTGGACAAGGGCTTGAGTGGATGGGATGGATCAACCCTGACAGTGGTG GCACAAACTATGCACAGAAGTTTCAGGGCAGGGTCACCATGACCAGGGACACGTCC ATCAGCACAGCCTACATGGAGCTGAACAGGCTGAGATCTGACGACACGGCCGTGTA TTACTGTGCGAGAGATCAGCCCCTAGGATATTGTACTAATGGTGTATGCTCCTACTTT GACTACTGGGGCCAGGGAACCCTGGTCACCGTCTCCTCAGCCTCCACCAAGGGCCCA TCGGTCTTCCCCCTGGCGCCCTGCTCCAGGAGCACCTCCGAGAGCACAGCGGCCCTG GGCTGCCTGGTCAAGGACTACTTCCCCGAACCGGTGACGGTGTCGTGGAACTCAGGC GCTCTGACCAGCGGCGTGCACACCTTCCCAGCTGTCCTACAGTCCTCAGGACTCTAC TCCCTCAGCAGCGTGGTGACCGTGCCCTCCAGCAACTTCGGCACCCAGACCTACACC TGCAACGTAGATCACAAGCCCAGCAACACCAAGGTGGACAAGACAGTTGAGCCCAA ATCTTGTGACAAAACTCACACATGCCCACCGTGCCCAGCACCTGAACTCGTGGGGGG ACCGTCAGTCTTCCTCCTGCCCCCAAAACCCAAGGACACCCTCATGATCTCCCGGAC CCCTGAGGTCACATGCGTGGTGGTGGACGTGAGCCACGAAGACCCTGAGGTCAAGT TCAACTGGTACGTGGACGGCGTGGAGGTGCATAATGCCAAGACAAAGCCGCCTGAG GAGCAGTACAACAGCACGCTGCGTGTGGTCAGCGTCCTCACCGTCCTGCACCAGGAC TGGCTGAATGGCAAGGAGTACAAGTGCAAGGTCTCCAACAAAGCCCTCCCAGCCCC CATCGAGAAAACCATCTCCAAAGCCAAAGGGCAGCCCCGAGAACCACAGGTGTACA CCCTGCCCCCATCCCGGGAGGAGATGACCAAGAACCAGGTCAGCCTGACCTGCCTG GTCAAAGGCTTCTATCCCAGCGACATCGCCGTGGAGTGGGAGAGCAATGGGCAGCC GGAGAACAACTACAAGACCACGCCTCTGGTGCTGGACTCCGACGGCTCCTTCTTCCT CTATAGCAAGCTCACCGTGGACAAGAGCAGGTGGCAGCAGGGGAACGTCTTCTCAT GCTCCGTGATGCATGAGGCTCTGCACAACCACTACACGCAGAAGAGCCTCTCCCTGT CCCCGGGTAAATGA (SEQ ID NO: 9) wherein the DNA sequence comprises DNA nucleotide modifications that correspond to L235V, F243L, R292P, Y300L and P396L amino acid residue modifications compared to a wild-type DNA sequence. SBT-040-G1VLPLL can be expressed from a DNA sequence comprising greater than 70%, greater than 75%, greater than 80%, greater than 85%, greater than 90%, greater than 95% or greater than 99% homology to SEQ ID NO: 9. A variable region of SBT-040-G1VLPLL can be expressed from a DNA sequence comprising SEQ ID NO: 13. A variable region of SBT-040-G1VLPLL can be expressed from a DNA sequence comprising greater than 70%, greater than 75%, greater than 80%, greater than 85%, greater than 90%, greater than 95% or greater than 99% homology to SEQ ID NO: 13. Additionally, anti-CD40 antibodies comprising SBT-040-G1VLPLL expressed from SEQ ID NO: 9, or expressed from a DNA sequence comprising greater than 70% homology to SEQ ID NO: 9 can have a dissociation constant (K_{d}) for CD40 that is less than 10 nM. Anti-CD40 antibodies comprising SBT-040-G1VLPLL expressed from DNA sequence comprising SEQ ID NO: 9, or comprising greater than 70% homology to SEQ ID NO: 9 can have a dissociation constant (K_{d}) for CD40 that is less than 1 nM, less than 100 pM, less than 10 pM, less than 1 pM, or less than 0.1 pM. SBT-040-G1VLPLL can be expressed with any anti-CD40 light chain or fragment thereof. SBT-040-G1VLPLL can also be expressed with any anti-CD40 light chain or fragment thereof to form an anti-CD40 antibody or fragment thereof. The anti-CD40 antibody or fragment thereof can be purified, and can be combined with a pharmaceutically acceptable carrier. The anti-CD40 antibody can be a construct. Additionally, one skilled in the art would recognize that these same concepts could apply to constructs comprising anti-CD40 antibodies created for use in the veterinary sciences and/or in laboratory animals.

SBT-040-G1VLPLL can comprise an amino acid sequence MDWTWRILFLVAAATGAHSQVQLVQSGAEVKKPGASVKVSCKASGYTFTGYYMHWV RQAPGQGLEWMGWINPDSGGTNYAQKFQGRVTMTRDTSISTAYMELNRLRSDDTAVY YCARDQPLGYCTNGVCSYFDYWGQGTLVTVSSASTKGPSVFPLAPCSRSTSESTAALGC LVKDYFPEPVTVSWNSGALTSGVHTFPAVLQSSGLYSLSSVVTVPSSNFGTQTYTCNVD HKPSNTKVDKTVEPKSCDKTHTCPPCPAPELVGGPSVFLLPPKPKDTLMISRTPEVTCVV VDVSHEDPEVKFNWYVDGVEVHNAKTKPPEEQYNSTLRVVSVLTVLHQDWLNGKEYK CKVSNKALPAPIEKTISKAKGQPREPQVYTLPPSREEMTKNQVSLTCLVKGFYPSDIAVE WESNGQPENNYKTTPLVLDSDGSFFLYSKLTVDKSRWQQGNVFSCSVMHEALHNHYTQ KSLSLSPGK (SEQ ID NO: 16) wherein the amino acid sequence comprises L235V, F243L, R292P, Y300L, and P396L amino acid residue modifications compared to a wild-type amino acid sequence. SBT-040-G1VLPLL can comprise an amino acid sequence with greater than 70%, greater than 75%, greater than 80%, greater than 85%, greater than 90%, greater than 95% or greater than 99% homology to SEQ ID NO: 16. SBT-040-G1VLPLL can comprise an amino acid sequence SEQ ID NO: 20. A variable region of SBT-040-G1VLPLL can comprise an amino acid sequence with greater than 70%, greater than 75%, greater than 80%, greater than 85%, greater than 90%, greater than 95% or greater than 99% homology to SEQ ID NO: 20. Additionally, anti-CD40 antibodies comprising SBT-040-G1VLPLL with SEQ ID NO: 16 or with an amino acid sequence with greater than 70% homology to SEQ ID NO: 16 can have a dissociation constant (K_{d}) for CD40 that is less than 10 nM. Anti-CD40 antibodies comprising SBT-040-G1VLPLL with SEQ ID NO: 16 or with an amino acid sequence with greater than 70% homology to SEQ ID NO: 16 can have a dissociation constant (K_{d}) for CD40 that is less than 1 nM, less than 100 pM, less than 10 pM, less than 1 pM, or less than 0.1 pM. SBT-040-G1VLPLL can be purified. SBT-040-G1VLPLL can be combined with any anti-CD40 light chain or fragment thereof to form an anti-CD40 antibody or fragment thereof. The anti-CD40 antibody or fragment thereof can be purified, and can be combined with a pharmaceutically acceptable carrier. The anti-CD40 antibody can be a construct. Additionally, one skilled in the art would recognize that these same concepts could apply to constructs comprising anti-CD40 antibodies created for use in the veterinary sciences and/or in laboratory animals.

A heavy chain of an anti-CD40 antibody can be SBT-040-G1DE. SBT-040-G1DE be expressed from a DNA sequence comprising ATGGACTGGACCTGGAGGATCCTCTTCTTGGTGGCAGCCACAGGAGCCCACTCC CAGGTGCAGCTGGTGCAGTCTGGGGCTGAGGTGAAGAAGCCTGGGGCCTCAGTGAA GGTCTCCTGCAAGGCTTCTGGATACACCTTCACCGGCTACTATATGCACTGGGTGCG ACAGGCCCCTGGACAAGGGCTTGAGTGGATGGGATGGATCAACCCTGACAGTGGTG GCACAAACTATGCACAGAAGTTTCAGGGCAGGGTCACCATGACCAGGGACACGTCC ATCAGCACAGCCTACATGGAGCTGAACAGGCTGAGATCTGACGACACGGCCGTGTA TTACTGTGCGAGAGATCAGCCCCTAGGATATTGTACTAATGGTGTATGCTCCTACTTT GACTACTGGGGCCAGGGAACCCTGGTCACCGTCTCCTCAGCCTCCACCAAGGGCCCA TCGGTCTTCCCCCTGGCGCCCTGCTCCAGGAGCACCTCCGAGAGCACAGCGGCCCTG GGCTGCCTGGTCAAGGACTACTTCCCCGAACCGGTGACGGTGTCGTGGAACTCAGGC GCTCTGACCAGCGGCGTGCACACCTTCCCAGCTGTCCTACAGTCCTCAGGACTCTAC TCCCTCAGCAGCGTGGTGACCGTGCCCTCCAGCAACTTCGGCACCCAGACCTACACC TGCAACGTAGATCACAAGCCCAGCAACACCAAGGTGGACAAGACAGTTGAGCCCAA ATCTTGTGACAAAACTCACACATGCCCACCGTGCCCAGCACCTGAACTCCTGGGGGG ACCGGATGTCTTCCTCTTCCCCCCAAAACCCAAGGACACCCTCATGATCTCCCGGAC CCCTGAGGTCACATGCGTGGTGGTGGACGTGAGCCACGAAGACCCTGAGGTCAAGT TCAACTGGTACGTGGACGGCGTGGAGGTGCATAATGCCAAGACAAAGCCGCGGGAG GAGCAGTACAACAGCACGTACCGTGTGGTCAGCGTCCTCACCGTCCTGCACCAGGAC TGGCTGAATGGCAAGGAGTACAAGTGCAAGGTCTCCAACAAAGCCCTCCCAGCCCC CGAGGAGAAAACCATCTCCAAAGCCAAAGGGCAGCCCCGAGAACCACAGGTGTACA CCCTGCCCCCATCCCGGGAGGAGATGACCAAGAACCAGGTCAGCCTGACCTGCCTG GTCAAAGGCTTCTATCCCAGCGACATCGCCGTGGAGTGGGAGAGCAATGGGCAGCC GGAGAACAACTACAAGACCACGCCTCCCGTGCTGGACTCCGACGGCTCCTTCTTCCT CTATAGCAAGCTCACCGTGGACAAGAGCAGGTGGCAGCAGGGGAACGTCTTCTCAT GCTCCGTGATGCATGAGGCTCTGCACAACCACTACACGCAGAAGAGCCTCTCCCTGT CCCCGGGTAAATGA (SEQ ID NO: 10) wherein the DNA sequence comprises DNA nucleotide modifications that correspond to S239D and 1332E amino acid residue modifications compared to a wild-type DNA sequence. SBT-040-G1DE can be expressed from a DNA sequence comprising greater than 70%, greater than 75%, greater than 80%, greater than 85%, greater than 90%, greater than 95% or greater than 99% homology to SEQ ID NO: 10. A variable region of SBT-040-G1DE can be expressed from a DNA sequence comprising SEQ ID NO: 13. A variable region of SBT-040-G1DE can be expressed from a DNA sequence comprising greater than 70%, greater than 75%, greater than 80%, greater than 85%, greater than 90%, greater than 95% or greater than 99% homology to SEQ ID NO: 13. Additionally, anti-CD40 antibodies comprising SBT-040-G1DE expressed from SEQ ID NO: 10, or expressed from a DNA sequence comprising greater than 70% homology to SEQ ID NO: 10 can have a dissociation constant (K_{d}) for CD40 that is less than 10 nM. Anti-CD40 antibodies comprising SBT-040-G1DE expressed from DNA sequence comprising SEQ ID NO: 10, or comprising greater than 70% homology to SEQ ID NO: 10 can have a dissociation constant (K_{d}) for CD40 that is less than 1 nM, less than 100 pM, less than 10 pM, less than 1 pM, or less than 0.1 pM. SBT-040-G1DE can be expressed with any anti-CD40 light chain or fragment thereof. SBT-040-G1DE can also be expressed with any anti-CD40 light chain or fragment thereof to form an anti-CD40 antibody or fragment thereof. The anti-CD40 antibody or fragment thereof can be purified, and can be combined with a pharmaceutically acceptable carrier. The anti-CD40 antibody can be a construct. Additionally, one skilled in the art would recognize that these same concepts could apply to constructs comprising anti-CD40 antibodies created for use in the veterinary sciences and/or in laboratory animals.

SBT-040-G1DE can comprise an amino acid sequence MDWTWRILFLVAAATGAHSQVQLVQSGAEVKKPGASVKVSCKASGYTFTGYYMHWV RQAPGQGLEWMGWINPDSGGTNYAQKFQGRVTMTRDTSISTAYMELNRLRSDDTAVY YCARDQPLGYCTNGVCSYFDYWGQGTLVTVSSASTKGPSVFPLAPCSRSTSESTAALGC LVKDYFPEPVTVSWNSGALTSGVHTFPAVLQSSGLYSLSSVVTVPSSNFGTQTYTCNVD HKPSNTKVDKTVEPKSCDKTHTCPPCPAPELLGGPDVFLFPPKPKDTLMISRTPEVTCVV VDVSHEDPEVKFNWYVDGVEVHNAKTKPREEQYNSTYRVVSVLTVLHQDWLNGKEY KCKVSNKALPAPEEKTISKAKGQPREPQVYTLPPSREEMTKNQVSLTCLVKGFYPSDIAV EWESNGQPENNYKTTPPVLDSDGSFFLYSKLTVDKSRWQQGNVFSCSVMHEALHNHYT QKSLSLSPGK (SEQ ID NO: 17) wherein the amino acid sequence comprises S239D and 1332E amino acid residue modifications compared to a wild-type amino acid sequence. SBT-040-G1DE can comprise an amino acid sequence with greater than 70%, greater than 75%, greater than 80%, greater than 85%, greater than 90%, greater than 95% or greater than 99% homology to SEQ ID NO: 17. SBT-040-G1DE can comprise an amino acid sequence SEQ ID NO: 20. A variable region of SBT-040-G1DE can comprise an amino acid sequence with greater than 70%, greater than 75%, greater than 80%, greater than 85%, greater than 90%, greater than 95% or greater than 99% homology to SEQ ID NO: 20. Additionally, anti-CD40 antibodies comprising SBT-040-G1DE with SEQ ID NO: 17 or with an amino acid sequence with greater than 70% homology to SEQ ID NO: 17 can have a dissociation constant (K_{d}) for CD40 that is less than 10 nM. Anti-CD40 antibodies comprising SBT-040-G1DE with SEQ ID NO: 17 or with an amino acid sequence with greater than 70% homology to SEQ ID NO: 17 can have a dissociation constant (K_{d}) for CD40 that is less than 1 nM, less than 100 pM, less than 10 pM, less than 1 pM, or less than 0.1 pM. SBT-040-G1DE can be purified. SBT-040-G1DE can be combined with any anti-CD40 light chain or fragment thereof to form an anti-CD40 antibody or fragment thereof. The anti-CD40 antibody or fragment thereof can be purified, and can be combined with a pharmaceutically acceptable carrier. The anti-CD40 antibody can be a construct. Additionally, one skilled in the art would recognize that these same concepts could apply to constructs comprising anti-CD40 antibodies created for use in the veterinary sciences and/or in laboratory animals.

A heavy chain of an anti-CD40 antibody can be SBT-040-G1AAA. SBT-040-G1AAA be expressed from a DNA sequence comprising ATGGACTGGACCTGGAGGATCCTCTTCTTGGTGGCAGCCACAGGAGCCCACTCC CAGGTGCAGCTGGTGCAGTCTGGGGCTGAGGTGAAGAAGCCTGGGGCCTCAGTGAA GGTCTCCTGCAAGGCTTCTGGATACACCTTCACCGGCTACTATATGCACTGGGTGCG ACAGGCCCCTGGACAAGGGCTTGAGTGGATGGGATGGATCAACCCTGACAGTGGTG GCACAAACTATGCACAGAAGTTTCAGGGCAGGGTCACCATGACCAGGGACACGTCC ATCAGCACAGCCTACATGGAGCTGAACAGGCTGAGATCTGACGACACGGCCGTGTA TTACTGTGCGAGAGATCAGCCCCTAGGATATTGTACTAATGGTGTATGCTCCTACTTT GACTACTGGGGCCAGGGAACCCTGGTCACCGTCTCCTCAGCCTCCACCAAGGGCCCA TCGGTCTTCCCCCTGGCGCCCTGCTCCAGGAGCACCTCCGAGAGCACAGCGGCCCTG GGCTGCCTGGTCAAGGACTACTTCCCCGAACCGGTGACGGTGTCGTGGAACTCAGGC GCTCTGACCAGCGGCGTGCACACCTTCCCAGCTGTCCTACAGTCCTCAGGACTCTAC TCCCTCAGCAGCGTGGTGACCGTGCCCTCCAGCAACTTCGGCACCCAGACCTACACC TGCAACGTAGATCACAAGCCCAGCAACACCAAGGTGGACAAGACAGTTGAGCCCAA ATCTTGTGACAAAACTCACACATGCCCACCGTGCCCAGCACCTGAACTCCTGGGGGG ACCGTCAGTCTTCCTCTTCCCCCCAAAACCCAAGGACACCCTCATGATCTCCCGGAC CCCTGAGGTCACATGCGTGGTGGTGGACGTGAGCCACGAAGACCCTGAGGTCAAGT TCAACTGGTACGTGGACGGCGTGGAGGTGCATAATGCCAAGACAAAGCCGCGGGAG GAGCAGTACAACGCCACGTACCGTGTGGTCAGCGTCCTCACCGTCCTGCACCAGGAC TGGCTGAATGGCAAGGAGTACAAGTGCAAGGTCTCCAACAAAGCCCTCCCAGCCCC CATCGCCGCTACCATCTCCAAAGCCAAAGGGCAGCCCCGAGAACCACAGGTGTACA CCCTGCCCCCATCCCGGGAGGAGATGACCAAGAACCAGGTCAGCCTGACCTGCCTG GTCAAAGGCTTCTATCCCAGCGACATCGCCGTGGAGTGGGAGAGCAATGGGCAGCC GGAGAACAACTACAAGACCACGCCTCCCGTGCTGGACTCCGACGGCTCCTTCTTCCT CTATAGCAAGCTCACCGTGGACAAGAGCAGGTGGCAGCAGGGGAACGTCTTCTCAT GCTCCGTGATGCATGAGGCTCTGCACAACCACTACACGCAGAAGAGCCTCTCCCTGT CCCCGGGTAAATGA (SEQ ID NO: 11) wherein the DNA sequence comprises DNA nucleotide modifications that correspond to S298A, E333A, and K334A amino acid residue modifications compared to a wild-type DNA sequence. SBT-040-G1AAA can be expressed from a DNA sequence comprising greater than 70%, greater than 75%, greater than 80%, greater than 85%, greater than 90%, greater than 95% or greater than 99% homology to SEQ ID NO: 11. A variable region of SBT-040-G1AAA can be expressed from a DNA sequence comprising SEQ ID NO: 13. A variable region of SBT-040-G1AAA can be expressed from a DNA sequence comprising greater than 70%, greater than 75%, greater than 80%, greater than 85%, greater than 90%, greater than 95% or greater than 99% homology to SEQ ID NO: 13. Additionally, anti-CD40 antibodies comprising SBT-040-G1AAA expressed from SEQ ID NO: 11, or expressed from a DNA sequence comprising greater than 70% homology to SEQ ID NO: 11 can have a dissociation constant (K_{d}) for CD40 that is less than 10 nM. Anti-CD40 antibodies comprising SBT-040-G1AAA expressed from DNA sequence comprising SEQ ID NO: 11, or comprising greater than 70% homology to SEQ ID NO: 11 can have a dissociation constant (K_{d}) for CD40 that is less than 1 nM, less than 100 pM, less than 10 pM, less than 1 pM, or less than 0.1 pM. SBT-040-G1AAA can be expressed with any anti-CD40 light chain or fragment thereof. SBT-040-G1AAA can also be expressed with any anti-CD40 light chain or fragment thereof to form an anti-CD40 antibody or fragment thereof. The anti-CD40 antibody or fragment thereof can be purified, and can be combined with a pharmaceutically acceptable carrier. The anti-CD40 antibody can be a construct. Additionally, one skilled in the art would recognize that these same concepts could apply to constructs comprising anti-CD40 antibodies created for use in the veterinary sciences and/or in laboratory animals. SBT-040-G1AAA can comprise an amino acid sequence MDWTWRILFLVAAATGAHSQVQLVQSGAEVKKPGASVKVSCKASGYTFTGYYMHWV RQAPGQGLEWMGWINPDSGGTNYAQKFQGRVTMTRDTSISTAYMELNRLRSDDTAVY YCARDQPLGYCTNGVCSYFDYWGQGTLVTVSSASTKGPSVFPLAPCSRSTSESTAALGC LVKDYFPEPVTVSWNSGALTSGVHTFPAVLQSSGLYSLSSVVTVPSSNFGTQTYTCNVD HKPSNTKVDKTVEPKSCDKTHTCPPCPAPELLGGPSVFLFPPKPKDTLMISRTPEVTCVV VDVSHEDPEVKFNWYVDGVEVHNAKTKPREEQYNATYRVVSVLTVLHQDWLNGKEY KCKVSNKALPAPIAATISKAKGQPREPQVYTLPPSREEMTKNQVSLTCLVKGFYPSDIAV EWESNGQPENNYKTTPPVLDSDGSFFLYSKLTVDKSRWQQGNVFSCSVMHEALHNHYT QKSLSLSPGK (SEQ ID NO: 18) wherein the amino acid sequence comprises S298A, E333A, and K334A amino acid residue modifications compared to a wild-type amino acid sequence. SBT-040-G1AAA can comprise an amino acid sequence with greater than 70%, greater than 75%, greater than 80%, greater than 85%, greater than 90%, greater than 95% or greater than 99% homology to SEQ ID NO: 18. SBT-040-G1AAA can comprise an amino acid sequence SEQ ID NO: 20. A variable region of SBT-040-G1AAA can comprise an amino acid sequence with greater than 70%, greater than 75%, greater than 80%, greater than 85%, greater than 90%, greater than 95% or greater than 99% homology to SEQ ID NO: 20. Additionally, anti-CD40 antibodies comprising SBT-040-G1AAA with SEQ ID NO: 18 or with an amino acid sequence with greater than 70% homology to SEQ ID NO: 18 can have a dissociation constant (K_{d}) for CD40 that is less than 10 nM. Anti-CD40 antibodies comprising SBT-040-G1AAA with SEQ ID NO: 18 or with an amino acid sequence with greater than 70% homology to SEQ ID NO: 18 can have a dissociation constant (K_{d}) for CD40 that is less than 1 nM, less than 100 pM, less than 10 pM, less than 1 pM, or less than 0.1 pM. SBT-040-G1AAA can be purified. SBT-040-G1AAA can be combined with any anti-CD40 light chain or fragment thereof to form an anti-CD40 antibody or fragment thereof. The anti-CD40 antibody or fragment thereof can be purified, and can be combined with a pharmaceutically acceptable carrier. The anti-CD40 antibody can be a construct. Additionally, one skilled in the art would recognize that these same concepts could apply to anti-CD40 antibodies created for use in the veterinary sciences and/or in laboratory animals.

A construct can comprise an antibody heavy chain. A heavy chain can be a heavy chain of an anti-HER2 monoclonal antibody which can bind a HER2 antigen. A heavy chain of an anti-HER2 antibody can be an IgG1 isotype. A heavy chain of an anti-HER2 antibody can be SBT-050 VH-hIgG1 wt (pertuzumab). SBT-050 VH-hIgG1 wt can comprise an amino acid sequence EVQLVESGGGLVQPGGSLRLSCAASGFTFTDYTMDWVRQAPGKGLEWVADVNPNSGG SIYNQRFKGRFTLSVDRSKNTLYLQMNSLRAEDTAVYYCARNLGPSFYFDYWGQGTLV TVSSASTKGPSVFPLAPSSKSTSGGTAALGCLVKDYFPEPVTVSWNSGALTSGVHTFPAV LQSSGLYSLSSVVTVPSSSLGTQTYICNVNHKPSNTKVDKKVEPKSCDKTHTCPPCPAPE LLGGPSVFLFPPKPKDTLMISRTPEVTCVVVDVSHEDPEVKFNWYVDGVEVHNAKTKPR EEQYNSTYRVVSVLTVLHQDWLNGKEYKCKVSNKALPAPIEKTISKAKGQPREPQVYTL PPSREEMTKNQVSLTCLVKGFYPSDIAVEWESNGQPENNYKTTPPVLDSDGSFFLYSKLT VDKSRWQQGNVFSCSVMHEALHNHYTQKSLSLSPGK (SEQ ID NO: 30). SBT-050 VH-hIgG1 wt can comprise an amino acid sequence with greater than 70%, greater than 75%, greater than 80%, greater than 85%, greater than 90%, greater than 95% or greater than 99% homology to SEQ ID NO: 30. A heavy chain of an anti-HER2 antibody can comprise a CDR. A heavy chain of an anti-HER2 antibody can comprise a CDR with an amino acid sequence GFTFTDYT (SEQ ID NO: 31). A heavy chain of an anti-HER2 antibody can comprise a CDR with an amino acid sequence VNPNSGGS (SEQ ID NO: 32). A heavy chain of an anti-HER2 antibody can comprise a CDR with an amino acid sequence ARNLGPSFYFDY (SEQ ID NO: 33). A heavy chain CDR of an anti-HER2 antibody can comprise an amino acid sequence with greater than 70%, greater than 75%, greater than 80%, greater than 85%, greater than 90%, greater than 95% or greater than 99% homology to SEQ ID NO: 31. A heavy chain CDR of an anti-HER2 antibody can comprise an amino acid sequence with greater than 70%, greater than 75%, greater than 80%, greater than 85%, greater than 90%, greater than 95% or greater than 99% homology to SEQ ID NO: 32. A heavy chain CDR of an anti-HER2 antibody can comprise an amino acid sequence with greater than 70%, greater than 75%, greater than 80%, greater than 85%, greater than 90%, greater than 95% or greater than 99% homology to SEQ ID NO: 33.

A construct can comprise an antibody light chain. A light chain can be a light chain of a HER2 monoclonal antibody which can bind a HER2 antigen. A light chain of an anti-HER2 antibody can be SBT-050 VL-Ck (pertuzumab). SBT-050 VL-Ck can comprise an amino acid sequence DIQMTQSPSSLSASVGDRVTITCKASQDVSIGVAWYQQKPGKAPKLLIYSASYRYTGVPS RFSGSGSGTDFTLTISSLQPEDFATYYCQQYYIYPYTFGQGTKVEIKRTVAAPSVFIFPPSD EQLKSGTASVVCLLNNFYPREAKVQWKVDNALQSGNSQESVTEQDSKDSTYSLSSTLTL SKADYEKHKVYACEVTHQGLSSPVTKSFNRGEC (SEQ ID NO: 34). SBT-050 VL-Ck can comprise an amino acid sequence with greater than 70%, greater than 75%, greater than 80%, greater than 85%, greater than 90%, greater than 95% or greater than 99% homology to SEQ ID NO: 34. A light chain of an anti-HER2 antibody can comprise a CDR. A light chain of an anti-HER2 antibody can comprise a CDR with an amino acid sequence QDVSIG (SEQ ID NO: 35). A light chain of an anti-HER2 antibody can comprise a CDR with an amino acid sequence SAS (SEQ ID NO: 36). A light chain of an anti-HER2 antibody can comprise a CDR with an amino acid sequence QQYYIYPYT (SEQ ID NO: 37). A light chain CDR of an anti-HER2 antibody can comprise an amino acid sequence with greater than 70%, greater than 75%, greater than 80%, greater than 85%, greater than 90%, greater than 95% or greater than 99% homology to SEQ ID NO: 35. A light chain CDR of an anti-HER2 antibody can comprise an amino acid sequence with greater than 70%, greater than 75%, greater than 80%, greater than 85%, greater than 90%, greater than 95% or greater than 99% homology to SEQ ID NO: 36. A light chain CDR of an anti-HER2 antibody can comprise an amino acid sequence with greater than 70%, greater than 75%, greater than 80%, greater than 85%, greater than 90%, greater than 95% or greater than 99% homology to SEQ ID NO: 37.

While a construct of the present disclosure can comprise an anti-CD40 antibody with wild-type or modified amino acid sequences encoding the Fc region or Fc domain, the modifications of the Fc region or the Fc domain from the wild-type sequence may not significantly alter binding and/or affinity of the anti-CD40 antibody for CD40. For example, binding and/or affinity of SBT-040-G1WT, SBT-040-G1VLPLL, SBT-040-G1DE, and SBT-040-G1AAA may not be significantly altered by modification of an Fc region or Fc domain amino acid sequence compared to a wild-type sequence. Modifications of an Fc region or Fc domain from a wild-type sequence may not alter binding and/or affinity of antibodies that bind to CD40 in a construct. Additionally, the binding and/or affinity of the antibodies described herein that bind to CD40 and are constructs, for example SBT-040-G1WT, SBT-040-G1VLPLL, SBT-040-G1DE, and SBT-040-G1AAA, may be comparable to the binding and/or affinity of wild-type antibodies that can bind to CD40.

Sequences that can be used to produce antibodies for constructs can include leader sequences. Any of the sequences provided herein can be used with or without a leader sequence in a construct described herein. Leader sequences can be signal sequences. Leader sequences useful with the compositions and methods described herein can include, but are not limited to, a DNA sequence comprising ATGAGGCTCCCTGCTCAGCTCCTGGGGCTCCTGCTGCTCTGGTTCCCAGGTTCCAGAT GC (SEQ ID NO: 2) or ATGGACTGGACCTGGAGGATCCTCTTCTTGGTGGCAGCCACAGGAGCCCACTCC (SEQ ID NO: 12), or an amino acid sequence comprising MRLPAQLLGLLLLWFPGSRC (SEQ ID NO: 5) and MDWTWRILFLVAAATGAHS (SEQ ID NO: 19). Leader sequence can comprise a DNA sequence with greater than 70%, greater than 75%, greater than 80%, greater than 85%, greater than 90%, greater than 95% or greater than 99% homology to SEQ ID NO: 2 or SEQ ID NO: 12. Leader sequence can comprise an amino acid sequence with greater than 70%, greater than 75%, greater than 80%, greater than 85%, greater than 90%, greater than 95% or greater than 99% homology to SEQ ID NO: 5 or SEQ ID NO: 19. Additionally, one skilled in the art would recognize that these same concepts can apply to constructs comprising anti-CD40 antibodies created for use in the veterinary sciences and/or in laboratory animals.

In some aspects, a composition may comprise an immune-stimulatory compound connected to a construct by a first linker. The construct may comprise an antigen binding domain, wherein the antigen binding domain may specifically bind a target antigen. The construct may additionally comprise an Fc domain, wherein a K_{d} for binding of the Fc domain to an Fc receptor in the presence of the immune-stimulatory compound may be no greater than about 100 times a K_{d} for binding of the Fc domain to the Fc receptor in the absence of the immune stimulatory compound. The construct may also comprise a peptide comprising an antigenic epitope of a cancer sequence, and wherein the peptide may be connected to the construct.

### Peptide of the Construct-Peptide Composition

A construct-peptide composition comprises a peptide. In some cases, the peptide can be from a peptide fragment of an antigen from a tumor cell. The peptide can be immunogenic and can generate or stimulate an immune response. The peptide can be an antigenic peptide. A peptide can be presented in a major histocompatibility complex by a cell. A presented peptide can be a tumor antigen fragment. The peptide can be a peptide fragment of an antigen from a cancer cell. For example, the peptide can be a protein fragment or antigen from Acute lymphoblastic leukemia (ALL); Acute myeloid leukemia; Adrenocortical carcinoma; Astrocytoma, childhood cerebellar or cerebral; Basal-cell carcinoma; Bladder cancer; Bone tumor, osteosarcoma/malignant fibrous histiocytoma; Brain cancer; Brain tumors, such as, cerebellar astrocytoma, malignant glioma, ependymoma, medulloblastoma, visual pathway and hypothalamic glioma; Brainstem glioma; Breast cancer; Bronchial adenomas/carcinoids; Burkitt's lymphoma; Cerebellar astrocytoma; Cervical cancer; Cholangiocarcinoma; Chondrosarcoma; Chronic lymphocytic leukemia; Chronic myelogenous leukemia; Chronic myeloproliferative disorders; Colon cancer; Cutaneous T-cell lymphoma; Endometrial cancer; Ependymoma; Esophageal cancer; Eye cancers, such as, intraocular melanoma and retinoblastoma; Gallbladder cancer; Glioma; Hairy cell leukemia; Head and neck cancer; Heart cancer; Hepatocellular (liver) cancer; Hodgkin lymphoma; Hypopharyngeal cancer; Islet cell carcinoma (endocrine pancreas); Kaposi sarcoma; Kidney cancer (renal cell cancer); Laryngeal cancer; Leukaemia, such as, acute lymphoblastic, acute myeloid, chronic lymphocytic, chronic myelogenous and, hairy cell; Lip and oral cavity cancer; Liposarcoma; Lung cancer, such as, non-small cell and small cell; Lymphoma, such as, AIDS-related, Burkitt; Lymphoma, cutaneous T-Cell, Hodgkin and Non-Hodgkin, Macroglobulinemia, Malignant fibrous histiocytoma of bone/osteosarcoma; Melanoma; Merkel cell cancer; Mesothelioma; Multiple myeloma/plasma cell neoplasm; Mycosis fungoides; Myelodysplastic syndromes; Myelodysplastic/myeloproliferative diseases; Myeloproliferative disorders, chronic; Nasal cavity and paranasal sinus cancer; Nasopharyngeal carcinoma; Neuroblastoma; Oligodendroglioma; Oropharyngeal cancer; Osteosarcoma/malignant fibrous histiocytoma of bone; Ovarian cancer; Pancreatic cancer; Parathyroid cancer; Pharyngeal cancer; Pheochromocytoma; Pituitary adenoma; Plasma cell neoplasia; Pleuropulmonary blastoma; Prostate cancer; Rectal cancer; Renal cell carcinoma (kidney cancer); Renal pelvis and ureter, transitional cell cancer; Rhabdomyosarcoma; Salivary gland cancer; Sarcoma, Ewing family of tumors; Sarcoma, Kaposi; Sarcoma, soft tissue; Sarcoma, uterine; Sezary syndrome; Skin cancer (non-melanoma); Skin carcinoma; Small intestine cancer; Soft tissue sarcoma; Squamous cell carcinoma; Squamous neck cancer with occult primary, metastatic; Stomach cancer; Testicular cancer; Throat cancer; Thymoma and thymic carcinoma; Thymoma; Thyroid cancer; Thyroid cancer, childhood; Uterine cancer; Vaginal cancer; Waldenstrom macroglobulinemia; or Wilms tumor. The construct can be directed to an antigen associated with a cancer or tumor, such as CD5, CD19, CD20, CD25, CD37, CD30, CD33, CD45, CAMPATH-I, HLD-DR, carcinoembryonic antigen (CEA), TAG-72, EpCAM, MUC1, MUC15, folate-binding protein, A33, G250, prostate-specific membrane antigen (PSMA), ferritin, GD2, GD3, GM2, Le^{y}, CA-125, CA19-9, epidermal growth factor, p185HER2, IL-2 receptor, fibroblast activation protein (FAP), tenascin, a metalloproteinase, endosialin, vascular endothelial growth factor, avB3, WT1, LMP2, HPV E6, HPV E7, EGFRvIII (de2-7 EGFR), Her-2/neu, idiotype, MAGE A3, p53 nonmutant, NY-ESO-1, MelanA/MART1, Ras mutant, gp100, p53 mutant, PR1, bcr-abl, tyrosinase, survivin, PSA, hTERT, a Sarcoma translocation breakpoint protein, EphA2, PAP, ML-IAP, AFP, ERG, NA17, PAX3, ALK, androgen receptor, cyclin B1, polysialic acid, MYCN, RhoC, TRP-2, fucosyl GM1, mesothelin (MSLN), PSCA, MAGE A1, sLe(animal), CYP1B1, PLAV1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, B7H3, Legumain, Tie 3, Page4, VEGFR2, MAD-CT-1, PDGFR-B, MAD-CT-2, ROR2, CMET, HER3, EPCAM, CA6, NAPI2B, TROP2, CLDN18.2, RON, LY6E, FRA, DLL3, PTK7, LIV1, ROR1, Fos-related antigen 1, VEGFR, endoglin, PD-L1, CD204, CD206, CD301, VTCN1, or VISTA. The antigen binding domain can bind to an antigen expressed on an immune cell. The antigen binding domain can bind to an antigen expressed on an antigen presenting cell.

The peptide can comprise, for example, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acid residues. The peptide can comprise 25 amino acids. The peptide can be 50 amino acid residues. The peptide can be less than 50 amino acid residues. The peptide may comprise an antigenic fragment. The peptide can have sequence identity or homology to a sequence found in a cancer, wherein the sequence contains a mutation (e.g., an insertion, deletion or substitution in the amino acid sequence) associated with a cancer, herein after referred to as a cancer sequence. The mutation associated with a cancer can be a mutation found in a cancer cell that is not found a normal cell. The mutation associated with a cancer can be a non-self mutation in which the mutation is not found in proteins of a non-cancer cell in the patient. This mutation can be a driver mutation, i.e., a mutation that can give a selective advantage to a cancer cells in its environment. A driver mutation can give selective advantage to a cancer cell by increasing survival or reproduction. The mutation can be a passenger mutation, i.e., a mutation that has no effect on the fitness of the cancer cell, but can be associated with the cancer because it is linked with a driver mutation. The mutation in the cancer sequence can be from a single clonal population of cells from a subject, and therefore can be a cancer sequence unique to the cancer of that subject. The mutation in the cancer sequence can be a mutation commonly found in different clonal populations of cancer cells from subjects with a type of cancer. In some embodiments, said peptide comprises a non-synonymous mutation, neoantigen, splice variant of a tumor specific epitope, or a tumor specific epitope. The peptide can comprise a cancer sequence with a non-synonymous or missense mutation at any position in the peptide. The peptide can comprise a cancer sequence with a non-synonymous or missense mutation at a central amino acid residue in the peptide. The peptide can comprise a cancer sequence with a non-synonymous or missense mutation from a cancer at a central amino acid residue in the peptide. The peptide can comprise a cancer sequence with a non-synonymous or missense mutation from a cancer of a subject being treated with a construct-peptide composition, with the non-synonymous mutation at a central amino acid residue in the peptide. The peptide can be a dodecapeptide or a nonapeptide derived from the peptide with the non-synonymous mutation.

The peptide can bind with a K_{d} that is no greater than 500 nM, no great than 100 nM, no greater than 50 nM, no great than 25 nM, no greater than 10 nM, no greater than 5 nM, no greater than 1 nM, or no greater than 0.1 nM in the groove of an MHC molecule of the subject to be treated. The peptide or a processed form of the peptide may be capable of binding to an MHC molecule and stimulating an immune response when displayed. The MHC molecule can be an MHC class I molecule. The MHC molecule can be an MHC class II molecule. In humans, the MHC molecule can be an HLA molecule. For an MHC class I molecule, the peptide can be a nonapeptide. For an MHC class I molecule, the peptide can be 7 amino acid residues in length, 8 amino acid residues in length, 9 amino acid residues in length, 10 amino acid residues in length, or 11 amino acid residues in length. For an MHC class II molecule, the peptide can be a dodecapeptide. For a peptide that binds to a MHC Class I molecule, the antigen bound by the antigen binding domain may be a tumor associated antigen or an antigen expressed on an immune cell. For an MHC class II molecule, the peptide can be 10 amino acid residues in length, 11 amino acid residues in length, 12 amino acid residues in length, 13 amino acid residues in length, 14 amino acid residues in length, 15 amino acid residues in length, 16 amino acid residues in length, 17 amino acid residues in length, 18 amino acid residues in length, 19 amino acid residues in length, 20 amino acid residues in length, 22 amino acid residues in length, 23 amino acid residues in length, 24 amino acid residues in length, or 25 amino acid residues in length. In a peptide that binds to a MHC Class II molecule, the antigen binding domain may be an antigen expressed on an immune cell. The peptide can bind tightly to an HLA from the subject harboring the cancer sequence. The peptide can bind to an HLA on an antigen presenting cell. The peptide can bind to an HLA on an antigen presenting cell derived from the subject harboring the cancer sequence. The peptide can bind to an HLA on an antigen presenting cell expressing at least one HLA antigen in common with the subject harboring the cancer sequence. The peptide bound to an HLA can be recognized by a T-cell. The peptide bound to an HLA can be recognized by a T-cell from the subject harboring the cancer sequence. The T-cell recognizing the peptide bound to an HLA can be a CD4⁺ T cell. The T cell recognizing the peptide bound to an HLA can be a CD8⁺ T cell. The T cell recognizing the peptide bound to an HLA can be a PD-1⁺ T cell.

In some embodiments, the peptide binds to an MHC class I molecule. In some embodiments, the target antigen is a tumor associated antigen or an antigen expressed on an immune cell. In some embodiments, the target antigen is a tumor associated antigen. In some embodiments, the peptide binds to an MHC class II molecule. In some embodiments, the target antigen is an antigen expressed on an immune cell.

The sequence identity or homology of the peptide to a cancer sequence can be, for example, about 10%, about 20%, about 30%, about 40%, about 50%, about 55%, about 60%, about 65%, about 70%, about 75%, about 80%, about 85%, about 90%, about 95%, or about 99%. The peptide can have, for example, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acid residues. The peptide can have, for example, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid residues.

The cancer sequence of the peptide can be determined by, for example, sequencing the transcriptome, exome, or genome of a cancer cell. After sequencing, the resulting sequence can be compared to a normal cell to find a mutation that is specific to the cancer cell or the cancer sequence. Then, a peptide that is at least, for example, about 80% homologous or identical to the cancer sequence can be generated. The sample used for sequencing can be from, for example, blood cells, saliva, epithelial cells, bone, primary tumor tissue, adjacent normal tissue, distal normal tissue, the tumor micro environment, fibroblasts, stromal cells, biopsy, or metastatic tumor sites. The peptide can be generated by, for example, liquid-phase synthesis or solid-phase synthesis.

In some embodiments, a method producing the peptide may comprise sequencing the genome or transcriptome of a cancer cell to produce a cancer cell sequence; comparing the cancer cell sequence to a sequence from a normal cell to identify a mutation in the cancer cell sequence; and generating the antigenic peptide with at least 80% sequence identity to the cancer cell sequence with the mutation, wherein the mutation is present in the peptide. In some embodiments, the cancer cell and the normal cell are from one subject. In some embodiments, the cancer cell sequence is clonally represented within a cancer from a patient. In some embodiments, the cancer cell sequence contains a driver mutation of a cancer. In some embodiments, the method is used to produce a peptide library for a cancer type.

The K_{d} for binding of an antigen-binding domain to an antigen in the presence of an the peptide can be about 2 times, about 3 times, about 4 times, about 5 times, about 6 times, about 7 times, about 8 times, about 9 times, about 10 times, about 15 times, about 20 times, about 25 times, about 30 times, about 35 times, about 40 times, about 45 times, about 50 times, about 60 times, about 70 times, about 80 times, about 90 times, about 100 times, about 110 times, or about 120 times greater than the K_{d} for binding of the antigen binding domain to the antigen in the absence of the peptide. The K_{d} for binding of an antigen-binding domain to an antigen in the presence of an the peptide can be no greater than 500 nM, no greater than 100 nM, no greater than 50 nM, no greater than 25 nM, no greater than 10 nM, no greater than 5 nM, no greater than 1 nM, or no greater than 0.1 nM.

In some embodiments, the K_{d} for binding of the Fc domain to the Fc receptor in the presence of the immune-stimulatory compound is no greater than about two times, five times, ten times, or fifty times a K_{d} for binding of the Fc domain to the Fc receptor in an absence of the immune-stimulatory compound. The K_{d} for binding of an Fc domain to a Fc receptor in the presence of the peptide can be about 2 times, about 3 times, about 4 times, about 5 times, about 6 times, about 7 times, about 8 times, about 9 times, about 10 times, about 15 times, about 20 times, about 25 times, about 30 times, about 35 times, about 40 times, about 45 times, about 50 times, about 60 times, about 70 times, about 80 times, about 90 times, about 100 times, about 110 times, or about 120 times greater than the K_{d} for binding of the Fc domain to the Fc receptor in the absence of the peptide. The K_{d} for binding of an Fc domain to a Fc receptor in the presence of the peptide can be no greater than no greater than 10 pM, no greater than 1 pM 500 nM, no greater than 100 nM, no greater than 50 nM, no greater than 25 nM, no greater than 10 nM, no greater than 5 nM, no greater than 1 nM, or no greater than 0.1 nM.

Affinity can be the strength of the sum total of noncovalent interactions between a single binding site of a molecule, for example, an antibody, and the binding partner of the molecule, for example, an antigen. The affinity can also measure the strength of an interaction between an Fc portion of an antibody and the Fc receptor. Unless indicated otherwise, as used herein, “binding affinity” refers to intrinsic binding affinity which reflects a 1:1 interaction between members of a binding pair (e.g., antibody and antigen or Fc domain and Fc receptor). The affinity of a molecule X for its partner Y can generally be represented by the dissociation constant (K_{d}). Affinity can be measured by common methods known in the art, including those described herein. Specific illustrative and exemplary embodiments for measuring binding affinity are described in the following.

In some embodiments, an antibody provided herein can have a dissociation constant (K_{d}) of about 1 pM, about 500 nM, about 100 nM, about 10 nM, about 5 nM, about 2 nM, about 1 nM, about 0.5 nM, about 0.1 nM, about 0.05 nM, about 0.01 nM, or about 0.001 nM or less (e.g., 10⁻⁸ M or less, e.g., from 10⁻⁸ M to 10⁻¹³ M, e.g., from 10⁻⁹ M to 10⁻¹³ M). An affinity matured antibody can be an antibody with one or more alterations in one or more complementarity determining regions (CDRs), compared to a parent antibody, which may not possess such alterations, such alterations resulting in an improvement in the affinity of the antibody for antigen. These antibodies can bind to their antigen with a K_{d} of about 5×10^{0.9} M, about 2×10^{0.9} M, about 1×10⁻⁹ M, about 5×10⁻¹ M, about 2×10⁻⁹ M, about 1×10⁻¹⁰ M, about 5×10⁻¹¹ M, about 1×10⁻¹¹ M, about 5×10⁻¹² M, about 1×10⁻¹² M, or less. In some embodiments, the construct can have an increased affinity of at least 1.5-fold, 2-fold, 2.5-fold, 3-fold, 4-fold, 5-fold, 10-fold, 20-fold, or greater as compared to a construct without alterations in one or more complementarity determining regions.

K_{d} can be measured by any suitable assay. For example, K_{d} can be measured by a radiolabeled antigen binding assay (RIA). For example, K_{d} can be measured using surface plasmon resonance assays (e.g., using a BIACORE®-2000 or a BIACORE®-3000).

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

### Immune-Stimulatory Compounds Conjugated to the Construct-Peptide Composition

Pattern recognition receptors (PRRs) can recognize pathogen-associated molecular patterns (PAMPs) and damage-associated molecular patterns (DAMPs). A PRR can be membrane bound. A PRR can be cytosolic. A PRR can be a toll-like receptor (TLR). A PRR can be RIG-I-like receptor. A PRR can be a receptor kinase. A PRR can be a C-type lectin receptor. A PRR can be a NOD-like receptor. A PRR can be TLR1, TLR2, TLR3, TLR4, TLR5, TLR6, TLR7, TLR8, TLR9, TLR10, TLR11, TLR12, or TLR13.

In some embodiments, the immune-stimulatory compound is a damage-associated molecular pattern molecule or a pathogen-associated molecular pattern molecule. In some embodiments, the damage-associated molecular pattern molecule is HMGP1, S100A8/S100A9, ATP, uric acid, APP, SAA, granulysin, or eosinophil-derived neurotoxin. In some embodiments, the immune-stimulatory compound is a toll-like receptor agonist, STING agonist, or RIG-I agonist. In some embodiments, the immune-stimulatory compound is a TLR1 agonist, a TLR2 agonist, a TLR3 agonist, a TLR4 agonist, a TLR5 agonist, a TLR6 agonist, a TLR7 agonist, a TLR8 agonist, a TLR9 agonist, or a TLR10 agonist.

In some embodiments, the immune-stimulatory compound is selected from a group consisting of: CpG oligonucleotide, Poly G10, Poly G3, Poly I:C, Lipopolysaccharide, zymosan, Flagellin, Pam3CSK4, PamCysPamSK4, dsRNA, a diacylated lipopeptide, a triacylated lipoprotein, lipoteichoic acid, a peptidoglycan, a cyclic dinucleotide, a 5′ppp-dsRNA, S-27609, CL307, UC-IV150, imiquimod, gardiquimod, resiquimod, motolimod, VTS-1463GS-9620, GSK2245035, TMX-101, TMX-201, TMX-202, isatoribine, AZD8848, MEDI9197, 3M-051, 3M-852, 3M-052, 3M-854A, S-34240, KU34B, CL663, SB9200, SB11285, or 8-substituted 2-amino-3H-benzo[b]azepine-4-carbozamide.

A PRR agonist can be pathogen-associated molecular pattern (PAMP) molecule. A PAMP molecule can be a toll-like receptor agonist. A PRR agonist can be a toll-like receptor agonist. A toll-like receptor agonist can be any molecule that acts as an agonist to at least one toll-like receptor. A toll-like receptor agonist can be bacterial lipoprotein. A toll-like receptor agonist can be bacterial peptidoglycans. A toll-like receptor agonist can be double stranded RNA. A toll-like receptor agonist can be lipopolysaccharides. A toll-like receptor agonist can be bacterial flagella. A toll-like receptor agonist can be single stranded RNA. A toll-like receptor can be CpG DNA. A toll-like receptor agonist can be imiquimod. A toll-like receptor agonist can be CL307. A toll-like receptor agonist can be S-27609. A toll-like receptor agonist can be resiquimod. A toll-like receptor agonist can be UC-IV150. A toll-like receptor agonist can be gardiquimod. A toll-like receptor agonist can be motolimod. A toll-like receptor agonist can be VTX-1463. A toll-like receptor agonist can be GS-9620. A toll-like receptor agonist can be GSK2245035. A toll-like receptor agonist can be TMX-101. A toll-like receptor agonist can be TMX-201. A toll-like receptor agonist can be TMX-202. A toll-like receptor agonist can be isatoribine. A toll-like receptor agonist can be AZD8848. A toll-like receptor agonist can be MEDI9197. A toll-like receptor agonist can be 3M-051. A toll-like receptor agonist can be 3M-852. A toll-like receptor agonist can be 3M-052. A toll-like receptor agonist can be 3M-854A. A toll-like receptor agonist can be S-34240. A toll-like receptor agonist can be CL663. A RIG-I agonist can be KIN1148. A RIG-I agonist can be SB-9200. A RIG-I agonist can be KIN700, KIN600, KIN500, KIN100, KIN101, KIN400, or KIN2000. A toll-like receptor agonist can be KU34B. A toll-like receptor agonist can be SB9200. A toll-like receptor agonist can be SB11285. A toll-like receptor agonist can be 8-substituted 2-amino-3H-benzo[b]azepine-4-carbozamide.

A PRR agonist can be a damage-associated molecular pattern (DAMP) molecule. A DAMP molecule can be an intracellular protein. A DAMP molecule can be a heat-shock protein. A DAMP molecule can be an HMGB1 protein. A DAMP molecule can be a protein derived from the extracellular matrix that is generated after tissue injury. A DAMP molecule can be a hyaluronan fragment. A DAMP molecule can be DNA. A DAMP molecule can be RNA. A DAMP molecule can be an S100 molecule. A DAMP molecule can be nucleotides. A DAMP molecule can be an ATP. A DAMP molecule can be nucleosides. A DAMP molecule can be an adenosine. A DAMP molecule can be uric acid.

Additionally, stimulator of interferon genes (STING) can act as a cytosolic DNA sensor wherein cytosolic DNA and unique bacterial nucleic acids called cyclic dinucleotides are recognized by STING, and therefore STING agonists. Interferon Regulatory Factor (IRF) agonist can be KIN-100. Non-limiting examples of STING agonists include:

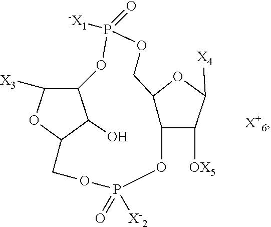

wherein in some embodiments, X₁═X₂═O; X₃=G; X₄=G; X₅=CO(CH₂)₁₂CH₃; X₆=2 TEAH; in some embodiments, X₁═X₂═S [RₚRₚ]; X₃=G; X₄=A; X₅═H; X₆=2 TEAH; in some embodiments, X₁═X₂═S [RₚRₚ,]; X₃=A; X₄=A; X₅═H; X₆=2 Na; in some embodiments, X₁═X₂═S [RₚRₚ]; X₃=A; X₄=A; X₅═H; X₆=2, NH₄; and in some embodiments, X₁═X₂O; X₃=G; X₄=A; X₅═H; X₆=2 TEAH,

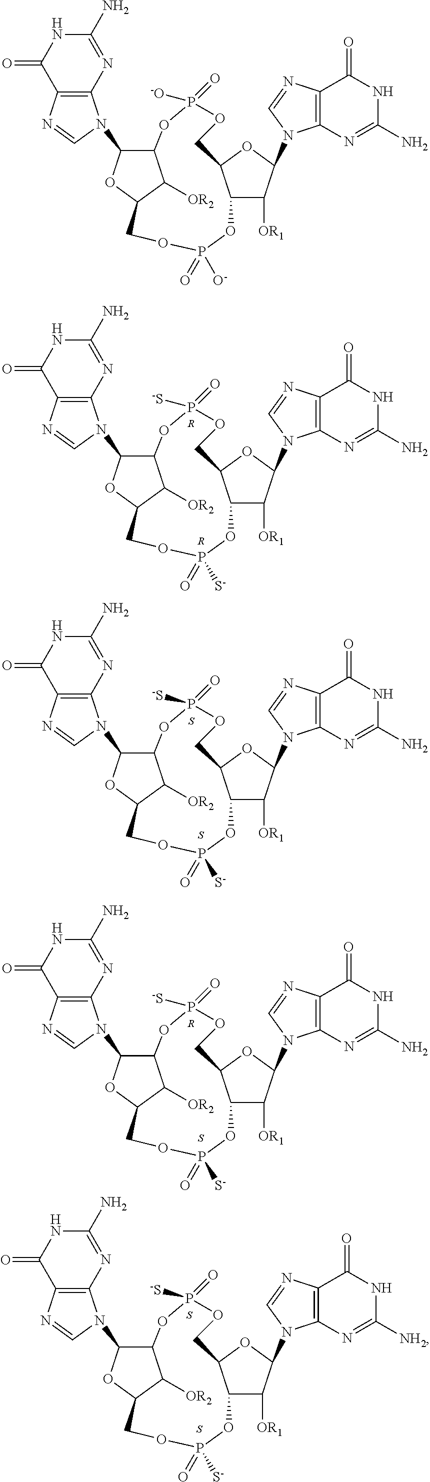

wherein R₁═R₂═H; R₁=propargyl, R₂═H; R₁═H, R₂=propargyl; R₁=allyl, R₂═H; R₁═H, R₂=allyl; R₁=methyl, R₂═H; R₁═H, R₂=methyl; R₁=ethyl, R₂═H; R₁═H, R₂=ethyl; R₁=propyl, R₂═H; R₁═H, R₂=propyl; R₁=benzyl, R₂═H; R₁═H, R₂=benzyl; R₁=myristoyl, R₂═H; R₁═H, R₂=myristoyl; R₁═R₂=heptanoyl; R₁═R₂=hexanoyl; or R₁═R₂=pentanoyl,

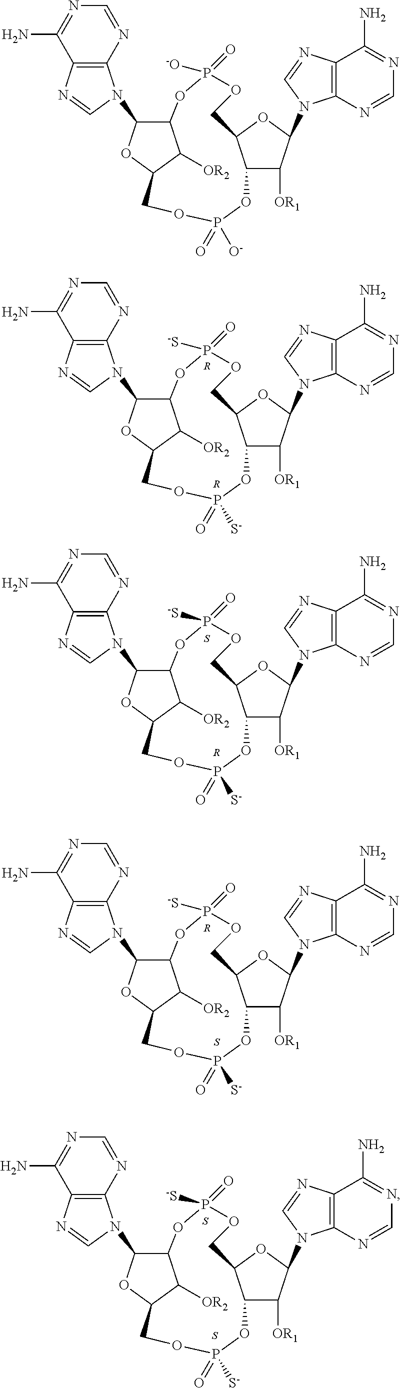

wherein R₁═R₂═H; R₁=propargyl, R₂═H; R₁═H, R₂=propargyl; R₁=allyl, R₂═H; R₁═H, R₂=allyl; R₁=methyl, R₂═H; R₁═H, R₂=methyl; R₁=ethyl, R₂═H; R₁═H, R₂=ethyl; R₁=propyl, R₂═H; R₁═H, R₂=propyl; R₁=benzyl, R₂═H; R₁═H, R₂=benzyl; R₁=myristoyl, R₂═H; R₁═H, R₂=myristoyl; R₁═R₂=heptanoyl; R₁═R₂=hexanoyl; or R₁═R₂=pentanoyl,

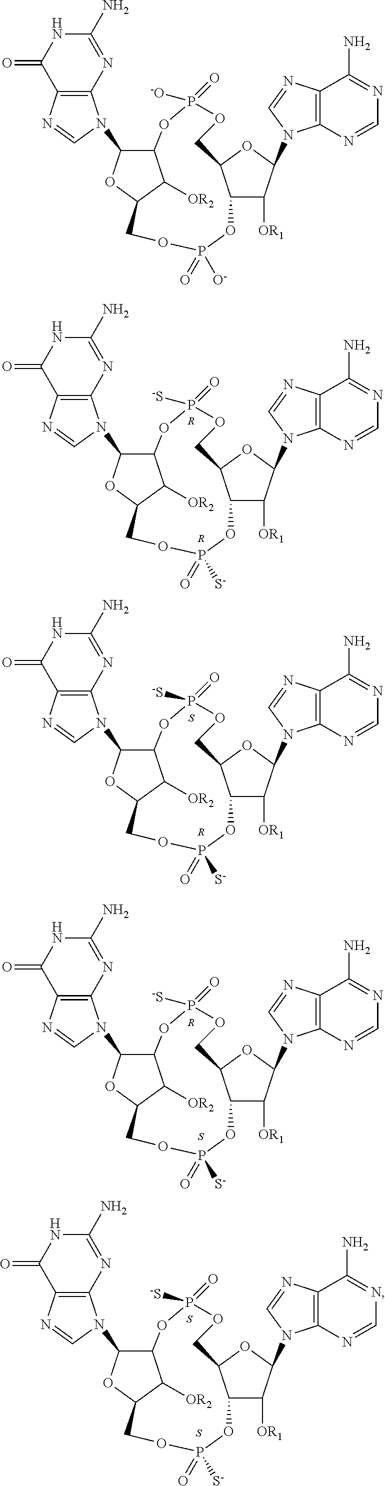

wherein R₁═R₂═H; R₁=propargyl, R₂═H; R₁═H, R₂=propargyl; R₁=allyl, R₂═H; R═H, R₂=allyl; R₁=methyl, R₂═H; R₁═H, R₂=methyl; R₁=ethyl, R₂═H; R₁═H, R₂=ethyl; R₁=propyl, R₂═H; R₁═H, R₂=propyl; R₁=benzyl R₂═H; R₁═H, R₂=benzyl; R₁=myristoyl R₂═H; R₁═H, R₂=myristoyl; R₁═R₂=heptanoyl; R₁═R₂=hexanoyl; or R₁═R₂=pentanoyl,

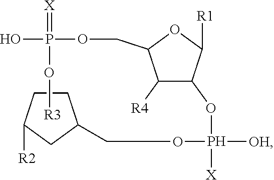

wherein each X is independently O or S, and R3 and R4 are each independently H or an optionally substituted straight chain alkyl of from 1 to 18 carbons and from 0 to 3 heteroatoms, an optionally substituted alkenyl of from 1-9 carbons, an optionally substituted alkynyl of from 1-9 carbons, or an optionally substituted aryl, wherein substitution(s), when present, may be independently selected from the group consisting of C₁₋₆ alkyl straight or branched chain, benzyl, halogen, trihalomethyl, C₁₋₆ alkoxy, —NO₂, —NH₂, —OH, =, —COOR′ where R′ is H or lower alkyl, —CH₂OH, and —CONH₂, wherein R3 and R4 are not both H,

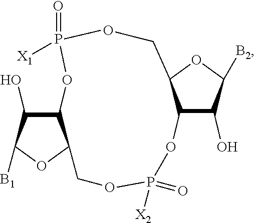

wherein X₁═X₂═O; X₁═X₂═S; or X₁═O and X₂═S,

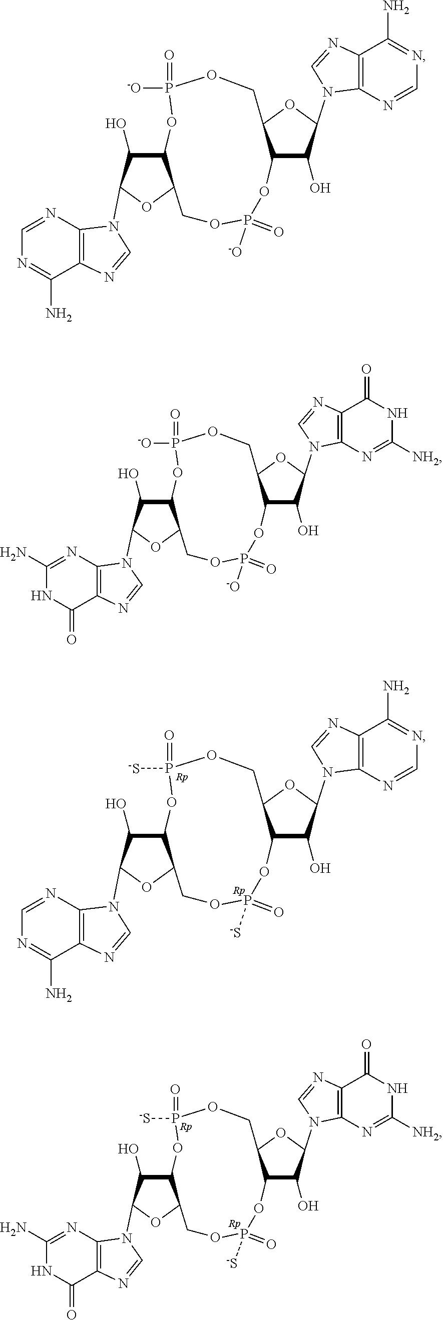

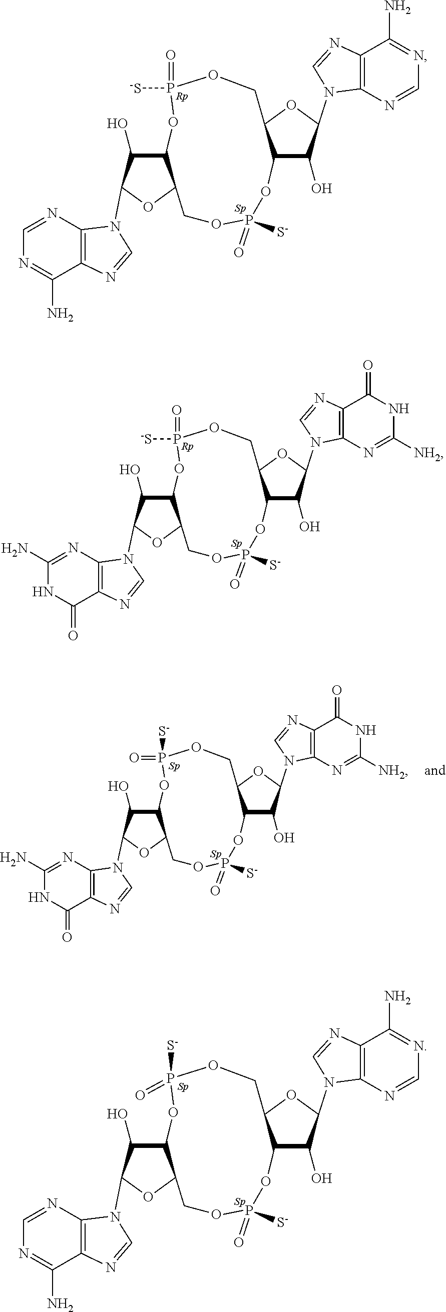

An immune-stimulatory compound can be a PRR agonist. An immune-stimulatory compound can be a PAMP. An immune-stimulatory compound can be a DAMP. An immune-stimulatory compound can be a TLR agonist. An immune-stimulatory compound can be a STING agonist. An immune-stimulatory compound can be a cyclic dinucleotide. In some aspects, an immune-stimulatory compound can be represented by the structure of Formula (I):

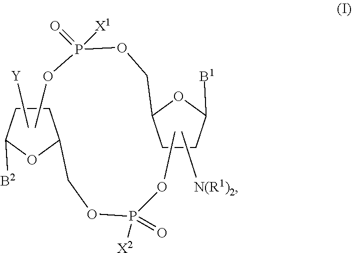

or a pharmaceutically acceptable salt thereof, wherein:

X¹ is selected from —OR² and —SR²;

X² is selected from —OR³ and —SR³;

B¹ and B² are independently selected from optionally substituted nitrogenous bases;

Y is selected from —OR⁴, —NR⁴R⁴, and halogen;

R¹, R², R³ and R⁴ are independently selected at each occurrence from hydrogen, —C(═O)R¹⁰⁰, —C(═O)OR¹⁰⁰ and —C(═O)NR¹⁰⁰; C₁₋₁₀ alkyl, C₂₋₁₀ alkenyl, C₂₋₁₀ alkynyl, each of which is independently optionally substituted at each occurrence with one or more substituents selected from halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, —S, ═N(R¹⁰⁰), —P(O)(OR¹⁰⁰)₂, —OP(O)(OR¹⁰⁰)₂, —CN, C₃₋₁₀ carbocycle and 3- to 10-membered heterocycle; and C₃₋₁₀ carbocycle and 3- to 10-membered heterocycle, wherein each C₃₋₁₀ carbocycle and 3- to 10-membered heterocycle in R¹, R², R³ and R⁴ is independently optionally substituted with one or more substituents selected from halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —P(O)(OR¹⁰⁰)₂, —OP(O)(OR¹⁰⁰)₂, —CN, C₁₋₆ alkyl, C₂₋₆ alkenyl, and C₂₋₆ alkynyl; and

R¹⁰⁰ at each occurrence is independently selected from hydrogen; and C₁₋₁₀ alkyl, C₂₋₁₀ alkenyl, C₂₋₁₀ alkynyl, C₃₋₁₀ carbocycle, and 3- to 10-membered heterocycle each of which is independently optionally substituted at each occurrence with one or more substituents selected from halogen, —CN, —NO₂, ═O, ═S, and haloalkyl.

In some embodiments, the compound of Formula (I) is represented by Formula (IA):

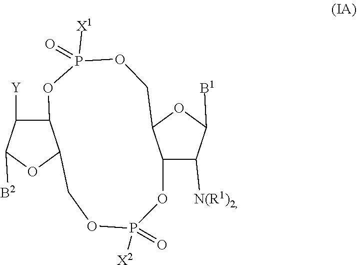

or pharmaceutically acceptable salts thereof.

In an alternative embodiment, the compound of Formula (I) is represented by Formula (IB):

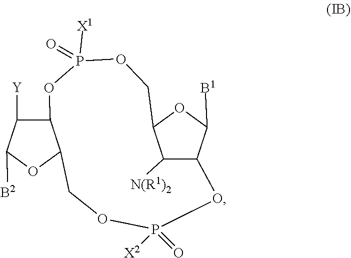

or a pharmaceutically acceptable salt thereof.

In various embodiments, B¹ and B² are independently selected from optionally substituted purines. In certain embodiments, B¹ and B² are independently selected from

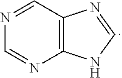

In certain embodiments, B¹ and B² are independently selected from optionally substituted pyrimidines.

In some embodiments, optionally substituted purines may include optionally substituted adenine, optionally substituted guanine, optionally substituted xanthine, optionally substituted hypoxaanthine, optionally substituted theobromine, optionally substituted caffeine, optionally substituted uric acid, and optionally substituted isoguanine. In certain embodiments, B¹ and B² are independently selected from:

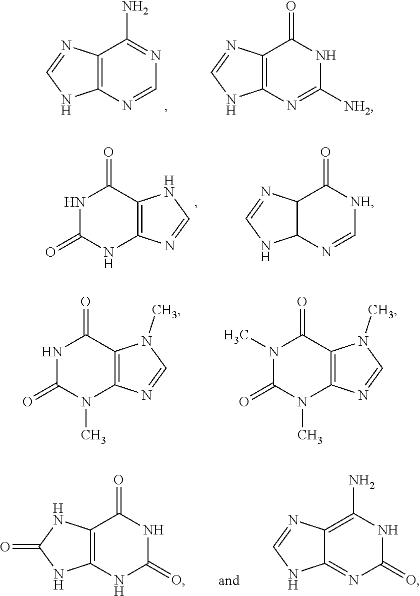

optionally substituted by one or more additional substituents.

In certain embodiments, B¹ and B² are independently selected from:

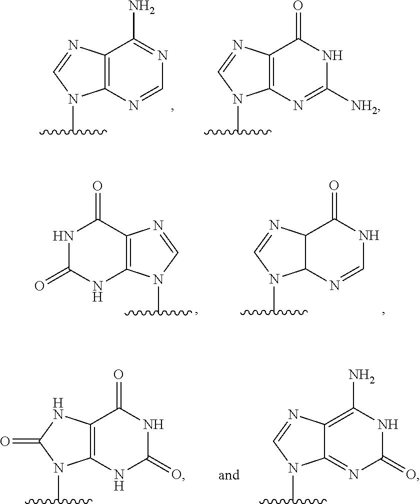

wherein the point of connectivity of B¹ to the remainder of the compound is represented by

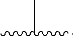

In a preferred embodiment, B¹ and B² are independently selected from optionally substituted adenine and optionally substituted guanine. In certain embodiments, B¹ and B² are independently selected from:

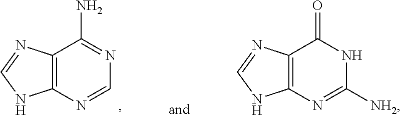 [SMILES: Nc1ncnc2[nH]cnc12 ; Nc1nc2[nH]cnc2c(=O)[nH]1 ; "," ; "and"]

optionally further substituted by one or more substituents. In certain embodiments, B¹ and B² are independently selected from:

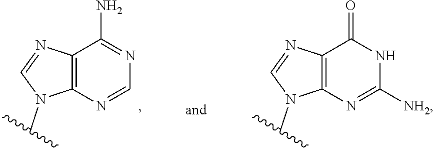 [SMILES: *n1cnc2c(N)ncnc21 ; "," ; "and" ; *n1cnc2c(=O)[nH]c(N)nc21]

In some embodiments, B¹ and B² are independently optionally substituted with one or more substituents, wherein the optional substituents on B¹ and B² are independently selected at each occurrence from halogen, ═O, ═S, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, —P(O)(OR¹⁰⁰)₂, —OP(O)(OR¹⁰⁰)₂ and —CN; C₁₋₁₀ alkyl, C₂₋₁₀ alkenyl, C₂₋₁₀ alkynyl, each of which is independently optionally substituted at each occurrence with one or more substituents selected from halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, —S, ═N(R¹⁰⁰), —P(O)(OR¹⁰⁰)₂, —OP(O)(OR¹⁰⁰)₂, —CN, C₃₋₁₀ carbocycle and 3- to 10-membered heterocycle; and C₃₋₁₀ carbocycle and 3- to 10-membered heterocycle, wherein each C₃₋₁₀ carbocycle and 3- to 10-membered heterocycle is independently optionally substituted with one or more substituents selected from halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —P(O)(OR¹⁰⁰)₂, —OP(O)(OR¹⁰⁰)₂, —CN, C₁₋₆ alkyl, C₂₋₆ alkenyl, and C₂₋₆ alkynyl.

In certain embodiments, B¹ and B² are independently optionally substituted with one or more substituents, wherein the optional substituents on B¹ and B² are independently selected at each occurrence from halogen, ═O, ═S, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, —P(O)(OR¹⁰⁰)₂, —OP(O)(OR¹⁰⁰)₂, —CN and C₁₋₁₀ alkyl.

In some embodiments, B¹ is an optionally substituted guanine. In certain embodiments, B¹ is

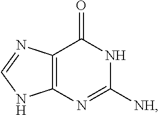

In certain embodiments, B¹ is

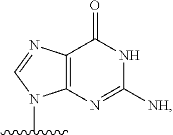

wherein the point of connectivity of B¹ to the remainder of the compound is represented by

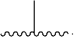

In some embodiments, B¹ is an optionally substituted adenine. In certain embodiments, B¹ is

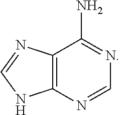

In certain embodiments, B¹ is

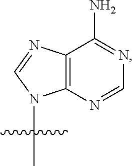 [SMILES: *n1cnc2c(N)ncnc21]

wherein the point of connectivity of B¹ to the remainder of the compound is represented by

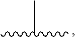

In some embodiments, B² is an optionally substituted guanine. In certain, embodiments, B² is

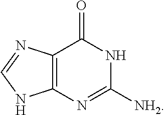 [SMILES: Nc1nc2[nH]cnc2c(=O)[nH]1]

In certain embodiments, B² is

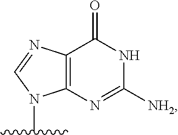 [SMILES: *n1cnc2c(=O)[nH]c(N)nc21]

wherein the point of connectivity on B² is represented by

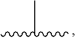

In some embodiments, B² is an optionally substituted adenine. In certain embodiments, B² is

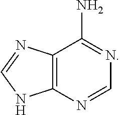

In certain embodiments, B² is

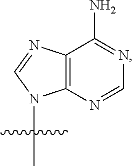 [SMILES: *n1cnc2c(N)ncnc21]

wherein the point of connectivity on B² is represented by

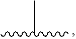

In some embodiments, B¹ is an optionally substituted guanine and B² is an optionally substituted guanine. In some embodiments, B¹ is an optionally substituted adenine and B² is an optionally substituted guanine.

In various embodiments, X¹ is selected from —OH and —SH. For example, X¹ may be —OH. In various embodiments, X² is selected from —OH and —SH. For example, X² may be —OH. In some embodiments, X¹ is —OH and X² is —OH. In some embodiments, X¹ is —SH and X² is —SH.

In various embodiments, Y is selected from —OH, —O—C₁₋₁₀alkyl, —NH(C₁₋₁₀alkyl), and —NH₂. For example, Y may be —OH.

In various embodiments, R¹⁰⁰ is independently selected at each occurrence from hydrogen and C₁₋₁₀ alkyl optionally substituted at each occurrence with one or more substituents selected from halogen, —CN, —NO₂, ═O, and ═S.

In various embodiments, the compound of Formula (I) is represented by Formula (IC):

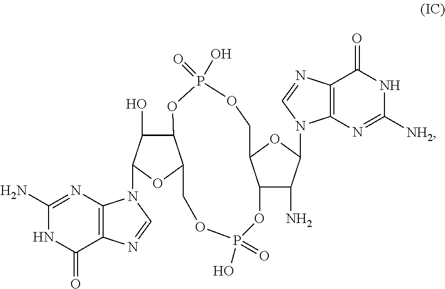 [SMILES: Nc1nc2c(ncn2C2OC3COP(=O)(O)OC4C(COP(=O)(O)OC3C2N)OC(n2cnc3c(=O)[nH]c(N)nc32)C4O)c(=O)[nH]1]

or a pharmaceutically acceptable salt thereof. In some embodiments, the compound of Formula (IC) is represented by Formula (ID):

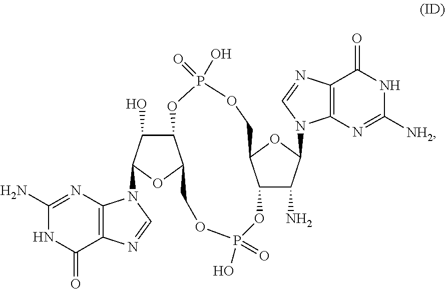 [SMILES: Nc1nc2c(ncn2[C@@H]2O[C@@H]3COP(=O)(O)O[C@H]4[C@@H](O)[C@H](n5cnc6c(=O)[nH]c(N)nc65)O[C@@H]4COP(=O)(O)O[C@H]3[C@H]2N)c(=O)[nH]1]

or a pharmaceutically acceptable salt thereof.

The compound or salt can be a modulator of a stimulator of interferon genes (STING). The compound or salt can agonize a stimulator of interferon genes (STING). The compound or salt can cause STING to coordinate multiple immune responses to infection, including the induction of interferons and STAT6-dependent response and selective autophagy response. The compound or salt can cause STING to mediate type I interferon production.

In some aspects, the present disclosure provides a construct-peptide immune-stimulatory compound conjugate, comprising a compound or salt previously described, a construct, and a linker group, wherein the compound or salt is linked, e.g., covalently bound, to the construct through the linker group. The construct can be an antibody. The linker group can be selected from a cleavable or non-cleavable linker. In some embodiments, the linker group is cleavable. In alternative embodiments, the linker group is non-cleavable. Linkers are further described in the present application in the subsequent section, any one of which may be used to connect a construct as described herein to a compound described herein.

In some aspects, the present disclosure provides a compound represented by the structure of Formula (II):

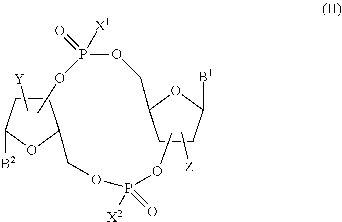

or a pharmaceutically acceptable salt thereof, wherein:

X¹ is selected from —OR² and —SR²;

X² is selected from —OR³ and —SR³;

B¹ and B² are independently selected from optionally substituted nitrogenous bases, wherein each optional substituent is independently selected from halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —CN, R⁶, and —X³;

Y is selected from —OR⁴, —SR⁴, —NR⁴R⁴, and halogen;

Z is selected from —OR^{5,} —SR⁵, and —NR⁵R⁵;

R¹, R², R³, R⁴, and R⁵ are independently selected from a —X³; hydrogen, —C(═O)R¹⁰⁰, —C(═O)OR¹⁰⁰ and —C(═O)NR¹⁰⁰; C₁₋₁₀ alkyl, C₂₋₁₀ alkenyl, C₂₋₁₀ alkynyl, each of which is independently optionally substituted at each occurrence with one or more substituents selected from halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —P(O)(OR¹⁰⁰)₂, —OP(O)(OR¹⁰⁰)₂, —CN, C₃₋₁₀ carbocycle and 3- to 10-membered heterocycle; and C₃₋₁₀ carbocycle and 3- to 10-membered heterocycle, wherein each C₃₋₁₀ carbocycle and 3- to 10-membered heterocycle in R¹, R², R³, R⁴, and R⁵ is optionally substituted with one or more substituents selected from halogen, —OR¹⁰⁰, —SR¹⁰⁰, —N(R¹⁰⁰)₂, —S(O)R¹⁰⁰, —S(O)₂R¹⁰⁰—C(O)R¹⁰⁰, —C(O)OR¹⁰⁰, —OC(O)R¹⁰⁰, —NO₂, —O, —S, ═N(R¹⁰⁰), —P(O)(OR¹⁰⁰)₂, —OP(O)(OR¹⁰⁰)₂, —CN, C₁₋₆ alkyl, C₂₋₆ alkenyl, C₂₋₆ alkynyl; R⁶ is independently selected from —C(═O)R¹⁰⁰, —C(═O)OR¹⁰⁰ and —C(═O)NR¹⁰⁰; C₁₋₁₀ alkyl, C₂₋₁₀ alkenyl, C₂₋₁₀ alkynyl, each of which is independently optionally substituted at each occurrence with one or more substituents selected from halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —P(O)(OR¹⁰⁰)₂, —OP(O)(OR¹⁰⁰)₂, —CN, C₃₋₁₀ carbocycle and 3- to 10-membered heterocycle; and C₃₋₁₀ carbocycle and 3- to 10-membered heterocycle, wherein each C₃₋₁₀ carbocycle and 3- to 10-membered heterocycle in R⁶ is optionally substituted with one or more substituents selected from halogen, —OR¹⁰⁰, —SR¹⁰⁰, —N(R¹⁰⁰)₂, —S(O)R¹⁰⁰, —S(O)₂R¹⁰⁰—C(O)R¹⁰⁰, —C(O)OR¹⁰⁰, —OC(O)R¹⁰⁰, —NO₂, ═O, ═S, ═N(R¹⁰⁰), —P(O)(OR¹⁰⁰)₂, —OP(O)(OR¹⁰⁰)₂, —CN, C₁₋₆ alkyl, C₂₋₆ alkenyl, C₂₋₆ alkynyl; R¹⁰⁰ at each occurrence is independently selected from hydrogen; and C₁₋₁₀ alkyl, C₂₋₁₀ alkenyl, C₂₋₁₀ alkynyl, C₃₋₁₀ carbocycle, and 3- to 10-membered heterocycle each of which is independently optionally substituted at each occurrence with one or more substituents selected from halogen, —CN, —NO₂, =0, ═S, and haloalkyl; and

X³ is a linker moiety, wherein at least one of R¹, R², R³, R⁴, R⁵, X¹, X², a B¹ substituent and a B² substituent is —X³.

In various embodiments, the compound of Formula (II) is represented by a structure of Formula (IIA):

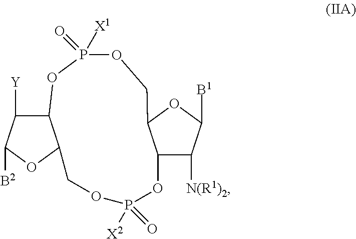

or pharmaceutically acceptable salts thereof.

In various embodiments, the compound of Formula (II) is represented by a structure of Formula (IIB):

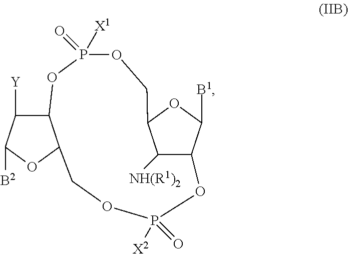

or a pharmaceutically acceptable salt thereof.

In various embodiments, B¹ and B² are independently selected from optionally substituted purines. B¹ and B² may be each, independently selected from one another, adenine, guanine, and derivatives thereof. B¹ and B² may be independently selected from optionally substituted adenine, optionally substituted guanine, optionally substituted xanthine, optionally substituted hypoxanthine, optionally substituted theobromine, optionally substituted caffeine, optionally substituted uric acid, and optionally substituted isoguanine. In a preferred embodiment, B¹ and B² are independently selected from optionally substituted adenine and optionally substituted guanine.

In various embodiments, B¹ is substituted by X³ and optionally one or more additional substituents independently selected from halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, =—S, ═N(R¹⁰⁰), —CN, and R⁶. For example, B¹ may be represented by:

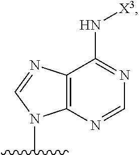

and wherein B¹ is optionally further substituted by one or more substituents.

In various embodiments, B² is substituted by X³ and optionally one or more additional substituents independently selected from halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —CN, and R⁶. For example, B² may be represented by:

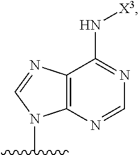

and wherein B² is optionally further substituted by one or more substituents.

In some embodiments, B¹ is represented by

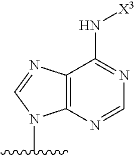

and B² is represented by

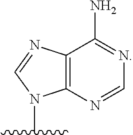 [SMILES: *n1cnc2c(N)ncnc21]

In some embodiments, B¹ is represented by

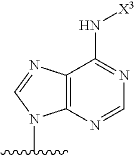

and B² is represented by

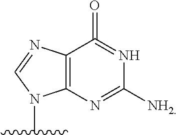

In various embodiments, X¹ is selected from —O— X³ and —S—X³. In some embodiments, X¹ is selected from —OH and —SH. In some embodiments, X¹ is —SH.

In various embodiments, X² is selected from —O— X³ and —S—X³. In some embodiments, X² is selected from —OH and —SH. In some embodiments, X² is —S—X³.

In some embodiments, X¹ is —SH and X² is —S—X³.

In certain embodiments, Y is selected from —NR⁴X³, —S—X³, and —O— X³. In some embodiments, Y is selected from —OH, —SH, —O—C₁₋₁₀alkyl, —NH(C₁₋₁₀alkyl), and —NH₂. In a preferred embodiment, Y is selected from —OH.

In various embodiments, Z is selected from —NR⁴X³, —S—X³, and —O— X³. In some embodiments, Z is selected from —OH, —SH, —O—C₁₋₁₀ alkyl, —NH(C₁₋₁₀ alkyl), and —NH₂.

In various embodiments, —X³ is represented by the formula:

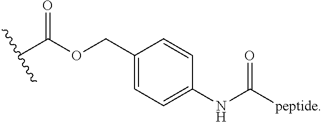

In some embodiments, —X³ is represented by the formula:

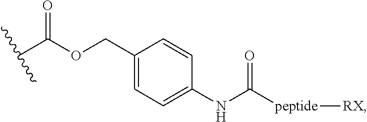

wherein RX comprises a reactive moiety, such a maleimide.

In some embodiments, —X³ is represented by the formula:

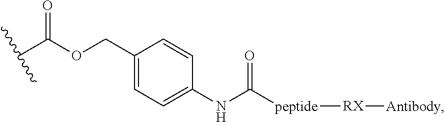

wherein RX* is a reactive moiety that has reacted with a moiety on a construct, such as an antibody, to form an antibody-immune stimulatory compound conjugate.

In some embodiments, —X³ is represented by the formula:

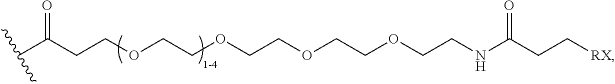

wherein RX is a reactive moiety, such as a maleimide.

In some embodiments, —X³ is represented by the formula:

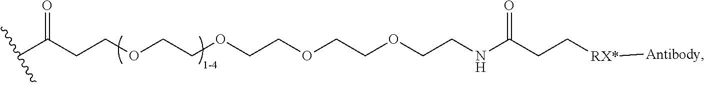

wherein RX* is a reactive moiety that has reacted with a moiety on a construct, such as antibody to form an antibody-immune stimulatory compound conjugate.

In some embodiments, the compound is represented by the formula:

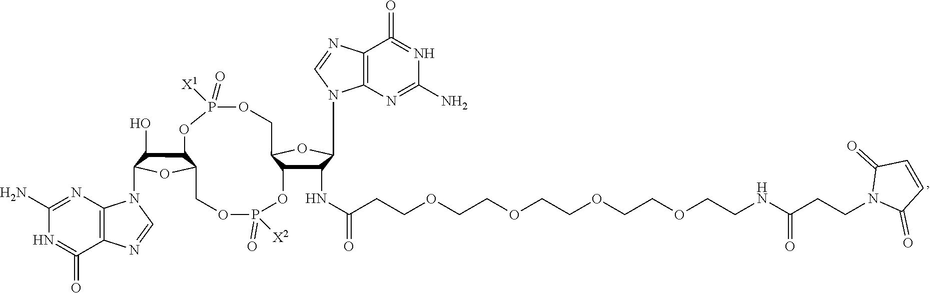

or a pharmaceutically acceptable salt thereof. The compound may be represented by the formula:

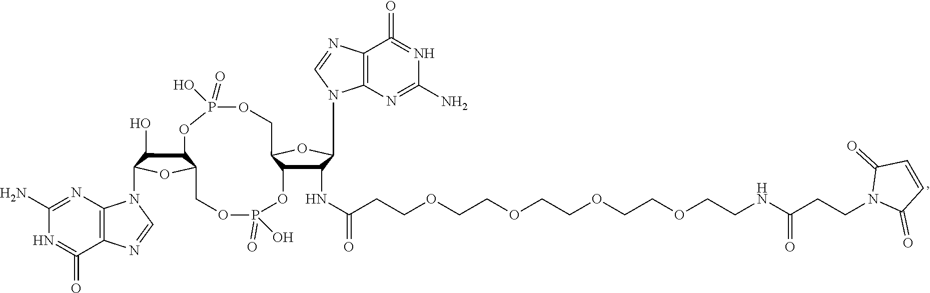

or a pharmaceutically acceptable salt thereof. The compound may be represented by the formula:

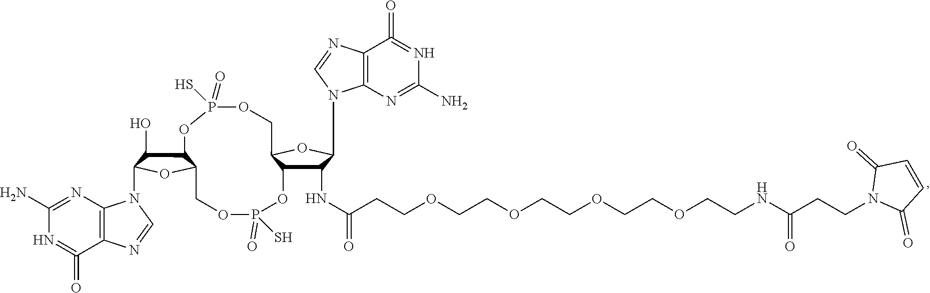

or a pharmaceutically acceptable salt thereof.

In some embodiments, the compound is represented by the formula:

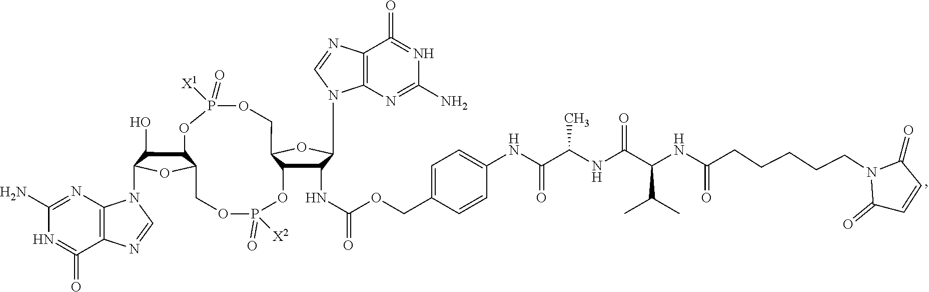

or a pharmaceutically acceptable salt thereof. The compound may be represented by the formula:

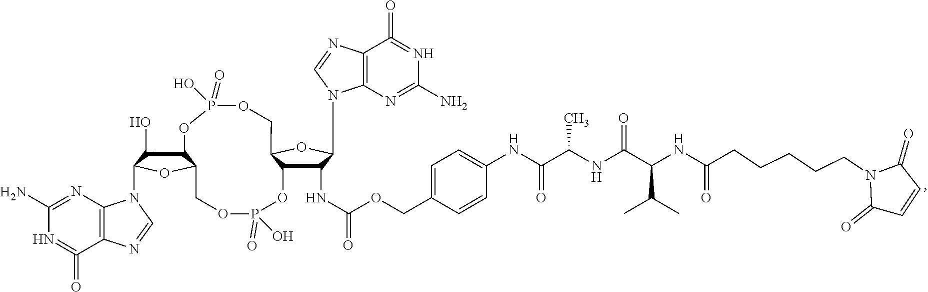

or a pharmaceutically acceptable salt thereof. The compound may be represented by the formula:

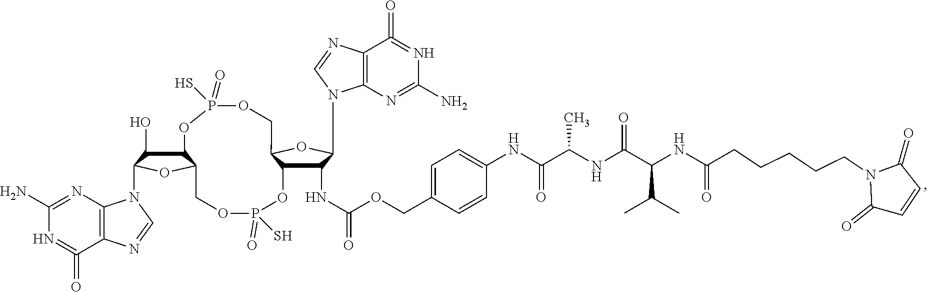

or a pharmaceutically acceptable salt thereof.

In some embodiments, the compound is represented by the formula:

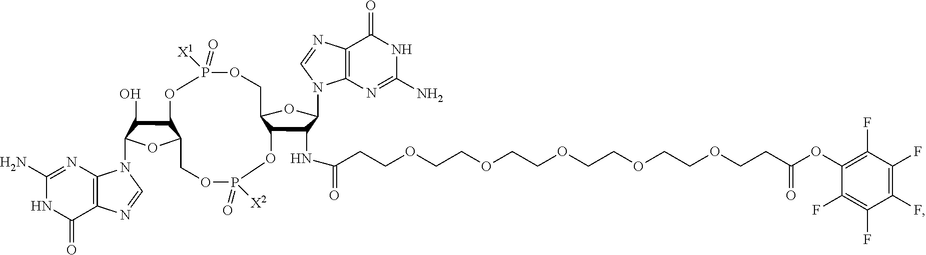

or a pharmaceutically acceptable salt thereof. The compound may be represented by the formula:

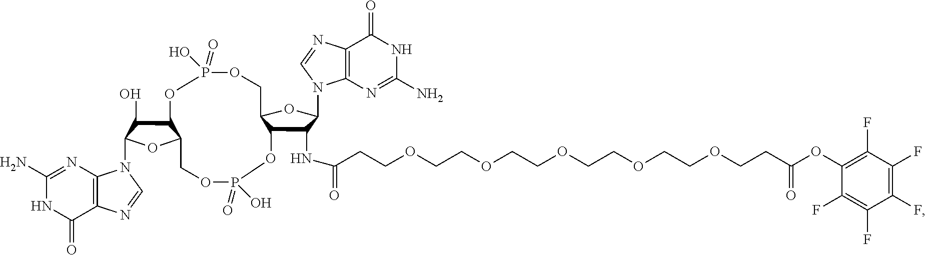

or a pharmaceutically acceptable salt thereof. The compound may be represented by the formula:

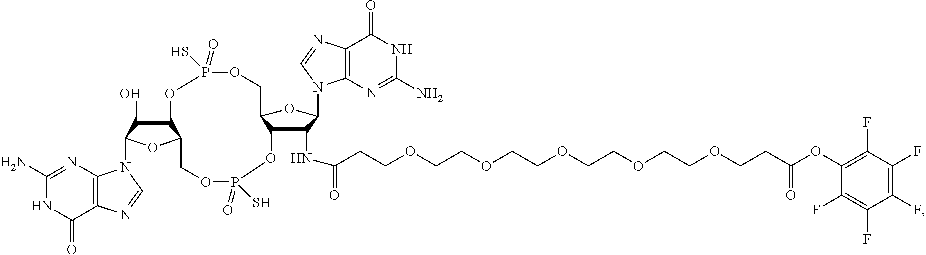

or a pharmaceutically acceptable salt thereof.

In some embodiments, the compound is represented by the formula:

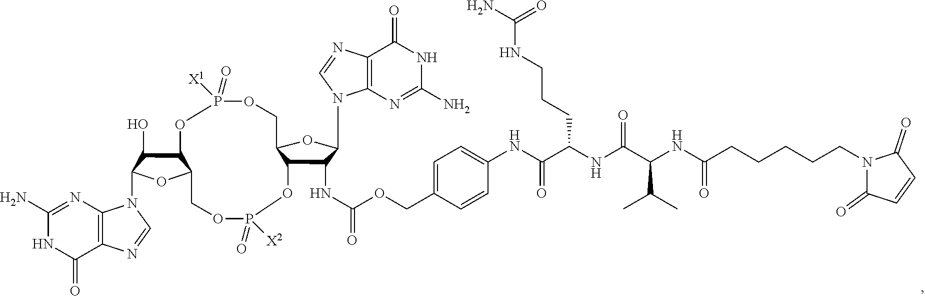

or a pharmaceutically acceptable salt thereof. The compound may be represented by the formula:

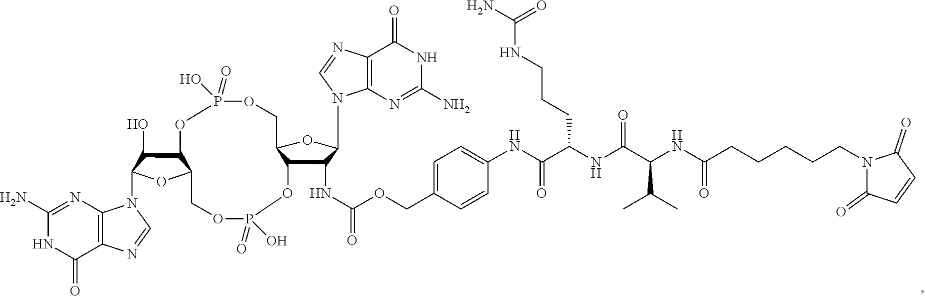

or a pharmaceutically acceptable salt thereof. The compound may be represented by the formula:

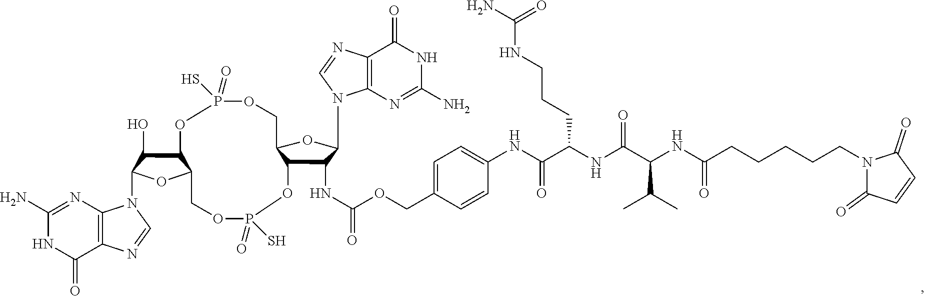

or a pharmaceutically acceptable salt thereof.

In some embodiments, the compound is represented by the formula:

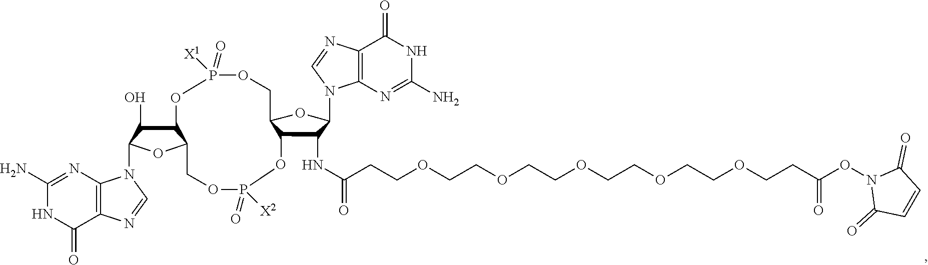

or a pharmaceutically acceptable salt thereof.

The compound is represented by the formula:

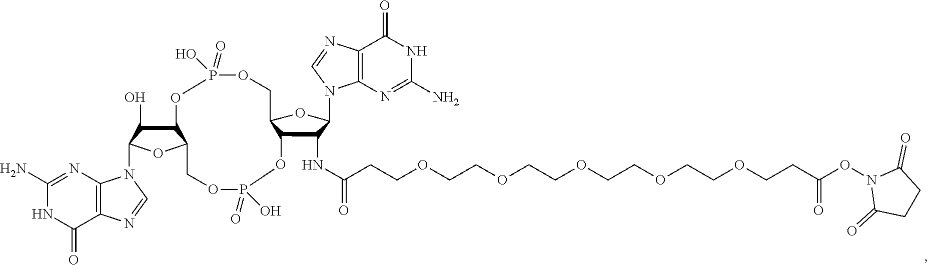

or a pharmaceutically acceptable salt thereof.

The compound may be represented by the formula:

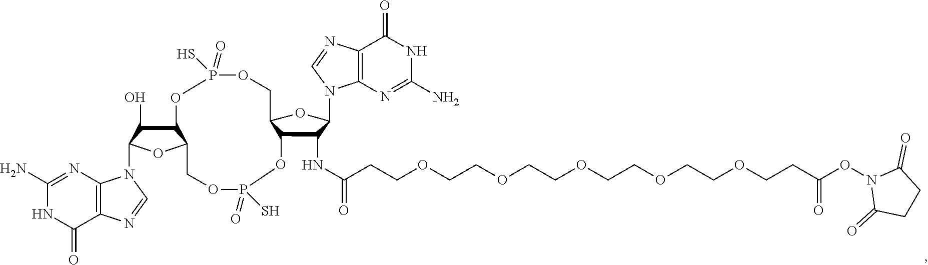

or a pharmaceutically acceptable salt thereof.

In some embodiments, the compound is represented by the formula:

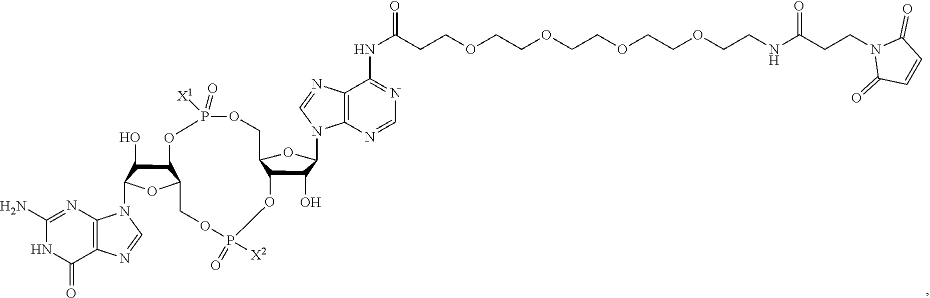

or a pharmaceutically acceptable salt thereof. The compound may be represented by the formula:

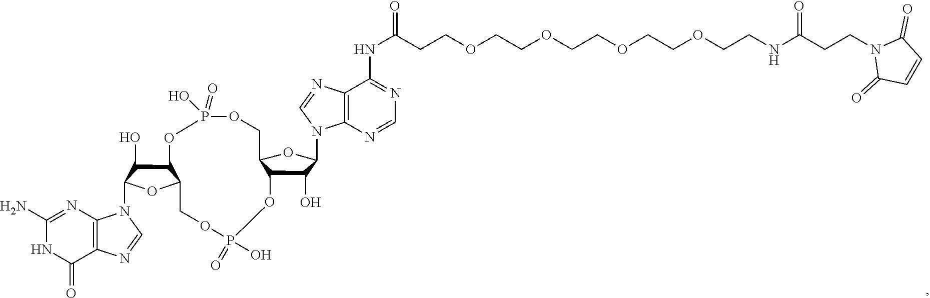

or a pharmaceutically acceptable salt thereof. The compound may be represented by the formula:

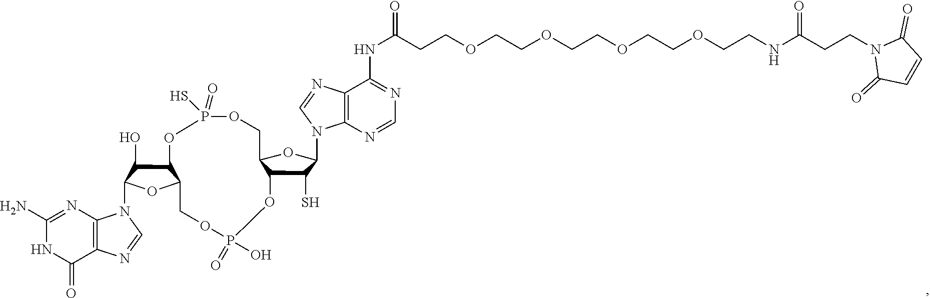

or a pharmaceutically acceptable salt thereof.

In some embodiments, the compound is represented by the formula:

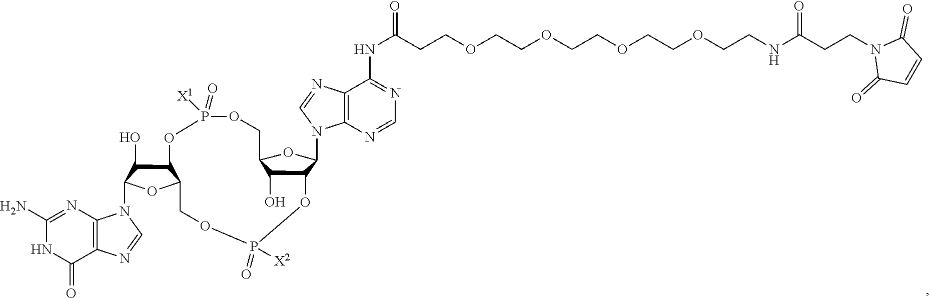

or a pharmaceutically acceptable salt thereof. The compound may be represented by the formula:

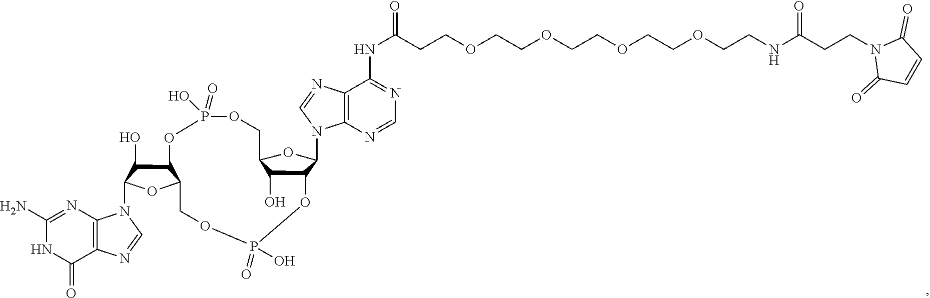

or a pharmaceutically acceptable salt thereof. The compound may be represented by the formula:

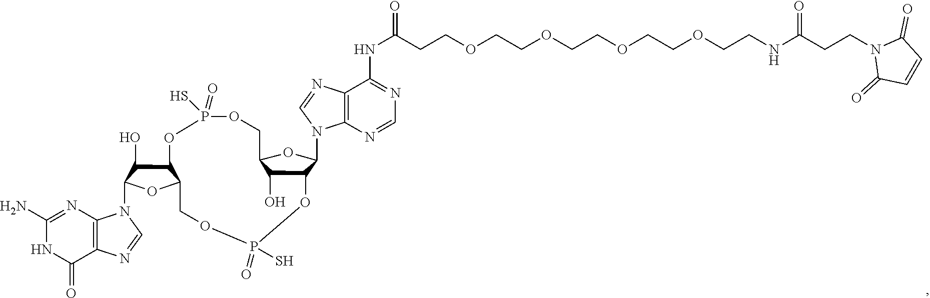

or a pharmaceutically acceptable salt thereof.

In some embodiments, the compound is represented by the formula:

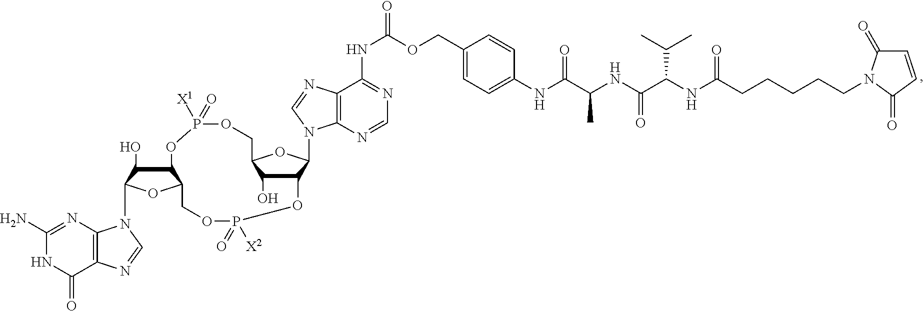

or a pharmaceutically acceptable salt thereof. The compound may be represented by the formula:

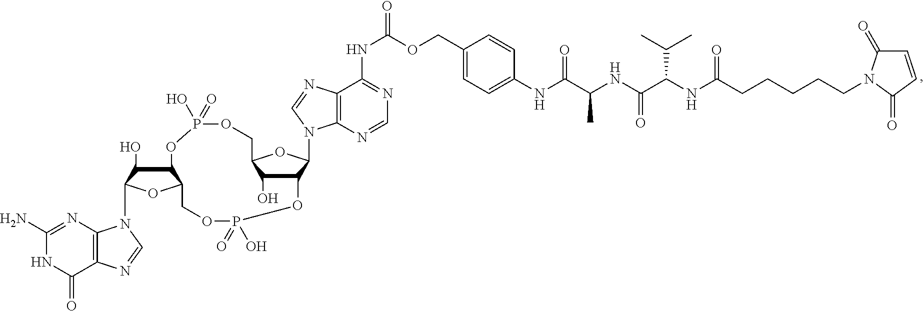

or a pharmaceutically acceptable salt thereof. The compound may be represented by the formula:

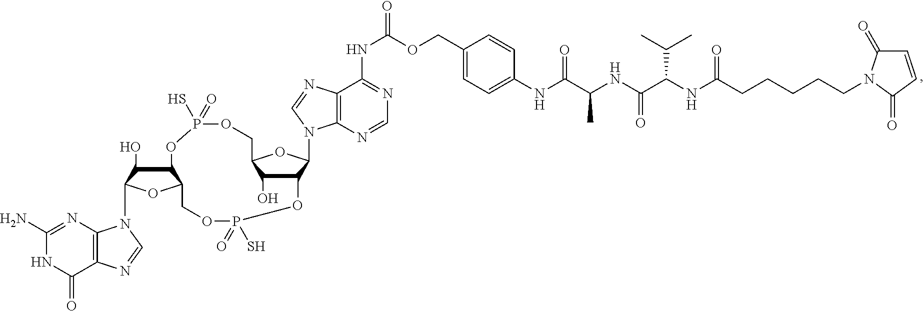

or a pharmaceutically acceptable salt thereof.

In some embodiments, the compound is represented by the formula:

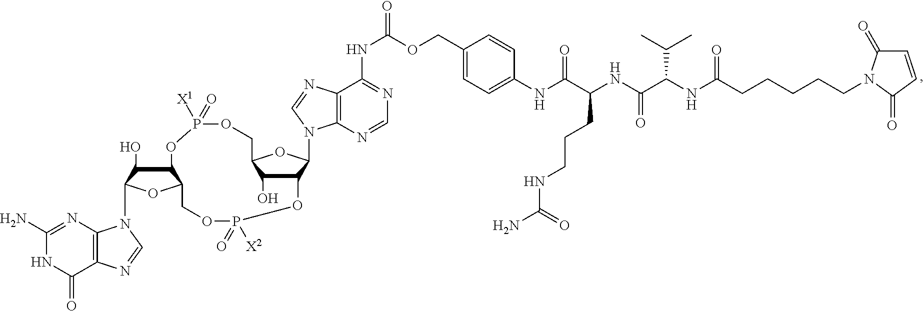

or a pharmaceutically acceptable salt thereof. The compound may be represented by the formula:

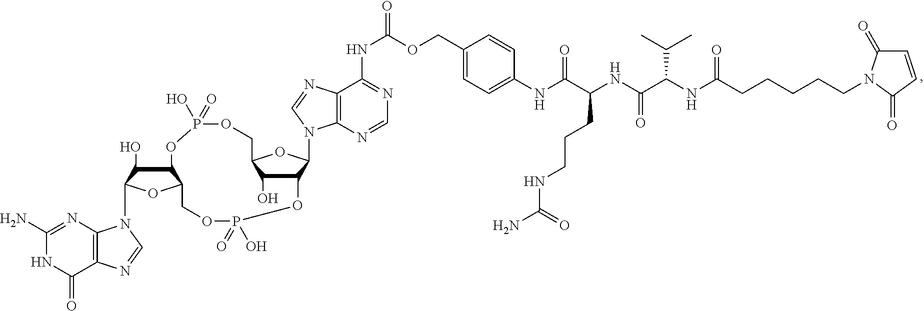

or a pharmaceutically acceptable salt thereof. The compound may be represented by the formula:

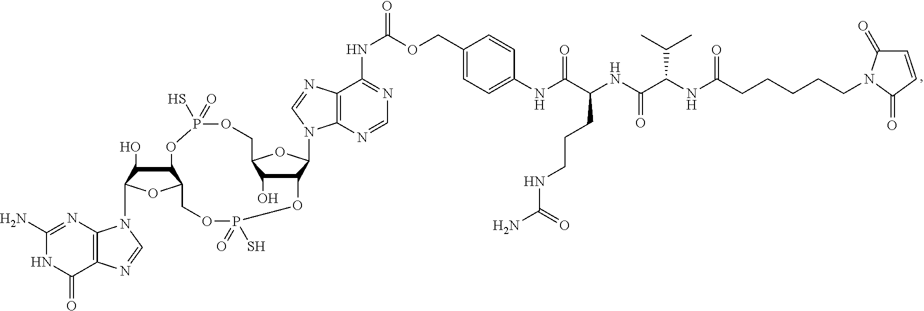

or a pharmaceutically acceptable salt thereof.

An immune-stimulatory compound can be an inhibitor of TGFβ. An immune-stimulatory compound can be β-Catenin. An immune-stimulatory compound can be PI3K-β. An immune-stimulatory compound can be STAT3. An immune-stimulatory compound can be IL-10. An immune-stimulatory compound can be IDO. An immune-stimulatory compound can be TDO. An immune-stimulatory compound can be LY2109761. An immune-stimulatory compound can be GSK263771. An immune-stimulatory compound can be iCRT3. An immune-stimulatory compound can be iCRT5. An immune-stimulatory compound can be iCRT14. An immune-stimulatory compound can be LY2090314. An immune-stimulatory compound can be CGX-1321. An immune-stimulatory compound can be PRI-724. An immune-stimulatory compound can be BC21. An immune-stimulatory compound can be ZINCO2092166. An immune-stimulatory compound can be LGK974. An immune-stimulatory compound can be IWP2. An immune-stimulatory compound can be LY3022859. An immune-stimulatory compound can be LY364947. An immune-stimulatory compound can be SB431542. An immune-stimulatory compound can be AZD8186. An immune-stimulatory compound can be SD-208. An immune-stimulatory compound can be indoximod (NLG8189). An immune-stimulatory compound can be F001287. An immune-stimulatory compound can be GDC-0919. An immune-stimulatory compound can be epacadostat (INCB024360). An immune-stimulatory compound can be RG70099. An immune-stimulatory compound can be 1-methyl-L-tryptophan. An immune-stimulatory compound can be methylthiohydantoin tryptophan. An immune-stimulatory compound can be brassinin. An immune-stimulatory compound can be annulin B. An immune-stimulatory compound can be exiguamine A. An immune-stimulatory compound can be PIM. An immune-stimulatory compound can be LM10. An immune-stimulatory compound can be INCB023843. An immune-stimulatory compound can be 8-substituted imidazo[1,5-a]pyridine.

In some embodiments, the immune-stimulatory compound is an inhibitor of TGFβ, β-Catenin, PI3K-3, STAT3, IL-10, IDO, or TDO. In some embodiments, the immune-stimulatory compound is LY2109761, GSK263771, iCRT3, iCRT5, iCRT14, LY2090314, CGX-1321, PRI-724, BC21, ZINCO2092166, LGK974, IWP2, LY3022859, LY364947, SB431542, AZD8186, SD-208, indoximod (NLG8189), F001287, GDC-0919, epacadostat (INCB024360), RG70099, 1-methyl-L-tryptophan, methylthiohydantoin tryptophan, brassinin, annulin B, exiguamine A, PIM, LM10, INCB023843, or 8-substituted imidazo[1,5-a]pyridine.

The K_{d} for binding of an antigen-binding domain to an antigen in the presence of an immune-stimulatory compound can be about 2 times, about 3 times, about 4 times, about 5 times, about 6 times, about 7 times, about 8 times, about 9 times, about 10 times, about 15 times, about 20 times, about 25 times, about 30 times, about 35 times, about 40 times, about 45 times, about 50 times, about 60 times, about 70 times, about 80 times, about 90 times, about 100 times, about 110 times, or about 120 times greater than the K_{d} for binding of the antigen binding domain to the antigen in the absence of the immune-stimulatory compound.

The K_{d} for binding of an Fc domain to a Fc receptor in the presence of an immune-stimulatory compound can be about 2 times, about 3 times, about 4 times, about 5 times, about 6 times, about 7 times, about 8 times, about 9 times, about 10 times, about 15 times, about 20 times, about 25 times, about 30 times, about 35 times, about 40 times, about 45 times, about 50 times, about 60 times, about 70 times, about 80 times, about 90 times, about 100 times, about 110 times, or about 120 times greater than the K_{d} for binding of the Fc domain to the Fc receptor in the absence of the immune-stimulator compound.

The K_{d} for binding of an antigen-binding domain to an antigen in the presence of an immune-stimulatory compound and a peptide can be about 2 times, about 3 times, about 4 times, about 5 times, about 6 times, about 7 times, about 8 times, about 9 times, about 10 times, about 15 times, about 20 times, about 25 times, about 30 times, about 35 times, about 40 times, about 45 times, about 50 times, about 60 times, about 70 times, about 80 times, about 90 times, about 100 times, about 110 times, or about 120 times greater than the K_{d} for binding of the antigen binding domain to the antigen in the absence of the immune-stimulatory compound and a peptide. The K_{d} for binding of an antigen-binding domain to an antigen in the presence of an immune-stimulatory compound and a peptide can be no greater than 500 nM, no greater than 100 nM, no greater than 50 nM, no greater than 25 nM, no greater than 10 nM, no greater than 5 nM, no greater than 1 nM, or no greater than 0.1 nM.

The K_{d} for binding of an Fc domain to a Fc receptor in the presence of an immune-stimulatory compound and a peptide can be about 2 times, about 3 times, about 4 times, about 5 times, about 6 times, about 7 times, about 8 times, about 9 times, about 10 times, about 15 times, about 20 times, about 25 times, about 30 times, about 35 times, about 40 times, about 45 times, about 50 times, about 60 times, about 70 times, about 80 times, about 90 times, about 100 times, about 110 times, or about 120 times greater than the K_{d} for binding of the Fc domain to the Fc receptor in the absence of the immune-stimulator compound and a peptide. The K_{d} for binding of an Fc domain to a Fc receptor in the presence of an immune-stimulatory compound and a peptide can be no greater than 500 nM, no greater than 100 nM, no greater than 50 nM, no greater than 25 nM, no greater than 10 nM, no greater than 5 nM, no greater than 1 nM, no greater than 0.1 nM, no greater than 10μM, or no greater than 1 μM.

Agonism can be described as the binding of a chemical to a receptor to induce a biological response. A chemical can be, for example, a small molecule, a compound, or a protein. An agonist causes a response, an antagonist can block the action of an agonist, and an inverse agonist can cause a response that is opposite to that of the agonist. A receptor can be activated by either endogenous or exogenous agonists.

The molar ratio of a construct immune-stimulatory compound conjugate refers to the average number of immune-stimulatory compounds conjugated to the construct or the construct-peptide composition in a preparation of a construct immune-stimulatory compound conjugate. The molar ratio can be determined, for example, by Liquid Chromatography/Mass Spectrometry (LC/MS), in which the number of immune-stimulatory compounds conjugated to the construct or construct-peptide composition can be directly determined. Additionally, as non-limiting examples, the molar ratio can be determined based on hydrophobic interaction chromatography (HIC) peak area, by liquid chromatography coupled to electrospray ionization mass spectrometry (LC-ESI-MS), by UV/Vis spectroscopy, by reversed-phase-HPLC (RP-HPLC), or by matrix-assisted laser desorption/ionization time of flight mass spectrometry (MALDI-TOF-MS).

In some embodiments, the molar ratio of immune-stimulatory compound to construct or construct-peptide composition can be less than 8. In other embodiments, the molar ratio of immune-stimulatory compound to construct or construct-peptide composition can be 8, 7, 6, 5, 4, 3, 2, or 1.

These conjugates can be made by various methods. It is understood that one skilled in the art will be able to make these compounds by similar methods or by combining other methods known to one skilled in the art. It is also understood that one skilled in the art will be able to make, in a similar manner as described herein by using the appropriate starting materials and modifying the synthetic route(s) as needed. Starting materials and reagents can be obtained from commercial vendors or synthesized according to sources known to those skilled in the art or prepared as described herein.

### Linkers

The construct-peptide compositions further comprising an immune-stimulatory compound and methods described herein can comprise a linker, e.g., a peptide linker. These conjugates can include a linker that attaches a construct-peptide composition to at least one myeloid agonist. The linker can be, for example, a cleavable or a non-cleavable linker. Linkers of the conjugates and methods described herein may not affect the binding of active portions of a conjugate (e.g., antigen binding domains, Fc domains, peptide, antibodies, agonists or the like) to a target, which can be a cognate binding partner such as an antigen or an Fc receptor. A linker sequence can form a linkage between different parts of a composition. A composition can be a conjugate. A conjugate can comprise multiple linkers. These linkers can be the same linkers or different linkers. A construct can be linked to a peptide. A construct can be linked to a peptide and an immune-stimulatory compound. In some embodiments the peptide is connected to the construct as a fusion protein or by a second linker at the C-terminus of the Fc domain. In some embodiments, the second linker is a polypeptide linker. The polypeptide linker can range from 10 to 25 amino acids and can, for example, comprise the sequence [G4S]n where n=2 to 5 (SEQ ID NO: 138). In some embodiments, the polypeptide linker can vary in length from 15 to 25 amino acids, and can, for example, comprise the sequence [G4S]n where n=3 to 5 (SEQ ID NO: 139).

Attachment via a linker can involve incorporation of a linker between parts of a construct-peptide composition or conjugate. A linker can be short, flexible, rigid, cleavable, non-cleavable, hydrophilic, or hydrophobic. A linker can contain segments that have different characteristics, such as segments of flexibility or segments of rigidity. The linker can be chemically stable to extracellular environments, for example, chemically stable in the blood stream, or may include linkages that are not stable. The linker can include linkages that are designed to cleave and/or immolate or otherwise breakdown specifically or non-specifically inside cells. A cleavable linker can be sensitive to (i.e., cleavable by) enzymes at a specific site. A cleavable linker can be cleaved by enzymes such as proteases. A cleavable linker can be a valine-citrulline peptide or a valine-alanine peptide. A valine-citrulline- or valine-alanine-containing linker can contain a pentafluorophenyl group. A valine-citrulline or valine-alanine-containing linker can contain a succimide or a maleimide group. A valine-citrulline- or valine-alanine-containing linker can contain a para aminobenzoic acid (PABA) group. A valine-citrulline- or valine-alanine-containing linker can contain a PABA group and a pentafluorophenyl group. A valine-citrulline- or valine-alanine-containing linker can contain a PABA group and a succinimide group. A valine-citrulline- or valine-alanine-containing linker can contain a PABA group and a maleimide group. A non-cleavable linker can be protease insensitive (i.e., non-cleavable). A non-cleavable linker can contain a maleimide group. A non-cleavable linker can contain a succinimide group. A non-cleavable linker can be maleimidocaproyl linker. A maleimidocaproyl linker can comprise N-maleimidomethylcyclohexane-1-carboxylate. A maleimidocaproyl linker can contain a succinimide group. A maleimidocaproyl linker can contain pentafluorophenyl group.

A linker can be a combination of a maleimidocaproyl linker containing a pentafluorophenyl group and one or more polyethylene glycol molecules. A linker can contain maleimides linked to polyethylene glycol molecules in which the polyethylene glycol can allow for more linker flexibility or can be used lengthen the linker. A linker can be a (maleimidocaproyl)-(valine-citrulline)-para-aminobenzyloxycarbonyl linker. A linker can also be an alkylene, alkenylene, alkynylene, polyether, polyester, polyamide, polyamino acids, polypeptides, cleavable peptides, or aminobenzylcarbamates. A linker can contain a maleimide at one end and an N-hydroxysuccinimidyl ester at the other end. A linker can contain a lysine with an N-terminal amine acetylated, and a valine-citrulline cleavage site. A linker can be a link created by a microbial transglutaminase, wherein the link can be created between an amine-containing moiety and a moiety engineered to contain glutamine as a result of the enzyme catalyzing a bond formation between the acyl group of a glutamine side chain and the primary amine of a lysine chain. A linker can contain a reactive primary amine. A linker can be a Sortase A linker. A Sortase A linker can be created by a Sortase A enzyme fusing an LPXTG recognition motif (SEQ ID NO: 21) to an N-terminal GGG motif to regenerate a native amide bond. The linker created can therefore link a moiety attached to the LPXTG recognition motif (SEQ ID NO: 21) with a moiety attached to the N-terminal GGG motif. A linker can be a link created between an unnatural amino acid on one moiety reacting with oxime bond that was formed by modifying a ketone group with an alkoxyamine on another moiety.

A moiety can be a part of the construct of the conjugate. A moiety can be a part of an antibody of the construct of the conjugate. A moiety can be a part of an immune-stimulatory compound. A moiety can be a part of a binding domain. A linker can be a portion of a linker can be unsubstituted or substituted, for example, with a substituent. A substituent can include, for example, hydroxyl groups, amino groups, nitro groups, cyano groups, azido groups, carboxyl groups, carboxaldehyde groups, imine groups, alkyl groups, alkenyl groups, alkynyl groups, alkoxy groups, acyl groups, acyloxy groups, amide groups, and ester groups.

In the construct-peptide conjugate described herein, the peptide can be linked to the construct by way of linkers. In the construct-peptide immune-stimulatory compound conjugate described herein, the peptide can be linked to the construct by way of linkers and/or the immune-stimulatory compound can be linked to the construct by way of linkers. The linker linking a peptide to the construct of a construct-peptide conjugate can be short, long, hydrophobic, hydrophilic, flexible or rigid, or may be composed of segments that each independently have one or more of the above-mentioned properties such that the linker may include segments having different properties. The linker linking an immune-stimulatory compound to the construct of a construct-peptide immune-stimulatory compound conjugate can be short, long, hydrophobic, hydrophilic, flexible or rigid, or may be composed of segments that each independently have one or more of the above-mentioned properties such that the linker may include segments having different properties. The linkers can be polyvalent such that they covalently link more than one immune-stimulatory compound to a single site on the antibody construct, or monovalent such that covalently they link a single immune-stimulatory compound to a single site on the antibody construct.

As will be appreciated by skilled artisans, the linkers can link the immune-stimulatory compound and/or the peptide to the construct by forming a covalent linkage to the immune-stimulatory compound at one location and a covalent linkage to the antibody construct at another. The covalent linkages can be formed by reaction between functional groups on the linker and functional groups on the inhibitors and antibody construct. As used herein, the expression “linker” can include (i) unconjugated forms of the linker that include a functional group capable of covalently linking the linker to a peptide and a functional group capable of covalently linking the linker to a construct; (ii) partially conjugated forms of the linker that include a functional group capable of covalently linking the linker to a construct and that is covalently linked to a peptide, or vice versa; and (iii) fully conjugated forms of the linker that is covalently linked to both a peptide compound and a construct. Additionally, the expression “linker” can include (i) unconjugated forms of the linker that include a functional group capable of covalently linking the linker to an immune-stimulatory compound and a functional group capable of covalently linking the linker to a construct; (ii) partially conjugated forms of the linker that include a functional group capable of covalently linking the linker to a construct and that is covalently linked to an immune-stimulatory compound, or vice versa; and (iii) fully conjugated forms of the linker that is covalently linked to both an immune-stimulatory compound and a construct. In some specific embodiments of intermediate synthons and construct-peptide immune-stimulatory compound conjugates described herein, moieties comprising the functional groups on the linker and covalent linkages formed between the linker and construct are specifically illustrated as Rx and LK, respectively. One embodiment pertains to a construct-peptide immune-stimulatory compound conjugate formed by contacting a construct that binds a cell surface receptor or tumor associated antigen expressed on a tumor cell with a synthon described herein under conditions in which the synthon covalently links to the construct. One embodiment pertains to a method of making a construct-peptide immune-stimulatory compound conjugate formed by contacting a synthon described herein under conditions in which the synthon covalently links to the construct. One embodiment pertains to a method of stimulating immune activity in a cell that expresses CD40, comprising contacting the cell with a construct-peptide immune-stimulatory compound conjugate described herein that is capable of binding the cell, under conditions in which the construct-peptide immune-stimulatory compound conjugate binds the cell.

Exemplary polyvalent linkers that may be used to link many immune-stimulatory compounds to a construct are described. For example, Fleximer® linker technology has the potential to enable high-DAR construct-peptide immune-stimulatory compound conjugate with good physicochemical properties. As shown below, the Fleximer® linker technology is based on incorporating drug molecules into a solubilizing poly-acetal backbone via a sequence of ester bonds. The methodology renders highly-loaded construct-peptide immune-stimulatory compound conjugates (DAR up to 20) whilst maintaining good physicochemical properties. This methodology could be utilized with an immune-stimulatory compound as shown in the Scheme below:

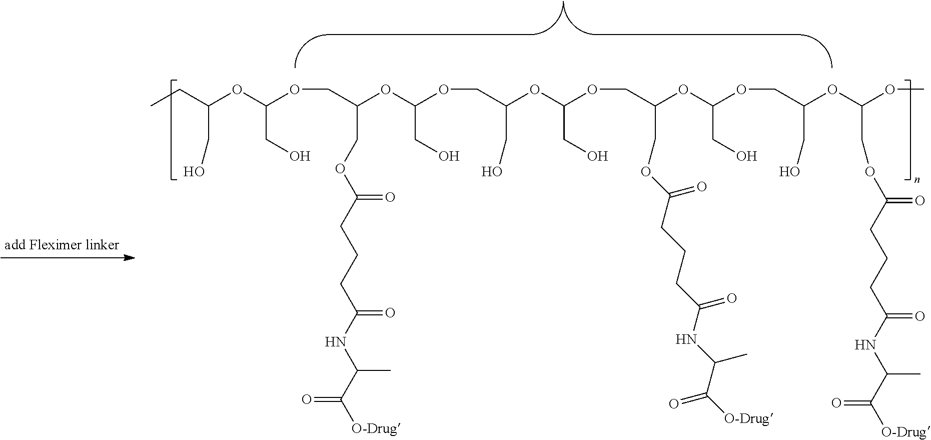

To utilize the Fleximer® linker technology depicted in the scheme above, an aliphatic alcohol can be present or introduced into the immune-stimulatory compound. The alcohol moiety is then conjugated to an alanine moiety, which is then synthetically incorporated into the Fleximer® linker. Liposomal processing of the construct-peptide immune-stimulatory compound conjugate in vitro releases the parent alcohol-containing drug.

By way of example and not limitation, some cleavable and noncleavable linkers that may be included in the construct-peptide immune-stimulatory compound conjugates described herein are described below.

Cleavable linkers can be cleavable in vitro and in vivo. Cleavable linkers can include chemically or enzymatically unstable or degradable linkages. Cleavable linkers can rely on processes inside the cell to liberate a peptide or an immune-stimulatory compound, such as reduction in the cytoplasm, exposure to acidic conditions in the lysosome, or cleavage by specific proteases or other enzymes within the cell. Cleavable linkers can incorporate one or more chemical bonds that are either chemically or enzymatically cleavable while the remainder of the linker can be non-cleavable.

A linker can contain a chemically labile group such as hydrazone and/or disulfide groups. Linkers comprising chemically labile groups can exploit differential properties between the plasma and some cytoplasmic compartments. The intracellular conditions that can facilitate immune-stimulatory compound release for hydrazone containing linkers can be the acidic environment of endosomes and lysosomes, while the disulfide containing linkers can be reduced in the cytosol, which can contain high thiol concentrations, e.g., glutathione. The plasma stability of a linker containing a chemically labile group can be increased by introducing steric hindrance using substituents near the chemically labile group.

Acid-labile groups, such as hydrazone, can remain intact during systemic circulation in the blood's neutral pH environment (pH 7.3-7.5) and can undergo hydrolysis and can release the immune-stimulatory compound once the construct-peptide immune-stimulatory compound conjugate is internalized into mildly acidic endosomal (pH 5.0-6.5) and lysosomal (pH 4.5-5.0) compartments of the cell. This pH dependent release mechanism can be associated with nonspecific release of the drug. To increase the stability of the hydrazone group of the linker, the linker can be varied by chemical modification, e.g., substitution, allowing tuning to achieve more efficient release in the lysosome with a minimized loss in circulation.

Hydrazone-containing linkers can contain additional cleavage sites, such as additional acid-labile cleavage sites and/or enzymatically labile cleavage sites. Construct-peptide compositions or construct-peptide immune-stimulatory compound conjugates including exemplary hydrazone-containing linkers can include, for example, the following structures:

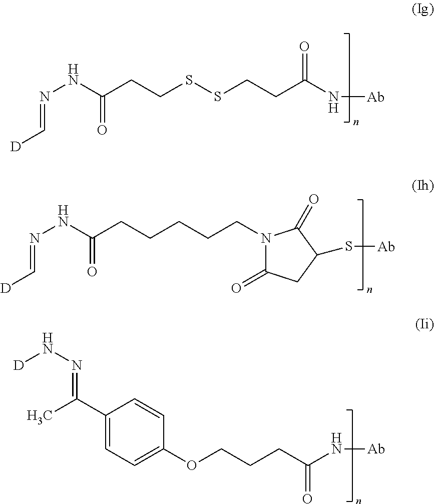

wherein D and Ab represent the immune-stimulatory compound or peptide and construct, respectively, and n represents the number of peptide or immune-stimulatory compound—linkers linked to the construct. In certain linkers such as linker (Ig), the linker can comprise two cleavable groups—a disulfide and a hydrazone moiety. For such linkers, effective release of the unmodified free peptide or immune-stimulatory compound can require acidic pH or disulfide reduction and acidic pH. Linkers such as (Ih) and (Ii) can be effective with a single hydrazone cleavage site.

Other acid-labile groups that can be included in linkers include cis-aconityl-containing linkers. cis-Aconityl chemistry can use a carboxylic acid juxtaposed to an amide bond to accelerate amide hydrolysis under acidic conditions.

Cleavable linkers can also include a disulfide group. Disulfides can be thermodynamically stable at physiological pH and can be designed to release the peptide or immune-stimulatory compound upon internalization inside cells, wherein the cytosol can provide a significantly more reducing environment compared to the extracellular environment. Scission of disulfide bonds can require the presence of a cytoplasmic thiol cofactor, such as (reduced) glutathione (GSH), such that disulfide-containing linkers can be reasonably stable in circulation, selectively releasing the peptide or immune-stimulatory compound in the cytosol. The intracellular enzyme protein disulfide isomerase, or similar enzymes capable of cleaving disulfide bonds, can also contribute to the preferential cleavage of disulfide bonds inside cells. GSH can be present in cells in the concentration range of 0.5-10 mM compared with a significantly lower concentration of GSH or cysteine, the most abundant low-molecular weight thiol, in circulation at approximately 5 pM. Tumor cells, where irregular blood flow can lead to a hypoxic state, can result in enhanced activity of reductive enzymes and therefore even higher glutathione concentrations. The in vivo stability of a disulfide-containing linker can be enhanced by chemical modification of the linker, e.g., use of steric hindrance adjacent to the disulfide bond.

Construct-peptide conjugates or construct-peptide immune-stimulatory compound conjugates including exemplary disulfide-containing linkers can include the following structures:

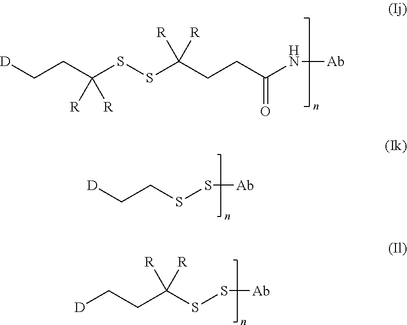

wherein D and Ab represent the peptide or immune-stimulatory compound and construct, respectively, n represents the number of peptide-linkers or immune-stimulatory compound-linkers linked to the construct and R is independently selected at each occurrence from hydrogen or alkyl, for example. Increasing steric hindrance adjacent to the disulfide bond can increase the stability of the linker. Structures such as (Ij) and (Il) can show increased in vivo stability when one or more R groups is selected from a lower alkyl such as methyl.

Another type of linker that can be used is a linker that is specifically cleaved by an enzyme. For example, the linker can be cleaved by a lysosomal enzyme. Such linkers can be peptide-based or can include peptidic regions that can act as substrates for enzymes. Peptide based linkers can be more stable in plasma and extracellular milieu than chemically labile linkers.

Peptide bonds can have good serum stability, as lysosomal proteolytic enzymes can have very low activity in blood due to endogenous inhibitors and the unfavorably high pH value of blood compared to lysosomes. Release of a peptide or an immune-stimulatory compound from a construct can occur due to the action of lysosomal proteases, e.g., cathepsin and plasmin. These proteases can be present at elevated levels in certain tumor tissues. The linker can be cleavable by a lysosomal enzyme. The lysosomal enzyme can be, for example, cathepsin B, β-glucuronidase, or β-galactosidase.

The cleavable peptide can be selected from tetrapeptides such as Gly-Phe-Leu-Gly (SEQ ID NO: 140), Ala-Leu-Ala-Leu (SEQ ID NO: 141) or dipeptides such as Val-Cit, Val-Ala, and Phe-Lys. Dipeptides can have lower hydrophobicity compared to longer peptides.

A variety of dipeptide-based cleavable linkers can be used in the construct-peptide conjugates or the construct-peptide immune-stimulatory compound conjugates described herein.

Enzymatically cleavable linkers can include a self-immolative spacer to spatially separate the peptide or immune-stimulatory compound from the site of enzymatic cleavage. The direct attachment of a peptide or immune-stimulatory compound to a peptide linker can result in proteolytic release of an amino acid adduct of the immune-stimulatory compound, thereby impairing its activity. The use of a self-immolative spacer can allow for the elimination of the fully active, chemically unmodified immune-stimulatory compound upon amide bond hydrolysis.

One self-immolative spacer can be a bifunctional para-aminobenzyl alcohol group, which can link to the linker peptide through the amino group, forming an amide bond, while amine containing immune-stimulatory compounds can be attached through carbamate functionalities to the benzylic hydroxyl group of the linker (to give a p-amidobenzylcarbamate, PABC). The resulting pro-immune-stimulatory compound can be activated upon protease-mediated cleavage, leading to a 1,6-elimination reaction releasing the unmodified immune-stimulatory compound, carbon dioxide, and remnants of the linker group. The following scheme depicts the fragmentation of p-amidobenzyl carbamate and release of the immune-stimulatory compound:

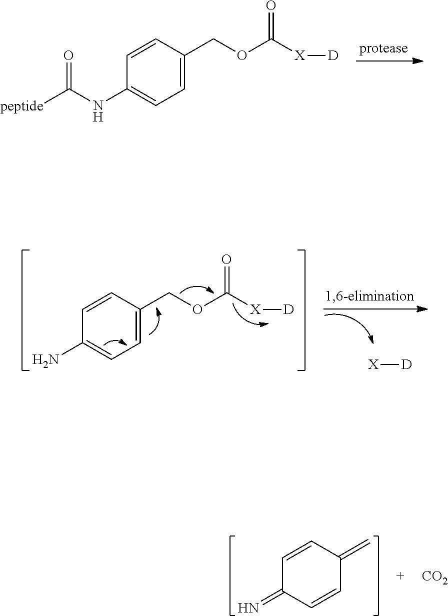

wherein X-D represents the unmodified immune-stimulatory compound. Heterocyclic variants of this self-immolative group have also been described.

The enzymatically cleavable linker can be a ß-glucuronic acid-based linker. Facile release of the immune-stimulatory compound or peptide can be realized through cleavage of the ß-glucuronide glycosidic bond by the lysosomal enzyme ß-glucuronidase. This enzyme can be abundantly present within lysosomes and can be overexpressed in some tumor types, while the enzyme activity outside cells can be low. ß-Glucuronic acid-based linkers can be used to circumvent the tendency of a construct-peptide conjugate or construct-peptide immune-stimulatory compound conjugate to undergo aggregation due to the hydrophilic nature of ß-glucuronides. In certain embodiments, ß-glucuronic acid-based linkers can link a construct to a hydrophobic immune-stimulatory compound. The following scheme depicts the release of an immune-stimulatory compound (D) from a construct (Ab) immune-stimulatory compound conjugate containing a ß-glucuronic acid-based linker:

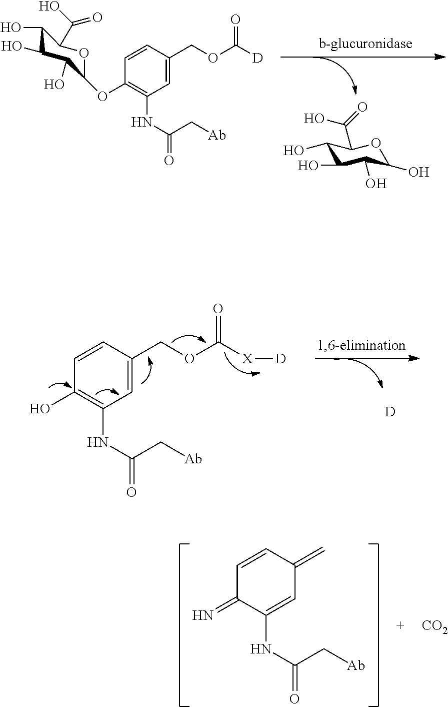

A variety of cleavable β-glucuronic acid-based linkers useful for linking drugs such as auristatins, camptothecin and doxorubicin analogues, CBI minor-groove binders, and psymberin to antibodies have been described. All of these β-glucuronic acid-based linkers may be used in the conjugates described herein. In certain embodiments, the enzymatically cleavable linker is a β-galactoside-based linker. β-Galactoside is present abundantly within lysosomes, while the enzyme activity outside cells is low.

Additionally, immune-stimulatory compounds containing a phenol group can be covalently bonded to a linker through the phenolic oxygen. One such linker relies on a methodology in which a diamino-ethane “Space Link” is used in conjunction with traditional “PABO”-based self-immolative groups to deliver phenols.

Cleavable linkers can include non-cleavable portions or segments, and/or cleavable segments or portions can be included in an otherwise non-cleavable linker to render it cleavable. By way of example only, polyethylene glycol (PEG) and related polymers can include cleavable groups in the polymer backbone. For example, a polyethylene glycol or polymer linker can include one or more cleavable groups such as a disulfide, a hydrazone or a dipeptide.

Other degradable linkages that can be included in linkers can include ester linkages formed by the reaction of PEG carboxylic acids or activated PEG carboxylic acids with alcohol groups on an immune-stimulatory compound, wherein such ester groups can hydrolyze under physiological conditions to release the peptide or immune-stimulatory compound. Hydrolytically degradable linkages can include, but are not limited to, carbonate linkages; imine linkages resulting from reaction of an amine and an aldehyde; phosphate ester linkages formed by reacting an alcohol with a phosphate group; acetal linkages that are the reaction product of an aldehyde and an alcohol; orthoester linkages that are the reaction product of a formate and an alcohol; and oligonucleotide linkages formed by a phosphoramidite group, including but not limited to, at the end of a polymer, and a 5′ hydroxyl group of an oligonucleotide.

A linker can contain an enzymatically cleavable peptide moiety, for example, a linker comprising structural formula (IVa), (IVb), (IVc), or (IVd):

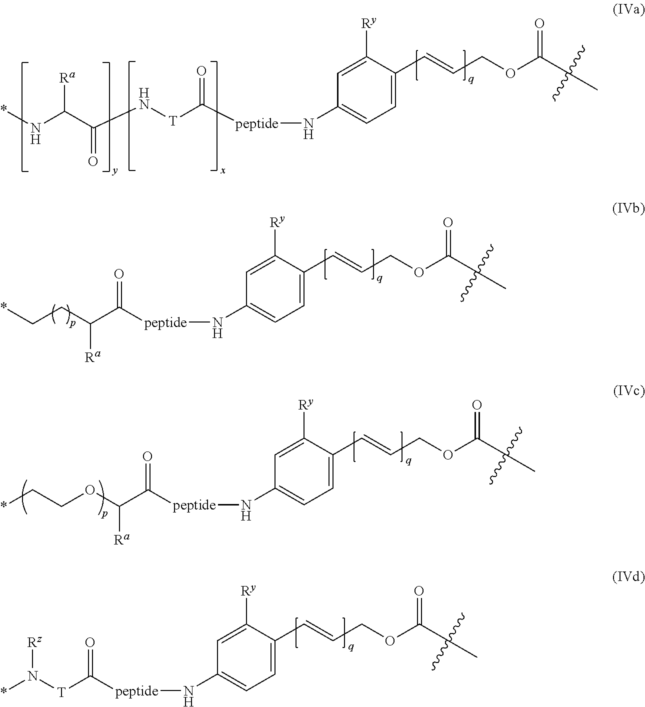

or a salt thereof, wherein: peptide represents a linker peptide (illustrated N→C, wherein peptide includes the amino and carboxy “termini”) a cleavable by a lysosomal enzyme; T represents a polymer comprising one or more ethylene glycol units or an alkylene chain, or combinations thereof; R^{a} is selected from hydrogen, alkyl, sulfonate and methyl sulfonate; R^{y} is hydrogen or C₁₋₄ alkyl-(O)ᵣ—(C₁₋₄ alkylene)ₛ-G¹ or C₁₋₄ alkyl-(N)—[(C₁₋₄ alkylene)-G¹]₂; R^{z} is C₁₋₄ alkyl-(O)ᵣ—(C₁₋₄ alkylene)ₛ-G²; G¹ is SO₃H, CO₂H, PEG 4-32, or sugar moiety; G² is SO₃H, CO₂H, or PEG 4-32 moiety; r is 0 or 1; s is 0 or 1; p is an integer ranging from 0 to 5; q is 0 or 1; x is 0 or 1; y is 0 or 1; [symbol] represents the point of attachment of the linker to the immune-stimulatory compound or peptide of the construct-peptide conjugate; and * represents the point of attachment to the remainder of the linker.

In certain embodiments, the linker peptide can be selected from a tripeptide or a dipeptide. In particular embodiments, the dipeptide can be selected from: Val-Cit; Cit-Val; Ala-Ala; Ala-Cit; Cit-Ala; Asn-Cit; Cit-Asn; Cit-Cit; Val-Glu; Glu-Val; Ser-Cit; Cit-Ser; Lys-Cit; Cit-Lys; Asp-Cit; Cit-Asp; Ala-Val; Val-Ala; Phe-Lys; Lys-Phe; Val-Lys; Lys-Val; Ala-Lys; Lys-Ala; Phe-Cit; Cit-Phe; Leu-Cit; Cit-Leu; Ile-Cit; Cit-Ile; Phe-Arg; Arg-Phe; Cit-Trp; and Trp-Cit, or salts thereof.

Exemplary embodiments of linkers according to structural formula (IVa) that can be included in the construct-peptide conjugates or construct-peptide immune-stimulatory compound conjugates described herein can include the linkers illustrated below (as illustrated, the linkers include a group suitable for covalently linking the linker to a construct):

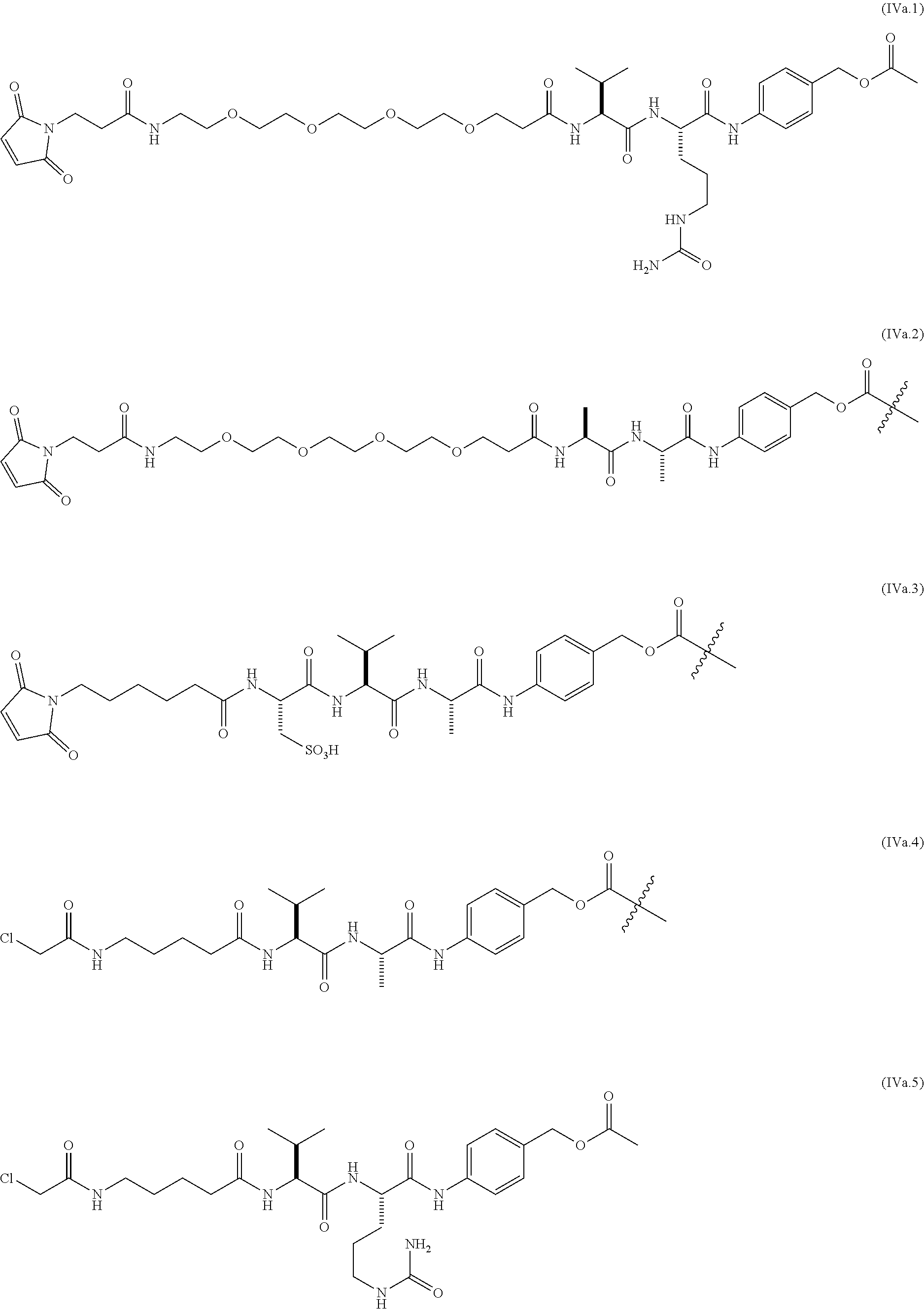 [SMILES: CC(=O)OCc1ccc(NC(=O)[C@H](CCCNC(N)=O)NC(=O)[C@@H](NC(=O)CCOCCOCCOCCOCCNC(=O)CCN2C(=O)C=CC2=O)C(C)C)cc1 ; *C(=O)OCc1ccc(NC(=O)[C@H](C)NC(=O)[C@H](C)NC(=O)CCOCCOCCOCCOCCNC(=O)CCN2C(=O)C=CC2=O)cc1 ; *C(=O)OCc1ccc(NC(=O)[C@H](C)NC(=O)[C@@H](NC(=O)[C@H](CS(=O)(=O)O)NC(=O)CCCCCN2C(=O)C=CC2=O)C(C)C)cc1 ; *C(=O)OCc1ccc(NC(=O)[C@H](C)NC(=O)[C@@H](NC(=O)CCCCNC(=O)CCl)C(C)C)cc1 ; CC(=O)OCc1ccc(NC(=O)[C@H](CCCNC(N)=O)NC(=O)[C@@H](NC(=O)CCCCNC(=O)CCl)C(C)C)cc1]

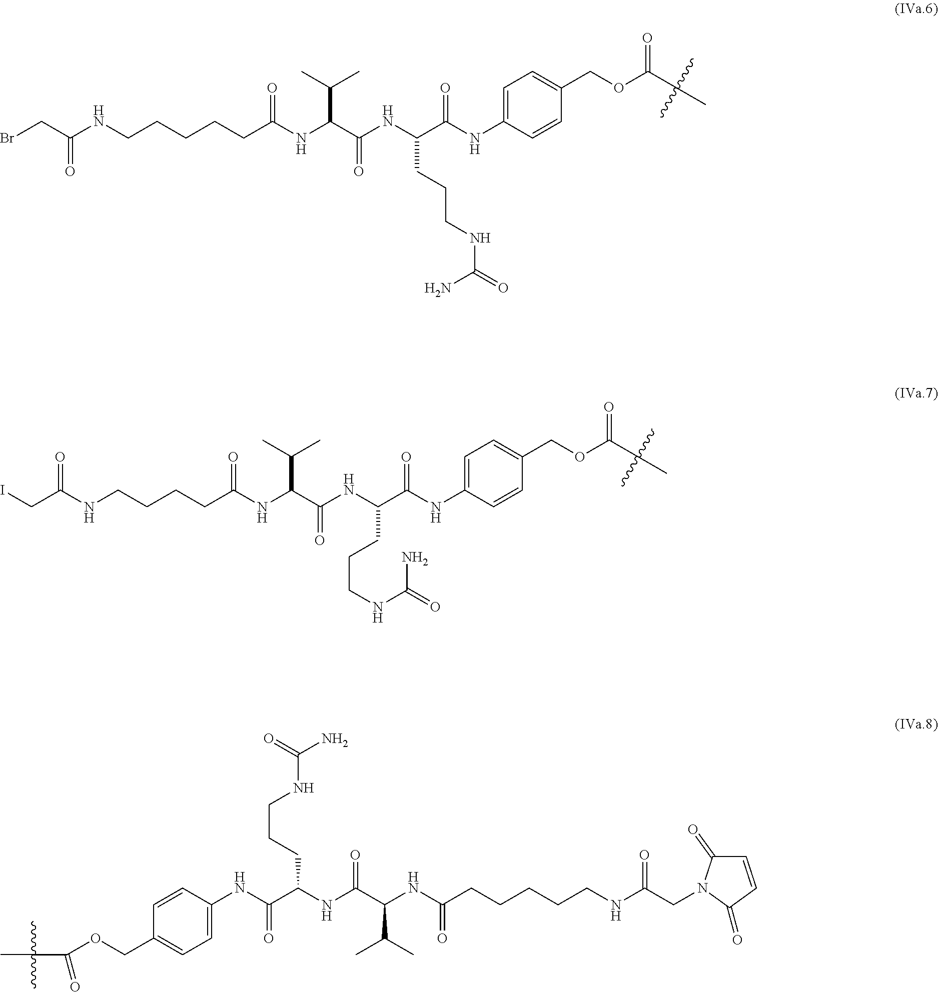 [SMILES: *C(=O)OCc1ccc(NC(=O)[C@H](CCCNC(N)=O)NC(=O)[C@@H](NC(=O)CCCCCNC(=O)CBr)C(C)C)cc1 ; *C(=O)OCc1ccc(NC(=O)[C@H](CCCNC(N)=O)NC(=O)[C@@H](NC(=O)CCCCNC(=O)CI)C(C)C)cc1 ; *C(=O)OCc1ccc(NC(=O)[C@H](CCCNC(N)=O)NC(=O)[C@@H](NC(=O)CCCCCNC(=O)CN2C(=O)C=CC2=O)C(C)C)cc1]

Exemplary embodiments of linkers according to structural formula (IVb), (IVc), or (IVd) that can be included in the construct-peptide conjugates or the construct-peptide immune-stimulatory compound conjugates described herein can include the linkers illustrated below (as illustrated, the linkers can include a group suitable for covalently linking the linker to an antibody construct):

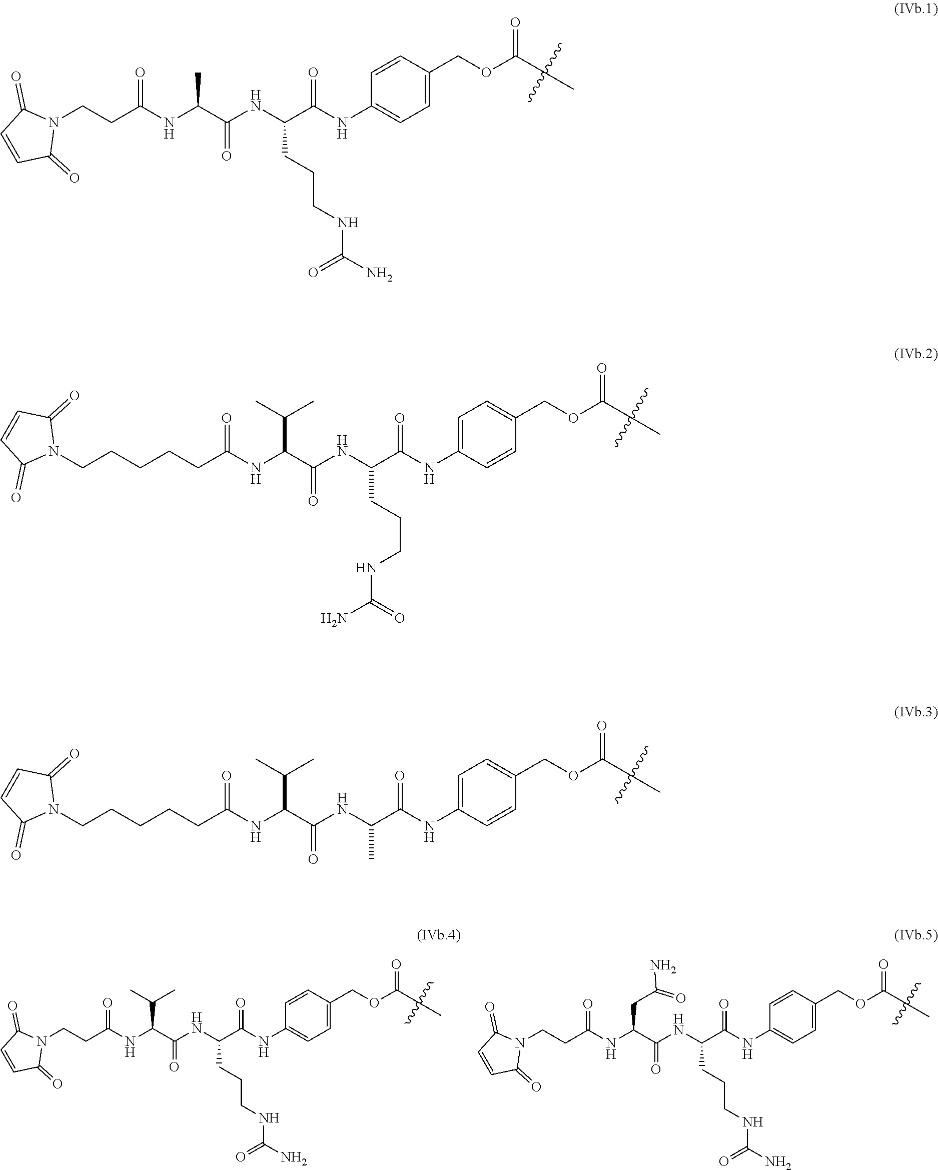 [SMILES: *C(=O)OCc1ccc(NC(=O)[C@H](CCCNC(N)=O)NC(=O)[C@H](C)NC(=O)CCN2C(=O)C=CC2=O)cc1 ; *C(=O)OCc1ccc(NC(=O)[C@H](CCCNC(N)=O)NC(=O)[C@@H](NC(=O)CCCCCN2C(=O)C=CC2=O)C(C)C)cc1 ; *C(=O)OCc1ccc(NC(=O)[C@H](C)NC(=O)[C@@H](NC(=O)CCCCCN2C(=O)C=CC2=O)C(C)C)cc1 ; *C(=O)OCc1ccc(NC(=O)[C@H](CCCNC(N)=O)NC(=O)[C@@H](NC(=O)CCN2C(=O)C=CC2=O)C(C)C)cc1 ; *C(=O)OCc1ccc(NC(=O)[C@H](CCCNC(N)=O)NC(=O)[C@H](CC(N)=O)NC(=O)CCN2C(=O)C=CC2=O)cc1]

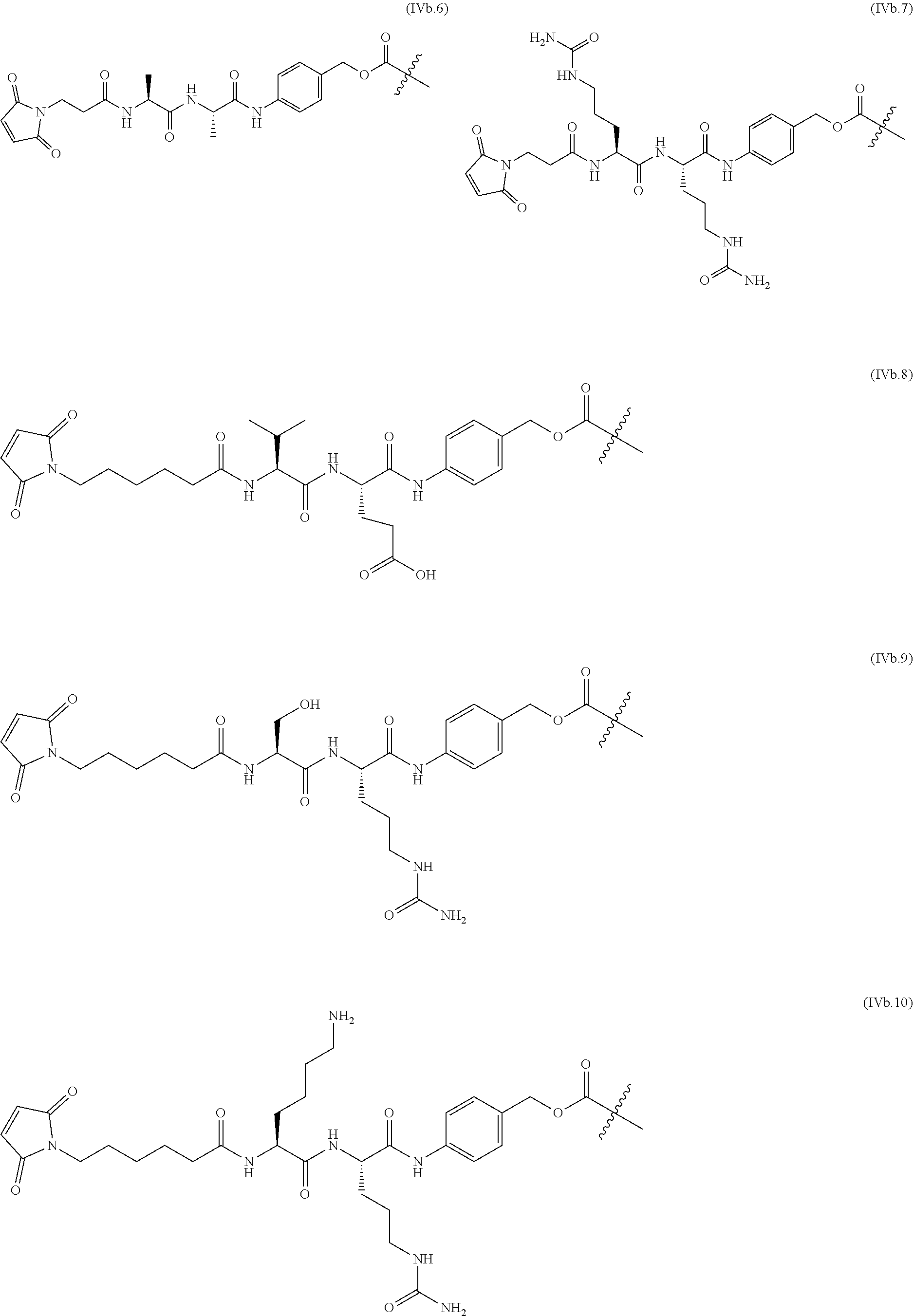 [SMILES: *C(=O)OCc1ccc(NC(=O)[C@H](C)NC(=O)[C@H](C)NC(=O)CCN2C(=O)C=CC2=O)cc1 ; *C(=O)OCc1ccc(NC(=O)[C@H](CCCNC(N)=O)NC(=O)[C@H](CCCNC(N)=O)NC(=O)CCN2C(=O)C=CC2=O)cc1 ; *C(=O)OCc1ccc(NC(=O)[C@H](CCC(=O)O)NC(=O)[C@@H](NC(=O)CCCCCN2C(=O)C=CC2=O)C(C)C)cc1 ; *C(=O)OCc1ccc(NC(=O)[C@H](CCCNC(N)=O)NC(=O)[C@H](CO)NC(=O)CCCCCN2C(=O)C=CC2=O)cc1 ; *C(=O)OCc1ccc(NC(=O)[C@H](CCCNC(N)=O)NC(=O)[C@H](CCCCN)NC(=O)CCCCCN2C(=O)C=CC2=O)cc1]

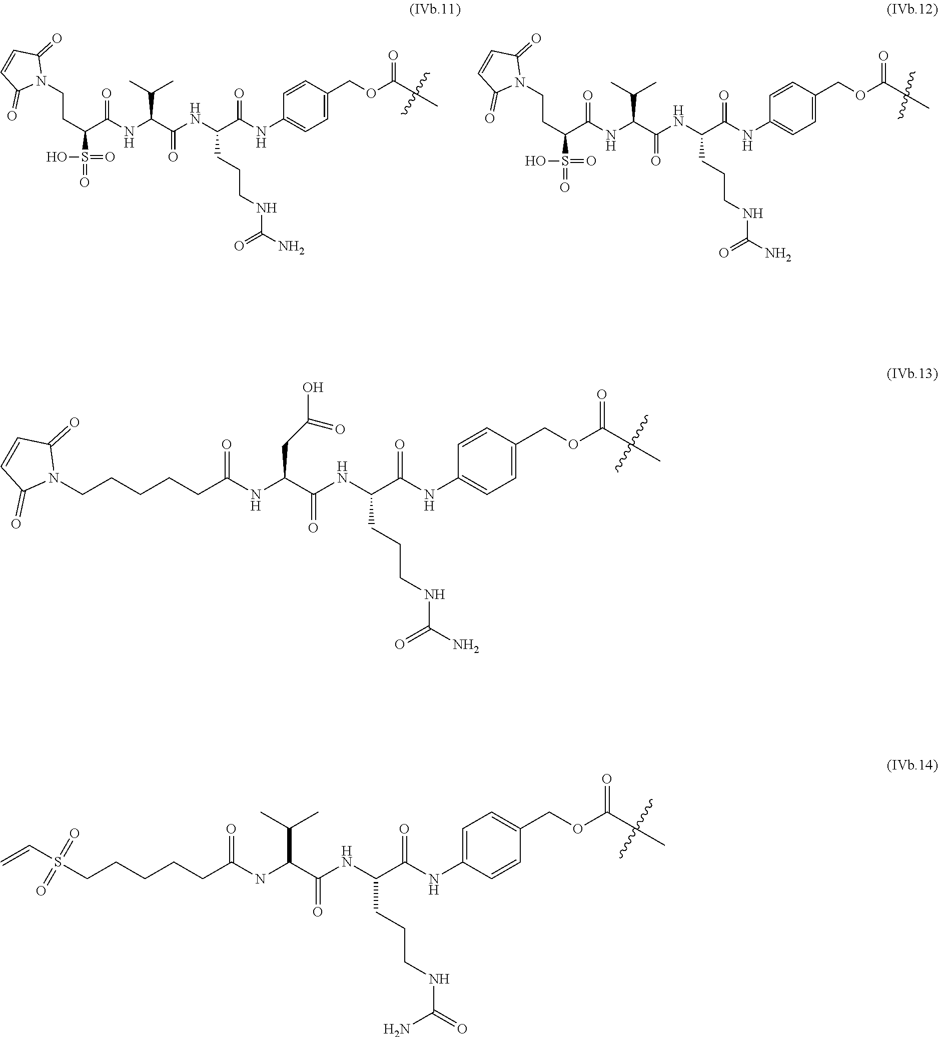 [SMILES: *C(=O)OCc1ccc(NC(=O)[C@H](CCCNC(N)=O)NC(=O)[C@@H](NC(=O)[C@H](CCN2C(=O)C=CC2=O)S(=O)(=O)O)C(C)C)cc1 ; *C(=O)OCc1ccc(NC(=O)[C@H](CCCNC(N)=O)NC(=O)[C@@H](NC(=O)[C@H](CCN2C(=O)C=CC2=O)S(=O)(=O)O)C(C)C)cc1 ; *C(=O)OCc1ccc(NC(=O)[C@H](CCCNC(N)=O)NC(=O)[C@H](CC(=O)O)NC(=O)CCCCCN2C(=O)C=CC2=O)cc1 ; *C(=O)OCc1ccc(NC(=O)[C@H](CCCNC(N)=O)NC(=O)[C@@H](NC(=O)CCCCCS(=O)(=O)C=C)C(C)C)cc1]

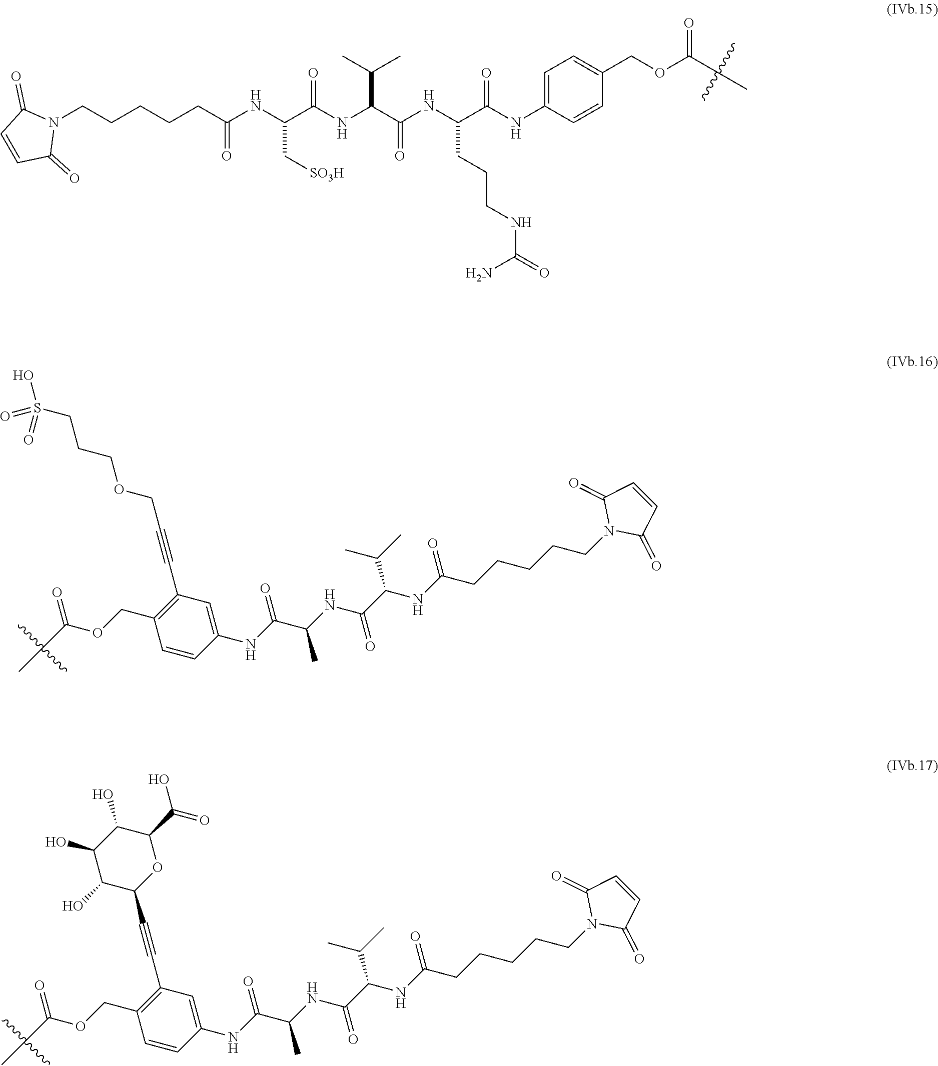 [SMILES: *C(=O)OCc1ccc(NC(=O)[C@H](CCCNC(N)=O)NC(=O)[C@@H](NC(=O)[C@H](CS(=O)(=O)O)NC(=O)CCCCCN2C(=O)C=CC2=O)C(C)C)cc1 ; *C(=O)OCc1ccc(NC(=O)[C@H](C)NC(=O)[C@@H](NC(=O)CCCCCN2C(=O)C=CC2=O)C(C)C)cc1C#CCOCCCS(=O)(=O)O ; *C(=O)OCc1ccc(NC(=O)[C@H](C)NC(=O)[C@@H](NC(=O)CCCCCN2C(=O)C=CC2=O)C(C)C)cc1C#C[C@@H]1O[C@H](C(=O)O)[C@@H](O)[C@H](O)[C@H]1O]

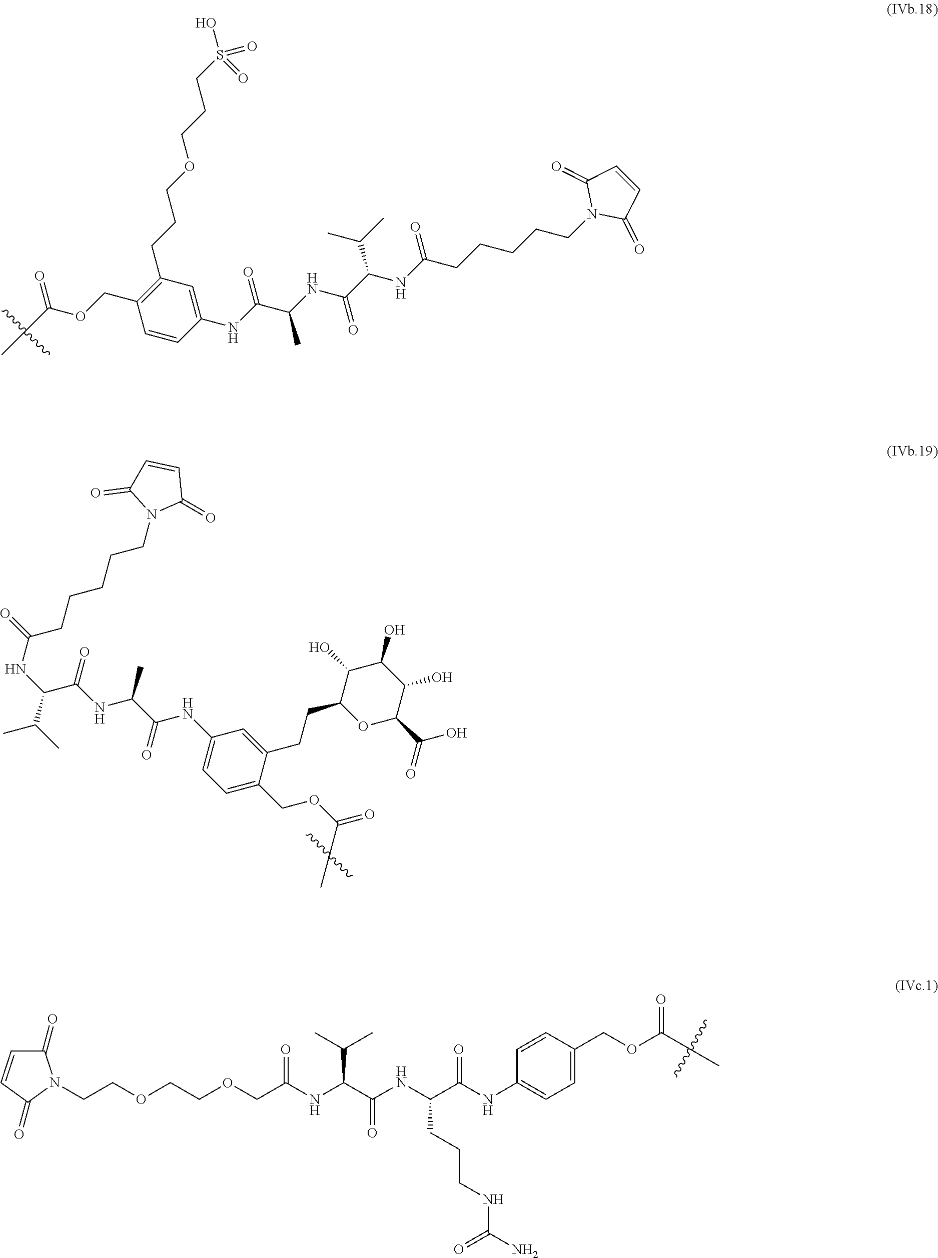 [SMILES: *C(=O)OCc1ccc(NC(=O)[C@H](C)NC(=O)[C@@H](NC(=O)CCCCCN2C(=O)C=CC2=O)C(C)C)cc1CCCOCCCS(=O)(=O)O ; *C(=O)OCc1ccc(NC(=O)[C@H](C)NC(=O)[C@@H](NC(=O)CCCCCN2C(=O)C=CC2=O)C(C)C)cc1CC[C@@H]1O[C@H](C(=O)O)[C@@H](O)[C@H](O)[C@H]1O ; *C(=O)OCc1ccc(NC(=O)[C@H](CCCNC(N)=O)NC(=O)[C@@H](NC(=O)COCCOCCN2C(=O)C=CC2=O)C(C)C)cc1]

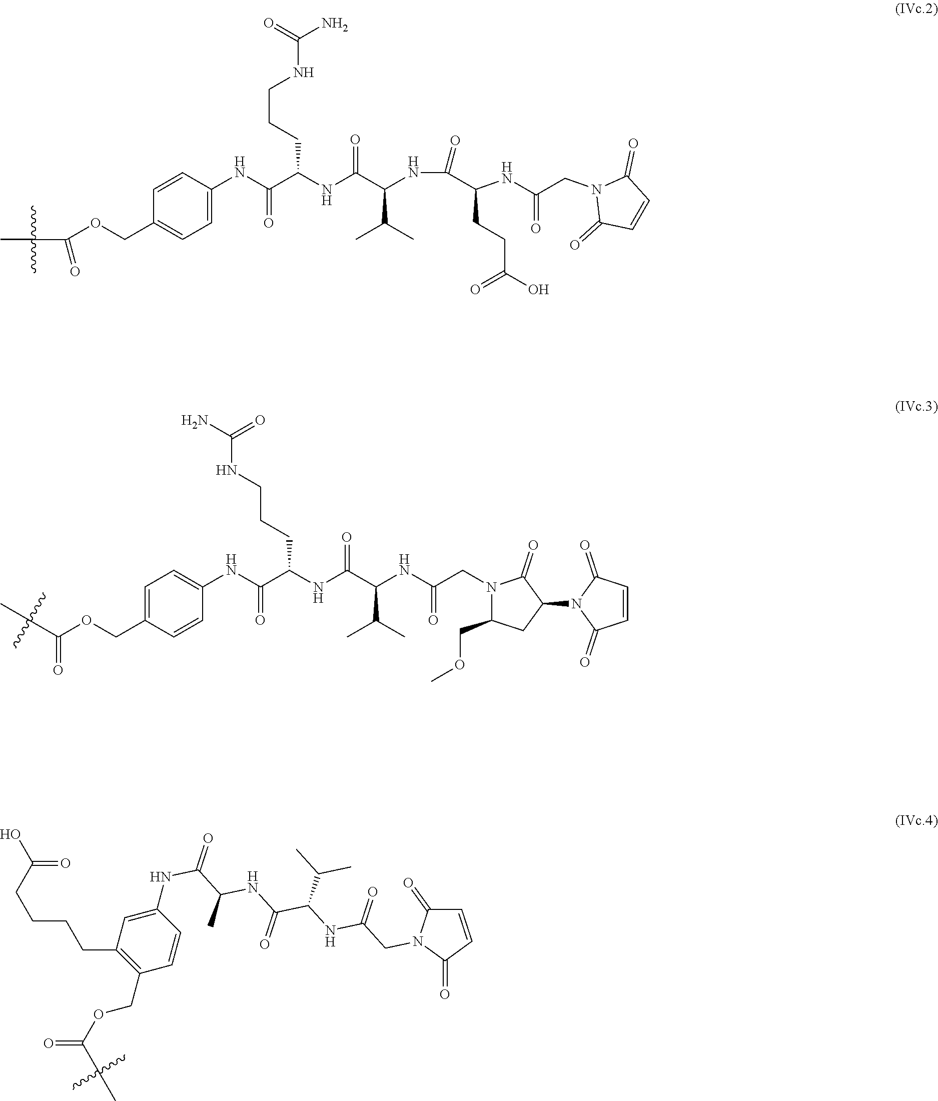 [SMILES: *C(=O)OCc1ccc(NC(=O)[C@H](CCCNC(N)=O)NC(=O)[C@@H](NC(=O)[C@H](CCC(=O)O)NC(=O)CN2C(=O)C=CC2=O)C(C)C)cc1 ; *C(=O)OCc1ccc(NC(=O)[C@H](CCCNC(N)=O)NC(=O)[C@@H](NC(=O)CN2C(=O)[C@@H](N3C(=O)C=CC3=O)C[C@H]2COC)C(C)C)cc1 ; *C(=O)OCc1ccc(NC(=O)[C@H](C)NC(=O)[C@@H](NC(=O)CN2C(=O)C=CC2=O)C(C)C)cc1CCCCC(=O)O]

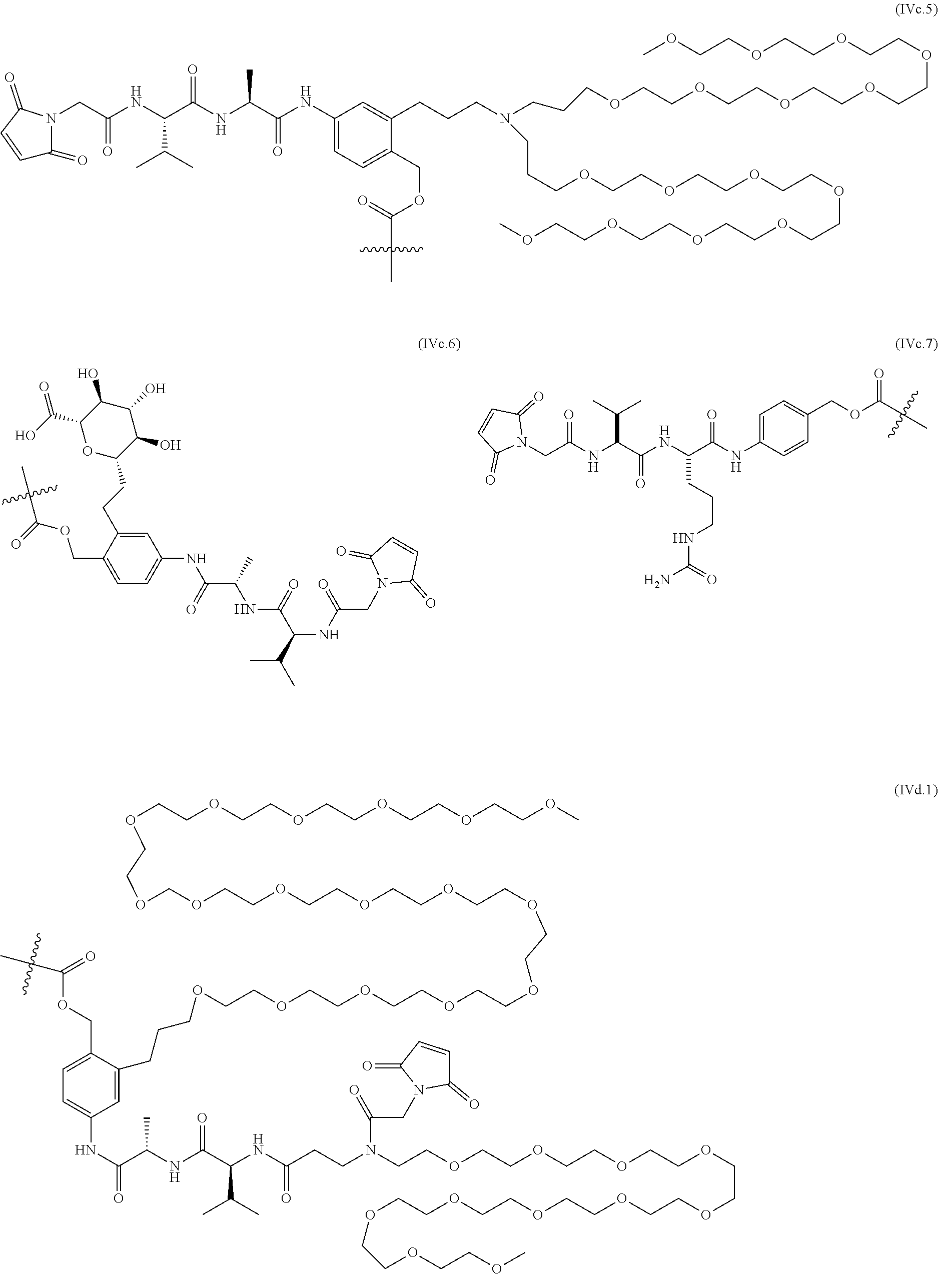

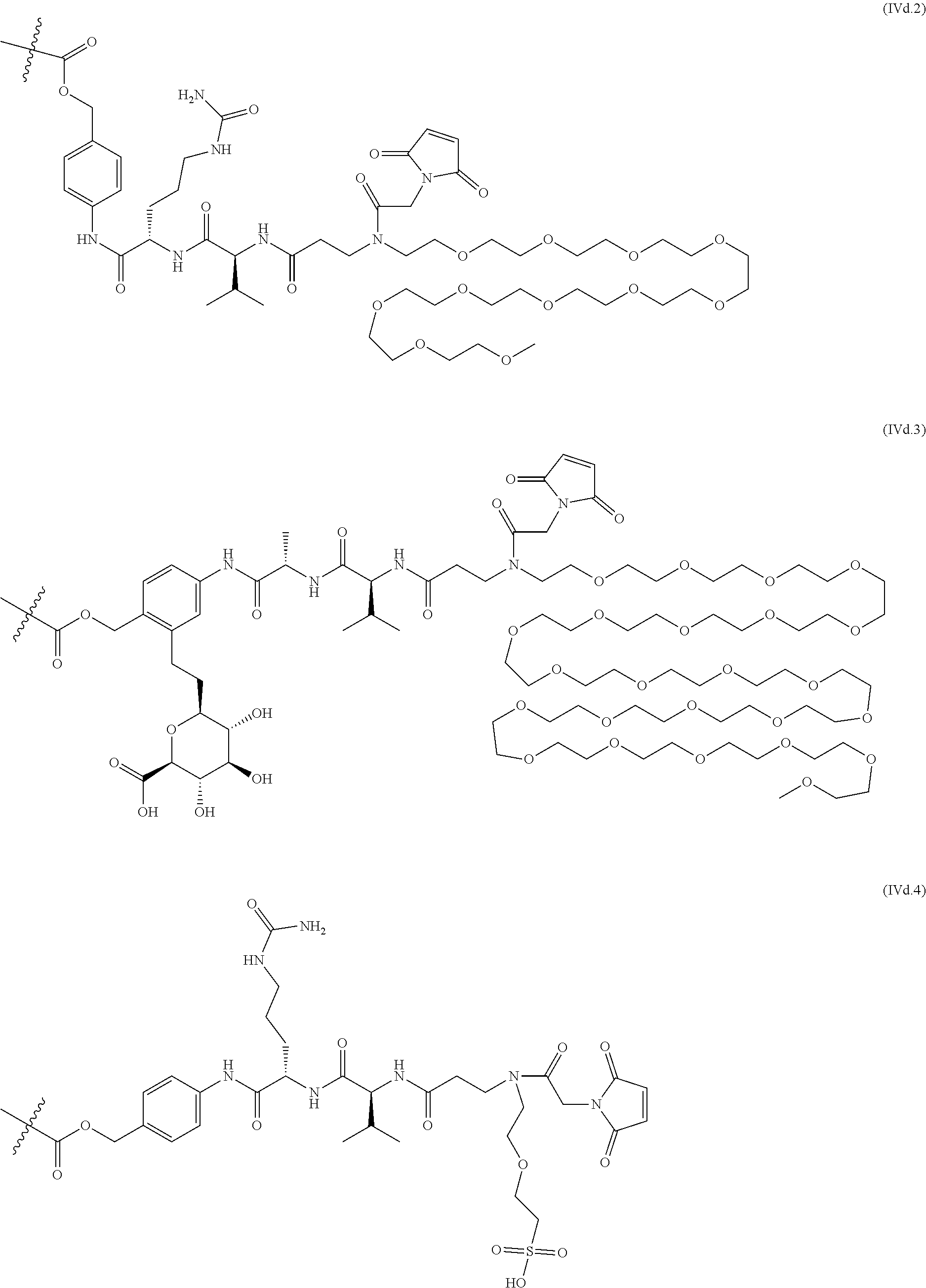 [SMILES: *C(=O)OCc1ccc(NC(=O)[C@H](CCCNC(N)=O)NC(=O)[C@@H](NC(=O)CCN(CCOCCOCCOCCOCCOCCOCCOCCOCCOCCOCCOC)C(=O)CN2C(=O)C=CC2=O)C(C)C)cc1 ; *C(=O)OCc1ccc(NC(=O)[C@H](C)NC(=O)[C@@H](NC(=O)CCN(CCOCCOCCOCCOCCOCCOCCOCCOCCOCCOCCOCCOCCOCCOCCOCCOCCOCCOCCOCCOCCOCCOCCOCCOC)C(=O)CN2C(=O)C=CC2=O)C(C)C)cc1CC[C@@H]1O[C@H](C(=O)O)[C@@H](O)[C@H](O)[C@H]1O ; *C(=O)OCc1ccc(NC(=O)[C@H](CCCNC(N)=O)NC(=O)[C@@H](NC(=O)CCN(CCOCCS(=O)(=O)O)C(=O)CN2C(=O)C=CC2=O)C(C)C)cc1]

The linker can contain an enzymatically cleavable sugar moiety, for example, a linker comprising structural formula (Va), (Vb), (Vc), (Vd), or (Ve):

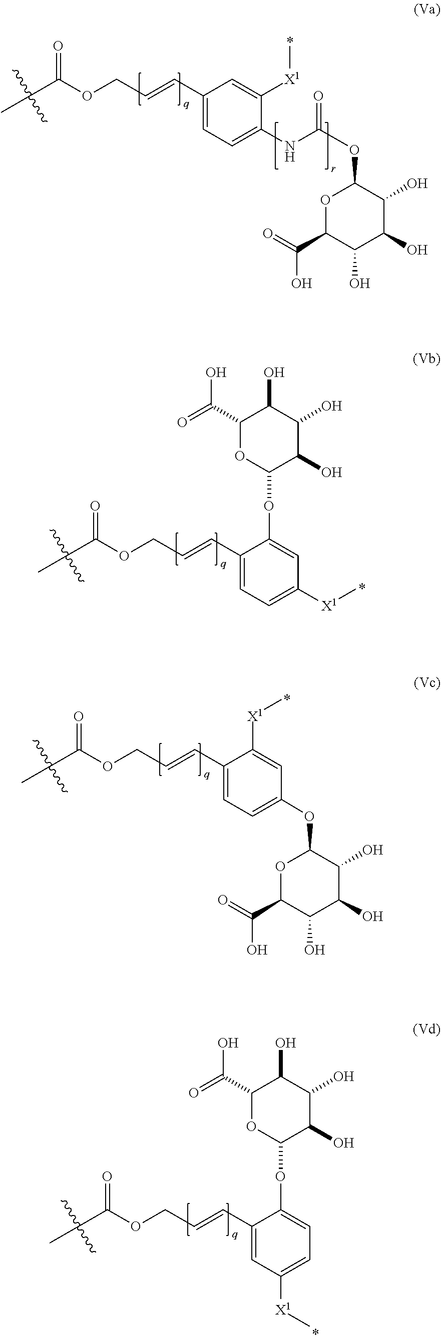

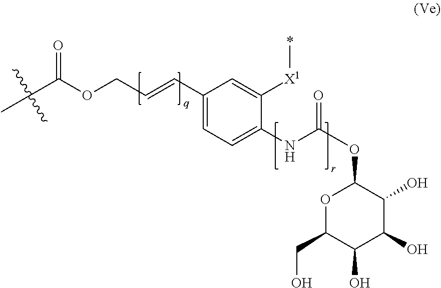

or a salt thereof, wherein: q is 0 or 1; r is 0 or 1; X¹ is CH₂, O or NH; [symbol] represents the point of attachment of the linker to the peptide or immune-stimulatory compound; and * represents the point of attachment to the remainder of the linker.

Exemplary embodiments of linkers according to structural formula (Va) that can be included in the construct-peptide conjugates or construct-peptide immune-stimulatory compound conjugates described herein can include the linkers illustrated below (as illustrated, the linkers include a group suitable for covalently linking the linker to a construct):

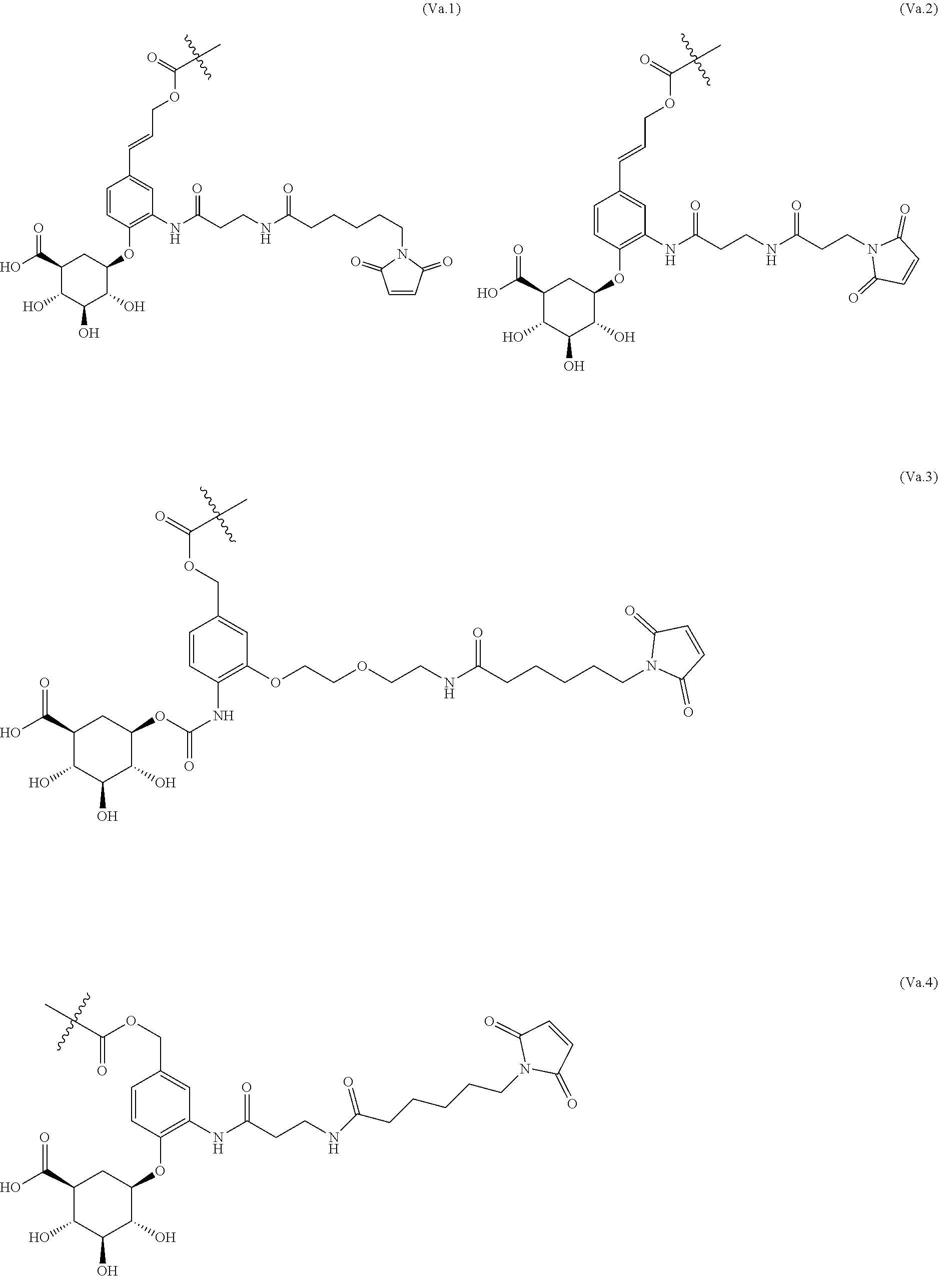 [SMILES: *C(=O)OC/C=C/c1ccc(O[C@@H]2C[C@H](C(=O)O)[C@@H](O)[C@H](O)[C@H]2O)c(NC(=O)CCNC(=O)CCCCCN2C(=O)C=CC2=O)c1 ; *C(=O)OC/C=C/c1ccc(O[C@@H]2C[C@H](C(=O)O)[C@@H](O)[C@H](O)[C@H]2O)c(NC(=O)CCNC(=O)CCN2C(=O)C=CC2=O)c1 ; *C(=O)OCc1ccc(NC(=O)O[C@@H]2C[C@H](C(=O)O)[C@@H](O)[C@H](O)[C@H]2O)c(OCCOCCNC(=O)CCCCCN2C(=O)C=CC2=O)c1 ; *C(=O)OCc1ccc(O[C@@H]2C[C@H](C(=O)O)[C@@H](O)[C@H](O)[C@H]2O)c(NC(=O)CCNC(=O)CCCCCN2C(=O)C=CC2=O)c1]

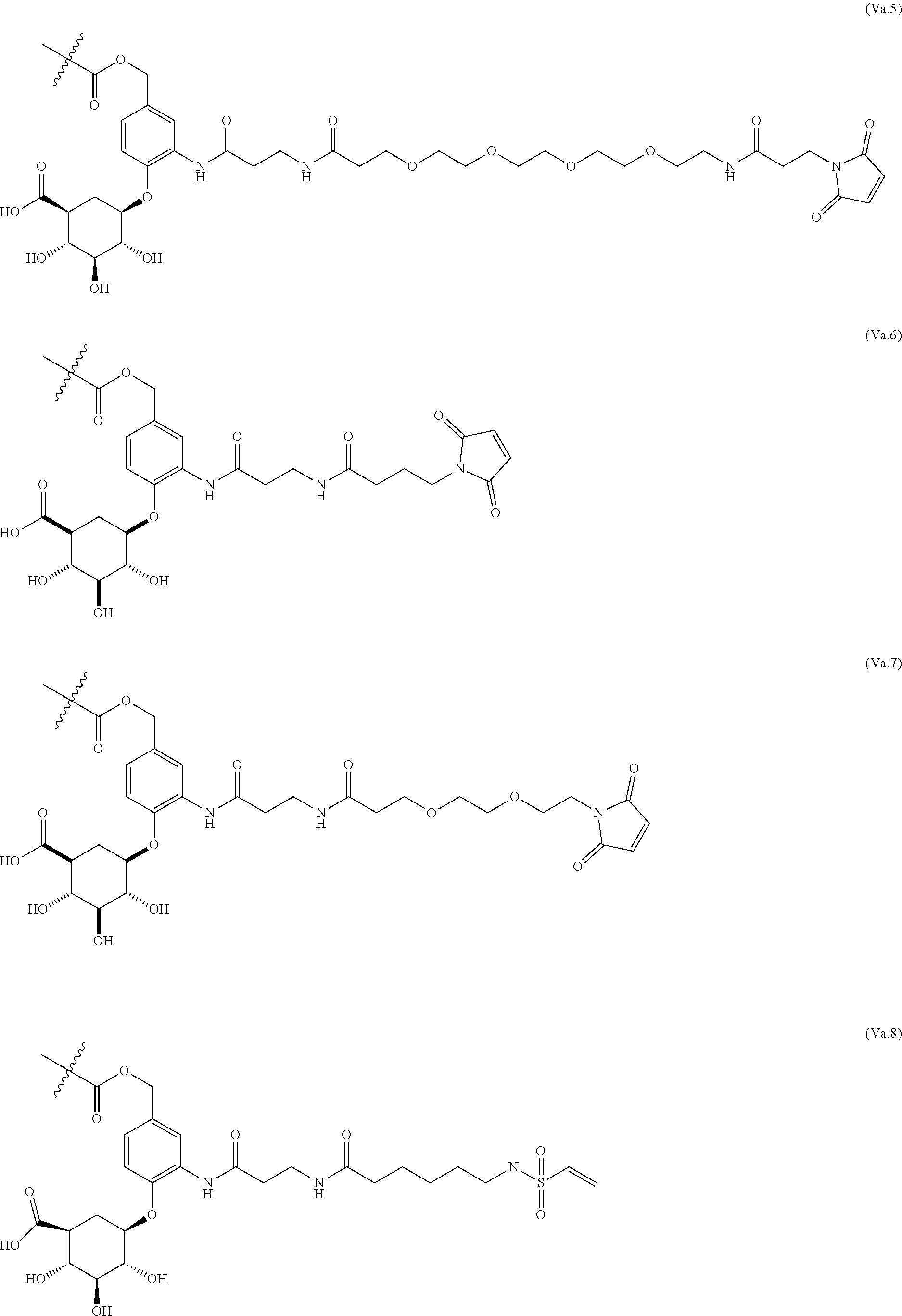 [SMILES: *C(=O)OCc1ccc(O[C@@H]2C[C@H](C(=O)O)[C@@H](O)[C@H](O)[C@H]2O)c(NC(=O)CCNC(=O)CCOCCOCCOCCOCCNC(=O)CCN2C(=O)C=CC2=O)c1 ; *C(=O)OCc1ccc(O[C@@H]2C[C@H](C(=O)O)[C@@H](O)[C@H](O)[C@H]2O)c(NC(=O)CCNC(=O)CCCN2C(=O)C=CC2=O)c1 ; *C(=O)OCc1ccc(O[C@@H]2C[C@H](C(=O)O)[C@@H](O)[C@H](O)[C@H]2O)c(NC(=O)CCNC(=O)CCOCCOCCN2C(=O)C=CC2=O)c1 ; *C(=O)OCc1ccc(O[C@@H]2C[C@H](C(=O)O)[C@@H](O)[C@H](O)[C@H]2O)c(NC(=O)CCNC(=O)CCCCCNS(=O)(=O)C=C)c1]

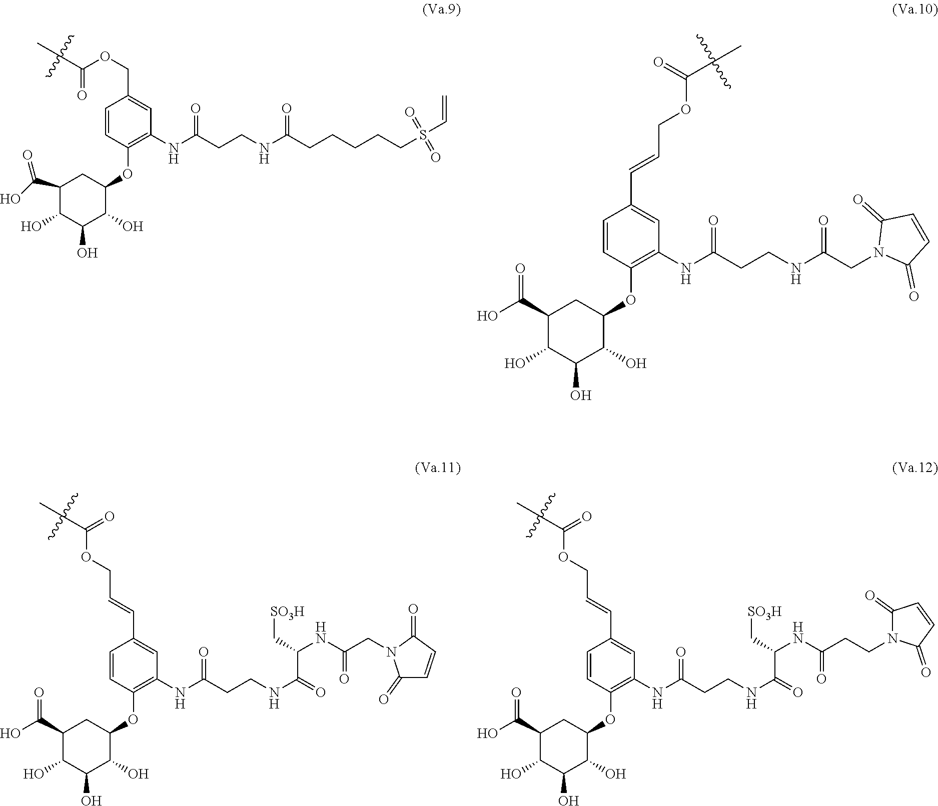 [SMILES: *C(=O)OCc1ccc(O[C@@H]2C[C@H](C(=O)O)[C@@H](O)[C@H](O)[C@H]2O)c(NC(=O)CCNC(=O)CCCCCS(=O)(=O)C=C)c1 ; *C(=O)OC/C=C/c1ccc(O[C@@H]2C[C@H](C(=O)O)[C@@H](O)[C@H](O)[C@H]2O)c(NC(=O)CCNC(=O)CN2C(=O)C=CC2=O)c1 ; *C(=O)OC/C=C/c1ccc(O[C@@H]2C[C@H](C(=O)O)[C@@H](O)[C@H](O)[C@H]2O)c(NC(=O)CCNC(=O)[C@H](CS(=O)(=O)O)NC(=O)CN2C(=O)C=CC2=O)c1 ; *C(=O)OC/C=C/c1ccc(O[C@@H]2C[C@H](C(=O)O)[C@@H](O)[C@H](O)[C@H]2O)c(NC(=O)CCNC(=O)[C@H](CS(=O)(=O)O)NC(=O)CCN2C(=O)C=CC2=O)c1]

Exemplary embodiments of linkers according to structural formula (Vb) that may be included in the construct-peptide conjugates or construct-peptides immune-stimulatory compound conjugates described herein include the linkers illustrated below (as illustrated, the linkers include a group suitable for covalently linking the linker to a construct):

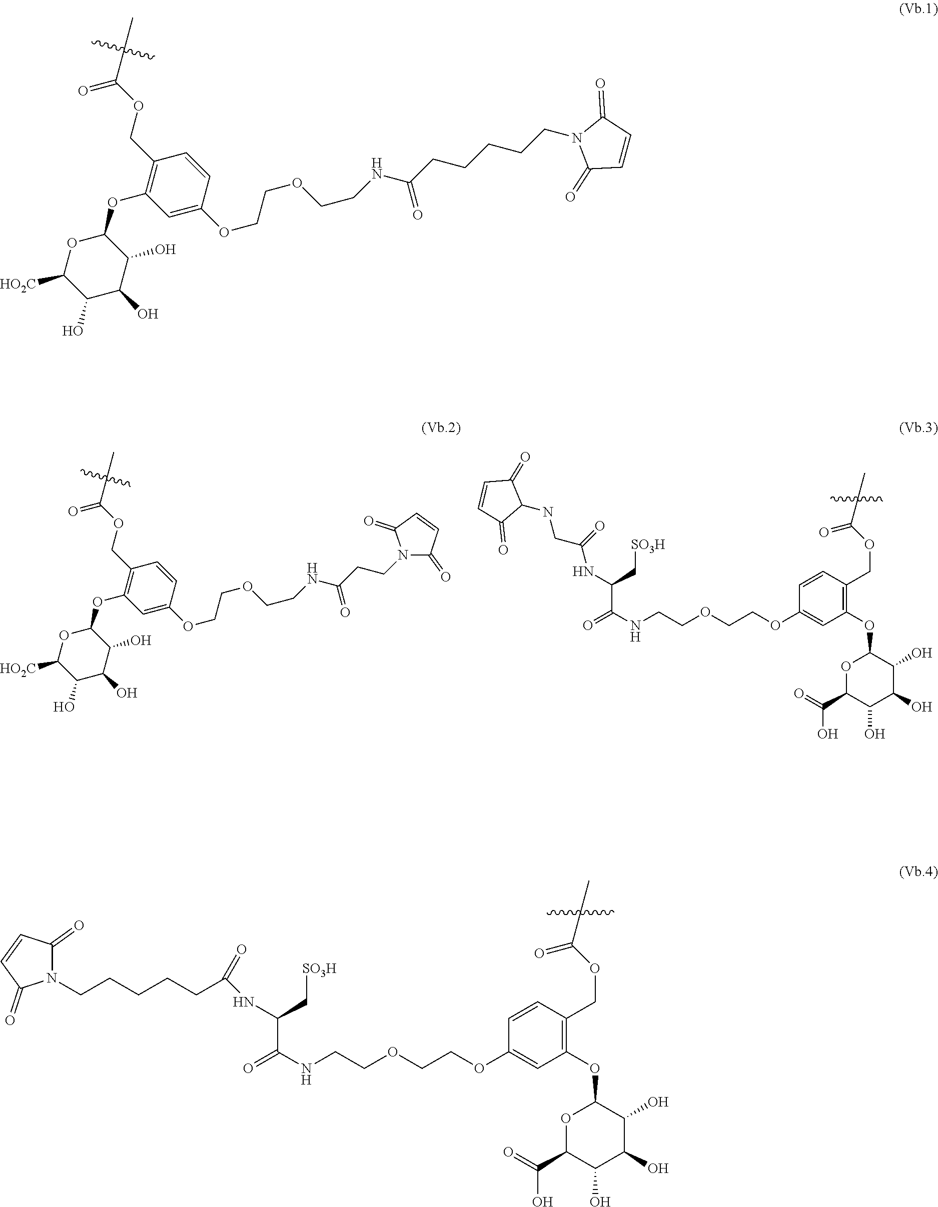

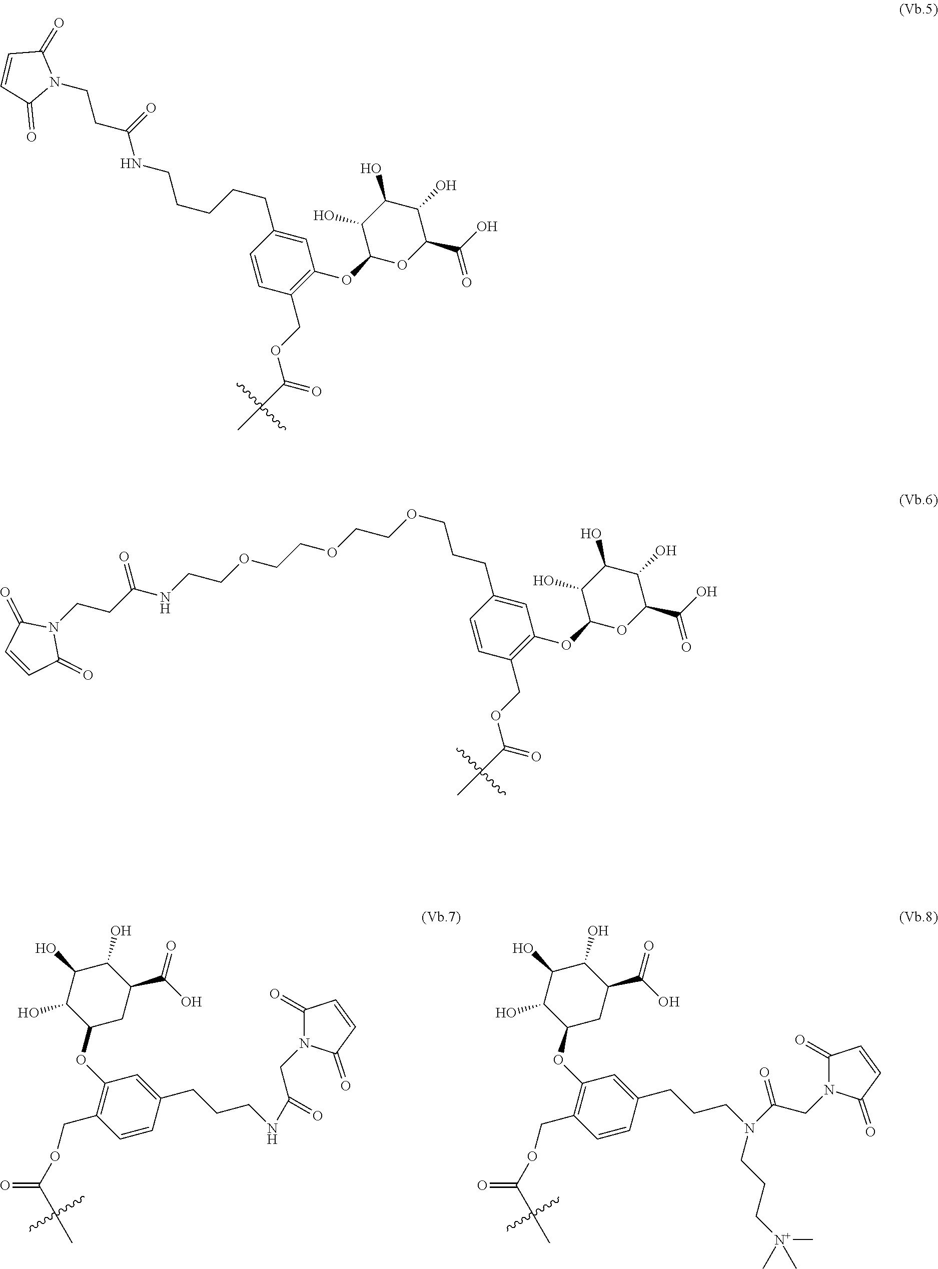 [SMILES: *C(=O)OCc1ccc(CCCCCNC(=O)CCN2C(=O)C=CC2=O)cc1O[C@@H]1O[C@H](C(=O)O)[C@@H](O)[C@H](O)[C@H]1O ; *C(=O)OCc1ccc(CCCOCCOCCOCCNC(=O)CCN2C(=O)C=CC2=O)cc1O[C@@H]1O[C@H](C(=O)O)[C@@H](O)[C@H](O)[C@H]1O ; *C(=O)OCc1ccc(CCCNC(=O)CN2C(=O)C=CC2=O)cc1O[C@@H]1C[C@H](C(=O)O)[C@@H](O)[C@H](O)[C@H]1O ; *C(=O)OCc1ccc(CCCN(CCC[N+](C)(C)C)C(=O)CN2C(=O)C=CC2=O)cc1O[C@@H]1C[C@H](C(=O)O)[C@@H](O)[C@H](O)[C@H]1O]

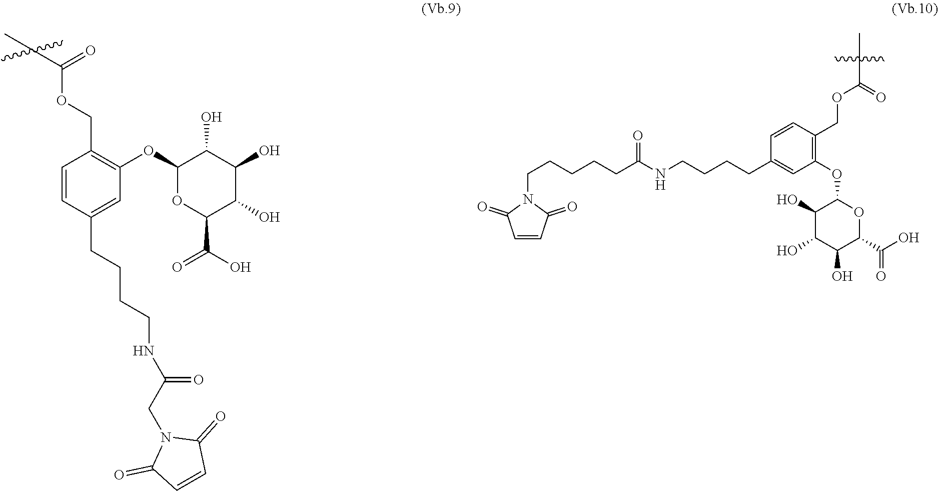 [SMILES: *C(=O)OCc1ccc(CCCCNC(=O)CN2C(=O)C=CC2=O)cc1O[C@@H]1O[C@H](C(=O)O)[C@@H](O)[C@H](O)[C@H]1O ; *C(=O)OCc1ccc(CCCCNC(=O)CCCCCN2C(=O)C=CC2=O)cc1O[C@@H]1O[C@H](C(=O)O)[C@@H](O)[C@H](O)[C@H]1O]

Exemplary embodiments of linkers according to structural formula (Vc) that may be included in the construct-peptide conjugates or peptide-construct immune-stimulatory compound conjugates described herein include the linkers illustrated below (as illustrated, the linkers include a group suitable for covalently linking the linker to a construct):

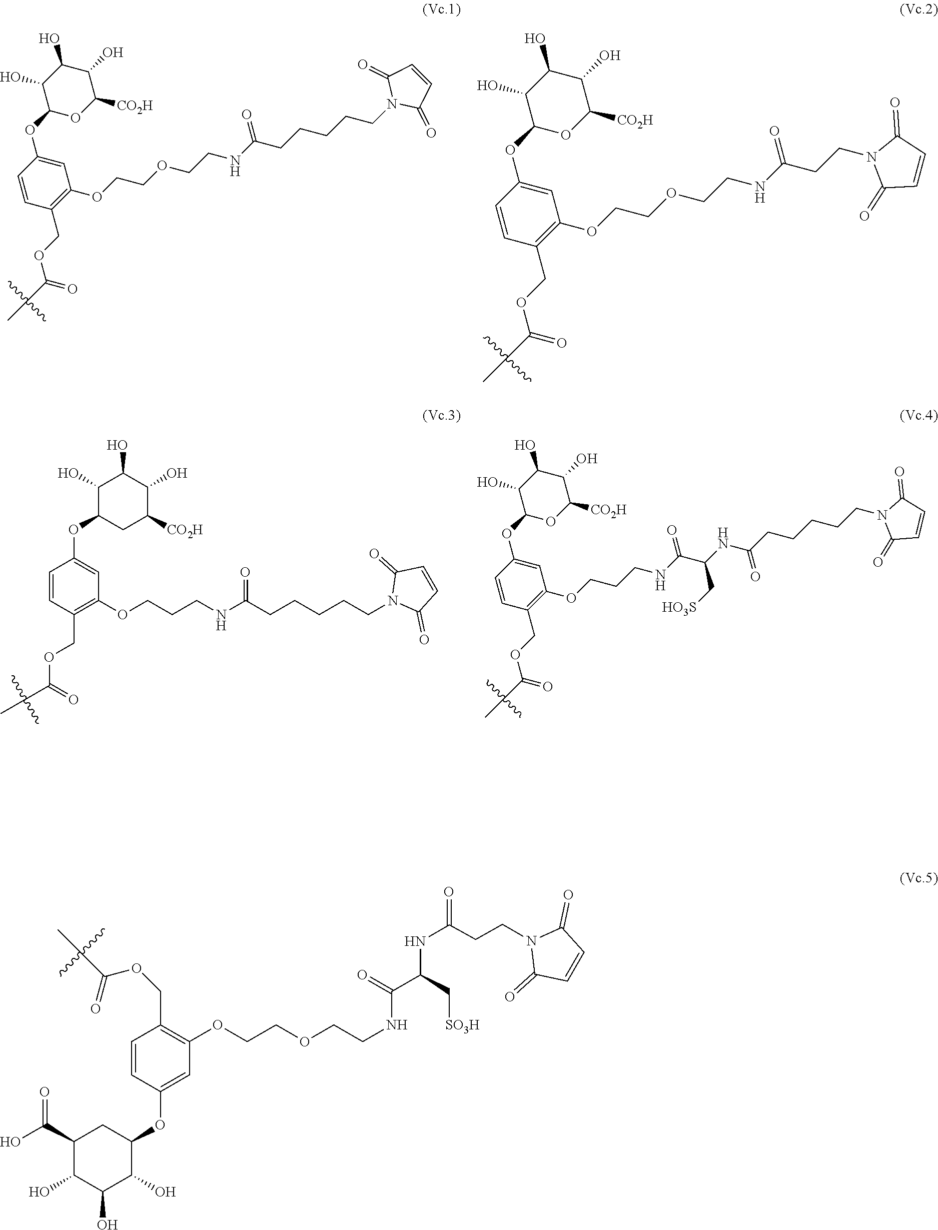

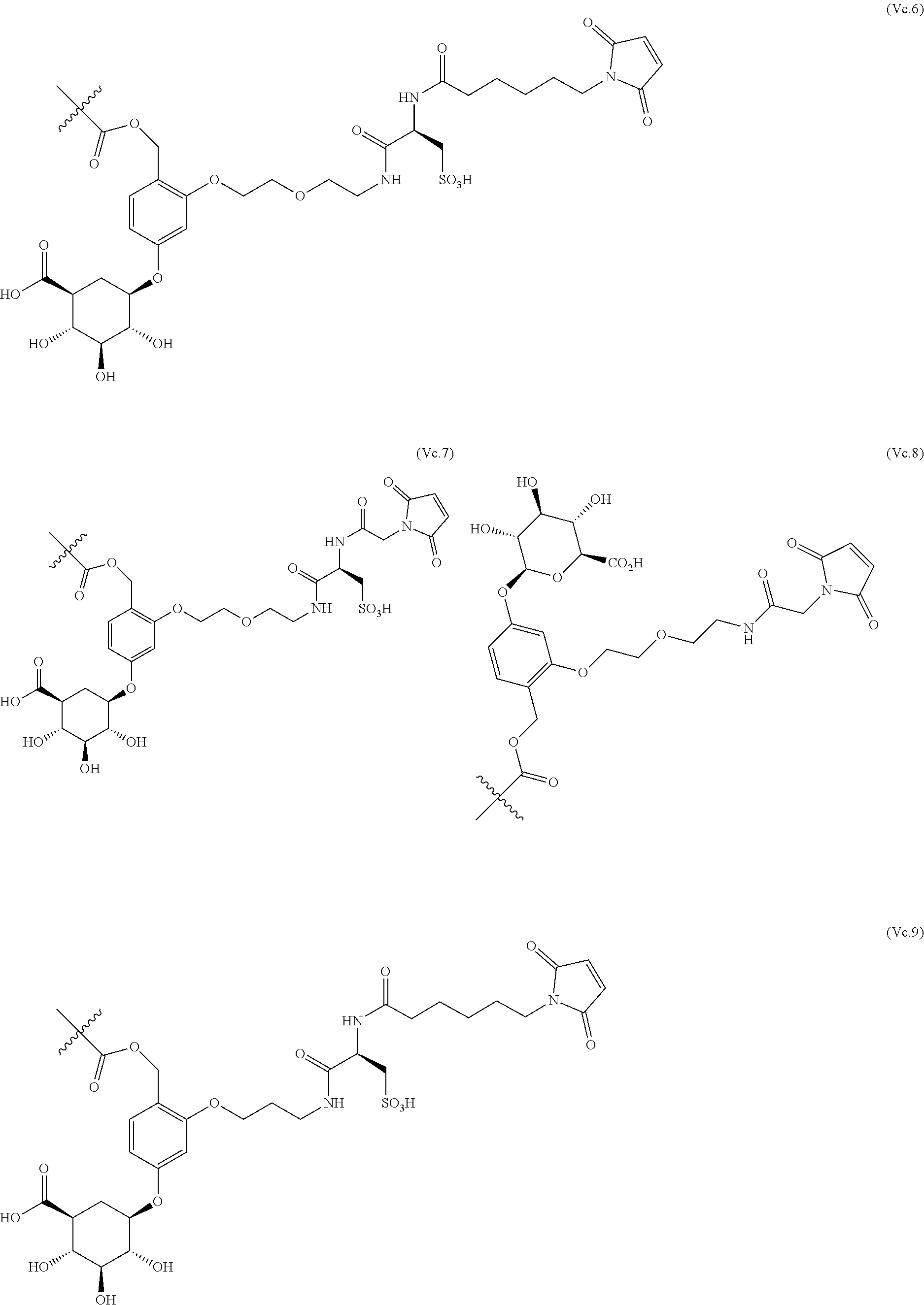

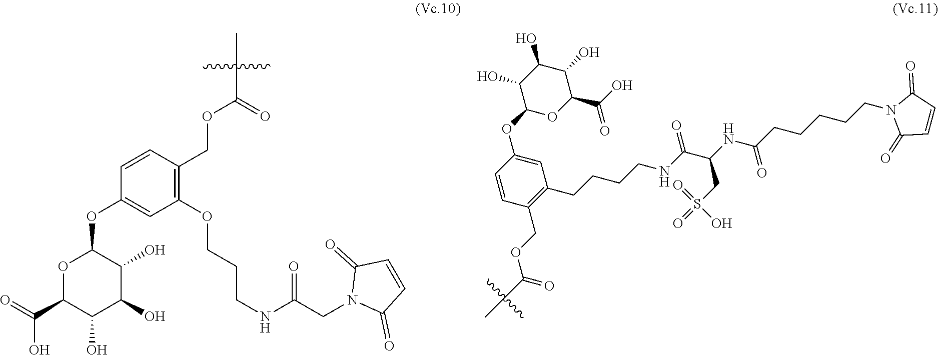 [SMILES: *C(=O)OCc1ccc(O[C@@H]2O[C@H](C(=O)O)[C@@H](O)[C@H](O)[C@H]2O)cc1OCCCNC(=O)CN1C(=O)C=CC1=O ; *C(=O)OCc1ccc(O[C@@H]2O[C@H](C(=O)O)[C@@H](O)[C@H](O)[C@H]2O)cc1CCCCNC(=O)[C@H](CS(=O)(=O)O)NC(=O)CCCCCN1C(=O)C=CC1=O]

Exemplary embodiments of linkers according to structural formula (Vd) that may be included in the construct-peptide conjugates or construct-peptide immune-stimulatory compound conjugates described herein include the linkers illustrated below (as illustrated, the linkers include a group suitable for covalently linking the linker to a construct):

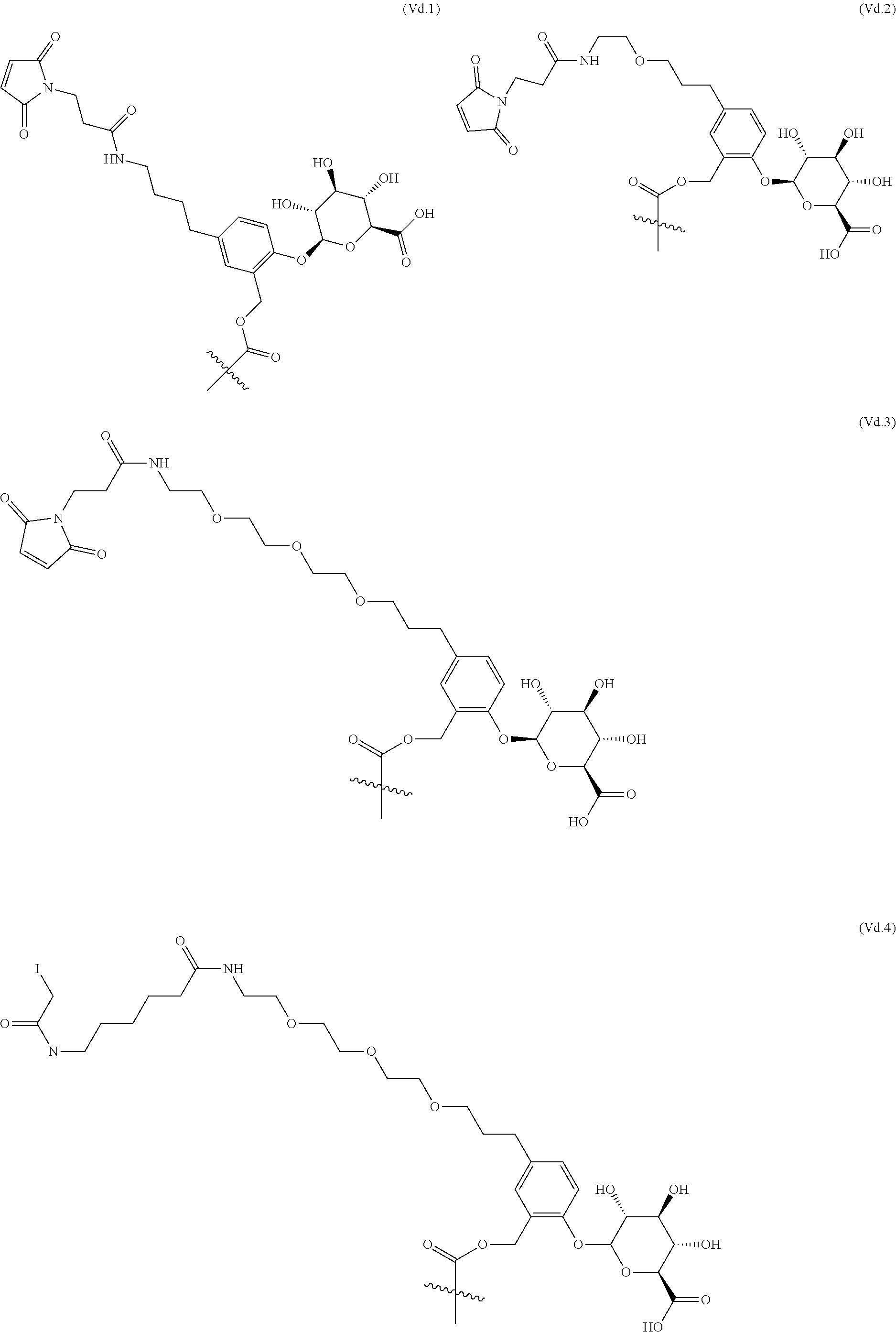 [SMILES: *C(=O)OCc1cc(CCCCNC(=O)CCN2C(=O)C=CC2=O)ccc1O[C@@H]1O[C@H](C(=O)O)[C@@H](O)[C@H](O)[C@H]1O ; *C(=O)OCc1cc(CCCOCCNC(=O)CCN2C(=O)C=CC2=O)ccc1O[C@@H]1O[C@H](C(=O)O)[C@@H](O)[C@H](O)[C@H]1O ; *C(=O)OCc1cc(CCCOCCOCCOCCNC(=O)CCN2C(=O)C=CC2=O)ccc1O[C@@H]1O[C@H](C(=O)O)[C@@H](O)[C@H](O)[C@H]1O ; *C(=O)OCc1cc(CCCOCCOCCOCCNC(=O)CCCCCNC(=O)CI)ccc1OC1O[C@H](C(=O)O)[C@@H](O)[C@H](O)[C@H]1O]

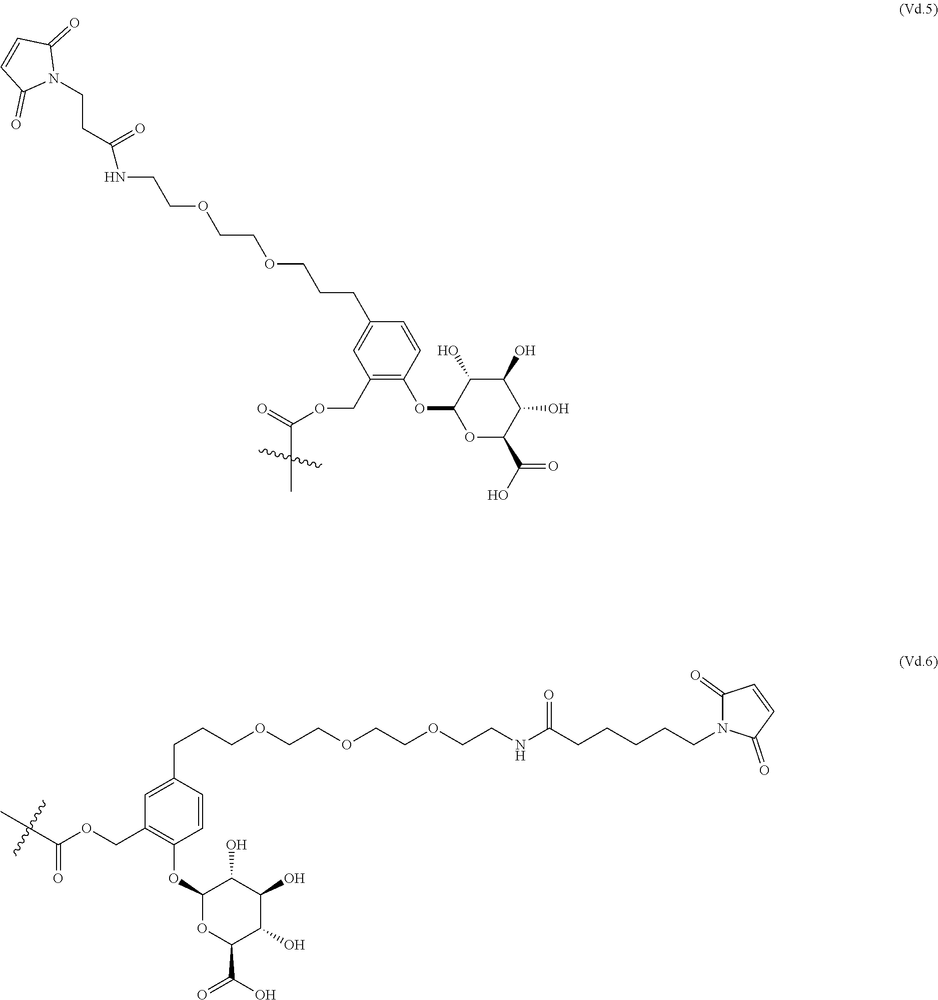 [SMILES: *C(=O)OCc1cc(CCCOCCOCCNC(=O)CCN2C(=O)C=CC2=O)ccc1O[C@@H]1O[C@H](C(=O)O)[C@@H](O)[C@H](O)[C@H]1O ; *C(=O)OCc1cc(CCCOCCOCCOCCNC(=O)CCCCCN2C(=O)C=CC2=O)ccc1O[C@@H]1O[C@H](C(=O)O)[C@@H](O)[C@H](O)[C@H]1O]

Exemplary embodiments of linkers according to structural formula (Ve) that may be included in the construct immune-stimulatory compound conjugates described herein include the linkers illustrated below (as illustrated, the linkers include a group suitable for covalently linking the linker to a construct):

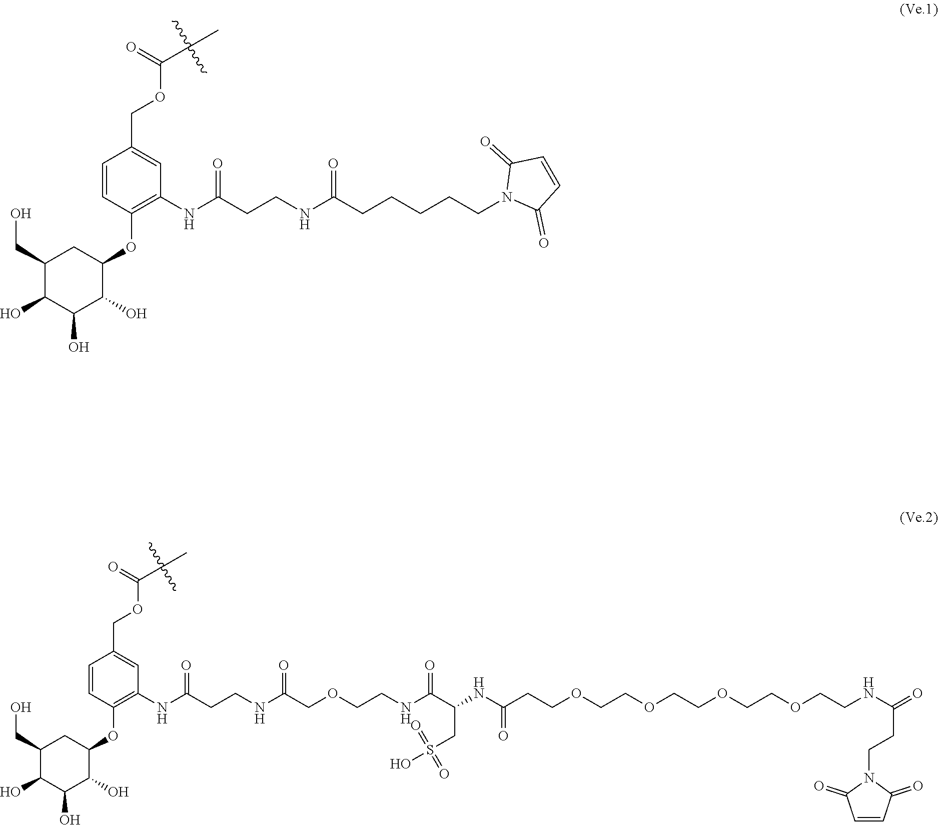 [SMILES: *C(=O)OCc1ccc(O[C@@H]2C[C@H](CO)[C@H](O)[C@H](O)[C@H]2O)c(NC(=O)CCNC(=O)CCCCCN2C(=O)C=CC2=O)c1 ; *C(=O)OCc1ccc(O[C@@H]2C[C@H](CO)[C@H](O)[C@H](O)[C@H]2O)c(NC(=O)CCNC(=O)COCCNC(=O)[C@@H](CS(=O)(=O)O)NC(=O)CCOCCOCCOCCOCCNC(=O)CCN2C(=O)C=CC2=O)c1]

Although cleavable linkers can provide certain advantages, the linkers comprising the conjugate described herein need not be cleavable. For non-cleavable linkers, the peptide or immune-stimulatory compound release may not depend on the differential properties between the plasma and some cytoplasmic compartments. The release of the peptide or immune-stimulatory compound can occur after internalization of the construct-peptide conjugate or construct-peptide immune-stimulatory compound conjugate via antigen-mediated endocytosis and delivery to lysosomal compartment, where the construct can be degraded to the level of amino acids through intracellular proteolytic degradation. This process can release a peptide and/or immune-stimulatory compound derivative, which is formed by the peptide or immune-stimulatory compound, the linker, and the amino acid residue to which the linker was covalently attached. The peptide or immune-stimulatory compound derivative from construct-peptide conjugates or construct-peptide immune-stimulatory compound conjugates with non-cleavable linkers can be more hydrophilic and less membrane permeable, which can lead to less bystander effects and less nonspecific toxicities compared to construct-peptide conjugates or construct-peptide immune-stimulatory compound conjugates with a cleavable linker. Construct-peptide conjugates or construct-peptide immune-stimulatory compound conjugates with non-cleavable linkers can have greater stability in circulation than construct-peptide immune-stimulatory compound conjugates with cleavable linkers. Non-cleavable linkers can be alkylene chains, or can be polymeric, such as, for example, based upon polyalkylene glycol polymers, amide polymers, or can include segments of alkylene chains, polyalkylene glycols and/or amide polymers. The linker can contain a polyethylene glycol segment having from 1 to 6 ethylene glycol units.

The linker can be non-cleavable in vivo, for example, a linker according to the formulations below:

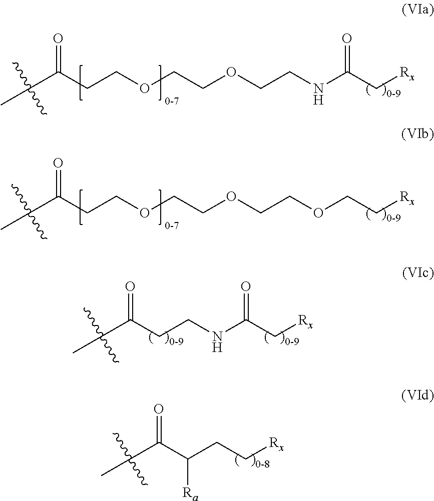

or salts thereof, wherein: R^{a} is selected from hydrogen, alkyl, sulfonate and methyl sulfonate; R^{x} is a moiety including a functional group capable of covalently linking the linker to a construct; and [symbol] represents the point of attachment of the linker to the peptide or immune-stimulatory compound.

Exemplary embodiments of linkers according to structural formula (VIa)-(VId) that may be included in the conjugates described herein include the linkers illustrated below (as illustrated, the linkers include a group suitable for covalently linking the linker to a construct, and [symbol] represents the point of attachment to a peptide or an immune-stimulatory compound):

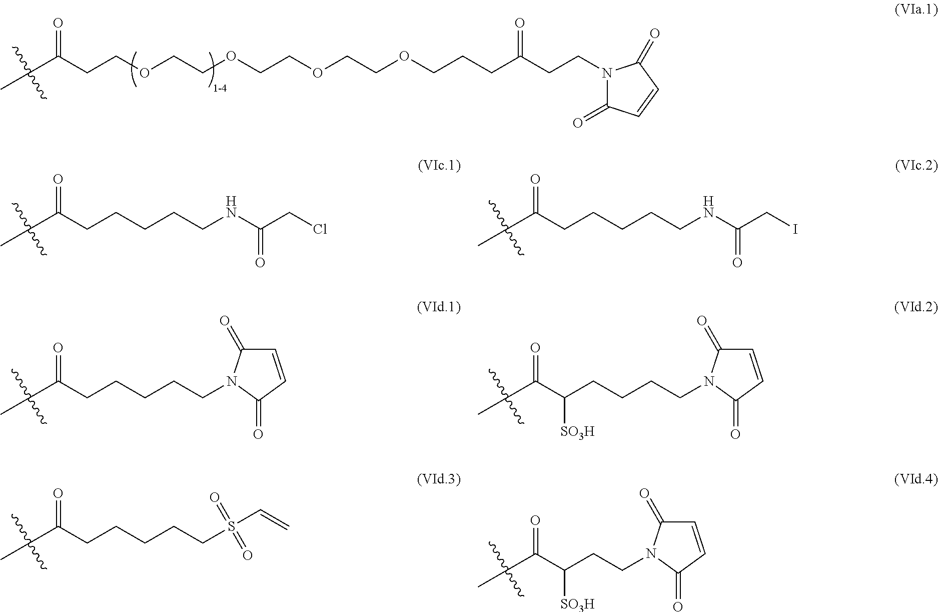

Attachment groups that are used to attach the linkers to a construct can be electrophilic in nature and include, for example, maleimide groups, activated disulfides, active esters such as NHS esters and HOBt esters, haloformates, acid halides, alkyl, and benzyl halides such as haloacetamides. There are also emerging technologies related to “self-stabilizing” maleimides and “bridging disulfides” that can be used in accordance with the disclosure.

One example of a “self-stabilizing” maleimide group that hydrolyzes spontaneously under construct conjugation conditions to give a construct-peptide conjugate or construct-peptide immune-stimulatory compound conjugate species with improved stability is depicted in the schematic below. Thus, the maleimide attachment group is reacted with a sulfhydryl of an antibody construct to give an intermediate succinimide ring. The hydrolyzed form of the attachment group is resistant to deconjugation in the presence of plasma proteins.

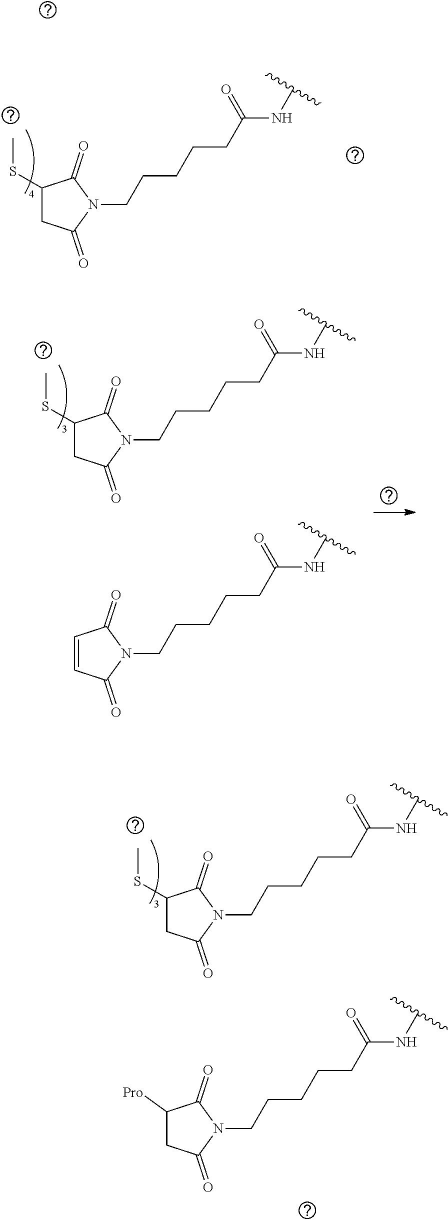

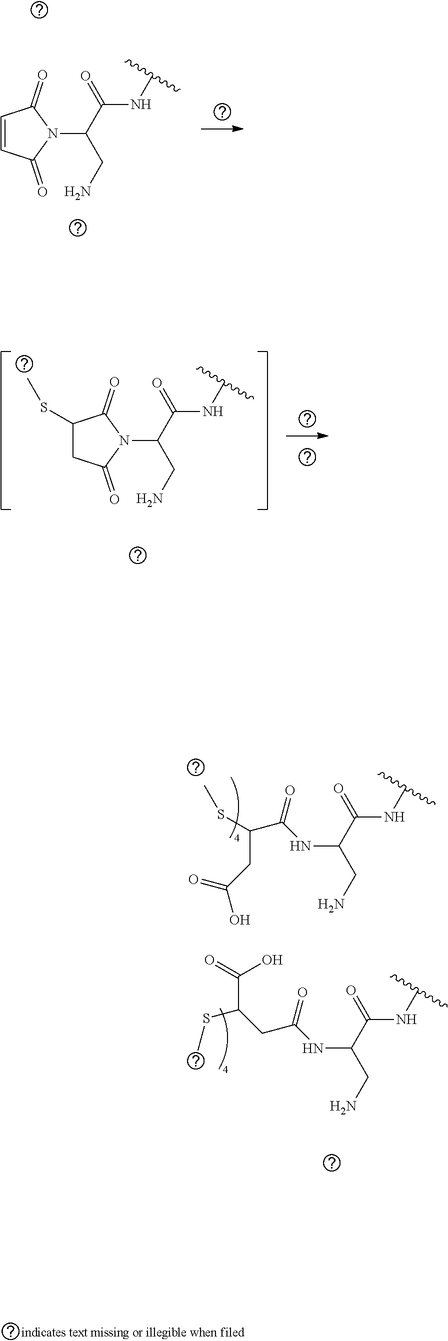

A method for bridging a pair of sulfhydryl groups derived from reductions of a native hinge disulfide bond has been disclosed and is depicted in the schematic below. An advantage of this methodology is the ability to synthesize homogenous DAR4 construct immune-stimulatory compound conjugates by full reduction of IgGs (to give 4 pairs of sulfhydryls) followed by reaction with 4 equivalents of the alkylating agent. Antibody construct immune-stimulatory compound conjugates containing “bridged disulfides” are also claimed to have increased stability.

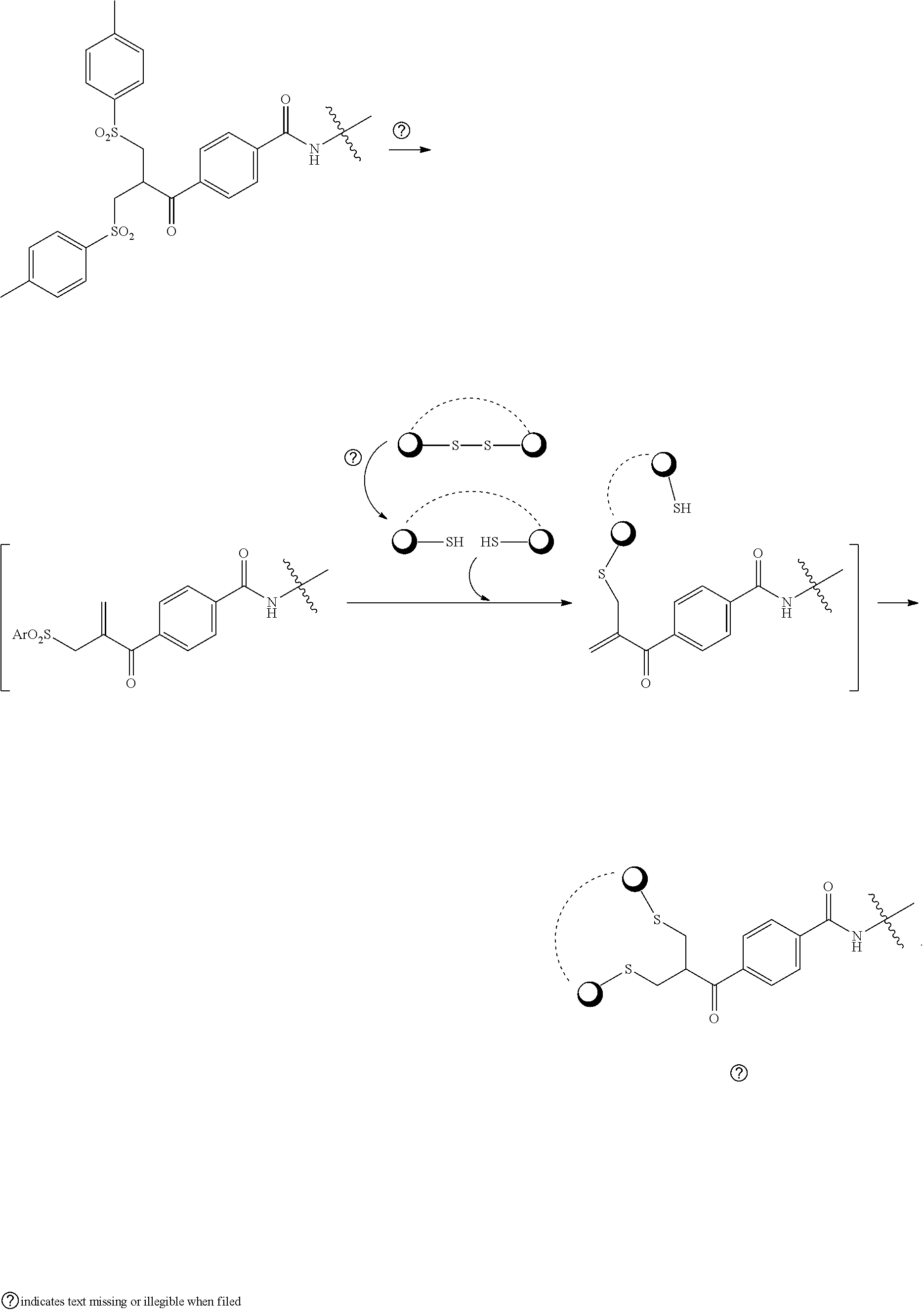

Similarly, as depicted below, a maleimide derivative that is capable of bridging a pair of sulfhydryl groups has been developed.

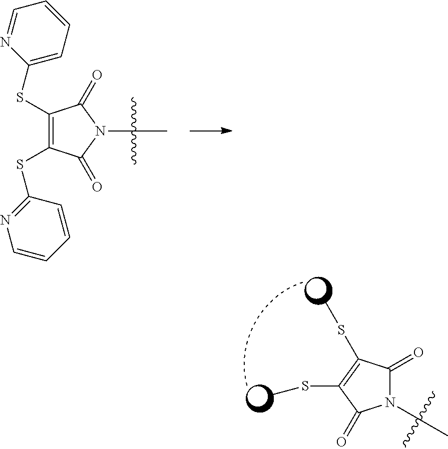 [SMILES: *N1C(=O)C(Sc2ccccn2)=C(Sc2ccccn2)C1=O ; *N1C(=O)C(SC)=C(SC)C1=O]

The attachment moiety can contain the following structural formulas (VIIa), (VIIb), or (VIIc):

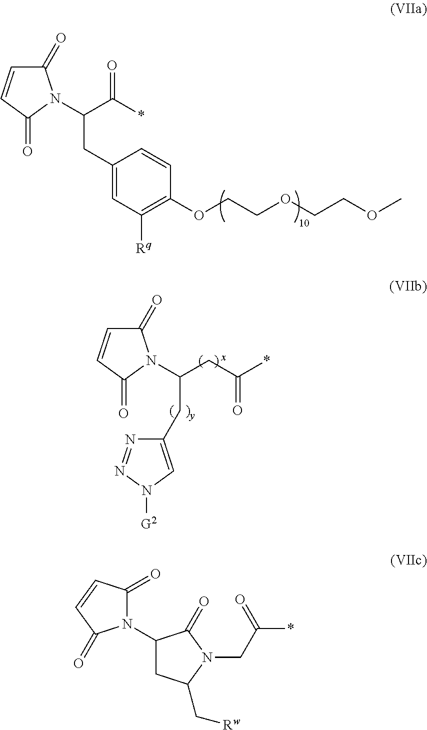

or salts thereof, wherein: R^{q} is H or —O—(CH₂CH₂O)₁₁—CH₃; x is 0 or 1; y is 0 or 1; G² is-CH₂CH₂CH₂SO₃H or —CH₂CH₂O—(CH₂CH₂O)_{II}—CH₃; R^{w} is-O—CH₂CH₂SO₃H or —NH(CO)—CH₂CH₂O—(CH₂CH₂O)₁₂—CH₃; and * represents the point of attachment to the remainder of the linker.

Exemplary embodiments of linkers according to structural formula (VIIa) and (VIIb) that can be included in the construct-peptide immune-stimulatory compound conjugates described herein can include the linkers illustrated below (as illustrated, the linkers can include a group suitable for covalently linking the linker to a construct):

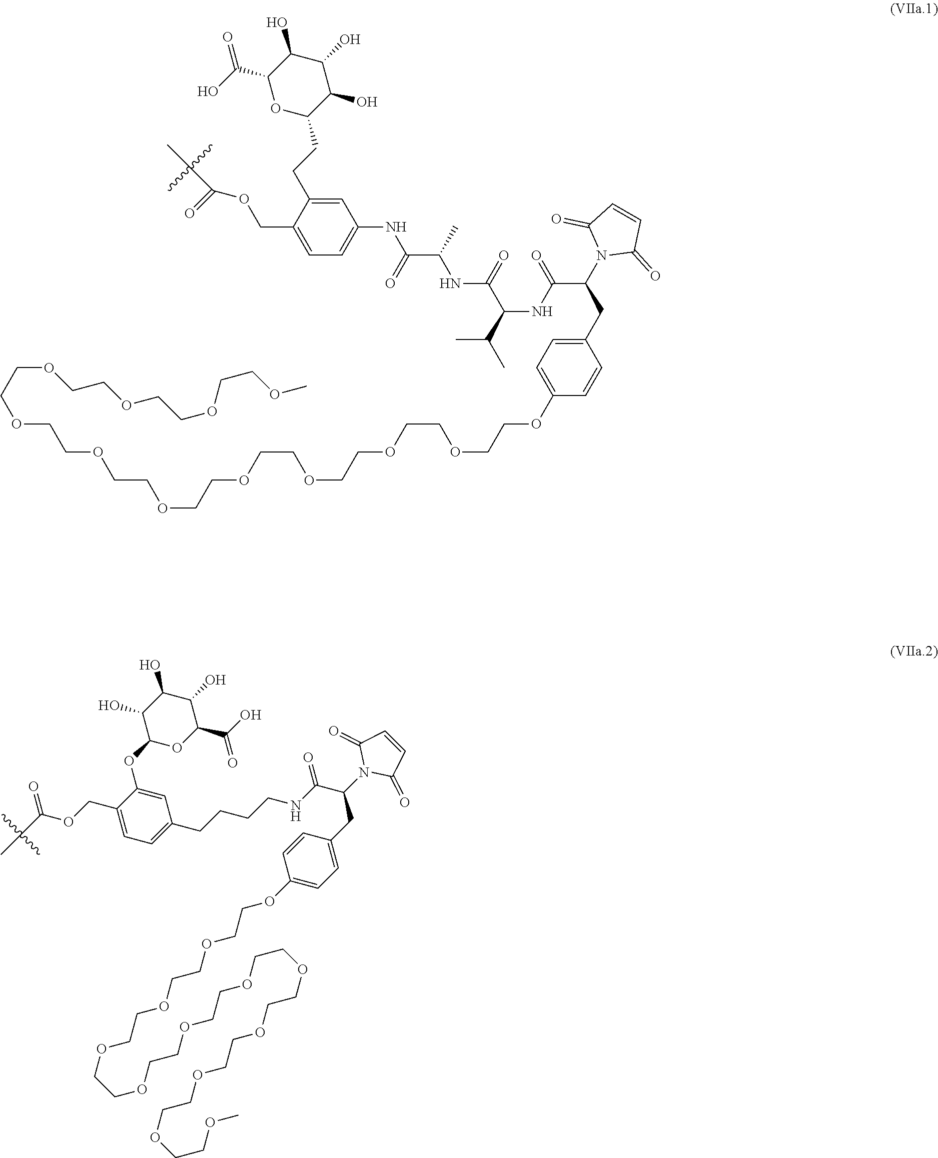 [SMILES: *C(=O)OCc1ccc(NC(=O)[C@H](C)NC(=O)[C@@H](NC(=O)[C@H](Cc2ccc(OCCOCCOCCOCCOCCOCCOCCOCCOCCOCCOCCOC)cc2)N2C(=O)C=CC2=O)C(C)C)cc1CC[C@@H]1O[C@H](C(=O)O)[C@@H](O)[C@H](O)[C@H]1O ; *C(=O)OCc1ccc(CCCCNC(=O)[C@H](Cc2ccc(OCCOCCOCCOCCOCCOCCOCCOCCOCCOCCOCCOC)cc2)N2C(=O)C=CC2=O)cc1O[C@@H]1O[C@H](C(=O)O)[C@@H](O)[C@H](O)[C@H]1O]

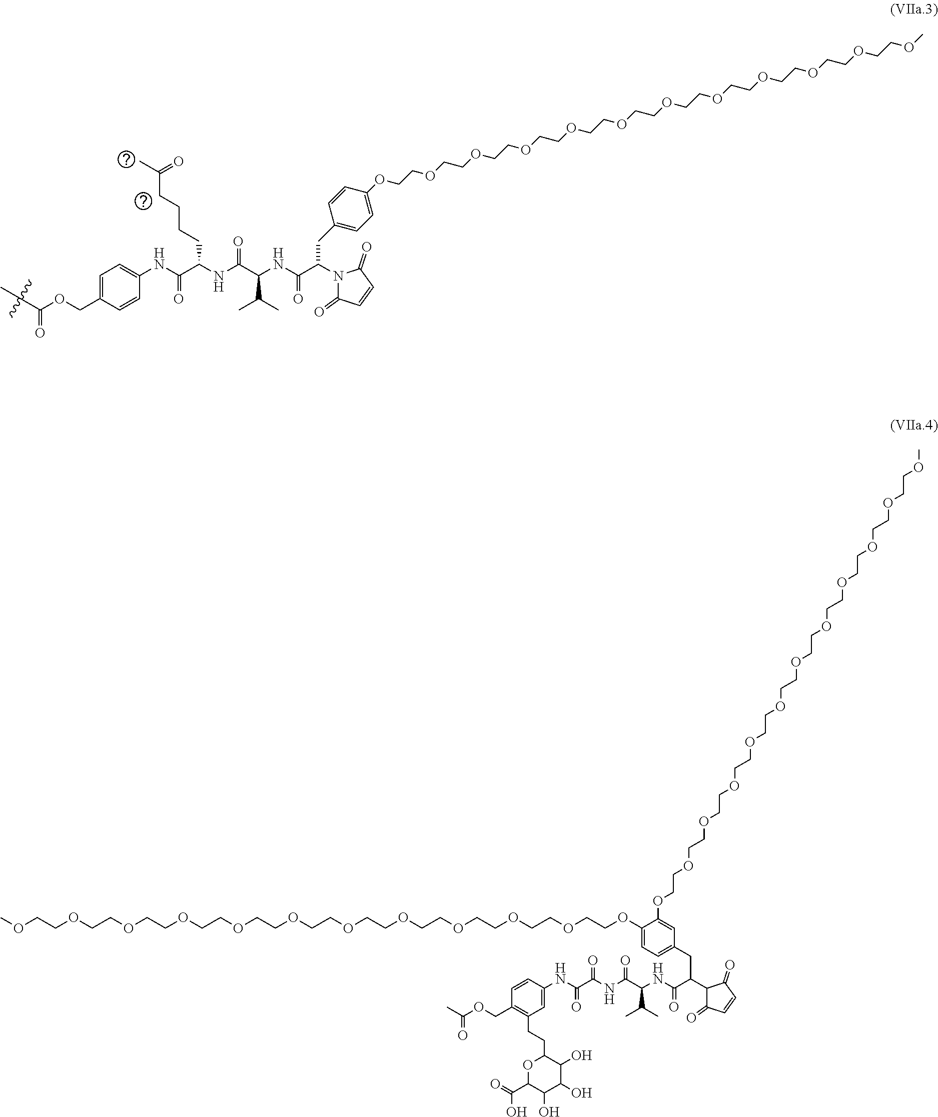

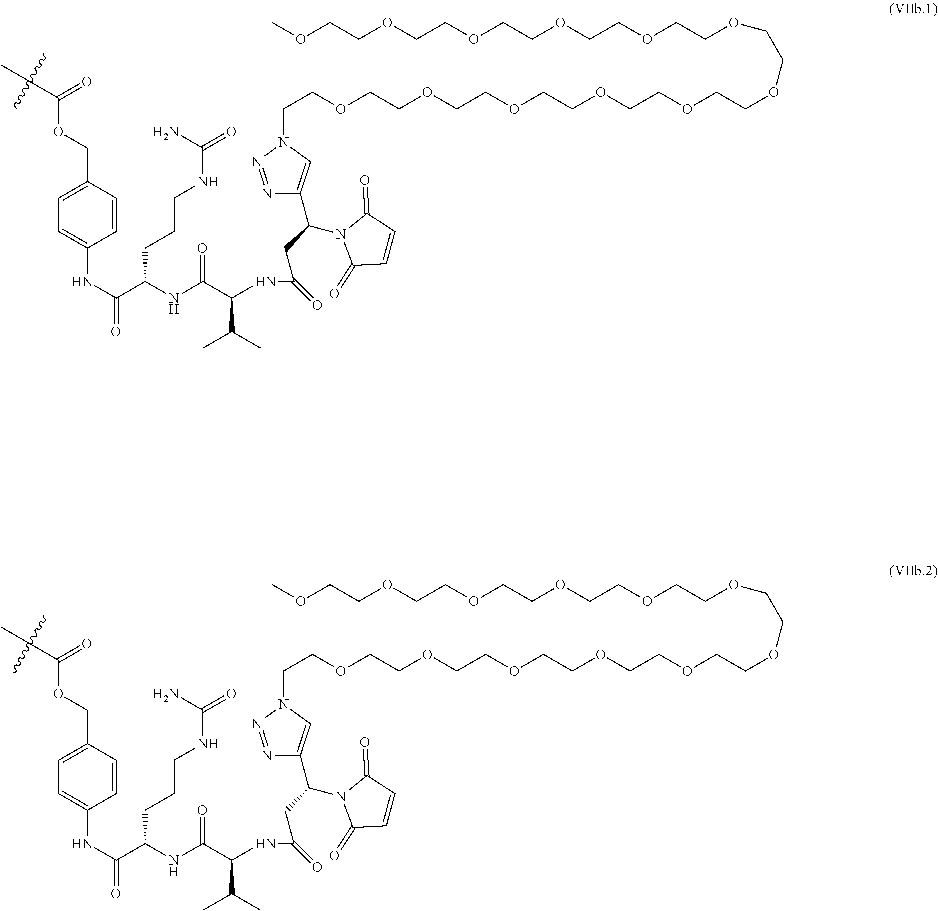 [SMILES: *C(=O)OCc1ccc(NC(=O)[C@H](CCCNC(N)=O)NC(=O)[C@@H](NC(=O)C[C@@H](c2cn(CCOCCOCCOCCOCCOCCOCCOCCOCCOCCOCCOCCOC)nn2)N2C(=O)C=CC2=O)C(C)C)cc1 ; *C(=O)OCc1ccc(NC(=O)[C@H](CCCNC(N)=O)NC(=O)[C@@H](NC(=O)C[C@H](c2cn(CCOCCOCCOCCOCCOCCOCCOCCOCCOCCOCCOCCOC)nn2)N2C(=O)C=CC2=O)C(C)C)cc1]

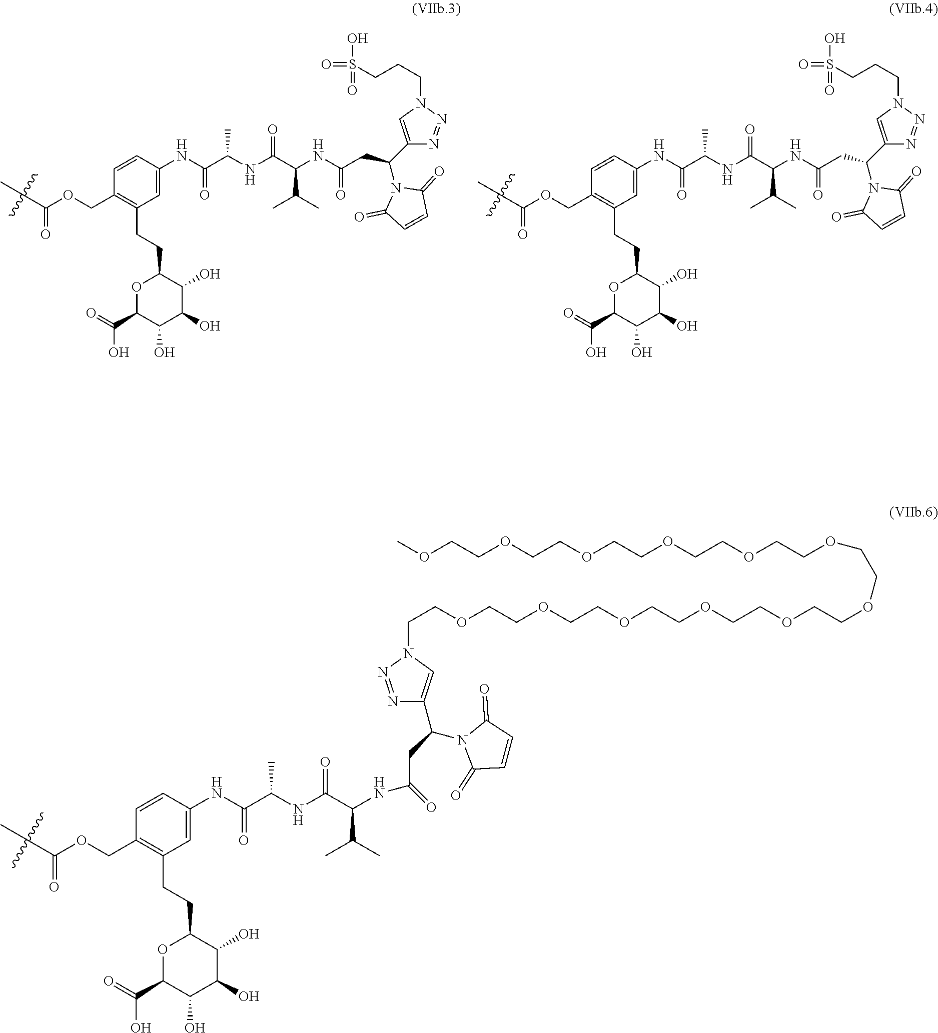 [SMILES: *C(=O)OCc1ccc(NC(=O)[C@H](C)NC(=O)[C@@H](NC(=O)C[C@@H](c2cn(CCCS(=O)(=O)O)nn2)N2C(=O)C=CC2=O)C(C)C)cc1CC[C@@H]1O[C@H](C(=O)O)[C@@H](O)[C@H](O)[C@H]1O ; *C(=O)OCc1ccc(NC(=O)[C@H](C)NC(=O)[C@@H](NC(=O)C[C@H](c2cn(CCCS(=O)(=O)O)nn2)N2C(=O)C=CC2=O)C(C)C)cc1CC[C@@H]1O[C@H](C(=O)O)[C@@H](O)[C@H](O)[C@H]1O ; *C(=O)OCc1ccc(NC(=O)[C@H](C)NC(=O)[C@@H](NC(=O)C[C@@H](c2cn(CCOCCOCCOCCOCCOCCOCCOCCOCCOCCOCCOCCOC)nn2)N2C(=O)C=CC2=O)C(C)C)cc1CC[C@@H]1O[C@H](C(=O)O)[C@@H](O)[C@H](O)[C@H]1O]

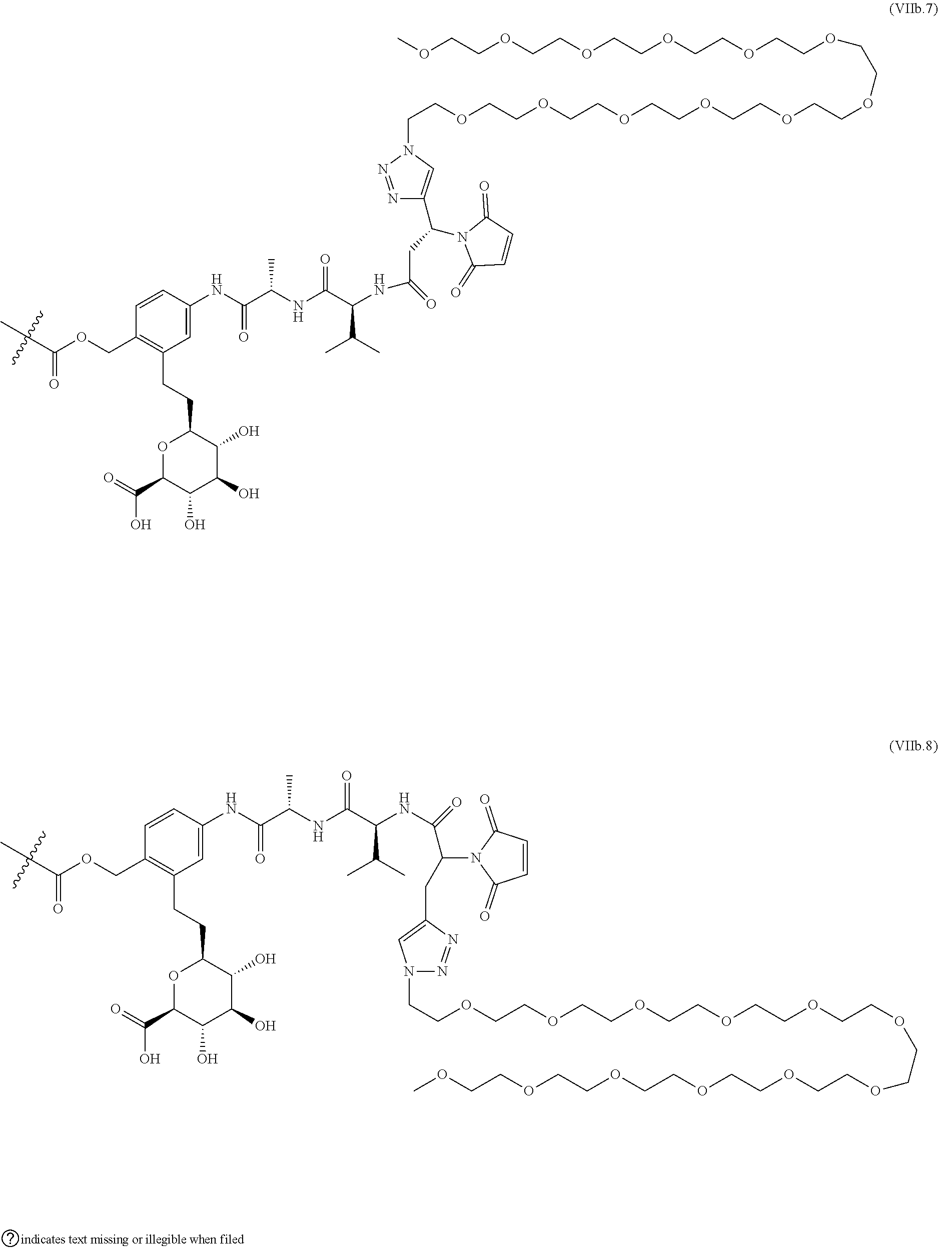 [SMILES: *C(=O)OCc1ccc(NC(=O)[C@H](C)NC(=O)[C@@H](NC(=O)C[C@H](c2cn(CCOCCOCCOCCOCCOCCOCCOCCOCCOCCOCCOCCOC)nn2)N2C(=O)C=CC2=O)C(C)C)cc1CC[C@@H]1O[C@H](C(=O)O)[C@@H](O)[C@H](O)[C@H]1O ; *C(=O)OCc1ccc(NC(=O)[C@H](C)NC(=O)[C@@H](NC(=O)C(Cc2cn(CCOCCOCCOCCOCCOCCOCCOCCOCCOCCOCCOCCOC)nn2)N2C(=O)C=CC2=O)C(C)C)cc1CC[C@@H]1O[C@H](C(=O)O)[C@@H](O)[C@H](O)[C@H]1O]

Exemplary embodiments of linkers according to structural formula (VIIc) that can be included in the construct immune-stimulatory compound conjugates described herein can include the linkers illustrated below (as illustrated, the linkers can include a group suitable for covalently linking the linker to a construct):

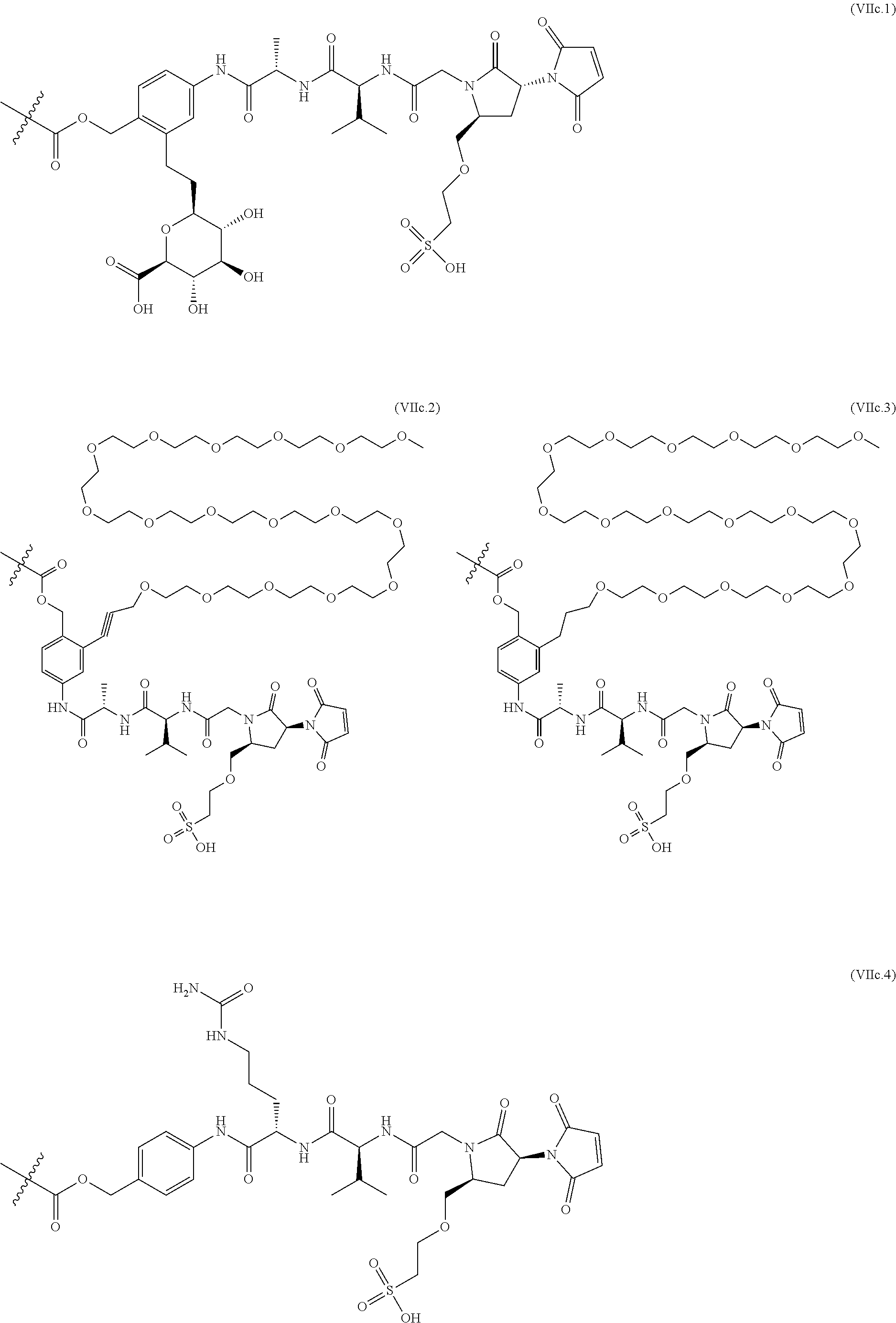

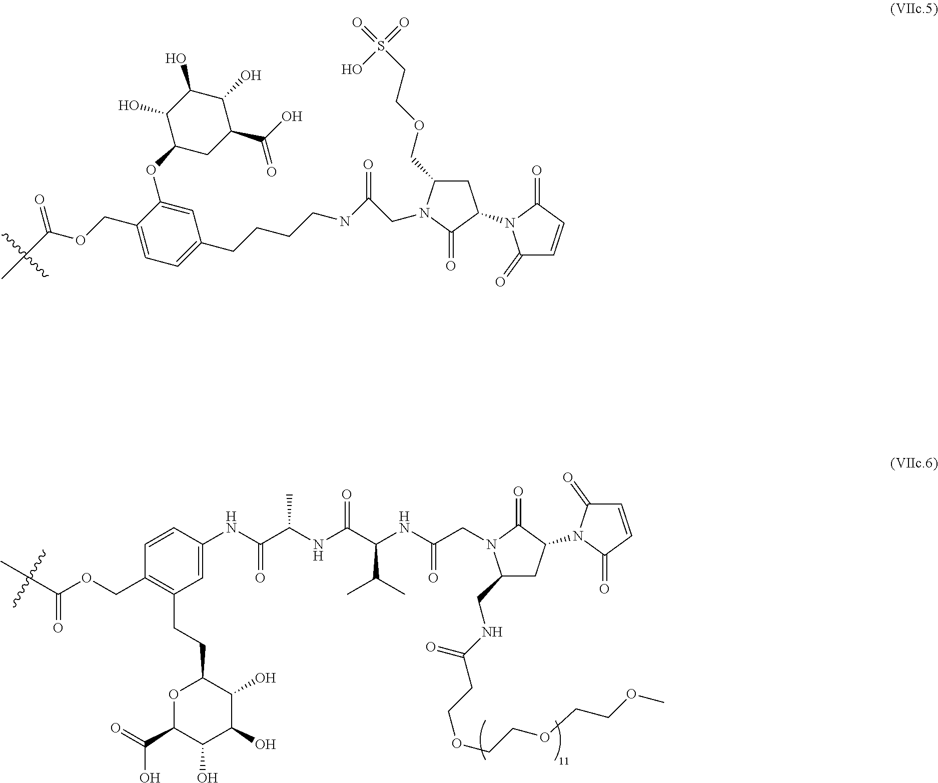

Construct-Peptide Compositions and Construct-Peptide Compositions Conjugated with Immune-Stimulatory Compounds

A construct-peptide composition can be a fusion protein. The fusion protein can be the construct fused with the peptide. The construct can be fused with the peptide through expression of a vector containing the sequence of the construct with the sequence of the peptide. The sequence of the construct and the sequence of the peptide are expressed from the same Open Reading Frame (ORF). The sequence of the construct and the sequence of the peptide can comprise a contiguous sequence. For example, the sequence of an anti-CD40 antibody and the sequence of a peptide can be expressed from a vector and translated into a contiguous amino acid sequence to create a fusion construct-peptide composition. The construct and the peptide can each retain similar functional capabilities in the fusion construct-peptide composition compared with their functional capabilities when expressed separately or when linked via a linker.

A construct-peptide composition can be in the form of a conjugate. A conjugate can be a construct directly linked to a peptide. A conjugate can be a construct indirectly linked to a peptide. A conjugate can comprise a construct linked to a peptide via a linker. Furthermore, a construct-peptide composition can be conjugated to an immune-stimulatory compound. A conjugate can comprise a construct, a peptide, an immune-stimulatory compound, and a linker. A conjugate can comprise a construct, a peptide, an inhibitor of TGFβ, and a linker. A conjugate can comprise a construct, a peptide, β-Catenin, and a linker. A conjugate can comprise a construct, a peptide, PI3K-β, and a linker. A conjugate can comprise a construct, a peptide, STAT3, and a linker. A conjugate can comprise a construct, a peptide, IL-10, and a linker. A conjugate can comprise a construct, a peptide, IDO, and a linker. A conjugate can comprise a construct, a peptide, TDO, and a linker. A conjugate can comprise a construct, a peptide, LY2109761, and a linker. A conjugate can comprise a construct, a peptide, GSK263771, and a linker. A conjugate can comprise a construct, a peptide, iCRT3, and a linker. A conjugate can comprise a construct, a peptide, iCRT5, and a linker. A conjugate can comprise a construct, a peptide, iCRT14, and a linker. A conjugate can comprise a construct, a peptide, LY2090314, and a linker. A conjugate can comprise a construct, a peptide, CGX-1321, and a linker. A conjugate can comprise a construct, a peptide, PRI-724, and a linker. A conjugate can comprise a construct, a peptide, BC21, and a linker. A conjugate can comprise a construct, a peptide, ZINCO2092166, and a linker. A conjugate can comprise a construct, a peptide, LGK974, and a linker. A conjugate can comprise a construct, a peptide, IWP2, and a linker. A conjugate can comprise a construct, a peptide, LY3022859, and a linker. A conjugate can comprise a construct, a peptide, LY364947, and a linker. A conjugate can comprise a construct, a peptide, SB431542, and a linker. A conjugate can comprise a construct, a peptide, AZD8186, and a linker. A conjugate can comprise a construct, a peptide, SD-208, and a linker. A conjugate can comprise a construct, a peptide, indoximod (NLG8189), and a linker. A conjugate can comprise a construct, a peptide, F001287, and a linker. A conjugate can comprise a construct, a peptide, GDC-0919, and a linker. A conjugate can comprise a construct, a peptide, epacadostat (INCB024360), and a linker. A conjugate can comprise a construct, a peptide, RG70099, and a linker. A conjugate can comprise a construct, a peptide, 1-methyl-L-tryptophan, and a linker. A conjugate can comprise a construct, a peptide, methylthiohydantoin tryptophan, and a linker. A conjugate can comprise a construct, a peptide, brassinin, and a linker. A conjugate can comprise a construct, a peptide, annulin B, and a linker. A conjugate can comprise a construct, a peptide, exiguamine A, and a linker. A conjugate can comprise a construct, a peptide, PIM, and a linker. A conjugate can comprise a construct, a peptide, LM10, and a linker. A conjugate can comprise a construct, a peptide, INCB023843, and a linker. A conjugate can comprise a construct, a peptide, 8-substituted imidazo[1,5-a]pyridine, and a linker. A conjugate can comprise a construct, a peptide, a pattern recognition receptor (PRR) agonist, and a linker. A conjugate can comprise a construct, a peptide, a pattern-associated molecular pattern (PAMP) molecule, and a linker. A conjugate can comprise a construct, a peptide, a damage-associated molecular pattern (DAMP) molecule, and a linker.

A conjugate can comprise a construct, a peptide, a STING agonist, and a linker. A conjugate can comprise a construct, a peptide, a toll-like receptor agonist molecule, and a linker. A conjugate can comprise a construct, a peptide, imiquimod, and a linker. A conjugate can comprise a construct, a peptide, S-27609, and a linker. A conjugate can comprise a construct, a peptide, CL307, and a linker. A conjugate can comprise a construct, a peptide, resiquimod, and a linker. A conjugate can comprise a construct, a peptide, gardiquimod, and a linker. A conjugate can comprise a construct, a peptide, UC-IV150, and a linker. A conjugate can comprise a construct, a peptide, motolimod, and a linker. A conjugate can comprise a construct, a peptide, VTX-1463, and a linker. A conjugate can comprise a construct, a peptide, GS-9620, and a linker. A conjugate can comprise a construct, a peptide, GSK2245035, and a linker. A conjugate can comprise a construct, a peptide, TMX-101, and a linker. A conjugate can comprise a construct, a peptide, TMX-201, and a linker. A conjugate can comprise a construct, a peptide, TMX-202, and a linker. A conjugate can comprise a construct, a peptide, isatoribine, and a linker. A conjugate can comprise a construct, a peptide, AZD8848, and a linker. A conjugate can comprise a construct, a peptide, MEDI9197, and a linker. A conjugate can comprise a construct, a peptide, 3M-051, and a linker. A conjugate can comprise a construct, a peptide, 3M-852, and a linker. A conjugate can comprise a construct, a peptide, 3M-052, and a linker. A conjugate can comprise a construct, a peptide, 3M-854A, and a linker. A conjugate can comprise a construct, a peptide, S-34240, and a linker. A conjugate can comprise a construct, a peptide, CL663, and a linker. A conjugate can comprise a construct, a peptide, KIN1148, and a linker. A conjugate can comprise a construct, a peptide, SB-9200, and a linker. A conjugate can comprise a construct, a peptide, KIN-100, and a linker. A conjugate can comprise a construct, a peptide, ADU-S100, and a linker. A conjugate can comprise a construct, a peptide, KU34B, and a linker. A conjugate can comprise a construct, a peptide, SB9200, and a linker. A conjugate can comprise a construct, a peptide, SB11285, and a linker. A conjugate can comprise a construct, a peptide, 8-substituted 2-amino-3H-benzo[b]azepine-4-carbozamide, and a linker.

The linker can be a linker as described herein. A linker can be cleavable, non-cleavable, hydrophilic, or hydrophobic. A cleavable linker can be sensitive to enzymes. A cleavable linker can be cleaved by enzymes such as proteases. A cleavable linker can be a valine-citrulline or a valine-alanine linker. A valine-citrulline or valine-alanine linker can contain a pentafluorophenyl group. A valine-citrulline or valine-alanine linker can contain a succimide group. A valine-citrulline or valine-alanine linker can contain a PABA group. A valine-citrulline or valine-alanine linker can contain a PABA group and a pentafluorophenyl group. A valine-citrulline or valine-alanine linker can contain a PABA group and a succinimide group. A non-cleavable linker can be protease insensitive. A non-cleavable linker can be maleimidocaproyl linker. A maleimidocaproyl linker can comprise N-maleimidomethylcyclohexane-1-carboxylate. A maleimidocaproyl linker can contain a succinimide group. A maleimidocaproyl linker can contain pentafluorophenyl group. A linker can be a combination of a maleimidocaproyl group and one or more polyethylene glycol molecules. A linker can be a maleimide-PEG4 linker. A linker can be a combination of a maleimidocaproyl linker containing a succinimide group and one or more polyethylene glycol molecules. A linker can be a combination of a maleimidocaproyl linker containing a pentafluorophenyl group and one or more polyethylene glycol molecules. A linker can contain maleimides linked to polyethylene glycol molecules in which the polyethylene glycol can allow for more linker flexibility or can be used lengthen the linker. A linker can be a (maleimidocaproyl)-(valine-citrulline)-(para-aminobenzyloxycarbonly)-(NH₂) linker. A linker can be a THIOMAB linker. A THIOMAB linker can be a (maleimidocaproyl)-(valine-citrulline)-(para-aminobenzyloxycarbonly)-(NH₂) linker. A linker can also be an alkylene, alkenylene, alkynylene, polyether, polyester, polyamide, polyamino acids, polypeptides, cleavable peptides, or aminobenzylcarbamates. A linker can contain a maleimide at one end and an N-hydroxysuccinimidyl ester at the other end. A linker can contain a lysine with an N-terminal amine acetylated, and a valine-citrulline cleavage site. A linker can be a link created by a microbial transglutaminase, wherein the link is created between an amine-containing moiety and a moiety engineered to contain glutamine as a result of the enzyme catalyzing a bond formation between the acyl group of a glutamine side chain and the primary amine of a lysine chain. A linker can contain a reactive primary amine. A linker can be a Sortase A linker. A Sortase A linker can be created by a Sortase A enzyme fusing an LPXTG recognition motif (SEQ ID NO: 21) to an N-terminal GGG motif to regenerate a native amide bond. The linker created can therefore link a moiety attached to the LPXTG recognition motif (SEQ ID NO: 21) with a moiety attached to the N-terminal GGG motif. A linker can be a link created between an unnatural amino acid on one moiety reacting with oxime bond that was formed by modifying a ketone group with an alkoxyamine on another moiety. A moiety can be a construct. A moiety can be a peptide. A moiety can be an antibody. A moiety can be an immune-stimulatory compound.

The construct-peptide composition can comprise any construct as described herein. The construct can be a single chain TCR. The construct can contain the antigen binding domain of a single chain TCR. The construct can be di-sulfide linked TCRs. The construct can be an anti-tumor antigen construct. The construct can be an anti-tumor antigen antibody. An antigen recognized by the construct can be CD5, CD19, CD20, CD25, CD37, CD30, CD33, CD45, CAMPATH-1, BCMA, CS-1, PD-L1, B7-H3, B7-DC, HLD-DR, carcinoembryonic antigen (CEA), TAG-72, EpCAM, MUC1, folate-binding protein, A33, G250, prostate-specific membrane antigen (PSMA), ferritin, GD2, GD3, GM2, Le^{y}, CA-125, CA19-9, epidermal growth factor, p185HER2, IL-2 receptor, fibroblast activation protein (FAP), tenascin, a metalloproteinase, endosialin, vascular endothelial growth factor, avB3, WT1, LMP2, HPV E6, HPV E7, EGFRvIII (de2-7 EGFR,), Her-2/neu, idiotype, MAGE A3, p53 nonmutant, NY-ESO-1, MelanA/MART1, Ras mutant, gp100, p53 mutant, PR1, bcr-abl, tyrosinase, survivin, PSA, hTERT, a Sarcoma translocation breakpoint fusion protein, EphA2, PAP, ML-IAP, AFP, ERG, NA17, PAX3, ALK, androgen receptor, cyclin B1, polysialic acid, MYCN, RhoC, TRP-2, fucosyl GM1, mesothelin (MSLN), PSCA, MAGE A1, sLe(animal), CYP1B1, PLAV1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, B7H3, Legumain, Tie 3, Page4, VEGFR2, MAD-CT-1, PDGFR-B, MAD-CT-2, ROR2, TRAIL1, MUC16, MAGE A4, MAGE C2, GAGE, EGFR, CMET, HER3, MUC1, MUC15, CA6, NAPI2B, TROP2, CLDN18.2, RON, LY6E, FRA, DLL3, PTK7, LIV1, ROR1, or Fos-related antigen 1. The construct can recognize an antigen that can be expressed on a cell. The construct can recognize an antigen that can be expressed by a cell. The construct can recognize an antigen that can be presented by a Major Histocompatibility Complex (MHC). An MHC can be a class I MHC. An MHC can be a class II MHC. The construct can recognize an antigen that can stimulate activity of a cell. The construct can recognize an antigen that can stimulate an immune response. The construct can recognize an antigen that can reduce an immune response. The construct can recognize an antigen that can reduce activity of a cell. The construct can recognize an antigen that can be expressed on an immune cell. The construct can recognize an antigen that can be expressed by an immune cell. The construct can recognize an antigen on a cell wherein the antigen can be involved in stimulating activity of a cell. The construct can recognize an antigen on an immune cell that can be involved in the costimulation of an immune cell. The construct can recognize an antigen on an immune cell that can be involved in the costimulation of an immune cell during an immune response. The construct can recognize a receptor. The construct can recognize a receptor on a cell. The construct can recognize a receptor ligand. The construct can recognize a receptor on a cell wherein the receptor can be involved in stimulating activity of a cell. The construct can recognize a receptor on an immune cell. The construct can recognize a receptor on an immune cell that can be involved in stimulating activity of an immune cell. The construct can recognize a receptor on an immune cell that can be involved in the costimulation of an immune cell. The construct can recognize a receptor on an immune cell that can be involved in the costimulation of an immune cell during an immune response. The construct can recognize an antigen that can be expressed on an immune cell and that can stimulate activity of an immune cell. The construct can recognize an antigen that can be expressed on an immune that can reduce activity of an immune cell. The construct can be an anti-CD40 antibody. The construct can comprise a light chain of an SBT-040 antibody. The construct can comprise an SBT-040-G1WT heavy chain. The construct can comprise an SBT-040-G1VLPLL heavy chain. The construct can comprise an SBT-040-G1DE heavy chain. The construct can comprise an SBT-040-G1AAA heavy chain. The construct can comprise an SBT-040-CDR sequence. The construct can be capable of recognizing a single antigen. The construct can be capable of recognizing two or more antigens. The construct can be capable of recognizing a single antigen. The construct can be capable of recognizing two or more antigens. The K_{d} for binding of an antigen-binding domain of a construct-peptide immune-stimulatory compound conjugate to an antigen in the presence of an immune-stimulatory compound can be about 2 times, about 3 times, about 4 times, about 5 times, about 6 times, about 7 times, about 8 times, about 9 times, about 10 times, about 15 times, about 20 times, about 25 times, about 30 times, about 35 times, about 40 times, about 45 times, about 50 times, about 60 times, about 70 times, about 80 times, about 90 times, about 100 times, about 110 times, or about 120 times greater than the K_{d} for binding of the antigen binding domain to the antigen of a construct in the absence of the immune-stimulatory compound. The K_{d} for binding of an antigen-binding domain of a construct-peptide immune-stimulatory compound conjugate to an antigen in the presence of the immune-stimulatory compound can be less than 10 nM. The K_{d} for binding of an antigen-binding domain of a construct-peptide immune-stimulatory compound conjugate to an antigen in the presence of the immune-stimulatory compound can be less than 100 nM, less than 50 nM, less than 20 nM, less than 5 nM, less than 1 nM, or less than 0.1 nM.

The any construct described herein can further comprise a peptide, thereby forming the construct-peptide composition. The peptide can contain, for example, sequence identity or homology to a sequence found in cancers. The sequence identity or homology to the cancer sequence can be, for example, about 10%, about 20%, about 30%, about 40%, about 50%, about 55%, about 60%, about 65%, about 70%, about 75%, about 80%, about 85%, about 90%, about 95%, or about 99%. The peptide can have, for example, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acid residues. The peptide can contain a non-synonymous or missense mutation at any point. In some embodiments, the peptide contains a non-synonymous mutation at a central amino acid residue in the peptide. The peptide can be conjugated at, for example, the Fc region of the antibody.

The peptide can be selected in order to be antigenic or immunogenic. The peptide can contain comprise a portion of a splice variant or a fragment of an endogenous protein that can be processed into a peptide and presented on the surface of a tumor cell. A peptide can contain a neo-antigen (neoAg), which can be portion of an endogenous mutated cancer protein that can be processed into a peptide and presented on the surface of a tumor cell. Once the neoAg is presented on the surface of the tumor cell, the neoAg can be recognized by the immune system as a foreign antigen. A neoAg can be identified by in silico methods. For example, the mutanome (mutational spectrum of individual tumors) can be used to identify nonsynonymous mutations. From the nonsynonymous mutations, neo-epitopes can be identified on a patient-by-patient basis. Neural network algorithms can be applied to predict high affinity neo-epitopes derived from mutated genes that can bind to a patient's own MHC molecules. These predicted peptides can be then synthesized and can be used to test a patient's immunity. This approach can used for long peptides or mRNA encoding for the mutations.

Alternatively, the MHC ligandome of a tumor cell can be analyzed, in which peptides from the MHC molecules derived from a tumor tissue of a patient can be eluted followed by reverse phase HPLC fractionation and mass spectrometry for identification.

Strategies to identify the neo-epitopes from the short, MHC class I or MHC class II restricted peptides can include, for example, using peptide-MHC multimer complexes and functional assays. Testing of a patient's immunity using the in silico identified peptides can be achieved by using the patient's own T cells. The T cell-based functional assays can use either in silico-predicted short peptides exclusively for CD8⁺ T cells, or long peptides and mRNA to analyze both CD4⁺ and CD8⁺ T cells. The short peptides, long peptides, and mRNA can be used to pulse and transduce antigen-presenting cells, and the antigen-presenting cells and the patient's cells are co-cultured to stimulate the production of neo-antigen-specific CD4⁺ or CD8⁺ T cells.

The PRR agonist can be a toll-like receptor agonist. The toll-like receptor agonist can be a TLR1 agonist, a TLR2 agonist, a TLR3 agonist, a TLR4 agonist, a TLR5 agonist, a TLR6 agonist, a TLR7 agonist, a TLR8 agonist, a TLR9 agonist, a TLR10 agonist, a TLR11 agonist, a TLR12 agonist or a TLR13 agonist. The toll-like receptor agonist can activate two or more TLRs. The PAMP molecule can be a RIG-I agonist.

A construct-peptide composition or conjugate can comprise of one or more peptides. In some cases, a construct-peptide composition or conjugate comprises one type of peptide, generating a monoclonal response. Alternatively, a construct-peptide composition or conjugate can comprise more than one type of peptide generating a polyclonal response. One or more peptides can be in the form of polymers. In some embodiments, the peptide is a polymer of peptides. In some embodiments, the polymer of peptides comprises at least two different peptides. In some embodiments, the peptide is immunogenic. Polymeric peptides can be processed together or they can be processed separately in the cell.

A conjugate can be formed by a linker that can connect a construct to a PRR agonist. A conjugate can be formed by a linker that can connect a construct to a PAMP molecule. A conjugate can be formed by a linker that can connect a construct and a DAMP molecule. A conjugate can be formed by a linker that can connect a construct to a PRR agonist, and a linker that can connect a construct and a targeting peptide. A conjugate can be formed by a linker that can connect a construct to a PAMP molecule, and a linker that can connect a construct and a peptide. A conjugate can be formed by a linker that can connect a construct and a DAMP molecule, and a linker that can connect a construct and a peptide.

A linker can be connected to a construct by a direct linkage between the construct and the linker. A linker can be connected to an anti-CD40 antibody construct by a direct linkage between the anti-CD40 antibody construct and the linker. A linker can be connected to an anti-CD40 antibody by a direct linkage between the anti-CD40 antibody and the linker. A linker can be connected to an anti-tumor antigen antibody construct by a direct linkage between the anti-tumor antigen antibody construct and the linker. A linker can be connected to an anti-tumor antigen antibody by a direct linkage between the anti-tumor antigen antibody and the linker. A direct linkage can be a covalent bond. For example, a linker can be attached to a terminus of an amino acid sequence of a construct, or could be attached to a side chain modification to the construct, such as the side chain of a lysine, serine, threonine, cysteine, tyrosine, aspartic acid, a non-natural amino acid residue, or glutamic acid residue.

An attachment can be via any of a number of bonds, for example but not limited to, an amide bond, an ester bond, an ether bond, a carbon-nitrogen bond, a carbon-carbon single double or triple bond, a disulfide bond, or a thioether bond. A linker can have at least one functional group, which can be linked to the antibody. Non-limiting examples of the functional groups can include those that form an amide bond, an ester bond, an ether bond, a carbonate bond, a carbamate bond, or a thioether bond, such functional groups can be, for example, amino groups; carboxyl groups; aldehyde groups; azide groups; alkyne and alkene groups; ketones; carbonates; carbonyl functionalities bonded to leaving groups such as cyano and succinimidyl and hydroxyl groups.

In some embodiments, the linker is not attached to an amino acid residue of the Fc domain of the antibody construct selected from a group consisting of: 221, 222, 224, 227, 228, 230, 231, 223, 233, 234, 235, 236, 237, 238, 239, 240, 241, 243, 244, 245, 246, 247, 249, 250, 258, 262, 263, 264, 265, 266, 267, 268, 269, 270, 271, 272, 273, 274, 275, 276, 278, 280, 281, 283, 285, 286, 288, 290, 291, 292, 293, 294, 295, 296, 297, 298, 299, 300, 302, 305, 313, 317, 318, 320, 322, 323, 324, 325, 326, 327, 328, 329, 330, 331, 332, 333, 334, 335 336, 396, or 428, wherein numbering of amino acid residues in said Fc domain is according to the EU index as in Kabat. In some embodiments, the linker is not attached to an amino acid residue of the Fc domain of the antibody construct selected from a group consisting of: 221, 224, 227, 230, 231, 232, 234, 235, 236, 237, 239, 240, 243, 244, 245, 247, 249, 258, 262, 263, 264, 265, 266, 267, 268, 269, 270, 271, 272, 273, 275, 278, 280, 281, 283, 285, 286, 291, 292, 293, 294, 295, 296, 297, 298, 299, 300, 305, 313, 323, 324, 325, 327, 328, 329, 330, 331, 332, 333, 335, 336, 396, or 428, wherein numbering of amino acid residues in said Fc domain is according to the EU index as in Kabat. In some embodiments, the linker is covalently bound to a residue of the antibody construct selected from the group consisting of a lysine residue, cysteine residue, and a glutamine residue, or is covalently bound to said antibody construct using a Sortase A linker.

A linker can be connected to a construct at a hinge cysteine. A linker can be connected to a construct at a light chain constant domain lysine. A linker can be connected to a construct at an engineered cysteine in the light chain. A linker can be connected to a construct at an engineered light chain glutamine. A linker can be connected to a construct at an unnatural amino acid engineered into the light chain. A linker can be connected to construct at an unnatural amino acid engineered into the heavy chain. Amino acids can be engineered into an amino acid sequence of a composition as described herein, for example, a linker of a conjugate. Engineered amino acids can be added to a sequence of existing amino acids. Engineered amino acids can be substituted for one or more existing amino acids of a sequence of amino acids. A linker can be conjugated to construct via a sulfhydryl group. A linker can be conjugated to a construct via a primary amine. A linker can be a link created between an unnatural amino acid on a construct reacting with oxime bond that was formed by modifying a ketone group with an alkoxyamine on an immune-stimulatory compound. When a linker is connected to a construct at the sites described herein, an Fc domain of the construct can bind to Fc receptors. When a linker is connected to a construct at the sites described herein, the antigen binding domain of the construct can bind its antigen. When a linker is connected to a construct at the sites described herein, a peptide of said construct can bind its antigen.

A linker can connect a construct to a peptide via THIOMAB linker. A linker can connect a construct to a peptide via Sortase A linker. A Sortase A linker can be created by a Sortase A enzyme fusing an LPXTG recognition motif (SEQ ID NO: 21) to an N-terminal GGG motif to regenerate a native amide bond. The linker created can therefore link a construct attached the LPXTG recognition motif (SEQ ID NO: 21) with a peptide attached to the N-terminal GGG motif. A peptide can be connected to a linker by a direct linkage. A direct linkage can be a covalent bond. For example, a linker can be attached to a terminus of an amino acid sequence of a peptide, or could be attached to a side chain modification to the targeting binding domain, such as the side chain of a lysine, serine, threonine, cysteine residue, an engineered cysteine residue, a lysine residue, a serine residue, a threonine residue, a tyrosine residue, an aspartic acid, residue, a glutamic acid residue, a glutamine residue, an engineered glutamine residue, a selenocysteine residue, or a non-natural amino acid. Non-natural amino acids can include para-azidomethyl-1-phenylalanine (pAMF). An attachment can also be at a residue containing an oxime bond that was formed by modifying a ketone group with an alkoxyamine on another moiety, and a reactive primary amine, such as a reactive primary amine at a C-terminal end of a protein or peptide. An attachment can be via any of a number of bonds, for example but not limited to, an amide bond, an ester bond, an ether bond, a carbon-nitrogen bond, a carbon-carbon single double or triple bond, a disulfide bond, or a thioether bond. A linker can have at least one functional group, which can be linked to the peptide. Non-limiting examples of the functional groups can include those which form an amide bond, an ester bond, an ether bond, a carbonate bond, a carbamate bond, or a thioether bond, such functional groups can be, for example, amino groups; carboxyl groups; aldehyde groups; azide groups; alkyne and alkene groups; ketones; carbonates; carbonyl functionalities bonded to leaving groups such as cyano and succinimidyl and hydroxyl groups. Amino acids can be engineered into an amino acid sequence of the peptide. Engineered amino acids can be added to a sequence of existing amino acids. Engineered amino acids can be substituted for one or more existing amino acids of a sequence of amino acids. A linker can be conjugated to a peptide via a sulfhydryl group. A linker can be conjugated to a peptide via a primary amine. A peptide can be conjugated to the C-terminal of an Fc domain of a construct.

An antibody with engineered reactive cysteine residues (THIOMAB) can be used to link an immune-stimulatory compound to the antibody. A linker can connect a construct to an immune-stimulatory compound via THIOMAB linker. A linker can connect a construct to an immune-stimulatory compound via Sortase A linker. A Sortase A linker can be created by a Sortase A enzyme fusing an LPXTG recognition motif (SEQ ID NO: 21) to an N-terminal GGG motif to regenerate a native amide bond. The linker created can therefore link a construct attached the LPXTG recognition motif (SEQ ID NO: 21) with an immune-stimulatory compound attached to the N-terminal GGG motif. A linker can be a link created between an unnatural amino acid on a construct reacting with oxime bond that was formed by modifying a ketone group with an alkoxyamine on an immune-stimulatory compound. The immune-stimulatory compound can comprise one or more rings selected from carbocyclic and heterocyclic rings. The immune-stimulatory compound can be covalently bound to a linker by a bond to an exocyclic carbon or nitrogen atom on said immune-stimulatory compound. A linker can be conjugated to an immune-stimulatory compound via an exocyclic nitrogen or carbon atom of an immune-stimulatory compound. A linker can be connected to a STING agonist, for example:

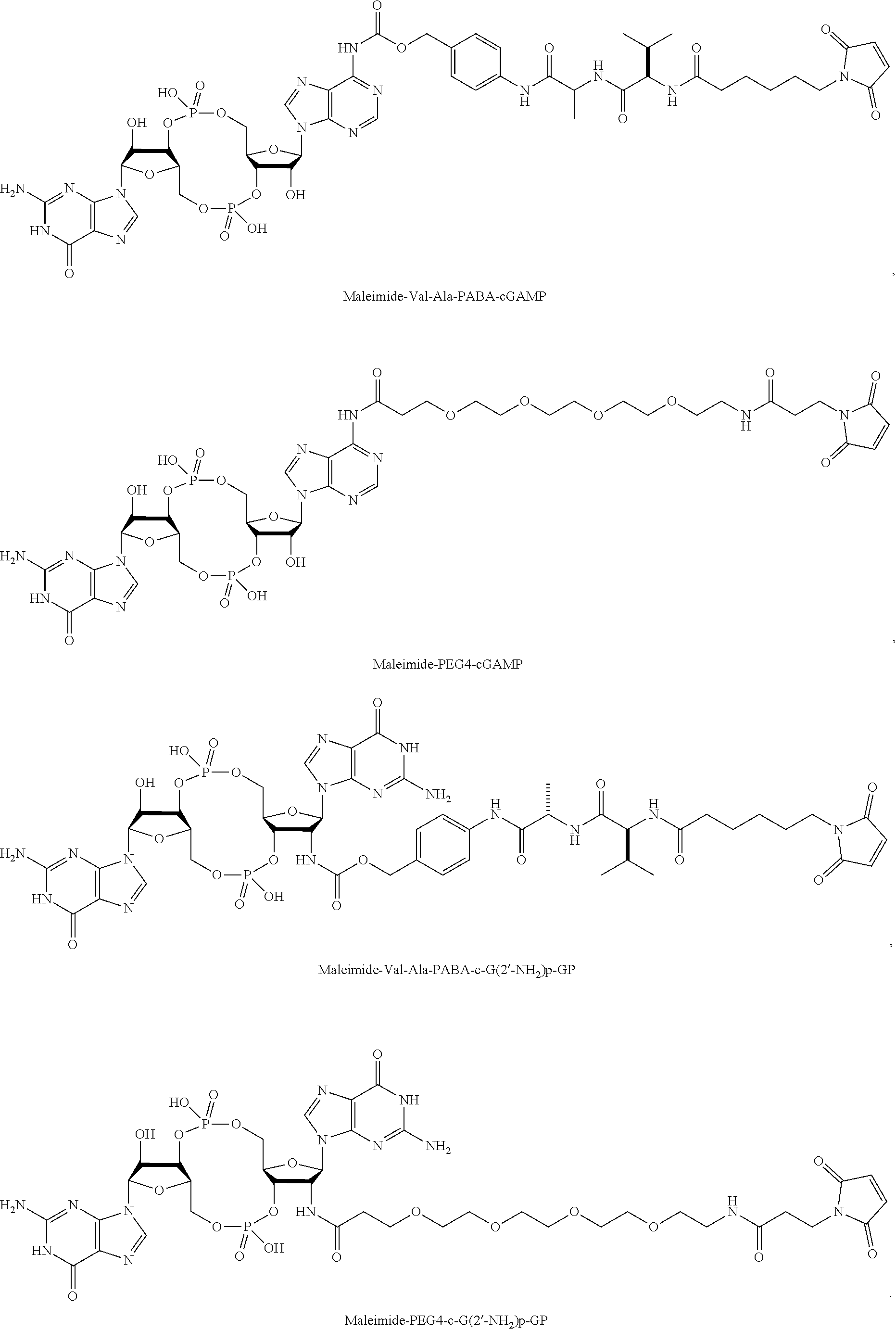

A linker agonist complex can dissociate under physiological conditions to yield an active agonist.

A linker can be connected to an immune-stimulatory compound by a direct linkage between the immune-stimulatory compound and the linker. A linker can be connected to an immune-stimulatory compound by a direct linkage between the immune-stimulatory compound and the linker.

A linker can be connected to a PRR agonist by a direct linkage between the PRR agonist and the linker. A linker can be connected to a PAMP molecule by a direct linkage between the PAMP molecule and the linker. A linker can be connected to a toll-like receptor agonist by a direct linkage between the toll-like receptor agonist and the linker. A linker can be connected to a DAMP molecule by a direct linkage between the DAMP molecule and the linker.

Examples of toll-like receptor agonists connected to a linker in a manner able to release an active toll-like receptor agonist under physiologic condition include:

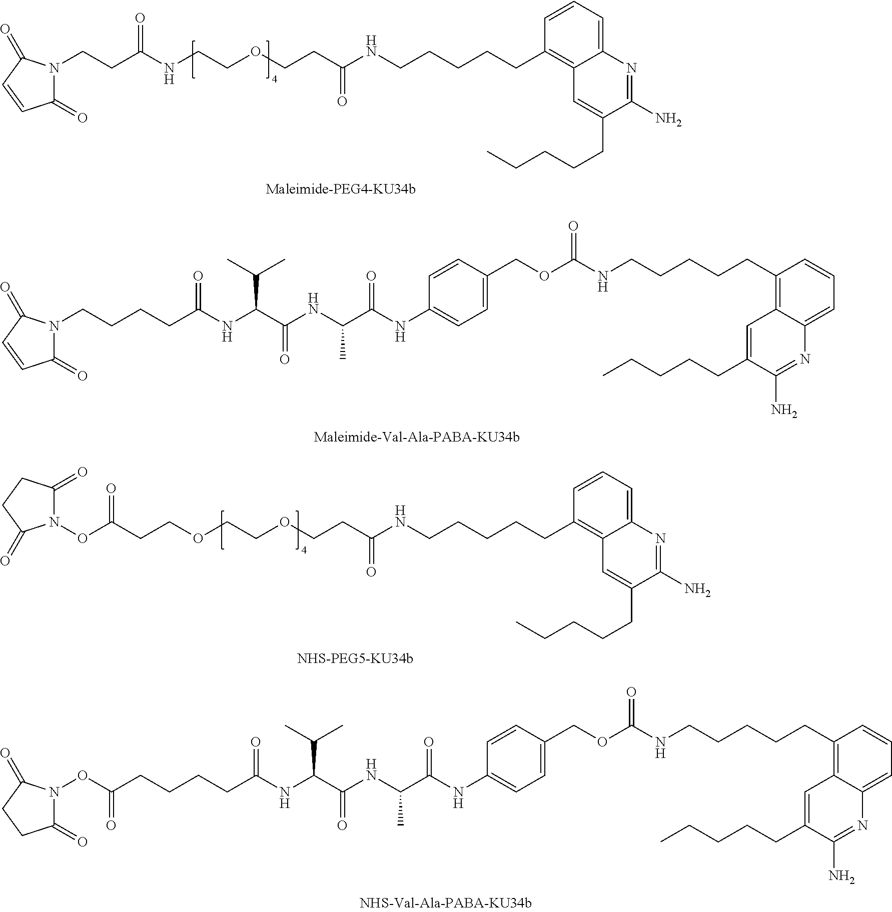

Examples of RIG-I agonists connected to a linker in a manner able to release an active toll-like receptor agonist under physiologic conditions include:

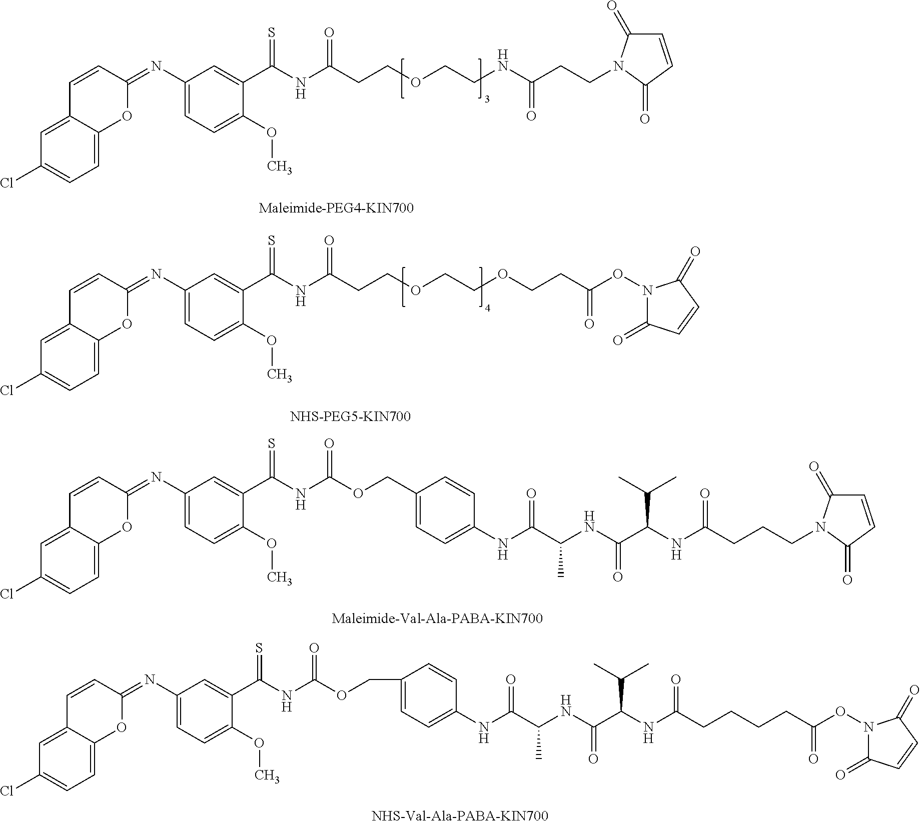

A linker can be connected to an immune-stimulatory compound by a direct linkage between the immune-stimulatory compound and the linker. A linker can be connected to a PAMP molecule by a direct linkage between the PAMP molecule and the linker. A direct linkage can be a covalent bond. A linker can be connected to a DAMP molecule by a direct linkage between the DAMP molecule and the linker. For example, a linker can be attached to a terminus of an amino acid sequence of an antibody, or could be attached to a side chain modification to the antibody, such as the side chain of a lysine, serine, threonine, cysteine, tyrosine, aspartic acid, a non-natural amino acid residue, or glutamic acid residue. An attachment can be via any of a number of bonds, for example but not limited to, an amide bond, an ester bond, an ether bond, a carbon-nitrogen bond, a carbon-carbon single double or triple bond, a disulfide bond, or a thioether bond. A linker can have at least one functional group, which can be linked to the construct. Non-limiting examples of the functional groups can include those that form an amide bond, an ester bond, an ether bond, a carbonate bond, a carbamate bond, or a thioether bond, such functional groups can be, for example, amino groups; carboxyl groups; aldehyde groups; azide groups; alkyne and alkene groups; ketones; carbonates; carbonyl functionalities bonded to leaving groups such as cyano and succinimidyl and hydroxyl groups.

The construct-peptide composition can have an Fc domain that can bind to an FcR when linked to an immune-stimulatory compound. The construct can have an Fc domain that can bind to an FcR to initiate FcR-mediated signaling when linked or fused to the peptide. The construct-peptide composition can have an Fc domain that can bind to an FcR to initiate FcR-mediated signaling when linked or fused to the peptide and when linked to an immune stimulatory compound. The construct-peptide composition can bind to its antigen when linked or fused with the peptide. The construct-peptide composition can bind to its antigen when linked or fused to the peptide and when linked to an immune-stimulatory compound. The construct-peptide composition can bind to its antigen when linked to an immune-stimulatory compound and the Fc domain of the construct can bind to an FcR when linked to an immune-stimulatory compound. The construct-peptide composition can bind to its antigen when linked to an immune-stimulatory compound and the Fc domain of the construct can bind to an FcR when linked to an immune-stimulatory compound. The construct-peptide composition can bind to its antigen when linked to an immune-stimulatory compound and the Fc domain of the antibody can bind to an FcR to initiate FcR-mediated signaling when linked to an immune stimulatory compound. The construct-peptide composition can bind to its antigen when linked to an immune-stimulatory compound and the Fc domain of the antibody can bind to an FcR to initiate FcR-mediated signaling when linked to an immune stimulatory compound.

A construct-peptide composition can comprise a peptide comprising a V157F, G154V, R176G, P278A, Y220C, G245S, R248Q, or R273H mutation in p53. A construct-peptide composition can comprise a peptide comprising a G466V or V600E mutation of BRAF. A construct-peptide composition can comprise a E79Q mutation in NFE2L2. A construct-peptide composition can comprise a G719A mutation in EGFR. A construct-peptide composition can comprise a G12D, G12V, G12C, or G12R mutation in KRAS. A construct-peptide composition can comprise a G12V, Q61L, or Q61R mutation in HRAS. A construct-peptide composition can comprise a G12D, G12S, G13D, Q61K, or Q61R mutation in NRAS. A construct-peptide composition can comprise a Q311E mutation in C3orf59. A construct-peptide composition can comprise a E805G mutation in ERBB2IP. A construct-peptide composition can comprise A359D mutation in NUP98. A construct-peptide composition can comprise a E426K mutation in GPD2. A construct-peptide composition can comprise a E1179K mutation in PLEC. A construct-peptide composition can comprise a P274S mutation in XPO7. A construct-peptide composition can comprise a Q418K mutation in AKAP2. A construct-peptide composition can comprise a F67V mutation in CASP8. A construct-peptide composition can comprise a S1002I mutation in ITGB4. A construct-peptide composition can comprise a P293L mutation in TUBGCP2. A construct-peptide composition can comprise a N1702S mutation in RNF213. A construct-peptide composition can comprise a R653H mutation in SKIV2L. A construct-peptide composition can comprise a A48T mutation in H3F3B. A construct-peptide composition can comprise a R243Q mutation in API5. A construct-peptide composition can comprise a E572K mutation in RNF10. A construct-peptide composition can comprise a G566E mutation in PHLPP1. A construct-peptide composition can comprise a R6H mutation in 2FYVE27. A construct peptide can comprise a mutated peptide disclosed in public neoAg databases such as the TANTIGEN: Tumor T-cell Antigen Database.

In some embodiments, the peptide comprises a mutation selected from the group consisting of: a V157F, G154V, R176G, P278A, Y220C, G245S, R248Q, or R273H mutation in p53; a G466V or V600E mutation in BRAF; a E79Q mutation in NFE2L2; a G719A mutation in EGFR; a G12D, G12V, G12C, or G12R mutation in KRAS; a G12V, Q61L, or Q61R mutation in HRAS; a G12D, G12S, G13D, Q61K, or Q61R mutation in NRAS; a Q311E mutation in C3orf59; a E805G mutation in ERBB2IP; a A359D mutation in NUP98; a E426K mutation in GPD2; a E1179K mutation in PLEC; a P274S mutation in XPO7; a Q418K mutation in AKAP2; a F67V mutation in CASP8; a S1002I mutation in ITGB4; a P293L mutation in TUBGCP2; a N1702S mutation in RNF213; a R653H mutation in SKIV2L; a A48T mutation in H3F3B; a R243Q mutation in API5; a E572K mutation in RNF10; a G566E mutation in PHLPP1; and a R6H mutation in 2FYVE27.

In a construct-peptide composition, a peptide can be linked or fused to the construct in such a way that an antigen-binding domain in the construct can still bind to an antigen and an Fc domain of the construct can still bind to an FcR. In a construct-peptide composition, a construct can be linked or fused to a peptide in such a way that the linking does not interfere with ability of an antigen-binding domain of the construct to bind to an antigen, the ability of an Fc domain of the construct to bind to an FcR, or FcR-mediated signaling resulting from the Fc domain of the construct from binding to an FcR. A construct-peptide composition can produce stronger immune stimulation than components of the composition alone.

In a construct-peptide composition conjugated to an immune-stimulatory compound, the construct-peptide composition can be linked to an immune-stimulatory compound in such a way that an antigen-binding domain of the construct can still bind to an antigen and an Fc domain of the construct can still bind to an FcR. In a construct-peptide composition conjugated to an immune-stimulatory compound, a construct is linked to an immune-stimulatory compound in such a way that the linking does not interfere with ability of an antigen-binding domain of the construct to bind to an antigen, the ability of an Fc domain of the construct to bind to an FcR, or FcR-mediated signaling resulting from the Fc domain of the construct from binding to an FcR. In a construct-peptide composition conjugated to an immune-stimulatory compound, an immune-stimulatory compound can be linked to a construct in such a way the linking does not interfere with the ability of the immune-stimulatory compound to bind to its receptor. A construct-peptide composition conjugated to an immune-stimulatory compound can produce stronger immune stimulation and a greater therapeutic window than components of the conjugate alone.

An ATAC can be formed by conjugating a noncleavable maleimide-PEG4 linker containing a succinimide group with an immune-stimulatory compound. For example, an ATAC can be N-((4-amino-1-(2-hydroxy-2-methylpropyl)-1H-imidazo[4,5-c]quinolin-2-yl)methyl)-1-(3-(2,5-dioxo-2,5-dihydro-1H-pyrrol-1-yl)propanamido)-N-ethyl-3,6,9,12-tetraoxapentadecan-15-amide (ATAC11); N-(5-(2-amino-3-pentylquinolin-5-yl)pentyl)-1-(3-(2,5-dioxo-2,5-dihydro-1H-pyrrol-1-yl)propanamido)-3,6,9,12-tetraoxapentadecan-15-amide (ATAC12); 1-(3-(2,5-dioxo-2,5-dihydro-1H-pyrrol-1-yl)propanamido)-N-(3-pentylquinolin-2-yl)-3,6,9,12-tetraoxapentadecan-15-amide (ATAC13); 1-(3-(2,5-dioxo-2,5-dihydro-1H-pyrrol-1-yl)propanamido)-N-(1-isobutyl-1H-imidazo[4,5-c]quinolin-4-yl)-3,6,9,12-tetraoxapentadecan-15-amide (ATAC14); 1-(3-(2,5-dioxo-2,5-dihydro-1H-pyrrol-1-yl)propanamido)-N-methyl-N-(2-(3-(7-methylbenzo[1,2-d:3,4-d′]bis(thiazole)-2-yl)ureido)ethyl)-3,6,9,12-tetraoxapentadecan-15-amide (ATAC15); (S)-1-(3-(2,5-dioxo-2,5-dihydro-1H-pyrrol-1-yl)propanamido)-N-(1-((7-methylbenzo[1,2-d:3,4-d′]bis(thiazole)-2-yl)amino)-1-oxo-3-phenylpropan-2-yl)-3,6,9,12-tetraoxapentadecan-15-amide (ATAC16); N-(benzo[d]thiazol-2-yl)-1-(3-(2,5-dioxo-2,5-dihydro-1H-pyrrol-1-yl)propanamido)-N-((8-hydroxyquinolin-7-yl)(4-(trifluoromethoxy)phenyl)methyl)-3,6,9,12-tetraoxapentadecan-15-amide (ATAC17); N-((2R,3R,3aS,7aR,9R,10R,10aS,14aR)-2,9-bis(2-amino-6-oxo-1H-purin-9(6H)-yl)-5,10,12-trihydroxy-5,12-dioxidodecahydrodifuro[3,2-d:3′,2′-j][1,3,7,9,2,8]tetra-oxadiphosphacyclododecin-3-yl)-1-(3-(2,5-dioxo-2,5-dihydro-1H-pyrrol-1-yl)propanamido)-3,6,9,12-tetraoxapentadecan-15-amide (ATAC18); N-((2R,3R,3aS,7aR,9R,10R,10aS,14aR)-2,9-bis(2-amino-6-oxo-1H-purin-9(6H)-yl)-10-hydroxy-5,12-dimercapto-5,12-dioxidodecahydrodifuro[3,2-d:3′,2′-j][1,3,7,9,2,8]tetraoxadiphosphacyclododecin-3-yl)-1-(3-(2,5-dioxo-2,5-dihydro-1H-pyrrol-1-yl)propanamido)-3,6,9,12-tetraoxapentadecan-15-amide (ATAC19); N-(9-((2R,3R,3aS,7aR,9R,10R,10aS,14aR)-9-(2-amino-6-oxo-1H-purin-9(6H)-yl)-3,5,10,12-tetrahydroxy-5,12-dioxidodecahydrodifuro[3,2-d:3′,2′-j][1,3,7,9,2,8]tetra-oxadiphosphacyclododecin-2-yl)-9H-purin-6-yl)-1-(3-(2,5-dioxo-2,5-dihydro-1H-pyrrol-1-yl)propanamido)-3,6,9,12-tetraoxapentadecan-15-amide (ATAC20); or N-(9-((2R,3R,3aS,7aR,9R,10R,10aS,14aR)-9-(2-amino-6-oxo-1H-purin-9(6H)-yl)-3,5,10,12-tetrahydroxy-5,12-dioxidodecahydrodifuro[3,2-d:3′,2′-j][1,3,7,9,2,8]tetraoxadiphosphacyclododecin-2-yl)-9H-purin-6-yl)-1-(3-(2,5-dioxo-2,5-dihydro-1H-pyrrol-1-yl)propanamido)-3,6,9,12-tetraoxapentadecan-15-amide (ATAC21).

An ATAC can be formed by conjugating a cleavable valine-alanine or valine-citrulline linker containing a PABA group and a succinimide group with an immune-stimulatory compound. For example, an ATAC can be 4-((S)-2-((S)-2-(6-(2,5-dioxo-2,5-dihydro-1H-pyrrol-1-yl)hexanamido)-3-methylbutanamido)propanamido)benzyl ((4-amino-1-(2-hydroxy-2-methyl-propyl)-1H-imidazo[4,5-c]quinolin-2-yl)methyl)(ethyl)carbamate (ATAC22); 4-((S)-2-((S)-2-(6-(2,5-dioxo-2,5-dihydro-1H-pyrrol-1-yl)hexanamido)-3-methyl-butanamido)propanamido)benzyl (5-(2-amino-3-pentylquinolin-5-yl)pentyl)-carbamate (ATAC23); 4-((S)-2-((S)-2-(6-(2,5-dioxo-2,5-dihydro-1H-pyrrol-1-yl)hexanamido)-3-methylbutan-amido)-5-ureidopentanamido)benzyl-(5-(2-amino-3-pentylquinolin-5-yl)pentyl)-carbamate (ATAC24); 4-((S)-2-((S)-2-(6-(2,5-dioxo-2,5-dihydro-1H-pyrrol-1-yl)hexanamido)-3-methylbutanamido)-5-ureidopentanamido)benzyl((4-amino-1-(2-hydroxy-2-methylpropyl)-1H-imidazo[4,5-c]quinolin-2-yl)methyl)(ethyl)carbamate TFA salt (ATAC25); 2-(3-{2-[N-Methyl({p-[(S)-2-{(S)-2-[6-(2,5-dioxo-1H-pyrrol-1-yl)hexanoylamino]-3-methylbutyrylamino}-5-ureidovalerylamino]phenyl}methoxycarbonyl)amino]ethyl}ureido)-7-methyl-1,6-dithia-3,8-diaza-as-indacene (ATAC26); 2-{[(8-Hydroxy-7-quinolyl)(p-trifluoromethoxyphenyl)methyl]({p-[(S)-2-{(S)-2-[6-(2,5-dioxo-1H-pyrrol-1-yl)hexanoylamino]-3-methylbutyrylamino}-5-ureidovalerylamino]phenyl}methoxycarbonyl)amino}-1,3-benzothiazole (ATAC27); (1R,6R,8R,9S,10S,15R,17R,18S)-18-({p-[(S)-2-{(S)-2-[6-(2,5-Dioxo-1H-pyrrol-1-yl)hexanoylamino]-3-methylbutyrylamino}-5-ureidovalerylamino]phenyl}methoxycarbonylamino)-8,17-bis(2-amino-6-oxo-1,9-dihydropurin-9-yl)-3,12-dihydroxy-9-hydroxy-2.4.7.11.13.16-hexaoxa-3λ5. 12λ5-diphosphatricyclo[13.3.0.06,10]octadecane-3,12-dione (ATAC28); (1R,6R,8R,9S,10S, 15R, 17R,18S)-18-({p-[(S)-2-{(S)-2-[6-(2,5-Dioxo-1H-pyrrol-1-yl)hexanoylamino]-3-methylbutyrylamino}propionylamino]phenyl}methoxycarbonylamino)-8,17-bis(2-amino-6-oxo-1,9-dihydropurin-9-yl)-3,12-dihydroxy-9-hydroxy-2.4.7.11.13.16-hexaoxa-3λ5. 12λ5-diphosphatricyclo[13.3.0.06,10]octadecane-3,12-dione (ATAC29); (1R,6R,8R,9S,10S, 15R, 17R,18S)-18-({p-[(S)-2-{(S)-2-[6-(2,5-Dioxo-1H-pyrrol-1-yl)hexanoylamino]-3-methylbutyrylamino}-5-ureidovalerylamino]phenyl}methoxycarbonylamino)-8,17-bis(2-amino-6-oxo-1,9-dihydropurin-9-yl)-9-hydroxy-3,12-dimercapto-2.4.7.11.13.16-hexaoxa-3λ5.12λ5-diphosphatricyclo[13.3.0.06,10]octadecane-3,12-dione (ATAC30); {p-[(S)-2-{(S)-2-[6-(2,5-Dioxo-1H-pyrrol-1-yl)hexanoylamino]-3-methylbutyrylamino}-5-ureidovalerylamino]phenyl}methyl 9-(1S,6R,8R,9S,10S,15R,17R,18S)-8-(2-amino-6-oxo-1,9-dihydropurin-9-yl)-3,12-dihydroxy-9,18-dihydroxy-3,12-dioxo-2.4.7.11.13.16-hexaoxa-3λ5.12λ5-diphosphatricyclo[13.2.1.06,10]octadec-17-yl}-9a-adenineecarboxylate (ATAC31; 1-{6-[({7-Amino-3-(2-hydroxy-2-methylpropyl)-3.5.8-triazatricyclo[7.4.0.02,6]trideca-1(9),2(6),4,7,10,12-hexaen-4-yl}methyl)-N-ethylamino]-6-oxohexyl}-1H-pyrrole-2,5-dione (ATAC32); 1-{[4-({6-[({7-Amino-3-(2-hydroxy-2-methylpropyl)-3.5.8-triazatricyclo[7.4.0.02,6]trideca-1(9),2(6),4,7,10,12-hexaen-4-yl}methyl)-N-ethylamino]-6-oxohexylamino}carbonyl)cyclohexyl]methyl}-1H-pyrrole-2,5-dione (ATAC33); or 1-[(4-{[({7-Amino-3-(2-hydroxy-2-methylpropyl)-3.5.8-triazatricyclo[7.4.0.02,6]trideca-1(9),2(6),4,7,10,12-hexaen-4-yl}methyl)-N-ethylamino]-carbonyl}cyclohexyl)methyl]-1H-pyrrole-2,5-dione (ATAC34).

An ATAC can be formed by conjugating a noncleavable maleimide-PEG4 linker containing an activated ester such as a pentafluorophenyl group or an N-hydroxysuccinimide group with an immune-stimulatory compound. For example, an ATAC can be pentafluorophenyl 25-(2-amino-3-pentylquinolin-5-yl)-19-oxo-4,7,10,13,16-pentaoxa-20-azapentacosanoate (ATAC1); perfluorophenyl 3-((4-amino-1-(2-hydroxy-2-methylpropyl)-1H-imidazo[4,5-c]quinolin-2-yl)methyl)-4-oxo-7,10,13,16,19-pentaoxa-3-azadocosan-22-oate (ATAC2); pentafluorophenyl 25-(2-amino-3-pentylquinolin-5-yl)-19-oxo-4,7,10,13,16-pentaoxa-20-azapentacosanoate (ATAC3); or 2,5-Dioxopyrrolidin-1-yl 3-((4-amino-1-(2-hydroxy-2-methylpropyl)-1H-imidazo-[4,5-c]quinolin-2-yl)methyl)-4-oxo-7,10,13,16,19-pentaoxa-3-azadocosan-22-oate (ATAC4).

An ATAC can be formed by conjugating a cleavable valine-alanine or valine-citrulline linker containing a PABA group and an activated ester such as a pentafluorophenyl group or an N-hydroxysuccinimde group to an immune-stimulatory compound. For example, an ATAC can be 2,5-dioxopyrrolidin-1-yl 6-(((S)-1-(((S)-1-((4-((((5-(2-amino-3-pentylquinolin-5-yl)pentyl)carbamoyl)oxy)methyl)phenyl)amino)-1-oxopropan-2-yl)amino)-3-methyl-1-oxobutan-2-yl)amino)-6-oxohexanoate (ATAC5); 2,5-dioxopyrrolidin-1-yl 7-(((S)-1-(((S)-1-((4-(((((4-amino-1-(2-hydroxy-2-methylpropyl)-1H-imidazo[4,5-c]quinolin-2-yl)methyl)(ethyl)carbamoyl)oxy)methyl)phenyl)amino)-1-oxopropan-2-yl)amino)-3-methyl-1-oxobutan-2-yl)amino)-7-oxoheptanoate (ATAC6); 2,5-dioxopyrrolidin-1-yl 7-(((S)-1-(((S)-1-((4-(((((4-amino-1-(2-hydroxy-2-methylpropyl)-1H-imidazo[4,5-c]quinolin-2-yl)methyl)(ethyl)carbamoyl)oxy)methyl)phenyl)amino)-1-oxo-5-ureidopentan-2-yl)amino)-3-methyl-1-oxobutan-2-yl)amino)-7-oxoheptanoate (ATAC7); perfluorophenyl 6-(((S)-1-(((S)-1-((4-((((5-(2-amino-3-pentylquinolin-5-yl)pentyl)carbamoyl)oxy)methyl)phenyl)amino)-1-oxopropan-2-yl)amino)-3-methyl-1-oxobutan-2-yl)amino)-6-oxohexanoate (ATAC8); perfluorophenyl 7-(((S)-1-(((S)-1-((4-(((((4-amino-1-(2-hydroxy-2-methylpropyl)-1H-imidazo[4,5-c]quinolin-2-yl)methyl)(ethyl)carbamoyl)oxy)methyl)phenyl)amino)-1-oxopropan-2-yl)amino)-3-methyl-1-oxobutan-2-yl)amino)-7-oxoheptanoate (ATAC9); or perfluorophenyl 7-(((S)-1-(((S)-1-((4-(((((4-amino-1-(2-hydroxy-2-methylpropyl)-1H-imidazo[4,5-c]quinolin-2-yl)methyl)(ethyl)carbamoyl)oxy)methyl)phenyl)amino)-1-oxo-5-ureidopentan-2-yl)amino)-3-methyl-1-oxobutan-2-yl)amino)-7-oxoheptanoate (ATAC10).

A construct-peptide composition can comprise an anti-CD40 antibody. An anti-CD40 antibody can comprise two SBT-040-G1WT heavy chains and two light chain from a SBT-040 antibody, which can be referred to as SBT-040-WT or as SBT-040-G1. An anti-CD40 antibody can comprise two SBT-040-G1VLPLL heavy chains and two light chains from a SBT-040 antibody, which can be referred to as SBT-040-VLPLL. An anti-CD40 antibody can comprise two SBT-040-G1DE heavy chains and two light chains from a SBT-040 antibody, which can be referred to as SBT-040-DE. An anti-CD40 antibody can comprise two SBT-040-G1AAA heavy chains and two light chains from a SBT-040 antibody, which can be referred to as SBT-040-AAA. An anti-CD40 antibody can comprise two IgG2 heavy chains and two light chain from a SBT-040 antibody, which can be referred to as SBT-040-G2.

A construct-peptide immune-stimulatory conjugate can comprise SBT-040/peptide-WT-ATAC1. A conjugate can comprise SBT-040/peptide-WT-ATAC2. A conjugate can comprise SBT-040/peptide-WT-ATAC3. A conjugate can comprise SBT-040/peptide-WT-ATAC4. A conjugate can comprise SBT-040/peptide-WT-ATAC5. A conjugate can comprise SBT-040/peptide-WT-ATAC6. A conjugate can comprise SBT-040/peptide-WT-ATAC7. A conjugate can comprise SBT-040/peptide-WT-ATAC8. A conjugate can comprise SBT-040/peptide-WT-ATAC9. A conjugate can comprise SBT-040/peptide-WT-ATAC10. A conjugate can comprise SBT-040/peptide-WT-ATAC11. A conjugate can comprise SBT-040/peptide-WT-ATAC12. A conjugate can comprise SBT-040/peptide-WT-ATAC13. A conjugate can comprise SBT-040/peptide-WT-ATAC14. A conjugate can comprise SBT-040/peptide-WT-ATAC15. A conjugate can comprise SBT-040/peptide-WT-ATAC16. A conjugate can comprise SBT-040/peptide-WT-ATAC17. A conjugate can comprise SBT-040/peptide-WT-ATAC18. A conjugate can comprise SBT-040/peptide-WT-ATAC19. A conjugate can comprise SBT-040/peptide-WT-ATAC20. A conjugate can comprise SBT-040/peptide-WT-ATAC21. A conjugate can comprise SBT-040/peptide-WT-ATAC22. A conjugate can comprise SBT-040/peptide-WT-ATAC23. A conjugate can comprise SBT-040/peptide-WT-ATAC24. A conjugate can comprise SBT-040/peptide-WT-ATAC25. A conjugate can comprise SBT-040/peptide-WT-ATAC26. A conjugate can comprise SBT-040/peptide-WT-ATAC27. A conjugate can comprise SBT-040/peptide-WT-ATAC28. A conjugate can comprise SBT-040/peptide-WT-ATAC29. A conjugate can comprise SBT-040/peptide-WT-ATAC30. A conjugate can comprise SBT-040/peptide-WT-ATAC31. A conjugate can comprise SBT-040/peptide-WT-ATAC32. A conjugate can comprise SBT-040/peptide-WT-ATAC33. A conjugate can comprise SBT-040/peptide-WT-ATAC34. A conjugate can comprise SBT-040/peptide-VLPLL-ATAC1. A conjugate can comprise SBT-040/peptide-VLPLL-ATAC2. A conjugate can comprise SBT-040/peptide-VLPLL-ATAC3. A conjugate can comprise SBT-040/peptide-VLPLL-ATAC4. A conjugate can comprise SBT-040/peptide-VLPLL-ATAC5. A conjugate can comprise SBT-040/peptide-VLPLL-ATAC6. A conjugate can comprise SBT-040/peptide-VLPLL-ATAC7. A conjugate can comprise SBT-040/peptide-VLPLL-ATAC8. A conjugate can comprise SBT-040/peptide-VLPLL-ATAC9. A conjugate can comprise SBT-040/peptide-VLPLL-ATAC10. A conjugate can comprise SBT-040/peptide-VLPLL-ATAC11. A conjugate can comprise SBT-040/peptide-VLPLL-ATAC12. A conjugate can comprise SBT-040/peptide-VLPLL-ATAC13. A conjugate can comprise SBT-040/peptide-VLPLL-ATAC14. A conjugate can comprise SBT-040/peptide-VLPLL-ATAC15. A conjugate can comprise SBT-040/peptide-VLPLL-ATAC16. A conjugate can comprise SBT-040/peptide-VLPLL-ATAC17. A conjugate can comprise SBT-040/peptide-VLPLL-ATAC18. A conjugate can comprise SBT-040/peptide-VLPLL-ATAC19. A conjugate can comprise SBT-040/peptide-VLPLL-ATAC20. A conjugate can comprise SBT-040/peptide-VLPLL-ATAC21. A conjugate can comprise SBT-040/peptide-VLPLL-ATAC22. A conjugate can comprise SBT-040/peptide-VLPLL-ATAC23. A conjugate can comprise SBT-040/peptide-VLPLL-ATAC24. A conjugate can comprise SBT-040-VLPLL-ATAC25. A conjugate can comprise SBT-040/peptide-VLPLL-ATAC26. A conjugate can comprise SBT-040/peptide-VLPLL-ATAC27. A conjugate can comprise SBT-040/peptide-VLPLL-ATAC28. A conjugate can comprise SBT-040/peptide-VLPLL-ATAC29. A conjugate can comprise SBT-040/peptide-VLPLL-ATAC30. A conjugate can comprise SBT-040/peptide-VLPLL-ATAC31. A conjugate can comprise SBT-040/peptide-VLPLL-ATAC32. A conjugate can comprise SBT-040/peptide-VLPLL-ATAC33. A conjugate can comprise SBT-040/peptide-VLPLL-ATAC34. A conjugate can comprise SBT-040/peptide-DE-ATAC1. A conjugate can comprise SBT-040/peptide-DE-ATAC2. A conjugate can comprise SBT-040/peptide-DE-ATAC3. A conjugate can comprise SBT-040/peptide-DE-ATAC4. A conjugate can comprise SBT-040/peptide-DE-ATAC5. A conjugate can comprise SBT-040/peptide-DE-ATAC6. A conjugate can comprise SBT-040/peptide-DE-ATAC7. A conjugate can comprise SBT-040/peptide-DE-ATAC8. A conjugate can comprise SBT-040/peptide-DE-ATAC9. A conjugate can comprise SBT-040/peptide-DE-ATAC10. A conjugate can comprise SBT-040/peptide-DE-ATAC11. A conjugate can comprise SBT-040/peptide-DE-ATAC12. A conjugate can comprise SBT-040/peptide-DE-ATAC13. A conjugate can comprise SBT-040/peptide-DE-ATAC14. A conjugate can comprise SBT-040/peptide-DE-ATAC15. A conjugate can comprise SBT-040/peptide-DE-ATAC16. A conjugate can comprise SBT-040/peptide-DE-ATAC17. A conjugate can comprise SBT-040/peptide-DE-ATAC18. A conjugate can comprise SBT-040/peptide-DE-ATAC19. A conjugate can comprise SBT-040/peptide-DE-ATAC20. A conjugate can comprise SBT-040/peptide-DE-ATAC21. A conjugate can comprise SBT-040/peptide-DE-ATAC22. A conjugate can comprise SBT-040/peptide-DE-ATAC23. A conjugate can comprise SBT-040-DE-ATAC24. A conjugate can comprise SBT-040/peptide-DE-ATAC25. A conjugate can comprise SBT-040/peptide DE-ATAC26. A conjugate can comprise SBT-040/peptide-DE-ATAC27. A conjugate can comprise SBT-040/peptide-DE-ATAC28. A conjugate can comprise SBT-040/peptide-DE-ATAC29. A conjugate can comprise SBT-040/peptide-DE-ATAC30. A conjugate can comprise SBT-040/peptide-DE-ATAC31. A conjugate can comprise SBT-040/peptide-DE-ATAC32. A conjugate can comprise SBT-040/peptide-DE-ATAC33. A conjugate can comprise SBT-040/peptide-DE-ATAC34. A conjugate can comprise SBT-040/peptide-AAA-ATAC1. A conjugate can comprise SBT-040/peptide-AAA-ATAC2. A conjugate can comprise SBT-040/peptide-AAA-ATAC3. A conjugate can comprise SBT-040/peptide-AAA-ATAC4. A conjugate can comprise SBT-040/peptide-AAA-ATAC5. A conjugate can comprise SBT-040/peptide-AAA-ATAC6. A conjugate can comprise SBT-040/peptide-AAA-ATAC7. A conjugate can comprise SBT-040/peptide-AAA-ATAC8. A conjugate can comprise SBT-040/peptide-AAA-ATAC9. A conjugate can comprise SBT-040/peptide-AAA-ATAC10. A conjugate can comprise SBT-040/peptide-AAA-ATAC11. A conjugate can comprise SBT-040/peptide-AAA-ATAC12. A conjugate can comprise SBT-040/peptide-AAA-ATAC13. A conjugate can comprise SBT-040/peptide-AAA-ATAC14. A conjugate can comprise SBT-040/peptide-AAA-ATAC15. A conjugate can comprise SBT-040/peptide-AAA-ATAC16. A conjugate can comprise SBT-040/peptide-AAA-ATAC17. A conjugate can comprise SBT-040/peptide-AAA-ATAC18. A conjugate can comprise SBT-040/peptide-AAA-ATAC19. A conjugate can comprise SBT-040/peptide-AAA-ATAC20. A conjugate can comprise SBT-040/peptide-AAA-ATAC21. A conjugate can comprise SBT-040/peptide-AAA-ATAC22. A conjugate can comprise SBT-040/peptide-AAA-ATAC23. A conjugate can comprise SBT-040/peptide-AAA-ATAC24. A conjugate can comprise SBT-040/peptide-AAA-ATAC25. A conjugate can comprise SBT-040/peptide-AAA-ATAC26. A conjugate can comprise SBT-040/peptide-AAA-ATAC27. A conjugate can comprise SBT-040/peptide-AAA-ATAC28. A conjugate can comprise SBT-040/peptide-AAA-ATAC29. A conjugate can comprise SBT-040/peptide-AAA-ATAC30. A conjugate can comprise SBT-040/peptide-AAA-ATAC31. A conjugate can comprise SBT-040/peptide-AAA-ATAC32. A conjugate can comprise SBT-040/peptide-AAA-ATAC33. A conjugate can comprise SBT-040/peptide-AAA-ATAC34. A conjugate can comprise SBT-040/peptide-AAA-ATAC33. The K_{d} for binding of the CD40 binding domain of any of these conjugates to CD40 can be about 2 times, about 3 times, about 4 times, about 5 times, about 6 times, about 7 times, about 8 times, about 9 times, about 10 times, about 15 times, about 20 times, about 25 times, about 30 times, about 35 times, about 40 times, about 45 times, about 50 times, about 60 times, about 70 times, about 80 times, about 90 times, about 100 times, about 110 times, or about 120 times greater than the K_{d} for binding of the CD40 binding domain to CD40 in the absence of the immune-stimulatory compound or ATAC. The K_{d} for binding of the CD40 binding domain of any of these conjugates to CD40 can be less than 10 nM. The K_{d} for binding of the CD40 binding domain of any of the conjugates to CD40 can be less than 100 nM, less than 50 nM, less than 20 nM, less than 5 nM, less than 1 nM, or less than 0.1 nM. The K_{d} for binding of the Fc domain of any of the conjugates to an Fc receptor can be about 2 times, about 3 times, about 4 times, about 5 times, about 6 times, about 7 times, about 8 times, about 9 times, about 10 times, about 15 times, about 20 times, about 25 times, about 30 times, about 35 times, about 40 times, about 45 times, about 50 times, about 60 times, about 70 times, about 80 times, about 90 times, about 100 times, about 110 times, or about 120 times greater than the K_{d} for binding of the Fc domain to the Fc receptor in the absence of the immune-stimulatory compound or ATAC. The K_{d} for binding of the Fc domain of any of the conjugates to an Fc receptor of an can be less than 10 nM. The K_{d} for binding of the Fc domain of any of the conjugates to an Fc receptor can be less than 100 nM, less than 50 nM, less than 20 nM, less than 5 nM, less than 1 nM, or less than 0.1 nM.

In a conjugate, an antibody can be linked to an immune-stimulatory compound in such a way that the antibody can still bind to an antigen and the Fc domain of the antibody can still bind to an FcR. In a conjugate, an antibody construct is linked to an immune-stimulatory compound in such a way that the linking does not interfere with ability of the antigen binding domain of the antibody construct to bind to antigen, the ability of the Fc domain of the antibody construct to bind to an FcR, or FcR-mediated signaling resulting from the Fc domain of the antibody construct from binding to an FcR. In a conjugate, an immune-stimulatory compound can be linked to an antibody construct in such a way the linking does not interfere with the ability of the immune-stimulatory compound to bind to its receptor. A conjugate can produce stronger immune stimulation and a greater therapeutic window than components of the conjugate alone. In an anti-CD40 antibody linked to a TLR agonist conjugate, the combination of CD40 agonism, TLR agonism, and an accessible Fc domain of the anti-CD40 antibody to allow FcR-mediated signaling can produce stronger immune stimulation and a greater therapeutic window than the CD40 agonism, TLR agonism, or the FcR-mediated signaling alone.

### Synthesis of ATAC Compounds

An ATAC compound can be synthesized by various methods. For example, ATAC compounds, such as ATAC1-ATAC4, can be synthesized as shown in Scheme B1.

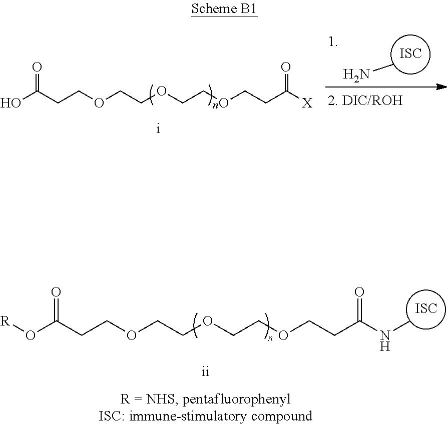

A PEGylated carboxylic acid (i) that has been activated for amide bond formation can be reacted with an appropriately substituted amine containing immune-stimulatory compound to afford an intermediate amide. Formation of an activated ester (ii) can be achieved by reaction the intermediate amide-containing carboxylic using a reagent such as N-hydroxysuccinimide or pentafluorophenol in the presence of a coupling agent such as diisopropylcarbodiimide (DIC) to provide compounds (ii).

An ATAC compound can be synthesized by various methods. For example, ATAC compounds, such as ATAC5-ATAC10, can be synthesized as shown in Scheme B2.

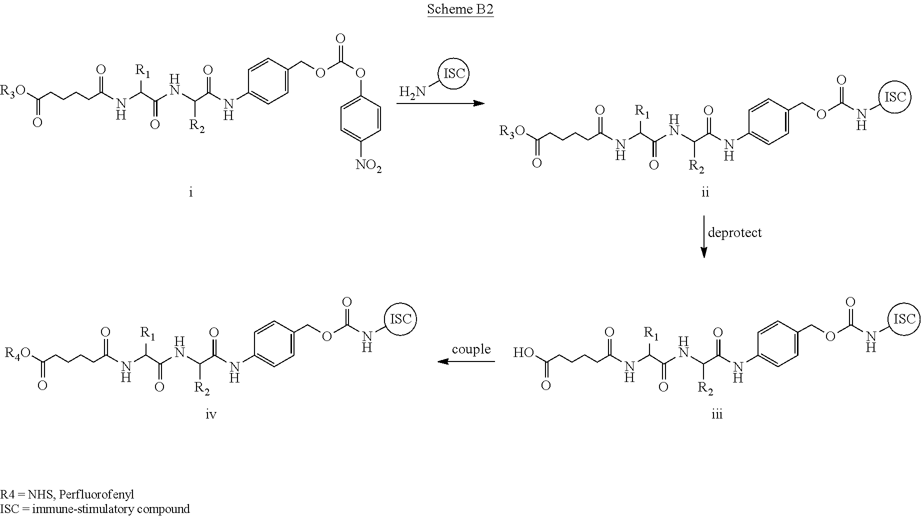

An activated carbonate such as (i) can be reacted with an appropriately substituted amine containing immune-stimulatory compound to afford carbamates (ii) which can be deprotected using standard methods based on the nature of the R₃ ester group. The resulting carboxylic acid (iii) can then by coupled with an activating agent such as N-hydroxysuccinimide or pentafluorophenol to provide compounds (iv).

An ATAC compound can be synthesized by various methods. For example, ATAC compounds, such as ATAC11-ATAC21, can be synthesized as shown in Scheme B3.

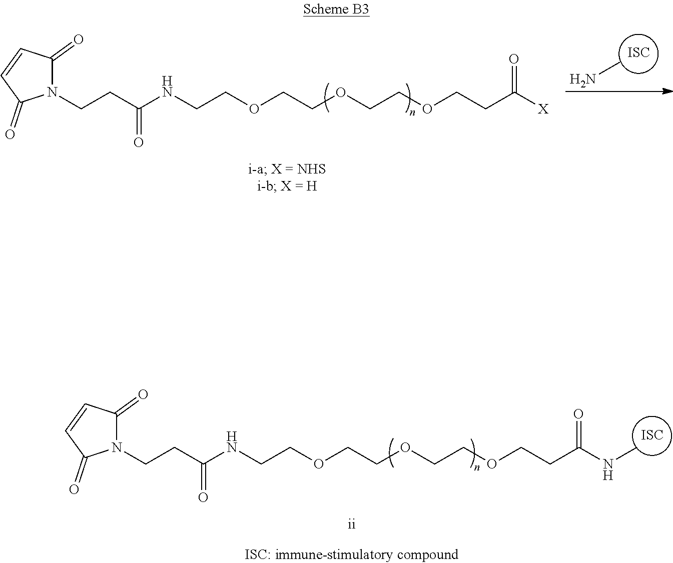

An activated carboxylic ester such as (i-a) can be reacted with an appropriately substituted amine containing immune-stimulatory compound to afford amides (ii). Alternatively, carboxylic acids of type (i-b) can be coupled to an appropriately substituted amine containing immune-stimulatory compound in the presence of an amide bond forming agent such as dicyclohexycarbodiimde (DCC) to provide the desired ATAC compounds.

An ATAC compound can be synthesized by various methods. For example, ATAC compounds, such as ATAC22-ATAC31, can be synthesized as shown in Scheme B4.

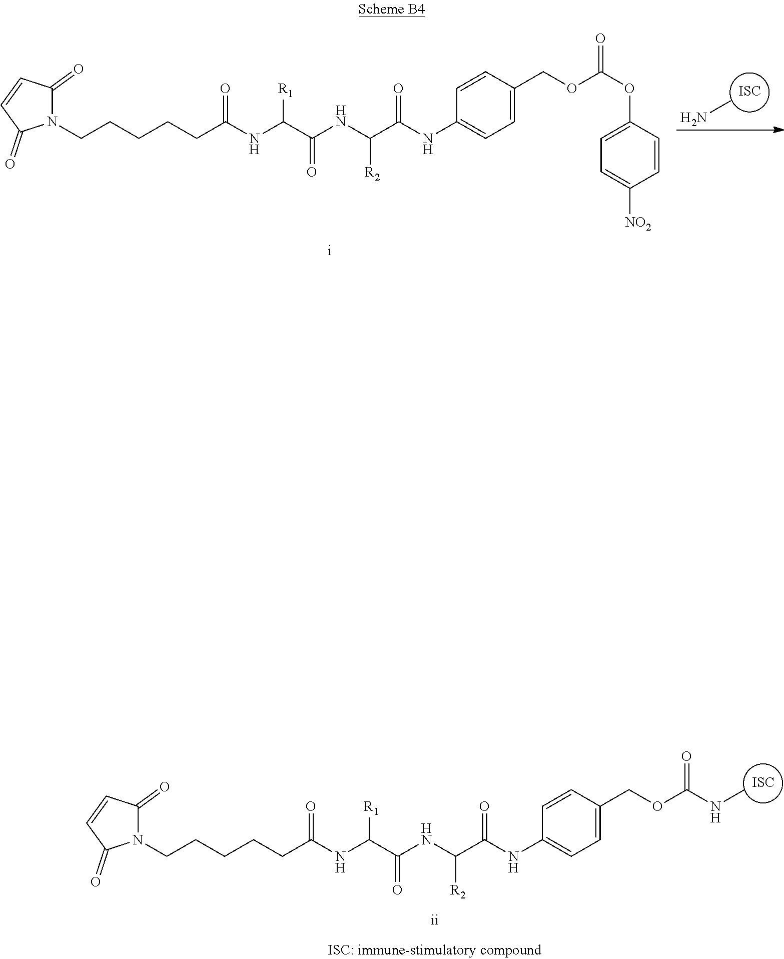

An activated carbonate such as (i) can be reacted with an appropriately substituted amine containing immune-stimulatory compound to afford carbamates (ii) as the target ATAC compounds.

An ATAC compound can be synthesized by various methods. For example, ATAC compounds, such as ATAC32-ATAC34, can be synthesized as shown in Scheme B5.

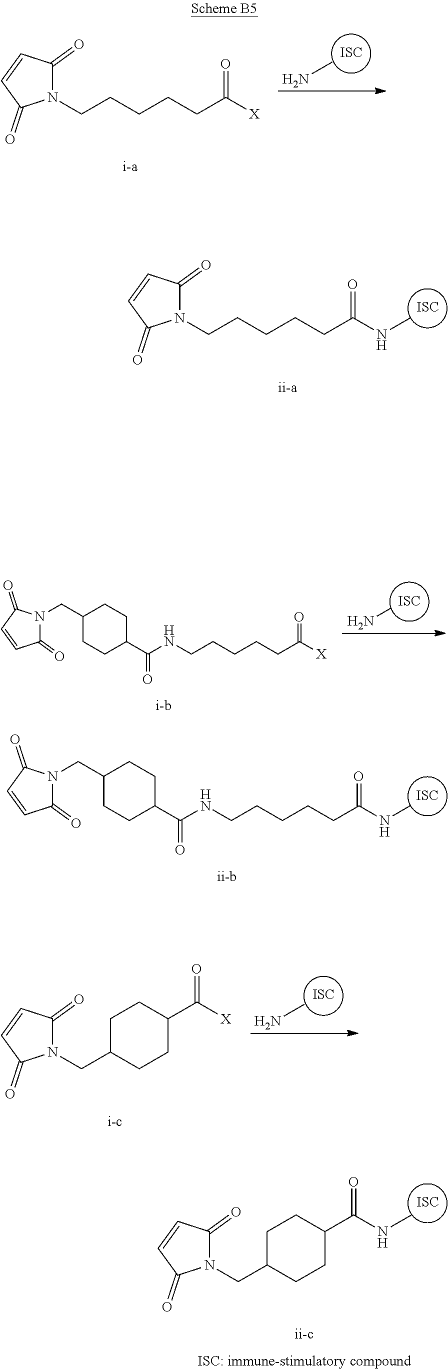

An activated carboxylic acid such as (i-a, i-b, i-c) can be reacted with an appropriately substituted amine containing immune-stimulatory compound to afford amides (ii-a, ii-b, ii-c) as the target ATAC compounds.

These construct-peptide immune-stimulatory conjugates can be made by various methods. It is understood that one skilled in the art may be able to make these compounds by similar methods or by combining other methods known to one skilled in the art. It is also understood that one skilled in the art would be able to make, in a similar manner as described herein by using the appropriate starting materials and modifying the synthetic route as needed. Starting materials and reagents can be obtained from commercial vendors or synthesized according to sources known to those skilled in the art or prepared as described herein.

### Pharmaceutical Formulations

The construct-peptide compositions and conjugates and methods described herein are useful as pharmaceutical compositions for administration to a subject in need thereof. Pharmaceutical compositions can comprise at least the construct-peptide composition or conjugate described herein and one or more pharmaceutically acceptable carriers, diluents, excipients, stabilizers, dispersing agents, suspending agents, and/or thickening agents. The pharmaceutical composition can comprise a construct-peptide composition. The pharmaceutical composition can comprise a construct-peptide composition wherein the construct is fused or linked with a peptide. The pharmaceutical composition can comprise a construct-peptide composition conjugated to an immune-stimulatory compound. A pharmaceutical composition can further comprise buffers, antibiotics, steroids, carbohydrates, drugs (e.g., chemotherapy drugs), radiation, polypeptides, chelators, adjuvants and/or preservatives.

Pharmaceutical compositions can be formulated using one or more physiologically-acceptable carriers comprising excipients and auxiliaries. Formulation can be modified depending upon the route of administration chosen. Pharmaceutical compositions comprising a composition as described herein can be manufactured, for example, by lyophilizing the conjugate, mixing, dissolving, emulsifying, encapsulating or entrapping the conjugate. The pharmaceutical compositions can also include the compositions described herein in a free-base form or pharmaceutically-acceptable salt form.

Methods for formulation of the pharmaceutical composition can include formulating any of the antibody constructs or conjugates described herein with one or more inert, pharmaceutically-acceptable excipients or carriers to form a solid, semi-solid, or liquid composition. Solid compositions can include, for example, powders, tablets, dispersible granules and capsules, and in some aspects, the solid compositions further contain nontoxic, auxiliary substances, for example wetting or emulsifying agents, pH buffering agents, and other pharmaceutically-acceptable additives. Alternatively, the pharmaceutical compositions described herein can be lyophilized or in powder form for re-constitution with a suitable vehicle, e.g., sterile pyrogen-free water, before use

Pharmaceutical compositions of the construct-peptide or conjugate described herein can comprise at least an active ingredient. The active ingredients can be entrapped in microcapsules prepared, for example, by coacervation techniques or by interfacial polymerization (e.g., hydroxymethylcellulose or gelatin microcapsules and poly-(methylmethacylate) microcapsules, respectively), in colloidal drug-delivery systems (e.g., liposomes, albumin microspheres, microemulsions, nano-particles and nanocapsules) or in macroemulsions.

Pharmaceutical compositions as described herein often further can comprise more than one active compound as necessary for the particular indication being treated. The active compounds can have complementary activities that do not adversely affect each other. For example, the pharmaceutical composition can also comprise a chemotherapeutic agent, cytotoxic agent, cytokine, growth-inhibitory agent, anti-hormonal agent, anti-angiogenic agent, and/or cardioprotectant. Such molecules can be present in combination in amounts that are effective for the purpose intended.

The pharmaceutical compositions and formulations can be sterilized. Sterilization can be accomplished by filtration through sterile filtration.

The pharmaceutical compositions described herein can be formulated for administration as an injection. Non-limiting examples of formulations for injection can include a sterile suspension, solution or emulsion in oily or aqueous vehicles. Suitable oily vehicles can include, but are not limited to, lipophilic solvents or vehicles such as fatty oils or synthetic fatty acid esters, or liposomes. Aqueous injection suspensions can contain substances which increase the viscosity of the suspension. The suspension can also contain suitable stabilizers. Injections can be formulated for bolus injection or continuous infusion. Alternatively, the pharmaceutical compositions described herein can be lyophilized or in powder form for reconstitution with a suitable vehicle, e.g., sterile pyrogen-free water, before use.

For parenteral administration construct-peptide or conjugate can be formulated in a unit dosage injectable form (e.g., use letter solution, suspension, emulsion) in association with a pharmaceutically acceptable parenteral vehicle. Such vehicles can be inherently nontoxic, and non-therapeutic. A vehicle can be water, saline, Ringer's solution, dextrose solution, and 5% human serum albumin. Nonaqueous vehicles such as fixed oils and ethyl oleate can also be used. Liposomes can be used as carriers. The vehicle can contain minor amounts of additives such as substances that enhance isotonicity and chemical stability (e.g., buffers and preservatives).

Sustained-release preparations can also be prepared. Examples of sustained-release preparations can include semipermeable matrices of solid hydrophobic polymers that can contain the antibody, and these matrices can be in the form of shaped articles (e.g., films or microcapsules). Examples of sustained-release matrices can include polyesters, hydrogels (e.g., poly(2-hydroxyethyl-methacrylate), or poly(vinylalcohol)), polylactides, copolymers of L-glutamic acid and y ethyl-L-glutamate, non-degradable ethylene-vinyl acetate, degradable lactic acid-glycolic acid copolymers such as the LUPRON DEPO™ (i.e., injectable microspheres composed of lactic acid-glycolic acid copolymer and leuprolide acetate), and poly-D-(−)-3-hydroxybutyric acid.

Pharmaceutical formulations of the compositions described herein can be prepared for storage by mixing a construct-peptide composition or conjugate with a pharmaceutically acceptable carrier, excipient, and/or a stabilizer. This formulation can be a lyophilized formulation or an aqueous solution. Acceptable carriers, excipients, and/or stabilizers can be nontoxic to recipients at the dosages and concentrations used. Acceptable carriers, excipients, and/or stabilizers can include buffers such as phosphate, citrate, and other organic acids; antioxidants including ascorbic acid and methionine; preservatives, polypeptides; proteins, such as serum albumin or gelatin; hydrophilic polymers; amino acids; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes; and/or non-ionic surfactants or polyethylene glycol.

### Therapeutic Applications

The construct-peptide composition, conjugates, pharmaceutical compositions, and methods of the present disclosure can be useful for treating a plurality of different subjects including, but are not limited to, a mammal, human, non-human mammal, a domesticated animal (e.g., laboratory animals, household pets, or livestock), non-domesticated animal (e.g., wildlife), dog, cat, rodent, mouse, hamster, cow, bird, chicken, fish, pig, horse, goat, sheep, rabbit, and any combination thereof. In some embodiments, a method of treating a subject in need thereof may comprise administering a therapeutic dose of the compositions or composition mixtures or the pharmaceutical compositions.

The construct-peptide composition, conjugates, pharmaceutical compositions, and methods described herein can be useful as a therapeutic, for example a treatment that can be administered to a subject in need thereof. A therapeutic effect of the present disclosure can be obtained in a subject by reduction, suppression, remission, or eradication of a disease state, including, but not limited to, a symptom thereof. A therapeutic effect in a subject having a disease or condition, or pre-disposed to have or is beginning to have the disease or condition, can be obtained by a reduction, a suppression, a prevention, a remission, or an eradication of the condition or disease, or pre-condition or pre-disease state.

In practicing the methods described herein, therapeutically-effective amounts of the construct-peptide composition, conjugates, or pharmaceutical compositions described herein can be administered to a subject in need thereof, often for treating and/or preventing a condition or progression thereof. A pharmaceutical composition can affect the physiology of the subject, such as the immune system, inflammatory response, or other physiologic affect. A therapeutically-effective amount can vary widely depending on the severity of the disease, the age and relative health of the subject, the potency of the compounds used, and other factors.

Treat and/or treating refers to any indicia of success in the treatment or amelioration of the disease or condition. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. Treat can be used herein to refer to a method that results in some level of treatment or amelioration of the disease or condition, and can contemplate a range of results directed to that end, including but not restricted to prevention of the condition entirely.

Prevent, preventing and the like refers to the prevention of the disease or condition, e.g., tumor formation, in the patient. For example, if an individual at risk of developing a tumor or other form of cancer is treated with the methods of the present disclosure and does not later develop the tumor or other form of cancer, then the disease has been prevented, at least over a period of time, in that individual.

A therapeutically effective amount can be the amount of a composition or an active component thereof sufficient to provide a beneficial effect or to otherwise reduce a detrimental non-beneficial event to the individual to whom the composition is administered. A therapeutically effective dose can be a dose that produces one or more desired or desirable (e.g., beneficial) effects for which it is administered, such administration occurring one or more times over a given period of time. An exact dose can depend on the purpose of the treatment, and can be ascertainable by one skilled in the art using known techniques.

The construct-peptide compositions, conjugates or pharmaceutical compositions described herein that can be used in therapy can be formulated and dosages established in a fashion consistent with good medical practice taking into account the disorder to be treated, the condition of the individual patient, the site of delivery of the conjugate or pharmaceutical composition, the method of administration and other factors known to practitioners. The construct-peptide compositions, conjugates or pharmaceutical compositions described herein can be prepared according to the description of preparation described herein.

Pharmaceutical compositions can be considered useful with the compositions and methods described herein can be administered to a subject in need thereof using a technique known to one of ordinary skill in the art which can be suitable as a therapy for the disease or condition affecting the subject. One of ordinary skill in the art would understand that the amount, duration and frequency of administration of a pharmaceutical composition described herein to a subject in need thereof depends on several factors including, for example but not limited to, the health of the subject, the specific disease or condition of the patient, the grade or level of a specific disease or condition of the patient, the additional therapeutics the subject is being or has been administered, and the like.

The methods, construct-peptide compositions, conjugates, or pharmaceutical compositions described herein can be for administration to a subject in need thereof. Often, administration of the construct-peptide compositions, conjugates, or pharmaceutical compositions described herein can include routes of administration, non-limiting examples of administration routes include intravenous, intraarterial, subcutaneous, subdural, intramuscular, intracranial, intrasternal, intratumoral, or intraperitoneally. Additionally, a pharmaceutical composition, construct-peptide composition, or conjugate can be administered to a subject by additional routes of administration, for example, by inhalation, oral, dermal, intranasal, or intrathecal administration. In some embodiments, the composition, composition mixture, or pharmaceutical composition is administered intravenously, cutaneously, subcutaneously, or injected at a site of affliction.

Pharmaceutical compositions, construct-peptide compositions, or conjugates of the present disclosure can be administered to a subject in need thereof in a first administration, and in one or more additional administrations. The one or more additional administrations can be administered to the subject in need thereof minutes, hours, days, weeks or months following the first administration. Any one of the additional administrations can be administered to the subject in need thereof less than 21 days, or less than 14 days, less than 10 days, less than 7 days, less than 4 days or less than 1 day after the first administration. The one or more administrations can occur more than once per day, more than once per week or more than once per month. The construct-peptide compositions, conjugates, or pharmaceutical compositions can be administered to the subject in need thereof in cycles of 21 days, 14 days, 10 days, 7 days, 4 days or daily over a period of one to seven days.

### Diseases, Conditions and the Like

The construct-peptide compositions, conjugates, or pharmaceutical compositions and methods provided herein can be useful for the treatment of a plurality of diseases, conditions, preventing a disease or a condition in a subject or other therapeutic applications for subjects in need thereof. Often the compositions and methods provided herein can be useful for treatment of hyperplastic conditions, including but not limited to, neoplasms, cancers, tumors and the like. In some embodiments, the subject has cancer. A condition, such as a cancer, can be associated with expression of a molecule on the cancer cells. Often, the molecule expressed by the cancer cells can comprise an extracellular portion capable of recognition by the antibody portion of the conjugate. A molecule expressed by the cancer cells can be a tumor antigen. An antibody portion of the conjugate can recognize a tumor antigen. A tumor antigen can include CD5, CD19, CD20, CD25, CD37, CD30, CD33, CD45, CAMPATH-1, HLD-DR, carcinoembryonic antigen (CEA), TAG-72, EpCAM, MUC1, MUC15, folate-binding protein, A33, G250, prostate-specific membrane antigen (PSMA), ferritin, GD2, GD3, GM2, Le^{y}, CA-125, CA19-9, epidermal growth factor, p185HER2, IL-2 receptor, fibroblast activation protein (FAP), tenascin, a metalloproteinase, endosialin, vascular endothelial growth factor, avB3, WT1, LMP2, HPV E6, HPV E7, EGFRvIII (de2-7 EGFR), Her-2/neu, idiotype, MAGE A3, p53 nonmutant, NY-ESO-1, MelanA/MART1, Ras mutant, gp100, p53 mutant, PR1, bcr-abl, tyrosinase, survivin, PSA, hTERT, a Sarcoma translocation breakpoint fusion protein, EphA2, PAP, ML-IAP, AFP, ERG, NA17, PAX3, ALK, androgen receptor, cyclin B1, polysialic acid, MYCN, RhoC, TRP-2, fucosyl GM1, mesothelin (MSLN), PSCA, MAGE A1, sLe(animal), CYP1B1, PLAV1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, B7H3, Legumain, Tie 3, Page4, VEGFR2, MAD-CT-1, PDGFR-B, MAD-CT-2, ROR2, CMET, HER3, EPCAM, CA6, NAPI2B, TROP2, CLDN18.2, RON, LY6E, FRA, DLL3, PTK7, LIV1, ROR1, Fos-related antigen 1, VEGFR, endoglin, PD-L1, CD204, CD206, CD301, VTCN1, or VISTA.

As described herein, an antigen binding domain portion of the construct, can be configured to recognize a molecule expressed by a cancer cell, such as for example, a disease antigen, tumor antigen or a cancer antigen. In some embodiments, the molecule is a disease antigen, tumor antigen or a cancer antigen, or fragment thereof. Often such antigens that are known to those of ordinary skill in the art, or newly found to be associated with such a condition, to be commonly associated with, and/or, specific to, such conditions. For example, a disease antigen, tumor antigen or a cancer antigen can be, but is not limited to, CD5, CD19, CD20, CD25, CD37, CD30, CD33, CD45, CAMPATH-1, HLD-DR, carcinoembryonic antigen (CEA), TAG-72, EpCAM, MUC1, MUC15, folate-binding protein, A33, G250, prostate-specific membrane antigen (PSMA), ferritin, GD2, GD3, GM2, Le^{y}, CA-125, CA19-9, epidermal growth factor, p185HER2, IL-2 receptor, fibroblast activation protein (FAP), tenascin, a metalloproteinase, endosialin, vascular endothelial growth factor, avB3, WT1, LMP2, HPV E6, HPV E7, EGFRvIII (de2-7 EGFR), Her-2/neu, idiotype, MAGE A3, p53 nonmutant, NY-ESO-1, MelanA/MART1, Ras mutant, gp100, p53 mutant, PR1, bcr-abl, tyrosinase, survivin, PSA, hTERT, a Sarcoma translocation breakpoint fusion protein, EphA2, PAP, ML-IAP, AFP, ERG, NA17, PAX3, ALK, androgen receptor, cyclin B1, polysialic acid, MYCN, RhoC, TRP-2, fucosyl GM1, mesothelin (MSLN), PSCA, MAGE A1, sLe(animal), CYP1B1, PLAV1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, B7H3, Legumain, Tie 3, Page4, VEGFR2, MAD-CT-1, PDGFR-B, MAD-CT-2, ROR2, CMET, HER3, EPCAM, CA6, NAPI2B, TROP2, CLDN18.2, RON, LY6E, FRA, DLL3, PTK7, LIV1, ROR1, Fos-related antigen 1, VEGFR, endoglin, PD-L1, CD204, CD206, CD301, VTCN1, or VISTA. Additionally, such tumor antigens can be derived from the following specific conditions and/or families of conditions, including but not limited to, cancers such as brain cancers, skin cancers, lymphomas, sarcomas, lung cancer, liver cancer, leukemias, uterine cancer, breast cancer, ovarian cancer, cervical cancer, bladder cancer, kidney cancer, hemangiosarcomas, bone cancers, blood cancers, testicular cancer, prostate cancer, stomach cancer, intestinal cancers, pancreatic cancer, and other types of cancers as well as pre-cancerous conditions such as hyperplasia or the like.

In some embodiments, the antigen binding domain of a construct-peptide compositions, conjugates, or pharmaceutical specifically binds to a target antigen or fragment thereof of a cancer cell, such as a cell from Acute lymphoblastic leukemia (ALL); Acute myeloid leukemia; Adrenocortical carcinoma; Astrocytoma, childhood cerebellar or cerebral; Basal-cell carcinoma; Bladder cancer; Bone tumor, osteosarcoma/malignant fibrous histiocytoma; Brain cancer; Brain tumors, such as, cerebellar astrocytoma, malignant glioma, ependymoma, medulloblastoma, visual pathway and hypothalamic glioma; Brainstem glioma; Breast cancer; Bronchial adenomas/carcinoids; Burkitt's lymphoma; Cerebellar astrocytoma; Cervical cancer; Cholangiocarcinoma; Chondrosarcoma; Chronic lymphocytic leukemia; Chronic myelogenous leukemia; Chronic myeloproliferative disorders; Colon cancer; Cutaneous T-cell lymphoma; Endometrial cancer; Ependymoma; Esophageal cancer; Eye cancers, such as, intraocular melanoma and retinoblastoma; Gallbladder cancer; Glioma; Hairy cell leukemia; Head and neck cancer; Heart cancer; Hepatocellular (liver) cancer; Hodgkin lymphoma; Hypopharyngeal cancer; Islet cell carcinoma (endocrine pancreas); Kaposi sarcoma; Kidney cancer (renal cell cancer); Laryngeal cancer; Leukaemia, such as, acute lymphoblastic, acute myeloid, chronic lymphocytic, chronic myelogenous and, hairy cell; Lip and oral cavity cancer; Liposarcoma; Lung cancer, such as, non-small cell and small cell; Lymphoma, such as, AIDS-related, Burkitt; Lymphoma, cutaneous T-Cell, Hodgkin and Non-Hodgkin, Macroglobulinemia, Malignant fibrous histiocytoma of bone/osteosarcoma; Melanoma; Merkel cell cancer; Mesothelioma; Multiple myeloma/plasma cell neoplasm; Mycosis fungoides; Myelodysplastic syndromes; Myelodysplastic/myeloproliferative diseases; Myeloproliferative disorders, chronic; Nasal cavity and paranasal sinus cancer; Nasopharyngeal carcinoma; Neuroblastoma; Oligodendroglioma; Oropharyngeal cancer; Osteosarcoma/malignant fibrous histiocytoma of bone; Ovarian cancer; Pancreatic cancer; Parathyroid cancer; Pharyngeal cancer; Pheochromocytoma; Pituitary adenoma; Plasma cell neoplasia; Pleuropulmonary blastoma; Prostate cancer; Rectal cancer; Renal cell carcinoma (kidney cancer); Renal pelvis and ureter, transitional cell cancer; Rhabdomyosarcoma; Salivary gland cancer; Sarcoma, Ewing family of tumors; Sarcoma, Kaposi; Sarcoma, soft tissue; Sarcoma, uterine; Sezary syndrome; Skin cancer (non-melanoma); Skin carcinoma; Small intestine cancer; Soft tissue sarcoma; Squamous cell carcinoma; Squamous neck cancer with occult primary, metastatic; Stomach cancer; Testicular cancer; Throat cancer; Thymoma and thymic carcinoma; Thymoma; Thyroid cancer; Thyroid cancer, childhood; Uterine cancer; Vaginal cancer; Waldenstrom macroglobulinemia; or Wilms tumor.

Non-limiting examples of cancers that can be treated by a construct-peptide composition can include Acute lymphoblastic leukemia (ALL); Acute myeloid leukemia; Adrenocortical carcinoma; Astrocytoma, childhood cerebellar or cerebral; Basal-cell carcinoma; Bladder cancer; Bone tumor, osteosarcoma/malignant fibrous histiocytoma; Brain cancer; Brain tumors, such as, cerebellar astrocytoma, malignant glioma, ependymoma, medulloblastoma, visual pathway and hypothalamic glioma; Brainstem glioma; Breast cancer; Bronchial adenomas/carcinoids; Burkitt's lymphoma; Cerebellar astrocytoma; Cervical cancer; Cholangiocarcinoma; Chondrosarcoma; Chronic lymphocytic leukemia; Chronic myelogenous leukemia; Chronic myeloproliferative disorders; Colon cancer; Cutaneous T-cell lymphoma; Endometrial cancer; Ependymoma; Esophageal cancer; Eye cancers, such as, intraocular melanoma and retinoblastoma; Gallbladder cancer; Glioma; Hairy cell leukemia; Head and neck cancer; Heart cancer; Hepatocellular (liver) cancer; Hodgkin lymphoma; Hypopharyngeal cancer; Islet cell carcinoma (endocrine pancreas); Kaposi sarcoma; Kidney cancer (renal cell cancer); Laryngeal cancer; Leukaemia, such as, acute lymphoblastic, acute myeloid, chronic lymphocytic, chronic myelogenous and, hairy cell; Lip and oral cavity cancer; Liposarcoma; Lung cancer, such as, non-small cell and small cell; Lymphoma, such as, AIDS-related, Burkitt; Lymphoma, cutaneous T-Cell, Hodgkin and Non-Hodgkin, Macroglobulinemia, Malignant fibrous histiocytoma of bone/osteosarcoma; Melanoma; Merkel cell cancer; Mesothelioma; Multiple myeloma/plasma cell neoplasm; Mycosis fungoides; Myelodysplastic syndromes; Myelodysplastic/myeloproliferative diseases; Myeloproliferative disorders, chronic; Nasal cavity and paranasal sinus cancer; Nasopharyngeal carcinoma; Neuroblastoma; Oligodendroglioma; Oropharyngeal cancer; Osteosarcoma/malignant fibrous histiocytoma of bone; Ovarian cancer; Pancreatic cancer; Parathyroid cancer; Pharyngeal cancer; Pheochromocytoma; Pituitary adenoma; Plasma cell neoplasia; Pleuropulmonary blastoma; Prostate cancer; Rectal cancer; Renal cell carcinoma (kidney cancer); Renal pelvis and ureter, transitional cell cancer; Rhabdomyosarcoma; Salivary gland cancer; Sarcoma, Ewing family of tumors; Sarcoma, Kaposi; Sarcoma, soft tissue; Sarcoma, uterine; Sezary syndrome; Skin cancer (non-melanoma); Skin carcinoma; Small intestine cancer; Soft tissue sarcoma; Squamous cell carcinoma; Squamous neck cancer with occult primary, metastatic; Stomach cancer; Testicular cancer; Throat cancer; Thymoma and thymic carcinoma; Thymoma; Thyroid cancer; Thyroid cancer, childhood; Uterine cancer; Vaginal cancer; Waldenstrom macroglobulinemia; Wilms tumor and any combination thereof.

### EXAMPLES

The following examples are included to further describe some embodiments of the present disclosure, and should not be used to limit the scope of the disclosure

### Example 1

### Peptide with a Cancer Sequence from a Subject's Cancer

This example describes a peptide with a cancer sequence that is from a subject's cancer. A subject is diagnosed with cancer, and a biopsy of the cancer is taken from the subject. The biopsy is sequenced. A mutation is identified in the biopsied cells by comparing the sequences of the subject's normal cells to the sequences of the cancer cells. A sequence with the mutation is produced for making a peptide with the cancer sequence. Cells are transfected with vectors containing the sequence for the peptide with the cancer sequence. The peptide with the cancer sequence is then purified from the transfected cells. The peptide with the cancer sequence is conjugated to an antibody.

Alternatively, the cancer sequence is inserted into a vector containing the sequence of an antibody. The cancer sequence is inserted into the vector to allow for the production of a fusion protein in which the fusion protein is the antibody and the peptide with the cancer sequence.

### Example 2

### Neo-Antigen Peptide with a Cancer Sequence from a Subject's Cancer

This example describes the identification of a peptide that is a neo-antigen (neoAg) from a subject. A mutanome (mutational spectrum of individual tumors) is used to identify nonsynonymous mutations in a cancer from a subject. From the nonsynonymous mutations, neo-epitopes are identified, which are specific to the subject's cancer. Neural network algorithms are applied to predict high affinity neo-epitopes derived from mutated genes that can bind to the subject's own MHC molecules. Alternatively, the MHC ligandome of a tumor cell is analyzed, in which peptides from the MHC molecules derived from a tumor tissue of a subject is eluted followed by reverse phase HPLC fractionation and mass spectrometry for identification.

After identification of a potential neoAg, the neoAg is further tested to determine if it is a suitable neoAg to induce an immune response in the subject. This is done by synthesizing the neoAg and then immunity of the subject is tested against the neoAgs using functional assays. The subject's own T cells are used in these functional assays, in which the neoAg or mRNA of the neoAg is used to pulse and transduce antigen-presenting cells. The antigen-presenting cells and the subject's T cells are co-cultured to test whether neoAg-specific CD4⁺ or CD8⁺ T cells are produced. These neo-Ag-specific CD4⁺ or CD8⁺ T cells are also tested for stimulation by their ability to expand and to produce cytokines after stimulation with the neoAg.

The sequence of the neoAg that successfully produces and stimulates CD4⁺ or CD8⁺ T cells is inserted into a vector, and then cells are transfected with these vectors. The neoAg is produced as a peptide and is then purified from the transfected cells. The peptide with the neoAg sequence is conjugated to an antibody.

Alternatively, the neoAg sequence is inserted into a vector containing the sequence of an antibody. The cancer sequence is inserted into the vector to allow for the production of a fusion protein in which the fusion protein is the antibody and the peptide with the cancer sequence.

### Example 3

### Peptide with a Common Cancer Sequence

This example describes a peptide with a cancer sequence contains a mutation that is common in a certain type of cancer. A mutation that is common in a certain type of cancer is identified. A sequence containing the mutation is produced that has the mutation in the middle of sequence. A sequence with the mutation is produced for making a peptide with the cancer sequence. Cells are transfected with vectors containing the sequence for the peptide with the cancer sequence. The peptide with the cancer sequence is then purified from the transfected cells. The peptide with the cancer sequence is conjugated to an antibody.

Alternatively, the cancer sequence is inserted into a vector containing the sequence of an antibody. The cancer sequence is inserted into the vector to allow for the production of a fusion protein in which the fusion protein is the antibody and the peptide with the cancer sequence.

### Example 4

### Anti-CD40 Antibody-Peptide Composition

This example illustrates an anti-CD40 antibody-peptide composition. An anti-CD40 antibody is comprised of two SBT-040-G1WT heavy chains and two light chains from a SBT-040 antibody, which is referred to as a SBT-040-WT antibody. An anti-CD40 antibody is comprised of two SBT-040-G1VLPLL heavy chains and two light chains from a SBT-040 antibody, which is referred to as a SBT-040-VLPLL antibody. An anti-CD40 antibody is comprised of two SBT-040-G1DE heavy chains and two light chains from a SBT-040 antibody, which is referred to as a SBT-040-DE antibody. An anti-CD40 antibody is comprised of two SBT-040-G1AAA heavy chains and two light chains from a SBT-040 antibody, which is referred to as a SBT-040-AAA antibody. A peptide with a cancer sequence as described in Example 1, Example 2, or Example 3 is conjugated to SBT-040-WT antibody, a SBT-040-VLPLL antibody, a SBT-040-DE antibody, or a SBT-040-AAA antibody.

### Example 5

### Affinity of Anti-CD40 Antibody-Peptide Compositions to CD40

This example illustrates the binding affinity of an antigen binding domain of an anti-CD40 antibody-peptide composition to CD40. Each anti-CD40 antibody-peptide composition as described in Example 4 is measured for affinity to CD40. These affinities are measured by experiments using surface plasmon resonance. In these experiments, biotinylated recombinant CD40 is immobilized on a streptavidin-coated surface. The ability of each anti-CD40 antibody-peptide composition to bind to recombinant CD40 is then measured by surface plasmon resonance using a Biacore instrument. Recombinant CD40 is bound by each anti-CD40 antibody-peptide composition. Therefore, the ability of each anti-CD40 antibody-peptide composition to bind to CD40 is not interfered with by peptide in the composition is shown by the surface plasmon resonance data.

### Example 6

### Fc Receptor Binding to Fc Domain of an Anti-CD40 Antibody-Peptide Composition

This example illustrates the binding of an Fc domain of an anti-CD40 antibody-peptide composition to an Fc Receptor. Each anti-CD40 antibody-peptide composition as described in Example 4 is characterized for the ability of their Fc domains to bind to and for their affinity for soluble glycosylated FcγR ectodomains from all human FcγRs. This is shown by performing surface plasmon resonance experiments. In these experiments, biotinylated soluble glycosylated FcγR ectodomains from all human FcγRs are immobilized on a streptavidin-coated surface. The ability of each anti-CD40 antibody-peptide composition to bind to soluble glycosylated FcγR ectodomains from all human FcγRs is then measured by surface plasmon resonance using a Biacore instrument. The soluble glycosylated FcγR ectodomains from all human FcγRs is bound by each of the antibody-peptide compositions. Therefore, the surface plasmon resonance experiments show that the ability of the Fc domain of the anti-CD40 antibody component of the anti-CD40 antibody-peptide composition to bind to all human FcγRs is not interfered with by linking the peptide to the anti-CD40 antibody. The affinity of each anti-CD40 antibody as described in Example 4 for each human FcγRs is also shown by the surface plasmon resonance experiments. These affinity measurements for each anti-CD40 antibody-peptide composition are compared with the affinity measurements for each anti-CD40 antibody. The similarity in affinity of each anti-CD40 antibody-peptide composition for soluble glycosylated FcγR ectodomains from all human FcγRs with the affinity of each corresponding anti-CD40 antibody alone for soluble glycosylated FcγR ectodomains from all human FcγRs is shown by this comparison.

### Example 7

### Anti-DEC205 Antibody-Peptide Composition

This example illustrates an anti-DEC205 antibody-peptide composition. An anti-DEC205 antibody is comprised of two heavy chains and two light chains from an anti-DEC205 antibody, which is referred to as an anti-DEC205-WT antibody. An anti-DEC205 antibody is comprised of two heavy chains with mutations in which CD32a binding is enhanced and two light chains from an anti-DEC205 antibody, which is referred to as an anti-DEC205-32a antibody. An anti-DEC205 antibody is comprised of two heavy chains with mutations in which CD32b binding is enhanced and two light chains from an anti-DEC205 antibody, which is referred to as an anti-DEC205-32b antibody. A peptide with a cancer sequence as described in Example 1, Example 2, or Example 3 is conjugated to an anti-DEC205-WT antibody, an anti-DEC205-32a antibody, or an anti-DEC205-32b antibody.

### Example 8

### Affinity of Anti-DEC205 Antibody-Peptide Compositions to DEC205

This example illustrates the binding affinity of an antigen binding domain of an anti-DEC205 antibody-peptide composition to DEC205. Each anti-DEC205 antibody-peptide composition as described in Example 7 is measured for affinity to DEC205. These affinities are measured by experiments using surface plasmon resonance. In these experiments, biotinylated recombinant DEC205 is immobilized on a streptavidin-coated surface. The ability of each anti-DEC205 antibody-peptide composition to bind to recombinant DEC205 is then measured by surface plasmon resonance using a Biacore instrument. Recombinant DEC205 is bound by each anti-DEC205 antibody-peptide composition. Therefore, the ability of each anti-DEC205 antibody-peptide composition to bind to DEC205 is not interfered with by peptide in the composition is shown by the surface plasmon resonance data.

### Example 9

### Fc Receptor Binding to Fc Domain of an Anti-DEC205 Antibody-Peptide Composition

This example illustrates the binding of an Fc domain of an anti-DEC205 antibody-peptide composition to an Fc Receptor. Each anti-DEC205 antibody-peptide composition as described in Example 7 is characterized for the ability of their Fc domains to bind to and for their affinity for soluble glycosylated FcγR ectodomains from all human FcγRs. This is shown by performing surface plasmon resonance experiments. In these experiments, biotinylated soluble glycosylated FcγR ectodomains from all human FcγRs are immobilized on a streptavidin-coated surface. The ability of each anti-DEC205 antibody-peptide composition to bind to soluble glycosylated FcγR ectodomains from all human FcγRs is then measured by surface plasmon resonance using a Biacore instrument. The soluble glycosylated FcγR ectodomains from all human FcγRs is bound by each of the antibody-peptide compositions. Therefore, the surface plasmon resonance experiments show that the ability of the Fc domain of the anti-DEC205 antibody component of the anti-DEC205 antibody-peptide composition to bind to all human FcγRs is not interfered with by linking the peptide to the anti-DEC205 antibody. The affinity of each anti-DEC205 antibody as described in Example 7 for each human FcγRs is also shown by the surface plasmon resonance experiments. These affinity measurements for each anti-DEC205 antibody-peptide composition are compared with the affinity measurements for each anti-DEC205 antibody. The similarity in affinity of each anti-DEC205 antibody-peptide composition for soluble glycosylated FcγR ectodomains from all human FcγRs with the affinity of each corresponding anti-DEC205 antibody alone for soluble glycosylated FcγR ectodomains from all human FcγRs is shown by this comparison.

### Example 10

### Anti-DICR Antibody-Peptide Composition

This example illustrates an anti-DICR antibody-peptide composition. An anti-DEC205 antibody is comprised of two heavy chains and two light chains from an anti-DICR antibody, which is referred to as an anti-DICR-WT antibody. An anti-DICR antibody is comprised of two heavy chains with mutations in which CD32a binding is enhanced and two light chains from an anti-DICR antibody, which is referred to as an anti-DICR-32a antibody. An anti-DICR antibody is comprised of two heavy chains with mutations in which CD32b binding is enhanced and two light chains from an anti-DICR antibody, which is referred to as an anti-DICR-32b antibody. A peptide with a cancer sequence as described in Example 1, Example 2, or Example 3 is conjugated to an anti-DICR-WT antibody, an anti-DICR-32a antibody, or an anti-DICR-32b antibody.

### Example 11

### Affinity of Anti-DICR Antibody-Peptide Compositions to DICR

This example illustrates the binding affinity of an antigen binding domain of an anti-DICR antibody-peptide composition to DICR. Each anti-DICR antibody-peptide composition as described in Example 10 is measured for affinity to DICR. These affinities are measured by experiments using surface plasmon resonance. In these experiments, biotinylated recombinant DICR is immobilized on a streptavidin-coated surface. The ability of each anti-DICR antibody-peptide composition to bind to recombinant DICR is then measured by surface plasmon resonance using a Biacore instrument. Recombinant DICR is bound by each anti-DICR antibody-peptide composition. Therefore, the ability of each anti-DICR antibody-peptide composition to bind to DICR is not interfered with by peptide in the composition is shown by the surface plasmon resonance data.

### Example 12

### Fc Receptor Binding to Fc Domain of an Anti-DICR Antibody-Peptide Composition

This example illustrates the binding of an Fc domain of an anti-DICR antibody-peptide composition to an Fc Receptor. Each anti-DICR antibody-peptide composition as described in Example 10 is characterized for the ability of their Fc domains to bind to and for their affinity for soluble glycosylated FcγR ectodomains from all human FcγRs. This is shown by performing surface plasmon resonance experiments. In these experiments, biotinylated soluble glycosylated FcγR ectodomains from all human FcγRs are immobilized on a streptavidin-coated surface. The ability of each anti-DICR antibody-peptide composition to bind to soluble glycosylated FcγR ectodomains from all human FcγRs is then measured by surface plasmon resonance using a Biacore instrument. The soluble glycosylated FcγR ectodomains from all human FcγRs is bound by each of the antibody-peptide compositions. Therefore, the surface plasmon resonance experiments show that the ability of the Fc domain of the anti-DICR antibody component of the anti-DICR antibody-peptide composition to bind to all human FcγRs is not interfered with by linking the peptide to the anti-DICR antibody. The affinity of each anti-DICR antibody as described in Example 10 for each human FcγRs is also shown by the surface plasmon resonance experiments. These affinity measurements for each anti-DICR antibody-peptide composition are compared with the affinity measurements for each anti-DICR antibody. The similarity in affinity of each anti-DICR antibody-peptide composition for soluble glycosylated FcγR ectodomains from all human FcγRs with the affinity of each corresponding anti-DICR antibody alone for soluble glycosylated FcγR ectodomains from all human FcγRs is shown by this comparison.

### Example 13

### Anti-DNGR-1 Antibody-Peptide Composition

This example illustrates an anti-DNGR-1 antibody-peptide composition. An anti-DNGR-1 antibody is comprised of two heavy chains and two light chains from an anti-DNGR-1 antibody, which is referred to as an anti-DNGR-1-WT antibody. An anti-DNGR-1 antibody is comprised of two heavy chains with mutations in which CD32a binding is enhanced and two light chains from an anti-DNGR-1 antibody, which is referred to as an anti-DNGR-1-32a antibody. An anti-DNGR-1 antibody is comprised of two heavy chains with mutations in which CD32b binding is enhanced and two light chains from an anti-DNGR-1 antibody, which is referred to as an anti-DNGR-1-32b antibody. A peptide with a cancer sequence as described in Example 1, Example 2, or Example 3 is conjugated to an anti-DNGR-1-WT antibody, an anti-DNGR-1-32a antibody, or an anti-DNGR-1-32b antibody.

### Example 14

### Affinity of Anti-DNGR-1 Antibody-Peptide Compositions to DNGR-1

This example illustrates the binding affinity of an antigen binding domain of an anti-DNGR-1 antibody-peptide composition to DNGR-1. Each anti-DNGR-1 antibody-peptide composition as described in Example 13 is measured for affinity to DNGR-1. These affinities are measured by experiments using surface plasmon resonance. In these experiments, biotinylated recombinant DNGR-1 is immobilized on a streptavidin-coated surface. The ability of each anti-DNGR-1 antibody-peptide composition to bind to recombinant DNGR-1 is then measured by surface plasmon resonance using a Biacore instrument. Recombinant DNGR-1 is bound by each anti-DNGR-1 antibody-peptide composition. Therefore, the ability of each anti-DNGR-1 antibody-peptide composition to bind to DNGR-1 is not interfered with by peptide in the composition is shown by the surface plasmon resonance data.

### Example 15

### Fc Receptor Binding to Fc Domain of an Anti-DNGR-1 Antibody-Peptide Composition

This example illustrates the binding of an Fc domain of an anti-CD40 antibody-peptide composition to an Fc Receptor. Each anti-CD40 antibody-peptide composition as described in Example 3 is characterized for the ability of their Fc domains to bind to and for their affinity for soluble glycosylated FcγR ectodomains from all human FcγRs. This is shown by performing surface plasmon resonance experiments. In these experiments, biotinylated soluble glycosylated FcγR ectodomains from all human FcγRs are immobilized on a streptavidin-coated surface. The ability of each anti-CD40 antibody-peptide composition to bind to soluble glycosylated FcγR ectodomains from all human FcγRs is then measured by surface plasmon resonance using a Biacore instrument. The soluble glycosylated FcγR ectodomains from all human FcγRs is bound by each of the antibody-peptide compositions. Therefore, the surface plasmon resonance experiments show that the ability of the Fc domain of the anti-CD40 antibody component of the anti-CD40 antibody-peptide composition to bind to all human FcγRs is not interfered with by linking the peptide to the anti-CD40 antibody. The affinity of each anti-CD40 antibody as described in Example 3 for each human FcγRs is also shown by the surface plasmon resonance experiments. These affinity measurements for each anti-CD40 antibody-peptide composition are compared with the affinity measurements for each anti-CD40 antibody. The similarity in affinity of each anti-CD40 antibody-peptide composition for soluble glycosylated FcγR ectodomains from all human FcγRs with the affinity of each corresponding anti-CD40 antibody alone for soluble glycosylated FcγR ectodomains from all human FcγRs is shown by this comparison.

### Example 16

### Anti-BDCA-2 Antibody-Peptide Composition

This example illustrates an anti-BDCA-2 antibody-peptide composition. An anti-BDCA-2 antibody is comprised of two heavy chains and two light chains from an anti-BDCA-2 antibody, which is referred to as an anti-BDCA-2-WT antibody. An anti-BDCA-2 antibody is comprised of two heavy chains with mutations in which CD32a binding is enhanced and two light chains from an anti-BDCA-2 antibody, which is referred to as an anti-BDCA-2-32a antibody. An anti-BDCA-2 antibody is comprised of two heavy chains with mutations in which CD32b binding is enhanced and two light chains from an anti-BDCA-2 antibody, which is referred to as an anti-BDCA-2-32b antibody. A peptide with a cancer sequence as described in Example 1, Example 2, or Example 3 is conjugated to an anti-BDCA-2 antibody, an anti-BDCA-2-32a antibody, or an anti-BDCA-2-32b antibody.

### Example 17

### Affinity of Anti-BDCA-2 Antibody-Peptide Compositions to BDCA-2

This example illustrates the binding affinity of an antigen binding domain of an anti-BDCA-2 antibody-peptide composition to BDCA-2. Each anti-BDCA-2 antibody-peptide composition as described in Example 16 is measured for affinity to BDCA-2. These affinities are measured by experiments using surface plasmon resonance. In these experiments, biotinylated recombinant BDCA-2 is immobilized on a streptavidin-coated surface. The ability of each anti-BDCA-2 antibody-peptide composition to bind to recombinant BDCA-2 is then measured by surface plasmon resonance using a Biacore instrument. Recombinant BDCA-2 is bound by each anti-BDCA-2 antibody-peptide composition. Therefore, the ability of each anti-BDCA-2 antibody-peptide composition to bind to BDCA-2 is not interfered with by peptide in the composition is shown by the surface plasmon resonance data.

### Example 18

### Fc Receptor Binding to Fc Domain of an Anti-BDCA-2 Antibody-Peptide Composition

This example illustrates the binding of an Fc domain of an anti-BDCA-2 antibody-peptide composition to an Fc Receptor. Each anti-BDCA-2 antibody-peptide composition as described in Example 16 is characterized for the ability of their Fc domains to bind to and for their affinity for soluble glycosylated FcγR ectodomains from all human FcγRs. This is shown by performing surface plasmon resonance experiments. In these experiments, biotinylated soluble glycosylated FcγR ectodomains from all human FcγRs are immobilized on a streptavidin-coated surface. The ability of each anti-BDCA-2 antibody-peptide composition to bind to soluble glycosylated FcγR ectodomains from all human FcγRs is then measured by surface plasmon resonance using a Biacore instrument. The soluble glycosylated FcγR ectodomains from all human FcγRs is bound by each of the antibody-peptide compositions. Therefore, the surface plasmon resonance experiments show that the ability of the Fc domain of the anti-BDCA-2 antibody component of the anti-BDCA-2 antibody-peptide composition to bind to all human FcγRs is not interfered with by linking the peptide to the anti-BDCA-2 antibody. The affinity of each anti-BDCA-2 antibody as described in Example 16 for each human FcγRs is also shown by the surface plasmon resonance experiments. These affinity measurements for each anti-BDCA-2 antibody-peptide composition are compared with the affinity measurements for each anti-BDCA-2 antibody. The similarity in affinity of each anti-BDCA-2 antibody-peptide composition for soluble glycosylated FcγR ectodomains from all human FcγRs with the affinity of each corresponding anti-BDCA-2 antibody alone for soluble glycosylated FcγR ectodomains from all human FcγRs is shown by this comparison.

### Example 19

### Lysine-Based Bioconjugation

The example illustrates lysine-based bioconjugation of an immune-stimulatory compound with a construct-peptide composition. The construct-peptide composition is exchanged into an appropriate buffer, for example, phosphate, borate, PBS, Tris-Acetate at a concentration of about 2 mg/mL to about 10 mg/mL. An appropriate number of equivalents of the immune stimulatory compound-linker are added as a solution with stirring. Dependent on the physical properties of the immune stimulatory compound-linker, a co-solvent can be introduced prior to the addition of the immune stimulatory compound-linker to facilitate solubility. The reaction is stirred at room temperature for 2 hours to about 12 hours depending on the observed reactivity. The progression of the reaction is monitored by LC-MS. Once the reaction has been deemed complete, the remaining immune stimulatory compound-linkers are removed by applicable methods and the construct-peptide composition conjugated with immune-stimulatory compound is exchanged into the desired formulation buffer.

### Example 20

### Cysteine-Based Bioconjugation

The example illustrates cysteine-based bioconjugation of an immune-stimulatory compound with a construct-peptide composition. The construct-peptide composition is exchanged into an appropriate buffer, for example, phosphate, borate, PBS, Tris-Acetate at a concentration of about 2 mg/mL to about 10 mg/mL with an appropriate number of equivalents of a reducing agent, for example, dithiothreitol or tris(2-carboxyethyl)phosphine. The resultant solution is stirred for an appropriate amount of time and temperature to effect the desired reduction. The immune stimulatory compound-linker construct is added as a solution with stirring. Dependent on the physical properties of the immune stimulatory compound-linker, a co-solvent can be introduced prior to the addition of the immune stimulatory compound-linker to facilitate solubility. The reaction is stirred at room temperature for about 1 hour to about 12 hours depending on the observed reactivity. The progression of the reaction is monitored by liquid chromatography-mass spectrometry (LC-MS). Once the reaction has been deemed complete, the remaining free immune stimulatory compound-linker is removed by applicable methods and the construct-peptide composition conjugated with immune-stimulatory compound is exchanged into the desired formulation buffer.

### Example 21

### Anti-CD40 Antibody-Peptide Composition Conjugated with Immune-Stimulatory Compound

This example describes an anti-CD40 antibody-peptide composition conjugated with an immune stimulatory compound. An anti-CD40 antibody-peptide composition as described in Example 4 is conjugated to an immune-stimulatory compound by lysine-based bioconjugation as described in Example 19. An anti-C40 antibody-peptide composition as described in Example 4 is conjugated to an immune-stimulatory compound by cysteine-based bioconjugation as described in Example 20.

### Example 22

### Affinity of Anti-CD40 Antibody-Peptide Composition Conjugated with Immune-Stimulatory Compound to CD40

This example illustrates the binding affinity of an antigen binding domain of an anti-CD40 antibody-peptide composition conjugated with immune stimulatory compound to CD40. Each anti-CD40 antibody-peptide composition conjugated with immune-stimulatory compound as described in Example 21 is measured for affinity to CD40. These affinities are measured by experiments using surface plasmon resonance. In these experiments, biotinylated recombinant CD40 is immobilized on a streptavidin-coated surface. The ability of each anti-CD40 antibody-peptide composition conjugated with immune-stimulatory compound to bind to recombinant CD40 is then measured by surface plasmon resonance using a Biacore instrument. Recombinant CD40 is bound by each anti-CD40 antibody-peptide composition conjugated with immune-stimulatory compound. Therefore, the ability of each anti-CD40 antibody-peptide composition conjugated with immune-stimulatory compound to bind to CD40 is not interfered with by peptide in the composition or by conjugation with immune-stimulatory compound is shown by the surface plasmon resonance data.

### Example 23

### Fc Receptor Binding to Fc Domain of an Anti-CD40 Antibody-Peptide Composition Conjugated with Immune-Stimulatory Compound

This example illustrates the binding of an Fc domain of an anti-CD40 antibody-peptide composition conjugated with immune-stimulatory compound to Fc receptors. Each anti-CD40 antibody-peptide composition conjugated with immune-stimulatory compound as described in Example 21 is characterized for the ability of their Fc domains to bind to and for their affinity for soluble glycosylated FcγR ectodomains from all human FcγRs. This is shown by performing surface plasmon resonance experiments. In these experiments, biotinylated soluble glycosylated FcγR ectodomains from all human FcγRs are immobilized on a streptavidin-coated surface. The ability of each anti-CD40 antibody-peptide composition conjugated with immune-stimulatory compound to bind to soluble glycosylated FcγR ectodomains from all human FcγRs is then measured by surface plasmon resonance using a Biacore instrument. The soluble glycosylated FcγR ectodomains from all human FcγRs is bound by each of the antibody-peptide compositions conjugated with immune-stimulatory compound. Therefore, the surface plasmon resonance experiments show that the ability of the Fc domain of the anti-CD40 antibody component of the anti-CD40 antibody-peptide composition conjugated with immune-stimulatory compound to bind to all human FcγRs is not interfered with by linking the peptide to the anti-CD40 antibody or by conjugation of immune-stimulatory compounds with the anti-CD40 antibody-peptide composition. The affinity of each anti-CD40 antibody as described in Example 21 for each human FcγRs is also shown by the surface plasmon resonance experiments. These affinity measurements for each anti-CD40 antibody-peptide composition conjugated with immune-stimulatory compound are compared with the affinity measurements for each anti-CD40 antibody. The similarity in affinity of each anti-CD40 antibody-peptide composition conjugated with immune-stimulatory compound for soluble glycosylated FcγR ectodomains from all human FcγRs with the affinity of each corresponding anti-CD40 antibody alone for soluble glycosylated FcγR ectodomains from all human FcγRs is shown by this comparison.

### Example 24

### Cytokine Production is Enhanced by Anti-CD40 Antibody-Peptide Compositions Conjugated with Immune-Stimulatory Compound

This example shows cytokine production is enhanced by anti-CD40-antibody peptide compositions conjugated with immune-stimulatory compound. Dendritic cells (DCs) are derived from peripheral blood mononuclear cells (PBMCs). DCs are obtained by putting human PBMCs into a culture dish. The resulting adherent cells are washed with RPMI containing 10% fetal calf serum, and then are incubated for 7 days in complete medium containing 10 ng/mL IL-4 and 100 ng/mL GM-CSF. The non-adherent cells are isolated and are washed. These isolated cells are run by a flow cytometer to ensure CD11c expression, in which the DCs identity as DCs is confirmed by CD11c expression. The DCs are then incubated with either anti-CD40 antibody-peptide compositions conjugated with immune stimulatory compounds as described in Example 21 or the corresponding anti-CD40 antibodies alone. A non-binding isotype control antibody is also incubated with DCs as a control. Each culture is then incubated for 24 hours and the supernatant of each culture is analyzed using a cytokine bead array assay. Cytokine expression levels of IFNγ, IL-8, IL-12 and IL-2 are measured by the cytokine bead array assay. The level of cytokine expression in the supernatant of the culture containing the non-binding isotype control is decreased compared to supernatants from the cultures containing the anti-CD40-peptide compositions conjugated with immune-stimulatory compound. Additionally, the level of cytokine expression in the supernatant from cultures containing anti-CD40 antibodies alone is decreased as compared to the supernatant from cultures containing the anti-CD40 antibody-peptide composition conjugated with immune-stimulatory compound.

### Example 25

### Anti-DEC205 Antibody-Peptide Composition Conjugated with Immune-Stimulatory Compound

This example describes an anti-DEC205 antibody-peptide composition conjugated with an immune stimulatory compound. An anti-DEC205 antibody-peptide composition as described in Example 7 is conjugated to an immune-stimulatory compound by lysine-based bioconjugation as described in Example 19. An anti-DEC205 antibody-peptide composition as described in Example 7 is conjugated to an immune-stimulatory compound by cysteine-based bioconjugation as described in Example 20.

### Example 26

### Affinity of Anti-DEC205 Antibody-Peptide Composition Conjugated with Immune-Stimulatory Compound to DEC205

This example illustrates the binding affinity of an antigen binding domain of an anti-DEC205 antibody-peptide composition conjugated with immune stimulatory compound to DEC205. Each anti-DEC205 antibody-peptide composition conjugated with immune-stimulatory compound as described in Example 25 is measured for affinity to DEC205. These affinities are measured by experiments using surface plasmon resonance. In these experiments, biotinylated recombinant DEC205 is immobilized on a streptavidin-coated surface. The ability of each anti-DEC205 antibody-peptide composition conjugated with immune-stimulatory compound to bind to recombinant DEC205 is then measured by surface plasmon resonance using a Biacore instrument. Recombinant DEC205 is bound by each anti-DEC205 antibody-peptide composition conjugated with immune-stimulatory compound. Therefore, the ability of each anti-DEC205 antibody-peptide composition conjugated with immune-stimulatory compound to bind to DEC205 is not interfered with by peptide in the composition or by conjugation with immune-stimulatory compound is shown by the surface plasmon resonance data.

### Example 27

### Fc Receptor Binding to Fc Domain of an Anti-DEC205 Antibody-Peptide Composition Conjugated with Immune-Stimulatory Compound

This example illustrates the binding of an Fc domain of an anti-DEC205 antibody-peptide composition conjugated with immune-stimulatory compound to Fc receptors. Each anti-DEC205 antibody-peptide composition conjugated with immune-stimulatory compound as described in Example 25 is characterized for the ability of their Fc domains to bind to and for their affinity for soluble glycosylated FcγR ectodomains from all human FcγRs. This is shown by performing surface plasmon resonance experiments. In these experiments, biotinylated soluble glycosylated FcγR ectodomains from all human FcγRs are immobilized on a streptavidin-coated surface. The ability of each anti-DEC205 antibody-peptide composition conjugated with immune-stimulatory compound to bind to soluble glycosylated FcγR ectodomains from all human FcγRs is then measured by surface plasmon resonance using a Biacore instrument. The soluble glycosylated FcγR ectodomains from all human FcγRs is bound by each of the antibody-peptide compositions conjugated with immune-stimulatory compound. Therefore, the surface plasmon resonance experiments show that the ability of the Fc domain of the anti-DEC205 antibody component of the anti-DEC205 antibody-peptide composition conjugated with immune-stimulatory compound to bind to all human FcγRs is not interfered with by linking the peptide to the anti-DEC205 antibody or by conjugation of immune-stimulatory compounds with the anti-DEC205 antibody-peptide composition. The affinity of each anti-DEC205 antibody as described in Example 25 for each human FcγRs is also shown by the surface plasmon resonance experiments. These affinity measurements for each anti-DEC205 antibody-peptide composition conjugated with immune-stimulatory compound are compared with the affinity measurements for each anti-DEC205 antibody. The similarity in affinity of each anti-DEC205 antibody-peptide composition conjugated with immune-stimulatory compound for soluble glycosylated FcγR ectodomains from all human FcγRs with the affinity of each corresponding anti-DEC205 antibody alone for soluble glycosylated FcγR ectodomains from all human FcγRs is shown by this comparison.

### Example 28

### Cytokine Production is Enhanced by Anti-DEC205 Antibody-Peptide Compositions Conjugated with Immune-Stimulatory Compound

This example shows cytokine production is enhanced by anti-DEC205-antibody peptide compositions conjugated with immune-stimulatory compound. Dendritic cells (DCs) are derived from peripheral blood mononuclear cells (PBMCs). DCs are obtained by putting human PBMCs into a culture dish. The resulting adherent cells are washed with RPMI containing 10% fetal calf serum, and then are incubated for 7 days in complete medium containing 10 ng/mL IL-4 and 100 ng/mL GM-CSF. The non-adherent cells are isolated and are washed. These isolated cells are run by a flow cytometer to ensure CD11c expression, in which the DCs identity as DCs is confirmed by CD11c expression. The DCs are then incubated with either anti-DEC205 antibody-peptide compositions conjugated with immune stimulatory compounds as described in Example 25, or the corresponding anti-DEC205 antibodies alone. A non-binding isotype control antibody is also incubated with DCs as a control. Each culture is then incubated for 24 hours and the supernatant of each culture is analyzed using a cytokine bead array assay. Cytokine expression levels of IFNγ, IL-8, IL-12 and IL-2 are measured by the cytokine bead array assay. The level of cytokine expression in the supernatant of the culture containing the non-binding isotype control is decreased compared to supernatants from the cultures containing the anti-DEC205-peptide compositions conjugated with immune-stimulatory compound. Additionally, the level of cytokine expression in the supernatant from cultures containing anti-DEC205 antibodies alone is decreased as compared to the supernatant from cultures containing the anti-DEC205 antibody-peptide composition conjugated with immune-stimulatory compound.

### Example 29

### Anti-DICR Antibody-Peptide Composition Conjugated with Immune-Stimulatory Compound

This example describes an anti-DICR antibody-peptide composition conjugated with an immune stimulatory compound. An anti-DICR antibody-peptide composition as described in Example 10 is conjugated to an immune-stimulatory compound by lysine-based bioconjugation as described in Example 19. An anti-DICR antibody-peptide composition as described in Example 10 is conjugated to an immune-stimulatory compound by cysteine-based bioconjugation as described in Example 20.

### Example 30

### Affinity of Anti-DICR Antibody-Peptide Composition Conjugated with Immune-Stimulatory Compound to DICR

This example illustrates the binding affinity of an antigen binding domain of an anti-DICR antibody-peptide composition conjugated with immune stimulatory compound to DICR. Each anti-DICR antibody-peptide composition conjugated with immune-stimulatory compound as described in Example 29 is measured for affinity to DICR. These affinities are measured by experiments using surface plasmon resonance. In these experiments, biotinylated recombinant DICR is immobilized on a streptavidin-coated surface. The ability of each anti-DICR antibody-peptide composition conjugated with immune-stimulatory compound to bind to recombinant DICR is then measured by surface plasmon resonance using a Biacore instrument. Recombinant DICR is bound by each anti-DICR antibody-peptide composition conjugated with immune-stimulatory compound. Therefore, the ability of each anti-DICR antibody-peptide composition conjugated with immune-stimulatory compound to bind to DICR is not interfered with by peptide in the composition or by conjugation with immune-stimulatory compound is shown by the surface plasmon resonance data.

### Example 31

### Fc Receptor Binding to Fc Domain of an Anti-DICR Antibody-Peptide Composition Conjugated with Immune-Stimulatory Compound

This example illustrates the binding of an Fc domain of an anti-DICR antibody-peptide composition conjugated with immune-stimulatory compound to Fc receptors. Each anti-DICR antibody-peptide composition conjugated with immune-stimulatory compound as described in Example 29 is characterized for the ability of their Fc domains to bind to and for their affinity for soluble glycosylated FcγR ectodomains from all human FcγRs. This is shown by performing surface plasmon resonance experiments. In these experiments, biotinylated soluble glycosylated FcγR ectodomains from all human FcγRs are immobilized on a streptavidin-coated surface. The ability of each anti-DICR antibody-peptide composition conjugated with immune-stimulatory compound to bind to soluble glycosylated FcγR ectodomains from all human FcγRs is then measured by surface plasmon resonance using a Biacore instrument. The soluble glycosylated FcγR ectodomains from all human FcγRs is bound by each of the antibody-peptide compositions conjugated with immune-stimulatory compound. Therefore, the surface plasmon resonance experiments show that the ability of the Fc domain of the anti-DICR antibody component of the anti-DICR antibody-peptide composition conjugated with immune-stimulatory compound to bind to all human FcγRs is not interfered with by linking the peptide to the anti-DICR antibody or by conjugation of immune-stimulatory compounds with the anti-DICR antibody-peptide composition. The affinity of each anti-DICR antibody as described in Example 29 for each human FcγRs is also shown by the surface plasmon resonance experiments. These affinity measurements for each anti-DICR antibody-peptide composition conjugated with immune-stimulatory compound are compared with the affinity measurements for each anti-DICR antibody. The similarity in affinity of each anti-DICR antibody-peptide composition conjugated with immune-stimulatory compound for soluble glycosylated FcγR ectodomains from all human FcγRs with the affinity of each corresponding anti-DICR antibody alone for soluble glycosylated FcγR ectodomains from all human FcγRs is shown by this comparison.

### Example 32

### Cytokine Production is Enhanced by Anti-DICR Antibody-Peptide Compositions Conjugated with Immune-Stimulatory Compound

This example shows cytokine production is enhanced by anti-DICR-antibody peptide compositions conjugated with immune-stimulatory compound. Dendritic cells (DCs) are derived from peripheral blood mononuclear cells (PBMCs). DCs are obtained by putting human PBMCs into a culture dish. The resulting adherent cells are washed with RPMI containing 10% fetal calf serum, and then are incubated for 7 days in complete medium containing 10 ng/mL IL-4 and 100 ng/mL GM-CSF. The non-adherent cells are isolated and are washed. These isolated cells are run by a flow cytometer to ensure CD11c expression, in which the DCs identity as DCs is confirmed by CD11c expression. The DCs are then incubated with either anti-DICR antibody-peptide compositions conjugated with immune stimulatory compounds as described in Example 29 or the corresponding anti-DICR antibodies alone. A non-binding isotype control antibody is also incubated with DCs as a control. Each culture is then incubated for 24 hours and the supernatant of each culture is analyzed using a cytokine bead array assay. Cytokine expression levels of IFNγ, IL-8, IL-12 and IL-2 are measured by the cytokine bead array assay. The level of cytokine expression in the supernatant of the culture containing the non-binding isotype control is decreased compared to supernatants from the cultures containing the anti-DICR-peptide compositions conjugated with immune-stimulatory compound. Additionally, the level of cytokine expression in the supernatant from cultures containing anti-DICR antibodies alone is decreased as compared to the supernatant from cultures containing the anti-DICR antibody-peptide composition conjugated with immune-stimulatory compound.

### Example 33

### Anti-DNGR-1 Antibody-Peptide Composition Conjugated with Immune-Stimulatory Compound

This example describes an anti-DNGR-1 antibody-peptide composition conjugated with an immune stimulatory compound. An anti-DNGR-1 antibody-peptide composition as described in Example 13 is conjugated to an immune-stimulatory compound by lysine-based bioconjugation as described in Example 19. An anti-C40 antibody-peptide composition as described in Example 13 is conjugated to an immune-stimulatory compound by cysteine-based bioconjugation as described in Example 20.

### Example 34

### Affinity of Anti-DNGR-1 Antibody-Peptide Composition Conjugated with Immune-Stimulatory Compound to DNGR-1

This example illustrates the binding affinity of an antigen binding domain of an anti-DNGR-1 antibody-peptide composition conjugated with immune stimulatory compound to DNGR-1. Each anti-DNGR-1 antibody-peptide composition conjugated with immune-stimulatory compound as described in Example 33 is measured for affinity to DNGR-1. These affinities are measured by experiments using surface plasmon resonance. In these experiments, biotinylated recombinant DNGR-1 is immobilized on a streptavidin-coated surface. The ability of each anti-DNGR-1 antibody-peptide composition conjugated with immune-stimulatory compound to bind to recombinant DNGR-1 is then measured by surface plasmon resonance using a Biacore instrument. Recombinant DNGR-1 is bound by each anti-DNGR-1 antibody-peptide composition conjugated with immune-stimulatory compound. Therefore, the ability of each anti-DNGR-1 antibody-peptide composition conjugated with immune-stimulatory compound to bind to DNGR-1 is not interfered with by peptide in the composition or by conjugation with immune-stimulatory compound is shown by the surface plasmon resonance data.

### Example 35

### Fc Receptor Binding to Fc Domain of an Anti-DNGR-1 Antibody-Peptide Composition Conjugated with Immune-Stimulatory Compound

This example illustrates the binding of an Fc domain of an anti-DNGR-1 antibody-peptide composition conjugated with immune-stimulatory compound to Fc receptors. Each anti-DNGR-1 antibody-peptide composition conjugated with immune-stimulatory compound as described in Example 33 is characterized for the ability of their Fc domains to bind to and for their affinity for soluble glycosylated FcγR ectodomains from all human FcγRs. This is shown by performing surface plasmon resonance experiments. In these experiments, biotinylated soluble glycosylated FcγR ectodomains from all human FcγRs are immobilized on a streptavidin-coated surface. The ability of each anti-DNGR-1 antibody-peptide composition conjugated with immune-stimulatory compound to bind to soluble glycosylated FcγR ectodomains from all human FcγRs is then measured by surface plasmon resonance using a Biacore instrument. The soluble glycosylated FcγR ectodomains from all human FcγRs is bound by each of the antibody-peptide compositions conjugated with immune-stimulatory compound. Therefore, the surface plasmon resonance experiments show that the ability of the Fc domain of the anti-DNGR-1 antibody component of the anti-DNGR-1 antibody-peptide composition conjugated with immune-stimulatory compound to bind to all human FcγRs is not interfered with by linking the peptide to the anti-DNGR-1 antibody or by conjugation of immune-stimulatory compounds with the anti-DNGR-1 antibody-peptide composition. The affinity of each anti-DNGR-1 antibody as described in Example 33 for each human FcγRs is also shown by the surface plasmon resonance experiments. These affinity measurements for each anti-DNGR-1 antibody-peptide composition conjugated with immune-stimulatory compound are compared with the affinity measurements for each anti-DNGR-1 antibody. The similarity in affinity of each anti-DNGR-1 antibody-peptide composition conjugated with immune-stimulatory compound for soluble glycosylated FcγR ectodomains from all human FcγRs with the affinity of each corresponding anti-DNGR-1 antibody alone for soluble glycosylated FcγR ectodomains from all human FcγRs is shown by this comparison.

### Example 36

### Cytokine Production is Enhanced by Anti-DNGR-1 Antibody-Peptide Compositions Conjugated with Immune-Stimulatory Compound

This example shows cytokine production is enhanced by anti-DNGR-1-antibody peptide compositions conjugated with immune-stimulatory compound. Dendritic cells (DCs) are derived from peripheral blood mononuclear cells (PBMCs). DCs are obtained by putting human PBMCs into a culture dish. The resulting adherent cells are washed with RPMI containing 10% fetal calf serum, and then are incubated for 7 days in complete medium containing 10 ng/mL IL-4 and 100 ng/mL GM-CSF. The non-adherent cells are isolated and are washed. These isolated cells are run by a flow cytometer to ensure CD11c expression, in which the DCs identity as DCs is confirmed by CD11c expression. The DCs are then incubated with either anti-DNGR-1 antibody-peptide compositions conjugated with immune stimulatory compounds as described in Example 8 or the corresponding anti-DNGR-1 antibodies alone. A non-binding isotype control antibody is also incubated with DCs as a control. Each culture is then incubated for 24 hours and the supernatant of each culture is analyzed using a cytokine bead array assay. Cytokine expression levels of IFNγ, IL-8, IL-12 and IL-2 are measured by the cytokine bead array assay. The level of cytokine expression in the supernatant of the culture containing the non-binding isotype control is decreased compared to supernatants from the cultures containing the anti-DNGR-1-peptide compositions conjugated with immune-stimulatory compound. Additionally, the level of cytokine expression in the supernatant from cultures containing anti-DNGR-1 antibodies alone is decreased as compared to the supernatant from cultures containing the anti-DNGR-1 antibody-peptide composition conjugated with immune-stimulatory compound.

### Example 37

### Anti-BDCA-2 Antibody-Peptide Composition Conjugated with Immune-Stimulatory Compound

This example describes an anti-BDCA-2 antibody-peptide composition conjugated with an immune stimulatory compound. An anti-BDCA-2 antibody-peptide composition as described in Example 16 is conjugated to an immune-stimulatory compound by lysine-based bioconjugation as described in Example 19. An anti-BDCA-2 antibody-peptide composition as described in Example 16 is conjugated to an immune-stimulatory compound by cysteine-based bioconjugation as described in Example 20.

### Example 38

### Affinity of Anti-BDCA-2 Antibody-Peptide Composition Conjugated with Immune-Stimulatory Compound to BDCA-2

This example illustrates the binding affinity of an antigen binding domain of an anti-BDCA-2 antibody-peptide composition conjugated with immune stimulatory compound to BDCA-2. Each anti-BDCA-2 antibody-peptide composition conjugated with immune-stimulatory compound as described in Example 37 is measured for affinity to BDCA-2. These affinities are measured by experiments using surface plasmon resonance. In these experiments, biotinylated recombinant BDCA-2 is immobilized on a streptavidin-coated surface. The ability of each anti-BDCA-2 antibody-peptide composition conjugated with immune-stimulatory compound to bind to recombinant BDCA-2 is then measured by surface plasmon resonance using a Biacore instrument. Recombinant BDCA-2 is bound by each anti-BDCA-2 antibody-peptide composition conjugated with immune-stimulatory compound. Therefore, the ability of each anti-BDCA-2 antibody-peptide composition conjugated with immune-stimulatory compound to bind to BDCA-2 is not interfered with by peptide in the composition or by conjugation with immune-stimulatory compound is shown by the surface plasmon resonance data.

### Example 39

### Fc Receptor Binding to Fc Domain of an Anti-BDCA-2 Antibody-Peptide Composition Conjugated with Immune-Stimulatory Compound

This example illustrates the binding of an Fc domain of an anti-BDCA-2 antibody-peptide composition conjugated with immune-stimulatory compound to Fc receptors. Each anti-BDCA-2 antibody-peptide composition conjugated with immune-stimulatory compound as described in Example 37 is characterized for the ability of their Fc domains to bind to and for their affinity for soluble glycosylated FcγR ectodomains from all human FcγRs. This is shown by performing surface plasmon resonance experiments. In these experiments, biotinylated soluble glycosylated FcγR ectodomains from all human FcγRs are immobilized on a streptavidin-coated surface. The ability of each anti-BDCA-2 antibody-peptide composition conjugated with immune-stimulatory compound to bind to soluble glycosylated FcγR ectodomains from all human FcγRs is then measured by surface plasmon resonance using a Biacore instrument. The soluble glycosylated FcγR ectodomains from all human FcγRs is bound by each of the antibody-peptide compositions conjugated with immune-stimulatory compound. Therefore, the surface plasmon resonance experiments show that the ability of the Fc domain of the anti-BDCA-2 antibody component of the anti-BDCA-2 antibody-peptide composition conjugated with immune-stimulatory compound to bind to all human FcγRs is not interfered with by linking the peptide to the anti-BDCA-2 antibody or by conjugation of immune-stimulatory compounds with the anti-BDCA-2 antibody-peptide composition. The affinity of each anti-BDCA-2 antibody as described in Example 37 for each human FcγRs is also shown by the surface plasmon resonance experiments. These affinity measurements for each anti-BDCA-2 antibody-peptide composition conjugated with immune-stimulatory compound are compared with the affinity measurements for each anti-BDCA-2 antibody. The similarity in affinity of each anti-BDCA-2 antibody-peptide composition conjugated with immune-stimulatory compound for soluble glycosylated FcγR ectodomains from all human FcγRs with the affinity of each corresponding anti-BDCA-2 antibody alone for soluble glycosylated FcγR ectodomains from all human FcγRs is shown by this comparison.

### Example 40

### Cytokine Production is Enhanced by Anti-BDCA-2 Antibody-Peptide Compositions Conjugated with Immune-Stimulatory Compound

This example shows cytokine production is enhanced by anti-BDCA-2-antibody peptide compositions conjugated with immune-stimulatory compound. Dendritic cells (DCs) are derived from peripheral blood mononuclear cells (PBMCs). DCs are obtained by putting human PBMCs into a culture dish. The resulting adherent cells are washed with RPMI containing 10% fetal calf serum, and then are incubated for 7 days in complete medium containing 10 ng/mL IL-4 and 100 ng/mL GM-CSF. The non-adherent cells are isolated and are washed. These isolated cells are run by a flow cytometer to ensure CD11c expression, in which the DCs identity as DCs is confirmed by CD11c expression. The DCs are then incubated with either anti-BDCA-2 antibody-peptide compositions conjugated with immune stimulatory compounds as described in Example 37 or the corresponding anti-BDCA-2 antibodies alone. A non-binding isotype control antibody is also incubated with DCs as a control. Each culture is then incubated for 24 hours and the supernatant of each culture is analyzed using a cytokine bead array assay. Cytokine expression levels of IFNγ, IL-8, IL-12 and IL-2 are measured by the cytokine bead array assay. The level of cytokine expression in the supernatant of the culture containing the non-binding isotype control is decreased compared to supernatants from the cultures containing the anti-BDCA-2-peptide compositions conjugated with immune-stimulatory compound. Additionally, the level of cytokine expression in the supernatant from cultures containing anti-BDCA-2 antibodies alone is decreased as compared to the supernatant from cultures containing the anti-BDCA-2 antibody-peptide composition conjugated with immune-stimulatory compound.

### Example 41

### Treatment of Cancer by Administering a Construct-Peptide Composition

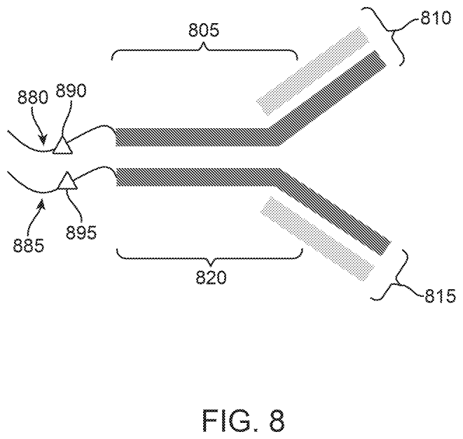

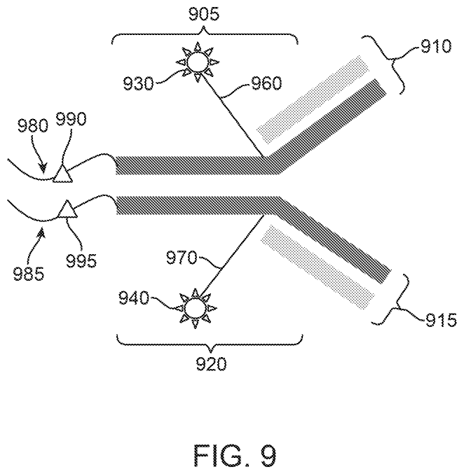

This example describes treatment of cancer with a construct-peptide composition. A human patient is diagnosed with a cancer. A construct-peptide composition as shown in the schematic of FIG. 8 is administered to the patient with a pharmaceutically acceptable carrier. FIG. 8 is a construct-peptide composition. The construct is an antibody. The antibody comprises two heavy chains as shown in gray and two light chains as shown in light gray. The antibody has two antigen binding sites (810 and 815), and a portion of the heavy chains contain Fc domains (805 and 820). The peptides form a composition with the antibody (880 and 885). The peptides contain a nonsynonymous (missense) mutation (890 and 895). As another example, a human patient is diagnosed with a cancer. A construct-peptide composition as shown in the schematic of FIG. 9 is administered to the patient with a pharmaceutically acceptable carrier. FIG. 9 is a construct-peptide composition conjugated to immune-stimulatory compounds. The construct is an antibody. The antibody comprises two heavy chains as shown in gray and two light chains as shown in light gray. The antibody has two antigen binding sites (910 and 915), and a portion of the heavy chains contain Fc domains (905 and 920). The immune-stimulatory compounds (930 and 940) are conjugated to the antibody by linkers (960 and 970). The peptides (980 and 985) form a composition with the antibody. The peptides contain a nonsynonymous (missense) mutation (990 and 995).

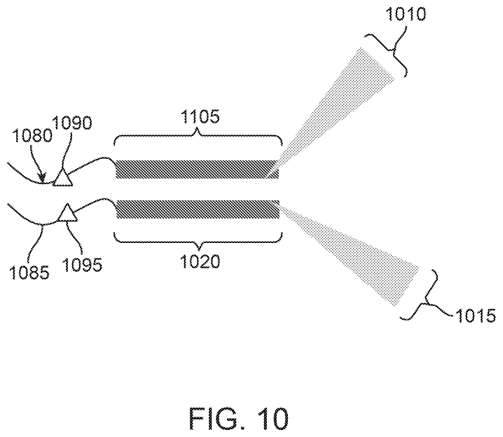

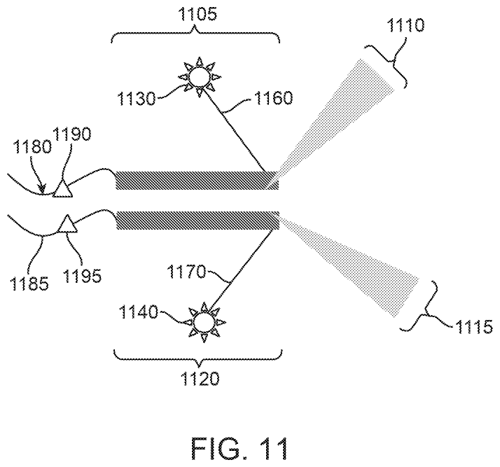

As an additional example, a human patient is diagnosed with a cancer. A construct-peptide composition as shown in the schematic of FIG. 10 is administered to the patient with a pharmaceutically acceptable carrier. FIG. 10 is a construct-peptide composition. The construct comprises Fc regions of an antibody with the heavy chains shown in gray, and two scaffolds as shown in light gray. The construct comprises two antigen binding sites (1010 and 1015) in the scaffolds, and a portion of the heavy chains contain Fc domains (1005 and 1020). The peptides (1080 and 1085) form a composition with the construct. The peptide contains a nonsynonymous (missense) mutation (1090 and 1095). As another example, a human patient is diagnosed with a cancer. A construct-peptide composition as shown in the schematic of FIG. 11 is administered to the patient with a pharmaceutically acceptable carrier. FIG. 11 is construct-peptide composition conjugated to immune-stimulatory compounds. The construct comprises Fc regions of an antibody with the heavy chains shown in gray, and two scaffolds as shown in light gray. The construct comprises two antigen binding sites (1110 and 1115) in the scaffolds, and a portion of the heavy chains contain Fc domains (1105 and 1120). The immune-stimulatory compounds (1130 and 1140) are conjugated to the construct by linkers (1160 and 1170). The peptides (1180 and 1185) form a composition with the construct. The peptides contain a nonsynonymous (missense) mutation (1190 and 1195).

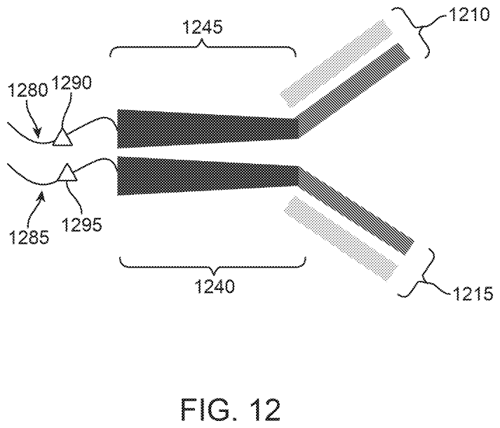

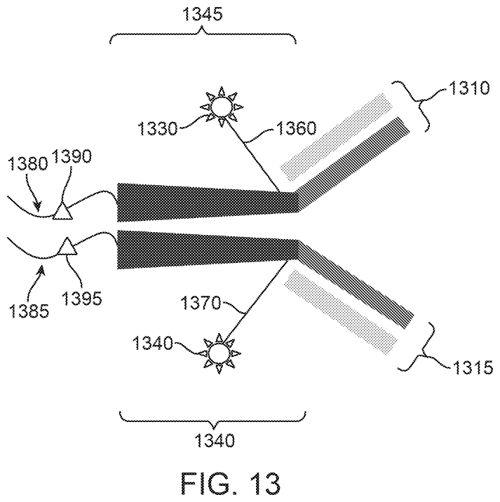

As another example, a human patient is diagnosed with a cancer. A construct-peptide composition as shown in the schematic of FIG. 12 is administered to the patient with a pharmaceutically acceptable carrier. FIG. 12 is a construct-peptide composition. The construct comprises the F(ab′)2 regions of an antibody with heavy chains shown in gray and light chains shown in light gray, and two scaffolds as shown in dark gray. The construct comprises two antigen binding sites (1210 and 1215), and a portion of two scaffolds contain Fc domains (1240 and 1245). The peptides (1280 and 1285) form a composition with the construct. The peptide contains a nonsynonymous (missense) mutation (1290 and 1295). As another example, a human patient is diagnosed with a cancer. A construct-peptide composition as shown in the schematic of FIG. 13 is administered to the patient with a pharmaceutically acceptable carrier. FIG. 13 is a construct-peptide composition conjugated to immune-stimulatory compounds. The construct contains the F(ab′)2 regions of an antibody with heavy chains shown in gray and light chains shown in light gray, and two scaffolds as shown in dark gray. The construct comprises two antigen binding sites (1310 and 1315), and a portion of two scaffolds contain Fc domains (1340 and 1345). The immune-stimulatory compounds (1330 and 1340) are conjugated to the construct by linkers (1360 and 1370). The peptides (1380 and 1385) form a composition with the construct. The peptide contains a nonsynonymous (missense) mutation (1390 and 1395).

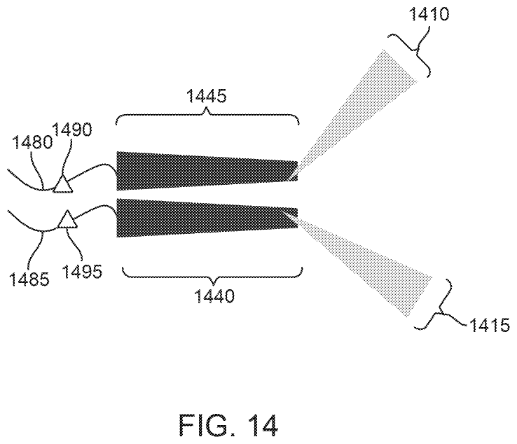

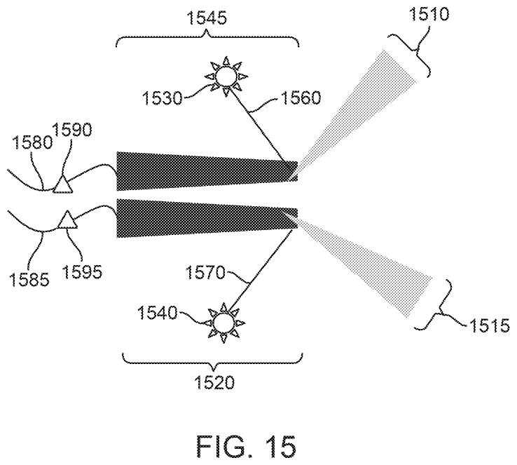

As another example, a human patient is diagnosed with a cancer. A construct-peptide composition as shown in the schematic of FIG. 14 is administered to the patient with a pharmaceutically acceptable carrier. FIG. 14 is a construct-peptide composition. The construct contains two scaffolds as shown in light gray and two scaffolds as shown in dark gray. The construct comprises two antigen binding sites (1410 and 1415) in the light gray scaffolds, and a portion of the two dark gray scaffolds contain Fc domains (1440 and 1445). The peptides (1480 and 1485) form a composition with the construct. The peptide contains a nonsynonymous (missense) mutation (1490 and 1495). As another example, a human patient is diagnosed with a cancer. A construct-peptide composition as shown in the schematic of FIG. 15 is administered to the patient with a pharmaceutically acceptable carrier. FIG. 15 is a construct-peptide composition conjugated to immune-stimulatory compounds. The construct contains two scaffolds as shown in light gray and two scaffolds as shown in dark gray. The construct comprises two antigen binding sites (1510 and 1515), and a portion of the two dark gray scaffolds contain Fc domains (1540 and 1545). The immune-stimulatory compounds (1530 and 1540) are conjugated to the construct by linkers (1560 and 1570). The peptides (1580 and 1585) form a composition with the construct. The peptide contains a nonsynonymous (missense) mutation (1590 and 1595).

### Example 42

### Determination of K_{d} Values

K_{d} is measured by a radiolabeled antigen binding assay (RIA) performed with the Fab version of an antibody of interest and its antigen as described by the following assay.

Solution binding affinity of Fabs for antigen is measured by equilibrating the Fab with a minimal concentration of (¹²⁵I)-labeled antigen in the presence of a titration series of unlabeled antigen, then capturing bound antigen with an anti-Fab antibody-coated plate (See, e.g., Chen et al., J. Mol. Biol. 293:865-881 (1999)). To establish conditions for the assay, multi-well plates are coated overnight with 5 pg/mL of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant is added, and the plates are counted on a TOPCOUNT™ gamma counter (Packard) for ten minutes. Concentrations of each Fab that give less than or equal to 20% of maximal binding are chosen for use in competitive binding assays.

### Example 43

### Determination of K_{d} Values

K_{d} is measured using surface plasmon resonance assays using a BIACORE®-2000 or a BIACORE®-3000 (BIAcore, Inc., Piscataway, N.J.) at 25° C. with immobilized antigen CM5 chips at ^{˜}10 response units (RU). Briefly, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/mL (^{˜}0.2 pM) before injection at a flow rate of 5 μL/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μL/Imin. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (K_{d}) is calculated as the ratio k_{off}/kₒₙ. See, e.g., Chen et al., J. Mol. Biol. 293:865-881 (1999). If the on-rate exceeds 106 M-1 s-1 by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

### Example 44

### Determination of Molar Ratio

This example illustrates one method by which the molar ratio is determined. One microgram of construct-peptide composition conjugated with an immune-stimulatory compound is injected into an LC/MS such as an Agilent 6550 iFunnel Q-TOF equipped with an Agilent Dual Jet Stream ESI source coupled with Agilent 1290 Infinity UHPLC system. Raw data is obtained and is deconvoluted with software such as Agilent MassHunter Qualitative Analysis Software with BioConfirm using the Maximum Entropy deconvolution algorithm. The average mass of intact construct-peptide composition conjugated with an immune-stimulatory compound is calculated by the software, which can use top peak height at 25% for the calculation. This data is then imported into another program to calculate the molar ratio of the immune-stimulatory compound in the construct-peptide composition conjugated with an immune-stimulatory compound such as Agilent molar ratio calculator.

Another method for determination of molar ratio is as follows. First, 10 μL of a 5 mg/mL solution of a construct-peptide composition conjugated with an immune-stimulatory compound is injected into an HPLC system set-up with a TOSOH TSKgel Butyl-NPR™ hydrophobic interaction chromatography (HIC) column (2.5 μM particle size, 4.6 mm×35 mm) attached. Then, over the course of 18 minutes, a method is run in which the mobile phase gradient is run from 100% mobile phase A to 100% mobile phase B over the course of 12 minutes, followed by a six minute re-equilibration at 100% mobile phase A. The flow rate is 0.8 mL/min and the detector is set at 280 nM. Mobile phase A is 1.5 M ammonium sulfate, 25 mM sodium phosphate (pH 7). Mobile phase B is 25% isopropanol in 25 mM sodium phosphate (pH 7). Post-run, the chromatogram is integrated and the molar ratio is determined by summing the weighted peak area.

### Example 45

### Cell Surface Molecules Important for Antigen Specific T Cell Activation and Expansion are Increased by Construct-Peptide Immune-Stimulatory Compound Conjugates

This example shows that cell surface molecules, whose expression are important for antigen specific T cell activation and expansion, are increased by construct-peptide immune stimulatory compound conjugates.

Increased levels of MHC and T cell costimulatory molecules on dendritic cells can promote T cell expansion and effector function. Therefore, anti-CD40/MART1 peptide fusions conjugated with a TLR8 agonist or STING agonist, anti-DEC205/MART1 peptide fusions conjugated with a TLR8 agonist or STING agonist, or anti-DNGR-1/MART 1 peptide fusions conjugated with a TLR8 agonist or STING agonist are tested for their ability to increase cell surface expression of MHC class II, CD86 and CD80 on in vitro derived human dendritic cells (mDCs). Antibody-peptide fusions without conjugated immune-stimulatory compounds or non-DC binding antibody-peptide fusions are used as controls. Human peripheral blood mononuclear cells (PBMCs) are isolated from normal donors by standard Ficoll-Hypaque density procedure. Dendritic cells are derived from human PBMCs by isolation of CD14⁺ monocytes followed by culture in RPMI containing 10% fetal calf serum for seven days in complete medium supplemented with 10 ng/mL IL-4 and 100 ng/mL GM-CSF. After 48 hours of further incubation in growth media, the cells are collected, washed by centrifugations, and then are stained for 30 minutes on ice using manufacturer's recommended concentrations of commercially available anti-CD80, anti-CD86, and anti-MHC class II monoclonal antibodies conjugated to laser sensitive fluors, and are stained for viability. A separate aliquot of each treatment is stained with IgG matched isotype control antibody for the anti-CD86, anti-CD80 and anti-MHC Class II. After washing to remove unbound antibody-fluor molecules, the stained cells are subjected to FACS analysis using a Celesta flow cytometer (BD Biosciences) with gating on live cells. The output is analyzed by FlowJo v10.2 software (FlowJo LLC) and curve fit with Prism 7.01 software (GraphPad Software, Inc.). Expression of MHC class II, CD86, and CD80 is increased on mDCs incubated with the construct/peptide fusions conjugated with a TLR8 agonist or STING agonist.

### Example 46

### Antigen from Construct-Peptide Immune-Stimulatory Compound Conjugates is Cross Presented to CD8⁺ T Cells

This example shows that antigen from a construct-peptide immune-stimulatory compound conjugate is cross presented to CD8⁺ T cells.

Tumor antigen cross presentation on MHC Class I molecules by dendritic cells to CD8⁺ T cells can be important for clinically relevant anti-tumor immune responses. Dendritic cells (DCs) are generated from HLA-A201⁺ donors as described in Example 45. From the same donors, CD8⁺ T cells with HER2 or MART1 tumor antigen positive TCRs are enriched for by isolation of CD8⁺ T cells from peripheral blood mononuclear cells (PBMCs) using CD8⁺ negative selection beads followed by incubation with MHC tetramers loaded with either HER2 (amino acid residues 369-377 of the HER2) or MART1 (amino acid residues 26-35 of MART1) peptides. The peptide reactive T cells are expanded in culture with beads coated with anti-CD3 and anti-CD28 for 7 days, are allowed to rest for 24 hours, and then are labeled with CellTrace Violet (ThermoFisher) according to the manufacturer's directions prior to placement into co-culture with the DCs. 1×10⁴ DCs and 2×10⁴ T cells are added to wells along with dose titrations of anti-CD40/HER2 fusion, anti-DEC205/HER2 fusion, anti-DNGR-1/HER2 fusion, anti-CD40/HER2 fusion conjugated with a TLR8 agonist or STING agonist, anti-DEC205/HER2 fusion conjugated with a TLR8 agonist or STING agonist, anti-DNGR-1/HER2 fusion conjugated with a TLR8 agonist or STING agonist, anti-CD40 antibody conjugated with a TLR8 agonist or STING agonist, anti-DEC205 antibody conjugated with a TLR8 agonist or STING agonist, or anti-DNGR-1 conjugated with a TLR8 agonist or STING agonist. After 24 hour incubation both supernatants and cells were harvested. The levels of interferon gamma (IFNγ) are determined by ELISA using electrochemiluminesence signal with commercially available reagents and plate reader (Meso Scale Discovery). The level T cell proliferation is determined by FACS analysis using a Celesta Flowcytometer (BD Biosciences), a commercial viable cell dye, and fluor-conjugated anti-CD8 antibody to identify viable CD8⁺ cells. T cell proliferation is determined by MFI level compared to non-proliferating and anti-CD3/CD28 cultured dye labeled T cells. Potent cross presentation is indicated by increased IFNγ production and CD8⁺ T cell proliferation after addition of the construct-peptide immune-stimulatory compound conjugates.

### Example 47

### Tumor Antigen Cross Presentation to CD8⁺ T Cells is Increased by Construct-Peptide Immune-Stimulatory Compound Conjugates with Enhanced ITAM FcγR Engagement

This example shows that tumor antigen cross presentation to CD8⁺ T cells is increased by construct-peptide immune-stimulatory compound conjugates with enhanced ITAM Fcγ Receptors (FcγRs) engagement.

Engagement of FcγRs that possess an ITAM signaling domain can be important for more effective dendritic cell antigen cross presentation to CD8⁺ T cells. Donor dendritic and tumor-peptide specific T cells are prepared and placed into co-culture as in Example 47. Dose titrations of anti-CD40/HER2 fusion conjugated with a TLR8 agonist or STING agonist, anti-DEC205/HER2 fusion conjugated with a TLR8 agonist or STING agonist, anti-DNGR-1/HER2 fusion conjugated with a TLR8 agonist or STING agonist are added to the co-cultures. Furthermore, a construct/peptide conjugated with a TLR8 agonist or STING agonist with an IgG2, IgG1_{Null} (in which the Fc domain does not bind to Fc receptors), or IgG1 VLPLL are added to co-cultures. The level of cross presentation is assessed by IFNγ secretion and CD8⁺ T cell proliferation as in Example 46. Tumor antigen cross presentation to CD8⁺ T cells is increased by construct-peptide immune-stimulatory compound conjugates with enhanced ITAM Fcγ Receptors (FcγRs) engagement, such as IgG1 VLPLL.

### Example 48

### Tumor Antigen is Effectively Cross-Presented from Construct-Peptide Immune-Stimulatory Compound Conjugates In Vivo

This example shows that tumor antigen is effectively cross-presented from construct-peptide immune-stimulatory compound conjugates in vivo.

The level of antigen cross-presented to human CD8⁺ T cells by human APCs in vivo can be assessed in the humanized NOD/SCID b-2 m^{-/-} immunocompromised mouse model, in which human antigen presenting cells (APCs) and T cells can be adoptively transferred and functionally assessed in vivo. CD34⁺ cells are isolated from normal HLA-A201⁺ donors by leukopheresis after standard G-CSF mobilization and peripheral blood mononuclear cells (PBMCs) isolation followed by CD34⁺ negative selection using beads (StemCell Technologies). 3×10⁶ human CD3⁴ cells is injected into sub-lethally irradiated mice. A 1:1 mixture of MART1 reactive enriched CD8⁺ and total peripheral blood CD4⁺ T cells from the same donor as the CD34⁺ cells are injected into mice 4 weeks post-CD34⁺ cell injection. One day after T cell transfer 10 ug/mouse of anti-CD40/HER2 fusion conjugated with a TLR8 agonist or STING agonist, anti-DEC205/HER2 fusion conjugated with a TLR8 agonist or STING agonist, anti-DNGR-1/HER2 fusion conjugated with a TLR8 agonist or STING agonist is injected intravenously into the mice. Peripheral blood is obtained at day 11 and 14 by retro-orbital bleed and the number of MART1 reactive CD8⁺ T cells/ul of blood are quantitated using FACS analysis and MART1 peptide loaded HLA-A201 tetramers with anti-biotin and anti-human CD8 fluor-conjugated antibodies. The number of tumor antigen CD8⁺ T cells is increased after administration of the construct-peptide immune-stimulatory compound conjugates, indicating effective antigen cross presentation.

### Example 49

### Anti-Tumor Activity is Increased by Construct-Peptide Immune-Stimulatory Compound Conjugates in a Mouse Syngeneic Model

This example shows that anti-tumor activity is increased by construct-peptide immune-stimulatory compound conjugates.

A mouse syngeneic tumor model with transgenic mice humanized for CD40 is used to test whether the increased anti-tumor antigen immune response after administration of construct-peptide immune-stimulatory conjugates can treat cancer. Mouse c57bl6 breast tumor cell line E0711 transfected to stably express human HER2 tumor antigen is used as the mouse syngeneic tumor model. Tumor cells (1×10⁶/mouse) are placed orthotopically in mammary fat pads and growth is tracked by measurement with calipers. At the time of tumor cell implant IV treatments are begun with cohorts of 10 mice receiving 3 weekly doses of anti-CD40/HER2 fusions conjugated with a STING agonist, in which the Fc domains are IgG1, IgG2, IgG1₀u (in which the Fc domain does bind to Fc receptors), or IgG1 VLPLL, or with control anti-CD40 antibody conjugated with STING agonists, in which the Fc domains are IgG1, IgG2, IgG1₀u, or IgG1 VLPLL. The treatment doses are ranged from 1 ug to 100 ug/dose. General health and weights of the tumor bearing mice and tumor sizes are monitored daily for 42 days or until 400 mm tumor size is reached, at which time the mice are euthanized. Kaplan-Meyer plots for survival and as plots of tumor size (mm) over time (days) are calculated. A lowered tumor burden and increased survive is found in mice treated with the anti-CD40/HER2 fusion with an IgG1, IgG2, or IgG1 VLPLL Fc domain and conjugated with a STING agonist versus the controls.

### Example 50

### Anti-Tumor Immunity and Tumor Epitope Spreading is Promoted by Construct-Peptide Immune-Stimulatory Compound Conjugates

This example shows that anti-tumor immunity and tumor epitope spreading is promoted by construct-peptide immune-stimulatory compound conjugates.

Because tumor antigen epitopes represented by the peptides in construct-peptide immune-stimulatory compound conjugates can be lost or altered by a patient's cancer thereby limiting therapeutic benefit, it is tested whether the increased anti-tumor response engendered by the construct-peptide immune-stimulatory compound conjugate can increase epitope spread anti-tumor responses. The syngeneic mouse model as described in Example 49 is used, except 1×10⁶ tumor cells are subcutaneously injected in each flank of the CD40 humanized mice. E0711 cells expressing the human HER2 tumor antigen are injected into both flanks for some mice, or human HER2 expressing E0711 is injected into one flank and non-transfected E0711 cells lacking human HER2 expression is injected into the second flank. As described in EXAMPLE 50, the mice are treated with anti-CD40/HER2 fusions conjugated with a STING agonist, in which the Fc domains are IgG1, IgG2, IgG1_{N}u (in which the Fc domain does bind to Fc receptors), or IgG1 VLPLL, or with control anti-CD40 antibody conjugated with STING agonists, in which the Fc domains are IgG1, IgG2, IgG11_{N}u (in which the Fc domain does bind to Fc receptors), or IgG1 VLPLL. Tumor burden is monitored as in Example 50. Tumor burden is lowered and survival is increased, but tumor burden for non-human HER2 expressing tumors is additionally diminished by an increased epitope spread anti-tumor response in mice after the anti-CD40/HER2 fusion with an IgG1, IgG2, or IgG1 VLPLL Fc domain and conjugated with a STING agonist is administered versus after the controls are administered.

### Example 51

### Stability of Construct-Peptide Immune-Stimulatory Compound Conjugate in IgG Depleted Human Serum

This example shows the stability of a construct-peptide conjugate and construct-peptide immune-stimulatory compound conjugate in IgG depleted human serum. Stability of construct-peptide conjugate and construct-peptide immune-stimulatory compound conjugate in human serum (IgG depleted) is measured over 96 hours at 37° C. using either a direct HIC-UV analysis approach (Method A) or an affinity capture approach (Method B). Construct-peptide conjugates or construct-peptide immune-stimulatory compound conjugates are spiked in IgG-depleted human serum (BBI solutions # SF142-2) in sterile tubes (75% final serum concentration) and samples are split into 4 aliquots of equal size then transferred to a 37° C. incubator. One of the aliquots of each sample is taken from the incubator at each time-point (T=0h, 24h, 48h, 96h) and the stability of the construct-peptide conjugate or the average immune-stimulatory molecule-construct peptide ratios (DAR) is recorded.

### Method A: Direct HIC-UV Analysis

At 0, 24, 48 and 96 hours after the beginning of incubation, the construct-peptide conjugate or construct-peptide immune-stimulatory compound conjugate spiked in IgG depleted human serum is analyzed by analytical hydrophobic interaction chromatography (HIC) using a TOSOH TSKgel Butyl-NPR 4.6 mm×35 mm HIC column (TOSOH Bioscience, #14947) connected to a Dionex Ultimate 3000RS HPLC system (ThermoFisher Scientific, Hemel Hemstead, UK). The stability of the construct-peptide conjugate is assessed and is found to remain stable at each time point. The average DAR of the construct-peptide immune-stimulatory compound conjugate is found to remain stable at each timepoint.

### Method B: Affinity Capture, De-Glycosylation and RP-ESI-MS Analysis

A construct-peptide conjugate or construct-peptide immune-stimulatory compound conjugate is immunocaptured from the IgG depleted human serum using an anti-Human IgG (Fc specific) biotin antibody immobilized on streptavidin beads at 0, 24, 48 and 96 hours after the beginning of incubation. After elution from the beads, the samples are de-glycosylated using agarose-immobilized EndoS (Genovis Inc, USA). The de-glycosylated construct-peptide conjugate or construct-peptide immune-stimulatory compound conjugate is analyzed by reverse phase chromatography hyphenated to electrospray ionization mass spectrometry (RP-ESI-MS) using an Acquity nano UPLC in line with a Xevo G2S Q-TOF (Waters, Elstree, UK). The separation is performed using an Acquity UPLC online coupled to an ESI-MS mass spectrometer. Mass spectrometric analysis is performed in positive ion mode, scanning from 1000 to 4000 m/z in high mass operating mode. The ion envelope produced by each sample is deconvoluted using the MaxEnt 1 algorithm provided within the MassLynx software (Waters, Elstree, UK). The stability of the construct-peptide conjugate is assessed and is found to remain stable at each time point. The average DAR of the construct-peptide immune-stimulatory compound conjugate is found to remain stable at each timepoint.

While aspects of the present disclosure have been shown and described herein, it will be apparent to those skilled in the art that such aspects are provided by way of example only. Numerous variations, changes, and substitutions will now occur to those skilled in the art without departing from the disclosure. It should be understood that various alternatives to the aspects of the disclosure described herein may be employed in practicing the disclosure. It is intended that the following claims define the scope of the disclosure and that methods and structures within the scope of these claims and their equivalents be covered thereby.

1. A composition, comprising:
(a) an immune-stimulatory compound connected to an antibody by a first linker, wherein the immune-stimulatory compound is a Toll-like receptor (TLR) agonist, a RIG-I agonist, a TGFβ agonist, a β-Catenin inhibitor, a PI3K-β inhibitor, or a STAT3 inhibitor,
(b) the antibody comprising:
(i) an antigen binding domain, wherein the antigen binding domain specifically binds a target antigen, wherein the target antigen is an antigen expressed on an immune cell selected from the group consisting of CD40, DEC-205, DICR DNGR-1, BDCA-2, CD36 mannose scavenger receptor 1, CLEC12A, DC-SIGN, OX40L, 4-1BBL, CD36, CD204, MARCO, CLEC9A, Dectin 1, Dectin 2, CLEC10A, CD206, CD64, CD32a, CD16a, HVEM, and CD32b, and
(ii) an Fc domain, wherein a K_{d} for binding of the Fc domain to an Fc receptor in the presence of the immune-stimulatory compound is no greater than about 100 times a K_{d} for binding of the Fc domain to the Fc receptor in the absence of the immune stimulatory compound; and
(c) a peptide comprising 10 to 55 amino acid residues of an antigenic epitope of a cancer sequence that is connected to the antibody.

2. The composition of claim of 1, wherein the peptide is connected to the antibody as a fusion protein or by a second linker at the C-terminus of the Fc domain.

3. The composition of claim 1, wherein the peptide comprises a neoantigen, a tumor specific epitope, or a splice variant of a tumor specific epitope.

4. (canceled)

5. The composition of claim 1, wherein the peptide is selected from the group consisting of:
a) p53 having a V157F, G154V, R176G, P278A, Y220C, G245S, R248Q, or R273H mutation;
b) BRAF having a G466V or V600E mutation;
c) NFE2L2 having a E79Q mutation;
d) EGFR having a G719A mutation;
e) KRAS having a G12D, G12V, G12C, or G12R mutation;
f) HRAS having a G12V, Q61L, or Q61R mutation;
g) NRAS having a G12D, G12S, G13D, Q61K, or Q61R mutation;
h) C3orf59 having Q311E mutation;
i) ERBB2IP having a E805G mutation;
j) NUP98 having a A359D mutation;
k) GPD2 having a E426K mutation;
1) PLEC having a E1179K mutation;
m) XPO7 having a P274S mutation;
n) AKAP2 having a Q418K mutation;
o) CASP8 having a F67V mutation;
p) ITBG4 having a S1002I mutation;
q) TUBGCP2 having a P293L mutation;
r) RNF213 having a N1702S mutation;
s) SKIV2L having a R653H mutation;
t) H3F3B having a A48T mutation;
u) API5 having a R243Q mutation;
v) RNF10 having a E572K mutation;
w) PHLPP1 having a G566E mutation; and
x) 2FYVE27 having a R6H mutation.

6. The composition of claim 1, wherein the peptide comprises 50 amino acids.

7-8. (canceled)

9. The composition of claim 1, wherein the peptide binds to an MHC class I molecule or an MHC class II molecule.

10-16. (canceled)

17. The composition of claim 1, wherein the peptide is immunogenic.

18. (canceled)

19. The composition of claim 1, wherein the immune cell is an antigen presenting cell.

20. (canceled)

21. The composition of claim 1, wherein the antigen binding domain is a CD40 agonist, a DEC-205 agonist, DICR agonist, DNGR-1 agonist, BDCA-2 agonist, CD36 mannose scavenger receptor 1 agonist, CLEC12A agonist, DC-SIGN agonist, OX40L agonist, 4-1BBL agonist, CD36 agonist, CD204 agonist, MARCO agonist, CLEC9A agonist, Dectin 1 agonist, Dectin 2 agonist, CLEC10A agonist, CD206 agonist, CD64 agonist, CD32a agonist, CD16a agonist, HVEM agonist, or CD32b agonist.

22. The composition of claim 1, wherein the antigen binding domain comprises:
(a) HC CDR1 comprising an amino acid sequence of SEQ ID NO: 23, HC CDR2 comprising an amino acid sequence of SEQ ID NO: 24, a HC CDR3 comprising an amino acid sequence of SEQ ID NO: 25, LC CDR1 comprising an amino acid sequence of SEQ ID NO: 27, LC CDR1 comprising an amino acid sequence of SEQ ID NO: 28, and LC CDR3 comprising an amino acid sequence of SEQ ID NO: 29;
(b) HC CDR1 comprising an amino acid sequence of SEQ ID NO: 88, HC CDR2 comprising an amino acid sequence of SEQ ID NO: 89, a HC CDR3 comprising an amino acid sequence of SEQ ID NO: 90, LC CDR1 comprising an amino acid sequence of SEQ ID NO: 93, LC CDR1 comprising an amino acid sequence of SEQ ID NO: 94, and LC CDR3 comprising an amino acid sequence of SEQ ID NO: 95;
(c) HC CDR1 comprising an amino acid sequence of SEQ ID NO: 98, HC CDR2 comprising an amino acid sequence of SEQ ID NO: 99, a HC CDR3 comprising an amino acid sequence of SEQ ID NO: 100, LC CDR1 comprising an amino acid sequence of SEQ ID NO: 103, LC CDR1 comprising an amino acid sequence of SEQ ID NO: 104, and LC CDR3 comprising an amino acid sequence of SEQ ID NO: 105;
(d) HC CDR1 comprising an amino acid sequence of SEQ ID NO: 106, HC CDR2 comprising an amino acid sequence of SEQ ID NO: 107, a HC CDR3 comprising an amino acid sequence of SEQ ID NO: 108, LC CDR1 comprising an amino acid sequence of SEQ ID NO: 115, LC CDR1 comprising an amino acid sequence of SEQ ID NO: 116, and LC CDR3 comprising an amino acid sequence of SEQ ID NO: 117;
(e) HC CDR1 comprising an amino acid sequence of SEQ ID NO: 109, HC CDR2 comprising an amino acid sequence of SEQ ID NO: 110, a HC CDR3 comprising an amino acid sequence of SEQ ID NO: 111, LC CDR1 comprising an amino acid sequence of SEQ ID NO: 118, LC CDR1 comprising an amino acid sequence of SEQ ID NO: 119, and LC CDR3 comprising an amino acid sequence of SEQ ID NO: 120; or
(f) HC CDR1 comprising an amino acid sequence of SEQ ID NO: 112, HC CDR2 comprising an amino acid sequence of SEQ ID NO: 113, a HC CDR3 comprising an amino acid sequence of SEQ ID NO: 114, LC CDR1 comprising an amino acid sequence of SEQ ID NO: 121, LC CDR1 comprising an amino acid sequence of SEQ ID NO: 122, and LC CDR3 comprising an amino acid sequence of SEQ ID NO: 123.

23. (canceled)

24. The composition of claim 1, wherein the K_{d} for binding of the Fc domain to the Fc receptor in the presence of the immune-stimulatory compound is no greater than about two times a K_{d} for binding of the Fc domain to the Fc receptor in an absence of the immune-stimulatory compound

25. The composition of claim 1, wherein the Fc domain is a human Fc domain.

26. The composition of claim 1, wherein the Fc domain is selected from a group consisting of a human IgG1 Fc domain, a human IgG2 Fc domain, a human IgG3 Fc domain, and a human IgG4 Fc domain.

27. The composition of claim 1, wherein the Fc domain binds an Fc receptor with the same affinity as a wild type IgG1 Fc domain.

28. The composition of claim 1, wherein the Fc domain comprises one or more amino acid substitutions that increase the affinity of the Fc domain to an Fc receptor compared to the affinity of a reference Fc domain to the Fc receptor in the absence of the one or more amino acid substitutions.

29. The composition of claim 1, wherein the Fc domain has at least one amino acid residue change as compared to a wildtype Fc domain, wherein the at least one amino acid residue is:
(a) F243L, R292P, Y300L, L235V, and P396L, wherein numbering of amino acid residues in the Fc domain is according to the EU index as in Kabat;
(b) S239D and I332E, wherein numbering of amino acid residues in the Fc domain is according to the EU index as in Kabat; or
(c) S298A, E333A, and K334A, wherein numbering of amino acid residues in the Fc domain is according to the EU index as in Kabat.

30-35. (canceled)

36. The composition of claim 1, wherein the immune-stimulatory compound is a TLR agonist selected from a TLR7 agonist, a TLR8 agonist, or both.

37. The composition of claim 1, wherein the immune-stimulatory compound is selected from a group consisting of: CpG oligonucleotide, Poly G10, Poly G3, Poly I:C, Lipopolysaccharide, zymosan, Flagellin, Pam3CSK4, PamCysPamSK4, dsRNA, a diacylated lipopeptide, a triacylated lipoprotein, lipoteichoic acid, a peptidoglycan, a cyclic dinucleotide, a 5′ppp-dsRNA, S-27609, CL307, UC-IV 150, imiquimod, gardiquimod, resiquimod, motolimod, VTS-1463GS-9620, GSK2245035, TMX-101, TMX-201, TMX-202, isatoribine, AZD8848, MEDI9197, 3M-051, 3M-852, 3M-052, 3M-854A, S-34240, KU34B, CL663, SB9200, SB11285, or 8-substituted 2-amino-3H-benzo[b]azepine-4-carbozamide.

38. (canceled)

39. The composition of claim 1, wherein the immune-stimulatory compound is LY2109761, GSK263771, iCRT3, iCRT5, iCRT14, LY2090314, CGX-1321, PRI-724, BC21, ZINCO2092166, LGK974, IWP2, LY3022859, LY364947, SB431542, AZD8186, SD-208, indoximod (NLG8189), F001287, GDC-0919, epacadostat (INCB024360), RG70099, 1-methyl-L-tryptophan, methylthiohydantoin tryptophan, brassinin, annulin B, exiguamine A, PIM, LM1O, INCB023843, or 8-substituted imidazo[1,5-a]pyridine.

40. The composition of claim 1, wherein a molar ratio of the immune-stimulatory compound to the antibody is less than 8.

41-42. (canceled)

43. A pharmaceutical composition comprising the composition of claim 1 and a pharmaceutically acceptable carrier.

44. A method of treating a subject having cancer, comprising administering a therapeutic dose of the composition of claim 1.

45. (canceled)

46. The method of claim 44, wherein the composition is administered intravenously, cutaneously, subcutaneously, or injected at a site of affliction.

47-55. (canceled)
